SEQUENCE LISTING (377 sequences)

SEQ ID NO: 1 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - protein, 226 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 4 - protein, 226 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 5 - protein, 243 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 6 - protein, 198 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 7 - protein, 189 aa, synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 8 - protein, 200 aa, synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 9 - protein, 225 aa, synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 10 - protein, 225 aa, synthetic construct; identical to SEQ ID NO: 9

SEQ ID NO: 11 - protein, 222 aa, synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 12 - protein, 6 aa, synthetic construct
SSNFYA

SEQ ID NO: 13 - skipped

SEQ ID NO: 14 - protein, 11 aa, synthetic construct
CALYNNNDMRF

SEQ ID NO: 15 - protein, 132 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKP

SEQ ID NO: 16 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 273 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 5 aa, synthetic construct
SGHNS

SEQ ID NO: 19 - protein, 6 aa, synthetic construct
FNNNVP

SEQ ID NO: 20 - protein, 13 aa, synthetic construct
CASSPGSTDTQYF

SEQ ID NO: 21 - protein, 132 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVL

SEQ ID NO: 22 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 311 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 6 aa, synthetic construct
DRGSQS

SEQ ID NO: 25 - skipped

SEQ ID NO: 26 - protein, 15 aa, synthetic construct
CAAVISNFGNEKLTF

SEQ ID NO: 27 - protein, 134 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIP

SEQ ID NO: 28 - protein, 141 aa, synthetic construct; identical to SEQ ID NO: 16

SEQ ID NO: 29 - protein, 275 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 5 aa, synthetic construct
SGHRS

SEQ ID NO: 31 - protein, 6 aa, synthetic construct
YFSETQ

SEQ ID NO: 32 - protein, 14 aa, synthetic construct
CASSPWDSPNEQYF

SEQ ID NO: 33 - protein, 132 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVT

SEQ ID NO: 34 - protein, 179 aa, synthetic construct; identical to SEQ ID NO: 22

SEQ ID NO: 35 - protein, 311 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - protein, 6 aa, synthetic construct
NSAFQY

SEQ ID NO: 37 - skipped

SEQ ID NO: 38 - protein, 13 aa, synthetic construct
CAMSEAAGNKLTF

SEQ ID NO: 39 - protein, 133 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKP

SEQ ID NO: 40 - protein, 141 aa, synthetic construct; identical to SEQ ID NO: 16

SEQ ID NO: 41 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 5 aa, synthetic construct
SGHAT

SEQ ID NO: 43 - protein, 6 aa, synthetic construct
FQNNGV

SEQ ID NO: 44 - protein, 13 aa, synthetic construct
CASSYTNQGEAFF

SEQ ID NO: 45 - protein, 132 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVV

SEQ ID NO: 46 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 309 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 48 - protein, 6 aa, synthetic construct
DRGSQS

SEQ ID NO: 49 - skipped

SEQ ID NO: 50 - protein, 13 aa, synthetic construct
CAVLNQAGTALIF

SEQ ID NO: 51 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSS

SEQ ID NO: 52 - protein, 141 aa, synthetic construct; identical to SEQ ID NO: 16

SEQ ID NO: 53 - protein, 273 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 5 aa, synthetic construct
SGDLS

SEQ ID NO: 55 - protein, 6 aa, synthetic construct
YYNGEE

SEQ ID NO: 56 - protein, 17 aa, synthetic construct
CASSAETGPWLGNEQFF

SEQ ID NO: 57 - protein, 135 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVL

SEQ ID NO: 58 - protein, 179 aa, synthetic construct; identical to SEQ ID NO: 22

SEQ ID NO: 59 - protein, 314 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 60 - protein, 7 aa, synthetic construct
TSESDYY

SEQ ID NO: 61 - protein, 4 aa, synthetic construct
QEAY

SEQ ID NO: 62 - protein, 15 aa, synthetic construct
CAYRWAQGGSEKLVF

SEQ ID NO: 63 - protein, 136 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNP

SEQ ID NO: 64 - protein, 141 aa, synthetic construct
YIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - protein, 277 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 5 aa, synthetic construct
MGHDK

SEQ ID NO: 67 - protein, 6 aa, synthetic construct
SYGVNS

SEQ ID NO: 68 - protein, 16 aa, synthetic construct
CATELWSSGGTGELFF

SEQ ID NO: 69 - protein, 134 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVL

SEQ ID NO: 70 - protein, 179 aa, synthetic construct; identical to SEQ ID NO: 22

SEQ ID NO: 71 - protein, 313 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 6 aa, synthetic construct
DRGSQS

SEQ ID NO: 73 - skipped

SEQ ID NO: 74 - protein, 13 aa, synthetic construct
CAVGPSGTYKYIF

SEQ ID NO: 75 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLA

SEQ ID NO: 76 - protein, 141 aa, synthetic construct; identical to SEQ ID NO: 16

SEQ ID NO: 77 - protein, 273 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 5 aa, synthetic construct
MNHEY

SEQ ID NO: 79 - protein, 6 aa, synthetic construct
SMNVEV

SEQ ID NO: 80 - protein, 14 aa, synthetic construct
CASSPGGSGNEQFF

SEQ ID NO: 81 - protein, 132 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVL

SEQ ID NO: 82 - protein, 179 aa, synthetic construct; identical to SEQ ID NO: 22

SEQ ID NO: 83 - protein, 311 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - protein, 6 aa, synthetic construct
DRGSQS

SEQ ID NO: 85 - skipped

SEQ ID NO: 86 - protein, 14 aa, synthetic construct
CAVVSGGGADGLTF

SEQ ID NO: 87 - protein, 133 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQP

SEQ ID NO: 88 - protein, 141 aa, synthetic construct; identical to SEQ ID NO: 64

SEQ ID NO: 89 - protein, 274 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 5 aa, synthetic construct
PRHDT

SEQ ID NO: 91 - protein, 6 aa, synthetic construct
FYEKMQ

SEQ ID NO: 92 - protein, 14 aa, synthetic construct
CASSLGRGGQPQHF

SEQ ID NO: 93 - protein, 142 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSIL

SEQ ID NO: 94 - protein, 177 aa, synthetic construct; identical to SEQ ID NO: 46

SEQ ID NO: 95 - protein, 319 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 96 - protein, 6 aa, synthetic construct
DSAIYN

SEQ ID NO: 97 - skipped

SEQ ID NO: 98 - protein, 15 aa, synthetic construct
CAVRPTSGGSYIPTF

SEQ ID NO: 99 - protein, 133 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 100 - protein, 141 aa, synthetic construct
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 101 - protein, 274 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 5 aa, synthetic construct
MNHEY

SEQ ID NO: 103 - protein, 6 aa, synthetic construct
SVGAGI

SEQ ID NO: 104 - protein, 14 aa, synthetic construct
CASSYVGNTGELFF

SEQ ID NO: 105 - protein, 132 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 106 - protein, 179 aa, synthetic construct; identical to SEQ ID NO: 22

SEQ ID NO: 107 - protein, 311 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 108 - protein, 6 aa, synthetic construct
SSNFYA

SEQ ID NO: 109 - skipped

SEQ ID NO: 110 - protein, 11 aa, synthetic construct
CALYNNNDMRF

SEQ ID NO: 111 - protein, 132 aa, synthetic construct
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKP

SEQ ID NO: 112 - protein, 141 aa, synthetic construct; identical to SEQ ID NO: 16

SEQ ID NO: 113 - protein, 273 aa, synthetic construct
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 114 - protein, 5 aa, synthetic construct
SGHNS

SEQ ID NO: 115 - protein, 6 aa, synthetic construct
FNNNVP

SEQ ID NO: 116 - protein, 13 aa, synthetic construct
CASSPGSTDTQYF

SEQ ID NO: 117 - protein, 132 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVL

SEQ ID NO: 118 - protein, 179 aa, synthetic construct; identical to SEQ ID NO: 22

SEQ ID NO: 119 - protein, 311 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 120 - protein, 7 aa, synthetic construct
MTLNGDE

SEQ ID NO: 121 - protein, 6 aa, synthetic construct
IYSNGD

SEQ ID NO: 122 - protein, 6 aa, synthetic construct
TYSSGN

SEQ ID NO: 123 - protein, 6 aa, synthetic construct
IYSNGD

SEQ ID NO: 124 - protein, 7 aa, synthetic construct
QEAYKQQ

SEQ ID NO: 125 - protein, 6 aa, synthetic construct
IYSNGD

SEQ ID NO: 126 - protein, 6 aa, synthetic construct
IYSNGD

SEQ ID NO: 127 - protein, 7 aa, synthetic construct
IQSSQRE

SEQ ID NO: 128 - protein, 7 aa, synthetic construct
MTLNGDE

SEQ ID NO: 129 - protein, 21 aa, synthetic construct
EPKSCDKTHTCPPCPAPELLG

SEQ ID NO: 130 - protein, 6 aa, synthetic construct
ELLGGP

SEQ ID NO: 131 - protein, 461 aa, synthetic construct
QKEVEQNSGPLSVPEGAIASLNCTYSDRGSQSFFWYRQYSGKSPELIMSIYSNGDKEDGRFTAQLNKASQYFSLLIRDSQPSDSATYLCAAVIDNSNGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 132 - protein, 477 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRSVSWYQQTPGQGLQFLFEYVHGAERNKGNFPGRFSGRQFSNSSSEMNISNLELGDSALYLCASSPWDSPNEQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 133 - protein, 463 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 479 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - protein, 6 aa, synthetic construct
DRGSQS

SEQ ID NO: 136 - protein, 6 aa, synthetic construct
DRGSQL

SEQ ID NO: 137 - protein, 6 aa, synthetic construct
IYSNGD

SEQ ID NO: 138 - protein, 6 aa, synthetic construct
IYQEGD

SEQ ID NO: 139 - protein, 15 aa, synthetic construct
CAAVINNPSGGMLTF

SEQ ID NO: 140 - protein, 15 aa, synthetic construct
CAAVIDNSNGGILTF

SEQ ID NO: 141 - protein, 15 aa, synthetic construct
CAAVIDNPSGGILTF

SEQ ID NO: 142 - protein, 15 aa, synthetic construct
CAAVIDNDQGGILTF

SEQ ID NO: 143 - protein, 15 aa, synthetic construct
CAAVIPNPPGGKLTF

SEQ ID NO: 144 - protein, 15 aa, synthetic construct
CAAVIPNPGGGALTF

SEQ ID NO: 145 - protein, 15 aa, synthetic construct
CAAVIPNSAGGRLTF

SEQ ID NO: 146 - protein, 15 aa, synthetic construct
CAAVIPNLEGGSLTF

SEQ ID NO: 147 - protein, 15 aa, synthetic construct
CAAVIPNRLGGYLTF

SEQ ID NO: 148 - protein, 15 aa, synthetic construct
CAAVIPNTDGGRLTF

SEQ ID NO: 149 - protein, 15 aa, synthetic construct
CAAVIPNQRGGALTF

SEQ ID NO: 150 - protein, 15 aa, synthetic construct
CAAVIPNVVGGILTF

SEQ ID NO: 151 - protein, 15 aa, synthetic construct
CAAVITNIAGGSLTF

SEQ ID NO: 152 - protein, 15 aa, synthetic construct
CAAVIPNNDGGYLTF

SEQ ID NO: 153 - protein, 15 aa, synthetic construct
CAAVIPNGRGGLLTF

SEQ ID NO: 154 - protein, 15 aa, synthetic construct
CAAVIPNTHGGPLTF

SEQ ID NO: 155 - protein, 15 aa, synthetic construct
CAAVIPNDVGGSLTF

SEQ ID NO: 156 - protein, 15 aa, synthetic construct
CAAVIENKPGGPLTF

SEQ ID NO: 157 - protein, 15 aa, synthetic construct
CAAVIDNPVGGPLTF

SEQ ID NO: 158 - protein, 15 aa, synthetic construct
CAAVIPNNNGGALTF

SEQ ID NO: 159 - protein, 15 aa, synthetic construct
CAAVIPNDQGGILTF

SEQ ID NO: 160 - protein, 15 aa, synthetic construct
CAAVIPNVVGGQLTF

SEQ ID NO: 161 - protein, 15 aa, synthetic construct
CAAVIPNSYGGLLTF

SEQ ID NO: 162 - protein, 15 aa, synthetic construct
CAAVIPNDDGGLLTF

SEQ ID NO: 163 - protein, 15 aa, synthetic construct
CAAVIPNAAGGLLTF

SEQ ID NO: 164 - protein, 15 aa, synthetic construct
CAAVIPNTIGGLLTF

SEQ ID NO: 165 - protein, 15 aa, synthetic construct
CAAVIPNTRGGLLTF

SEQ ID NO: 166 - protein, 5 aa, synthetic construct
SGHRS

SEQ ID NO: 167 - protein, 5 aa, synthetic construct
PGHRA

SEQ ID NO: 168 - protein, 5 aa, synthetic construct
PGHRS

SEQ ID NO: 169 - protein, 6 aa, synthetic construct
YFSETQ

SEQ ID NO: 170 - protein, 6 aa, synthetic construct
YVHGEE

SEQ ID NO: 171 - protein, 6 aa, synthetic construct
YVHGAE

SEQ ID NO: 172 - protein, 14 aa, synthetic construct
CASSPWDSPNEQYF

SEQ ID NO: 173 - protein, 14 aa, synthetic construct
CASSPWDSPNVQYF

SEQ ID NO: 174 - protein, 503 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGQKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGGSGGGGSGGGGSGGGGSGGGGSGSKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 175 - protein, 214 aa, synthetic construct
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 461 aa, synthetic construct
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 177 - protein, 477 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 178 - protein, 214 aa, synthetic construct; identical to SEQ ID NO: 175

SEQ ID NO: 179 - protein, 487 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPTREFQDLHWYRKETAKSPEFLFYFGPYGVEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGSTDTQYFGPGTRLTVL

SEQ ID NO: 180 - protein, 487 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPTKEFQDLHWYRKETAKSPEFLFYFGPYGREKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVL

SEQ ID NO: 181 - protein, 487 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPSSNFYNLHWYRKETAKSPEFLFYFGPYGVEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFNSETVIDDSGMPEDRFSAKMPNASFSTLKIQPSEPRDSAVYFCASSPGATDTQYFGPGTRLTVL

SEQ ID NO: 182 - protein, 487 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPNKEFQDLHWYRKETAKSPEFLFYFGPYGTEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGSTDTQYFGPGTRLTVL

SEQ ID NO: 183 - protein, 487 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVL

SEQ ID NO: 184 - protein, 454 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 185 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 186 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 187 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 188 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 189 - protein, 472 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 190 - protein, 454 aa, synthetic construct
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 191 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 192 - protein, 107 aa, synthetic construct
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIK

SEQ ID NO: 193 - protein, 122 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSS

SEQ ID NO: 194 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 195 - protein, 467 aa, synthetic construct
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 196 - protein, 120 aa, synthetic construct
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 197 - protein, 106 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIK

SEQ ID NO: 198 - protein, 120 aa, synthetic construct
EVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 199 - protein, 120 aa, synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSS

SEQ ID NO: 200 - protein, 120 aa, synthetic construct
EVQLVQSGAEVKKPGASVKVSCKASGYKFTRYVMHWVRQAPGQGLEWMGYINPYNDVTKYAEKFQGRVTLTSDTSTSTAYMELSSLRSEDTAVYYCARGSYYDYEGFVYWGQGTLVTVSS

SEQ ID NO: 201 - protein, 120 aa, synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSS

SEQ ID NO: 202 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 203 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 204 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 205 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 206 - protein, 454 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 207 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSS

SEQ ID NO: 208 - protein, 454 aa, synthetic construct; identical to SEQ ID NO: 190

SEQ ID NO: 209 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGESYISYWAYWGQGTLVTVSS

SEQ ID NO: 210 - protein, 454 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 211 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSS

SEQ ID NO: 212 - protein, 454 aa, synthetic construct
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 213 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 214 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETMMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 215 - protein, 470 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETMMRGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 216 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 217 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 218 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 219 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 220 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 221 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 222 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 223 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 224 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSAGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 225 - protein, 454 aa, synthetic construct; identical to SEQ ID NO: 223

SEQ ID NO: 226 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 227 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSAGSTDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 228 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSIDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 229 - protein, 467 aa, synthetic construct
ILNVEQSPQSLHVQEGDSTKFTCSFPVKEFQDIHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 230 - protein, 467 aa, synthetic construct; identical to SEQ ID NO: 222

SEQ ID NO: 231 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETMMRGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 232 - protein, 467 aa, synthetic construct; identical to SEQ ID NO: 187

SEQ ID NO: 233 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSAGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 234 - protein, 467 aa, synthetic construct; identical to SEQ ID NO: 218

SEQ ID NO: 235 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSTGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 236 - protein, 467 aa, synthetic construct; identical to SEQ ID NO: 220

SEQ ID NO: 237 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 238 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 239 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 240 - protein, 472 aa, synthetic construct; identical to SEQ ID NO: 189

SEQ ID NO: 241 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGESYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 242 - protein, 472 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 243 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 244 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 245 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSAGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 246 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 247 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 248 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSTGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 249 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 250 - protein, 472 aa, synthetic construct; identical to SEQ ID NO: 242

SEQ ID NO: 251 - protein, 472 aa, synthetic construct; identical to SEQ ID NO: 244

SEQ ID NO: 252 - protein, 472 aa, synthetic construct; identical to SEQ ID NO: 246

SEQ ID NO: 253 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNADMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 254 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNDDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 255 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNEDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 256 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNFDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 257 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNHDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 258 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNIDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 259 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 260 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNKDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 261 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNQDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 262 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNRDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 263 - protein, 467 aa, synthetic construct
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNVDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 264 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNESFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 265 - protein, 467 aa, synthetic construct; identical to SEQ ID NO: 218

SEQ ID NO: 266 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNRSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 267 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNKSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 268 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNQSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 269 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNNSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 270 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNSSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 271 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDRQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 272 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDHQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 273 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDEQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 274 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 275 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDQQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 276 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDNQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 277 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDFQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 278 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDYQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 279 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDIQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 280 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDVQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 281 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDRQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 282 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDHQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 283 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDEQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 284 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 285 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDQQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 286 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDNQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 287 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDFQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 288 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDYQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 289 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDIQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 290 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDVQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 291 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNESFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 292 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNRSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 293 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNKSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 294 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNQSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 295 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNNSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 296 - protein, 457 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNSSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 297 - protein, 454 aa, synthetic construct; identical to SEQ ID NO: 284

SEQ ID NO: 298 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 299 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 300 - protein, 472 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 301 - protein, 457 aa, synthetic construct; identical to SEQ ID NO: 239

SEQ ID NO: 302 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 303 - protein, 454 aa, synthetic construct; identical to SEQ ID NO: 247

SEQ ID NO: 304 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 305 - protein, 110 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKP

SEQ ID NO: 306 - protein, 111 aa, synthetic construct
GVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVL

SEQ ID NO: 307 - protein, 111 aa, synthetic construct
GVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVL

SEQ ID NO: 308 - protein, 111 aa, synthetic construct
GVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDAQYFGPGTRLTVL

SEQ ID NO: 309 - protein, 110 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKP

SEQ ID NO: 310 - protein, 9 aa, synthetic construct
SLLQHLIGL

SEQ ID NO: 311 - protein, 9 aa, synthetic construct
SLLMWITQV

SEQ ID NO: 312 - protein, 10 aa, synthetic construct
STASAITPSV

SEQ ID NO: 313 - protein, 509 aa, synthetic construct
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 314 - RNA, 1527 nt, synthetic construct
  source 1..1527: mol_type = other RNA
atggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaac

SEQ ID NO: 315 - RNA, 1527 nt, synthetic construct
  source 1..1527: mol_type = other RNA
atggaacgaaggcgcttgtggggctccatccagagccgatacatcagcatgagcgtgtggacaagcccacggagactcgtggagctggcagggcagagcctgctgaaggacgaggccctggccatcgccgccctggagttgctgcccagggagctcttcccgccactcttcatggcagccttcgacgggagacacagccagaccctgaaggcaatggtgcaggcctggcccttcacctgcctccccctgggagtgctgatgaagggacaacacctccacctggagaccttcaaagccgtgctcgacggactcgacgtgctcctcgcccaggaggtccgccccaggaggtggaaactccaagtgctggacttacggaagaactcccaccaggacttctggaccgtatggtccggaaacagggccagcctgtactcattcccagagccagaagcagcccagcccatgacaaagaagcgaaaagtagacggcttgagcacagaggcagagcagcccttcatcccagtagaggtgctcgtagacctgttcctcaaggaaggcgcctgcgacgaattgttctcctacctcatcgagaaagtgaagcgaaagaaaaacgtactacgcctgtgctgcaagaagctgaagatcttcgcaatgcccatgcaggacatcaagatgatcctgaaaatggtgcagctggactccatcgaagacttggaagtgacctgcacctggaagctacccaccttggcgaaattctccccctacctgggccagatgatcaacctgcgcagactcctcctctcccacatccacgcatcctcctacatctccccggagaaggaagagcagtacatcgcccagttcacctcccagttcctcagcctgcagtgcctgcaggccctctacgtggactccttattcttcctcagaggccgcctggaccagttgctcaggcacgtgatgaaccccttggaaaccctctcaataaccaactgccggctctcggaaggggacgtgatgcacctgtcccagagccccagcgtcagccagctaagcgtcctgagcctaagcggggtcatgctgaccgacgtaagccccgagcccctccaagccctgctggagagagcctccgccaccctccaggacctggtcttcgacgagtgcgggatcacggacgaccagctcctcgccctcctgccctccctgagccactgctcccagctcacaaccttaagcttctacgggaactccatctccatatccgccttgcagagcctcctgcagcacctcatcgggctgagcaacctgacccacgtgctgtaccccgtccccctggagagctacgaggacatccacggcaccctccacctggagaggctcgcctacctgcacgccaggctcagggagttgctgtgcgagttggggcggcccagcatggtctggctcagcgccaacccctgcccccactgcggggacagaaccttctacgacccggagcccatcctgtgcccctgcttcatgcccaac

SEQ ID NO: 316 - DNA, 1527 bp, synthetic construct
  source 1..1527: mol_type = other DNA
atggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaac

SEQ ID NO: 317 - RNA, 27 nt, synthetic construct
  source 1..27: mol_type = other RNA
agtctcctgcagcacctcatcgggctg

SEQ ID NO: 318 - RNA, 27 nt, synthetic construct
  source 1..27: mol_type = other RNA
agcctcctgcagcacctcatcgggctg

SEQ ID NO: 319 - DNA, 27 bp, synthetic construct
  source 1..27: mol_type = other DNA
agtctcctgcagcacctcatcgggctg

SEQ ID NO: 320 - protein, 6 aa, synthetic construct
VKEFQD

SEQ ID NO: 321 - protein, 7 aa, synthetic construct
FGPYGKE

SEQ ID NO: 322 - protein, 9 aa, synthetic construct
ALYNNYDMR

SEQ ID NO: 323 - protein, 110 aa, synthetic construct; identical to SEQ ID NO: 305

SEQ ID NO: 324 - protein, 467 aa, synthetic construct; identical to SEQ ID NO: 221

SEQ ID NO: 325 - protein, 5 aa, synthetic construct
SGHNS

SEQ ID NO: 326 - protein, 6 aa, synthetic construct
FQNTAV

SEQ ID NO: 327 - protein, 11 aa, synthetic construct
ASSPGATDKQY

SEQ ID NO: 328 - protein, 111 aa, synthetic construct; identical to SEQ ID NO: 307

SEQ ID NO: 329 - protein, 454 aa, synthetic construct; identical to SEQ ID NO: 247

SEQ ID NO: 330 - protein, 6 aa, synthetic construct
VKEFQD

SEQ ID NO: 331 - protein, 7 aa, synthetic construct
FGPYGKE

SEQ ID NO: 332 - protein, 9 aa, synthetic construct
ALYNNYDMR

SEQ ID NO: 333 - protein, 110 aa, synthetic construct; identical to SEQ ID NO: 305

SEQ ID NO: 334 - protein, 467 aa, synthetic construct; identical to SEQ ID NO: 299

SEQ ID NO: 335 - protein, 5 aa, synthetic construct
SGHNS

SEQ ID NO: 336 - protein, 6 aa, synthetic construct
FQNTAV

SEQ ID NO: 337 - protein, 11 aa, synthetic construct
ASSAGSTDAQY

SEQ ID NO: 338 - protein, 111 aa, synthetic construct
GVIQSPRHEVTEMGQEVTLRCKPISGHNSLFWYRETPMQGLELLIYFQNTAVIDDSGMPEDRFSAKMPNDSFSTLKIQPSEPRDSAVYFCASSAGSTDAQYFGPGTRLTVL

SEQ ID NO: 339 - protein, 454 aa, synthetic construct; identical to SEQ ID NO: 227

SEQ ID NO: 340 - protein, 6 aa, synthetic construct
VKEFQD

SEQ ID NO: 341 - protein, 7 aa, synthetic construct
FGPYGKE

SEQ ID NO: 342 - protein, 9 aa, synthetic construct
ALYNNYDMR

SEQ ID NO: 343 - protein, 110 aa, synthetic construct; identical to SEQ ID NO: 305

SEQ ID NO: 344 - protein, 472 aa, synthetic construct; identical to SEQ ID NO: 246

SEQ ID NO: 345 - protein, 5 aa, synthetic construct
SGHNS

SEQ ID NO: 346 - protein, 6 aa, synthetic construct
FQNTAV

SEQ ID NO: 347 - protein, 11 aa, synthetic construct
ASSPGATDKQY

SEQ ID NO: 348 - protein, 111 aa, synthetic construct; identical to SEQ ID NO: 307

SEQ ID NO: 349 - protein, 457 aa, synthetic construct; identical to SEQ ID NO: 249

SEQ ID NO: 350 - protein, 6 aa, synthetic construct
VKEFQD

SEQ ID NO: 351 - protein, 7 aa, synthetic construct
FGPYGKE

SEQ ID NO: 352 - protein, 9 aa, synthetic construct
ALYNNYDMR

SEQ ID NO: 353 - protein, 110 aa, synthetic construct; identical to SEQ ID NO: 305

SEQ ID NO: 354 - protein, 467 aa, synthetic construct; identical to SEQ ID NO: 299

SEQ ID NO: 355 - protein, 5 aa, synthetic construct
SGHNS

SEQ ID NO: 356 - protein, 6 aa, synthetic construct
FQNTAV

SEQ ID NO: 357 - protein, 11 aa, synthetic construct
ASSPGSTDAQY

SEQ ID NO: 358 - protein, 111 aa, synthetic construct; identical to SEQ ID NO: 308

SEQ ID NO: 359 - protein, 454 aa, synthetic construct; identical to SEQ ID NO: 284

SEQ ID NO: 360 - protein, 6 aa, synthetic construct
VKEFQD

SEQ ID NO: 361 - protein, 7 aa, synthetic construct
FGPYGKE

SEQ ID NO: 362 - protein, 9 aa, synthetic construct
ALYNNYDMR

SEQ ID NO: 363 - protein, 110 aa, synthetic construct; identical to SEQ ID NO: 305

SEQ ID NO: 364 - protein, 472 aa, synthetic construct; identical to SEQ ID NO: 241

SEQ ID NO: 365 - protein, 5 aa, synthetic construct
SGHNS

SEQ ID NO: 366 - protein, 6 aa, synthetic construct
FQNTAV

SEQ ID NO: 367 - protein, 11 aa, synthetic construct
ASSPGATDKQY

SEQ ID NO: 368 - protein, 111 aa, synthetic construct; identical to SEQ ID NO: 307

SEQ ID NO: 369 - protein, 457 aa, synthetic construct; identical to SEQ ID NO: 249

SEQ ID NO: 370 - protein, 9 aa, synthetic construct
IIINHVISV

SEQ ID NO: 371 - protein, 9 aa, synthetic construct
SLNLRETNL

SEQ ID NO: 372 - protein, 9 aa, synthetic construct
LTLGHLMGV

SEQ ID NO: 373 - protein, 9 aa, synthetic construct
KLLKNLIGI

SEQ ID NO: 374 - protein, 479 aa, synthetic construct; identical to SEQ ID NO: 134

SEQ ID NO: 375 - protein, 463 aa, synthetic construct; identical to SEQ ID NO: 133

SEQ ID NO: 376 - protein, 10 aa, synthetic construct
GLPSIPVHPI

SEQ ID NO: 377 - protein, 10 aa, synthetic construct
GLPSIPVHPV

(19) United States
(12) Patent Application Publication
(10) Pub. No.: US 2024/0075130 A1
(43) Pub. Date: Mar. 7, 2024

(54) METHODS OF IDENTIFYING METASTATIC LESIONS IN A PATIENT AND TREATING THEREOF

(71) Applicant: Immatics Biotechnologies GmbH, Tuebingen (DE)

(72) Inventors: Jens HUKELMANN, Tuebingen (DE); Heiko SCHUSTER, Tuebingen (DE); Jens FRITSCHE, Tuebingen (DE); Oliver SCHOOR, Tuebingen (DE); Frank SCHWOEBEL, Tuebingen (DE); Lena Katharina FREUDENMANN, Tuebingen (DE)

(21) Appl. No.: 18/505,361

(22) Filed: Nov. 9, 2023

Related U.S. Application Data
(63) Continuation of application No. 17/938,311, filed on Oct. 5, 2022.
(60) Provisional application No. 63/275,854, filed on Nov. 4, 2021.
(60) Provisional application No. 63/252,749, filed on Oct. 6, 2021.

(30) Foreign Application Priority Data
Oct. 6, 2021 (EP) 21201289.2
Feb. 8, 2022 (EP) 22155737.4
Aug. 2, 2022 (EP) 22188307.7
Aug. 31, 2022 (EP) 22193289.0

(51) Int. Cl. A61K 39/385; A61K 35/17; A61K 38/17; A61K 38/20; A61P 11/00; A61P 35/04; A61P 37/04; C07K 14/725; C07K 16/28; C12N 5/0783; C12N 5/10; G01N 33/574

(52) U.S. Cl. CPC A61K 39/385; A61K 35/17; A61K 38/1774; A61K 38/20; A61K 38/2046; A61K 38/2086; A61P 11/00; A61P 35/04; A61P 37/04; C07K 14/7051; C07K 16/2809; C12N 5/0638; C12N 5/10; G01N 33/57492; A61K 2039/53

(57) ABSTRACT

A method of treating a metastatic lesion that presents a peptide containing SLLQHLIGL (SEQ ID NO: 310) on a cell surface, including selecting a patient having a metastatic lesion and administering to the patient a composition containing recombinant T lymphocytes or activated T lymphocytes that express a T cell receptor, or a functional fragment thereof, that is reactive with, or binds to, an MHC ligand containing SLLQHLIGL (SEQ ID NO: 310).

### CROSS REFERENCE TO RELATED APPLICATIONS

This application is a continuation of U.S. patent application Ser. No. 17/938,311, filed on Oct. 5, 2022, which claims priority to European Patent Application No. 22193289.0, filed on Aug. 31, 2022, European Patent Application No. 22188307.7, filed on Aug. 2, 2022, European Patent Application No. 22155737.4, filed on Feb. 8, 2022, U.S. Provisional Patent Application No. 63/275,854, filed on Nov. 4, 2021, U.S. Provisional Patent Application No. 63/252,749, filed on Oct. 6, 2021, and European Patent Application No. 21201289.2, filed on Oct. 6, 2021. Each of these applications is incorporated by reference in its entirety.

### REFERENCE TO SEQUENCE LISTING SUBMITTED AS A COMPLIANT XML FORMAT FILE (.xml)

Pursuant to the EFS-Web legal framework and 37 CFR §§ 1.821-825 (see MPEP § 2442.03(a)), Rule 30 EPC, and § 11 PatV, an electronic sequence listing compliant with WIPO standard ST.26 in the form of an XML format file (entitled “2912919-109008_Sequence_Listing_ST26.xml” created on Nov. 8, 2023, and 403,655 bytes in size) is submitted concurrently with the instant application, and the entire contents of the sequence listing are incorporated herein by reference. For the avoidance of doubt, if discrepancies exist between the sequences mentioned in the specification and the electronic sequence listing, the sequences in the specification shall be deemed to be the correct ones.

The present invention relates to peptides, proteins, nucleic acids, and cells for use in immunotherapeutic methods. In particular, the present invention relates to the immunotherapy of cancer. The present invention furthermore relates to tumor-associated T cell peptide epitopes, alone or in combination with other tumor-associated peptides that can for example serve as active pharmaceutical ingredients of vaccine compositions that stimulate anti-tumor immune responses, or to stimulate T cells ex vivo and transfer into patients. Peptides bound to molecules of the major histocompatibility complex (MHC), or peptides as such, can also be targets of antibodies, soluble T cell receptors, and other binding molecules.

The present invention relates to several novel peptide sequences and their variants derived from HLA class I molecules of human tumor cells that can be used in vaccine compositions for eliciting anti-tumor immune responses, or as targets for the development of pharmaceutically/immunologically active compounds and cells.

### BACKGROUND OF THE INVENTION

According to the World Health Organization (WHO), cancer ranked among the four major non-communicable deadly diseases worldwide in 2012. For the same year, colorectal cancer, breast cancer, and respiratory tract cancers were listed within the top 10 causes of death in high income countries.

### Cancer Immunotherapy

Immunotherapy of cancer represents an option of specific targeting of cancer cells while minimizing side effects. Cancer immunotherapy makes use of the existence of tumor-associated antigens.

The current classification of tumor-associated antigens (TAAs) comprises the following major groups:
a) Cancer-testis antigens: The first TAAs ever identified that can be recognized by T cells belong to this class, which was originally called cancer-testis (CT) antigens. Since the cells of testis do not express class I and II HLA molecules, these antigens cannot be recognized by T cells in normal tissues and can therefore be considered as immunologically tumor-specific. Well-known examples for CT antigens are the MAGE family members, PRAME and NY-ESO-1.
b) Differentiation antigens: These TAAs are shared between tumors and the normal tissue from which the tumor arose. Most of the known differentiation antigens are found in melanomas and normal melanocytes. Examples include, but are not limited to, tyrosinase and Melan-A/MART-1 for melanoma or PSA for prostate cancer.
c) Overexpressed TAAs: Genes encoding widely expressed TAAs have been detected in histologically different types of tumors as well as in many normal tissues, generally with lower expression levels. It is possible that many of the epitopes processed and potentially presented by normal tissues are below the threshold level for T cell recognition, while their overexpression in tumor cells can trigger an anticancer response by breaking previously established tolerance. Prominent examples for this class of TAAs are Her-2/neu, survivin, telomerase, or WT1.
d) Tumor-specific antigens: These unique TAAs arise from mutations of normal genes (such as β-catenin, CDK4, BCR-ABL, etc.). Some of these molecular changes are associated with neoplastic transformation and/or tumor progression. Tumor-specific antigens are generally able to induce strong immune responses without bearing the risk for autoimmune reactions against normal tissues. On the other hand, these TAAs are in most cases only relevant to the exact tumor on which they were identified and are usually not shared between many individual tumors. Tumor specificity (or -association) of a peptide may also arise if the peptide originates from a tumor-specific (-associated) exon in case of proteins with tumor-specific (-associated) isoforms.
e) Oncoviral proteins: These TAAs are viral proteins that may play a critical role in the oncogenic process and, because they are foreign (not of human origin), they can evoke a T cell response. Examples of such proteins are the human papilloma type 16 virus proteins, E6 and E7, which are expressed in cervical carcinoma. Human endogenous retroviruses (HERVs) make up a significant portion (˜8%) of the human genome. These viral elements integrated into the genome millions of years ago and were since then vertically transmitted through generations. The huge majority of HERVs have lost functional activity through mutation or truncation, yet some endogenous retroviruses, such as the members of the HERV-K clade, still encode functional genes and have been shown to form retrovirus-like particles. Transcription of HERV proviruses is epigenetically controlled and remains silenced under normal physiological conditions. Reactivation and overexpression resulting in active translation of viral proteins has, however, been described in certain diseases and especially for different types of cancer. This tumor-specific expression of HERV-derived proteins can be harnessed for different types of cancer immunotherapy.
f) TAAs arising from abnormal post-translational modifications: Such TAAs may arise from proteins which are neither specific nor overexpressed in tumors but nevertheless become tumor-associated by post-translational processes primarily active in tumors. Examples for this class arise from altered glycosylation patterns leading to novel epitopes in tumors as for MUC1 or events like protein splicing during degradation which may or may not be tumor-specific.

T cell-based immunotherapy targets peptide epitopes derived from tumor-associated or tumor-specific proteins, which are presented by MHC molecules. The antigens that are recognized by the tumor-specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

There are two classes of MHC molecules, MHC class I and MHC class II. MHC class I molecules are composed of an alpha (heavy) chain and beta-2-microglobulin (light chain, β2m), MHC class II molecules of an alpha and a beta chain. Their three-dimensional conformation results in a binding groove, which is used for non-covalent interaction with peptides.

MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation in the literature (Rock, Gamble, and Rothstein 1990; Brossart and Bevan 1997). MHC class II molecules can be found predominantly on professional antigen-presenting cells (APCs), and primarily present peptides of exogenous or transmembrane proteins that are taken up by APCs e.g. during endocytosis and are subsequently processed.

Complexes of peptide and MHC class I are recognized by CD8-positive T cells bearing the appropriate T cell receptor (TCR), whereas complexes of peptide and MHC class II molecules are recognized by CD4-positive helper T cells bearing the appropriate TCR. It is well known that the TCR, the peptide and the MHC are thereby present in a stoichiometric amount of 1:1:1.

CD4-positive helper T cells play an important role in inducing and sustaining effective responses by CD8-positive cytotoxic T cells. The identification of CD4-positive T cell epitopes derived from tumor associated antigens (TAA) is of great importance for the development of pharmaceutical products for triggering anti-tumor immune responses. At the tumor site, T helper cells, support a cytotoxic T cell (CTL) friendly cytokine milieu and attract effector cells, e.g. CTLs, natural killer (NK) cells, macrophages, and granulocytes.

According to different sources, >90% of deaths from cancer are caused by lesions, including metastases (Hanahan and Weinberg 2000). There are so far only few therapeutic options that address such metastatic lesions.

Hence, there is an urgent need for new and effective treatment for such conditions. There is also a need to identify factors representing biomarkers for such metastatic lesions, leading to better diagnosis of such metastatic lesions, assessment of prognosis, and prediction of treatment success.

### DETAILED DESCRIPTION OF THE INVENTION

Before the invention is described in detail, it is to be understood that this invention is not limited to the particular component parts of the devices described or process steps of the methods described, as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, a peptide comprising the amino acid sequence of SEQ ID NO: 310 (SLLQHLIGL) or a pharmaceutically acceptable salt thereof is provided, said peptide being for use in the (manufacture of a medicament for the) treatment of a patient (i) being diagnosed for, (ii) suffering from or (iii) being at risk of developing, metastasis or a metastatic lesion.

This language is deemed to encompass both the swiss type claim language accepted in some countries (in this case, brackets are deemed absent) and EPC2000 language (in this case, brackets and content within the brackets is deemed absent).

Alternatively, or in addition, a method of treating a patient (i) being diagnosed for, (ii) suffering from or (iii) being at risk of developing, a metastasis or a metastatic lesion,

is provided.

The method comprises administering to the patient a peptide comprising the amino acid sequence of SEQ ID NO: 310 (SLLQHLIGL) or a pharmaceutically acceptable salt thereof, in one or more therapeutically effective doses.

Alternatively, or in addition, a pharmaceutical composition for treating metastasis or a metastatic lesion is provided, comprising a peptide comprising the amino acid sequence of SEQ ID NO: 310 (SLLQHLIGL) or a pharmaceutically acceptable salt as an effective ingredient.

In one embodiment, said treatment or composition does not encompass the co-administration (simultaneously or sequentially) with a peptide that is a fragment of the Prostate specific Membrane antigen (PSMA). The amino acid sequence of PSMA is disclosed under UniProt reference Q04609.

In particular, said treatment does not encompass the co-administration (simultaneously or sequentially) with PSMA₂₈₈₋₂₉₇ (GLPSIPVHPI, SEQ ID NO: 376) or PSMA₂₈₈₋₂₉₇ I297V (GLPSIPVHPV, SEQ ID NO: 377)

In one embodiment, the peptide used in that treatment does not comprise any N-terminal or C terminal residues that go beyond the sequence as set forth in SEQ ID NO: 1.

In one embodiment, the metastases or metastatic lesion is PRAME positive. As used herein, the term “metastasis or a metastatic lesion which is PRAME positive” relates to metastasis or a metastatic lesion that comprises cells that express PRAME. In one embodiment, the metastases or metastatic lesion displays, on the surface of at least one of its cells, a peptide comprising the amino acid sequence of SEQ ID NO: 310 (SLLQHLIGL), or said amino acid sequence bound to a major histocompatibility complex.

The term “metastasization” relates to the spread of cancerous cells or tissues from a primary tumor. Cancer occurs after cells are genetically altered to proliferate rapidly and indefinitely. The cells eventually undergo metaplasia, followed by dysplasia then anaplasia, resulting in a malignant phenotype, which is often called “primary tumor”. This malignancy allows for invasion into the circulation, followed by invasion to a second site for tumorigenesis.

Some cells from the primary tumor acquire the ability to penetrate the walls of lymphatic or blood vessels, after which they are able to circulate through the bloodstream to other sites and tissues in the body. This process is known as lymphatic or hematogenous spread. After the tumor cells come to rest at another site, they re-penetrate the vessel or walls and continue to multiply, eventually forming another clinically detectable tumor. This new tumor is known as a metastasis (plural: “metastases”, both terms can be used interchangeably herein), commonly causing metastatic lesions. Metastasization is one of the hallmarks of cancer, distinguishing it from benign tumors. Most cancers can metastasize, however some don't. Basal cell carcinoma for example rarely metastasizes.

Regarding nomenclature, the following rules apply:
(i) The term “metastatic Breast cancer”, relates to a Breast cancer as primary tumor, that releases cancer cells into the body, which may or may not settle and form metastases in the same or other organs or tissues.
(ii) The term “Breast cancer metastasis” relates to a metastasis in either the breast or another organ or tissue which has spread from a Breast cancer as primary tumor.

This nomenclature relates to all other tumor or cancer types or metastasis as well, like e.g.
(i) metastatic lung cancer, (ii) lung cancer metastasis, and/or
(ii) metastatic liver cancer, (ii) liver cancer metastasis, and so forth.

Hence, in diagnosis, a metastasis found somewhere in the body is oftentimes for example qualified as a lung cancer metastasis if the patient has been diagnosed for a primary lung tumor, or as a colon cancer metastasis if the patient has been diagnosed for a primary colon tumor.

This nomenclature will be used throughout the present application.

In one embodiment, the metastases or metastatic lesions according to the invention occur in one or more vital organs. In one embodiment, the vital organ is preferably at least one selected from the group consisting of brain, spinal cord, heart, lungs, liver, bone marrow, blood, trachea, skin, kidneys, pancreas, intestines.

In one embodiment, the metastases or metastatic lesions according to the invention have a diameter of 1 cm or more. In one embodiment thereof, such metastases or metastatic lesions occur in vital organs.

In one embodiment, 10 or more metastases or metastatic lesions are found in the patient, preferably 11 or more. In one embodiment thereof, such metastases or metastatic lesions occur in vital organs.

In one embodiment, the metastases or metastatic lesions have progressed beyond the lymphatic system.

In one embodiment, the metastases or metastatic lesions are not lymphatically confined.

Metastases can and will often acquire additional mutations and evolve independently of their original tumor at their metastatic site. As such, information gained from studying primary tumors is not necessarily applicable to their metastases and the independent development of the metastases can lead to several differences between primary tumors and metastases derived thereof that can affect the clinical outcome of the cancer.

Some of these differences can affect the presentation levels of pHLA and may include, but are not limited to:

(a) Differences in the Antigen Peptide Presentation Complex.

An overview of loss of MHC class I antigen presentation in cancer evolution can be found in (Dhatchinamoorthy, Colbert, and Rock 2021). In particular, down-regulation of the antigen processing presenting complex in metastases has been shown via reduced expression of TAP1 (Ling et al. 2017), HLA (McGranahan et al. 2017; Watkins et al. 2020) as well as b₂M (Campo et al. 2014).

(b) Down Regulation of Specific Genes and Antigens

Apart from the downregulation of MHC presentation pathway in metastases, reduced expression of tumor antigens used in clinical trials like TRPM8 (Fuessel et al. 2006) has also been reported (Yao et al. 2019)

Both mechanisms—the downregulation of the antigen processing pathway and the down regulation of specific antigens—may contribute to the effect seen in Figure. 42, which shows the presentation of the peptide KRT5-004 (STASAITPSV, SEQ ID NO: 312).

KRT5-004 is associated to the parental protein Keratin 5, also known as KRT5, K5, or CK5, which is a protein that is encoded in humans by the KRT5 gene. It dimerizes with keratin 14 and forms the intermediate filaments (IF) that make up the cytoskeleton of basal epithelial cells. This protein is involved in several diseases including epidermolysis bullosa simplex and breast and lung cancers.

The presentation of KRT5-004 is completely lost when comparing HNSCC (Head and neck squamous cell carcinoma) primary tumors with HNSCC metastases: While SEQ ID NO: 312 is detected in nearly 50% of primary HNSCC tumor samples, it is completely absent in the metastatic HNSCC tumor samples analyzed.

Furthermore, when comparing the chemosensitivity of primary and metastatic tumor samples from the same patients, differences in the chemosensitivity to common chemotherapeutic drugs have also been reported (Furukawa et al. 2000)

FIG. 40 shows that the peptide PRAME 004 (SLLQHLIGL, SEQ ID NO: 310) is presented on selected metastases, but not on healthy tissues.

FIG. 43 shows that the peptide PRAME 004 (SLLQHLIGL, SEQ ID NO: 310) is differently presented on selected metastases, and on selected primary tumors.

FIG. 45 shows that the peptide PRAME 004 (SLLQHLIGL, SEQ ID NO: 310) is differently presented on primary triple-negative breast cancer (TNBC) and metastatic triple-negative breast cancer (TNBC).

FIGS. 48, 49A, and 49B show experiment from patient-derived xenografts (PDX), where tumor metastases were xenografted into preclinical mouse models with tumor biology as close as possible to the in vivo situation in patients. Main genetic and histological properties of the patient's metastases remained unchanged over a certain period of time (passages in mice). For this reason, the PDX models used are superior over cell line-derived xenografts (CDX), which do not have, let alone preserve, the physiological properties, including the immunopeptidome, of metastases.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

Metastasis or a metastatic lesion can be analyzed whether it displays, on the surface of at least one of its cells, a peptide comprising the amino acid sequence of SEQ ID NO: 310 (SLLQHLIGL), or said amino acid bound to a major histocompatibility complex, by different means.

In one embodiment, one takes a biopsy of the tumor, or another sample that is diagnostically suitable (like blood, lymph, liquor, saliva or urine sample, comprising for example, floating cells, sHLA, exosomes, tumor-derived extracellular vesicles (Evs) etc), and subjects it to immunoprecipitation of peptide MHC complexes, with subsequent analysis of the peptidome thus obtained by means of mass spectrometry. Respective methods are e.g. disclosed in (Fritsche et al. 2018), the content of which is incorporated herein by reference.

Another possibility is to use a labelled T cell receptor or TCR mimetic antibody specific of the peptide MHC complex comprising the peptide of SEQ ID NO: 310 (SLLQHLIGL). In one embodiment, a biopsy or sample of the metastases is obtained, rated with routine immunological methods (sliced, homogenized, or the like) and then incubated with the T cell receptor of TCR mimectic antibody. See e.g. (Høydahl et al. 2019) for methods, the content of which is incorporated herein by reference.

In another embodiment, the mRNA encoding for the parental protein that gives rise to the peptide of interest, or encoding for the specific exon thereof, can be determined, for example by means of qRT-PCR or any other mRNA detection technique. Such methods are in the routine of the skilled artisan. See, for example, (Wong and Medrano 2005; Moon et al. 2020), the contents of which are incorporated herein by reference.

Another possibility is to apply RNA-Seq techniques to the metastasis. RNA-Seq (named as an abbreviation of “RNA sequencing”) is a sequencing technique which uses next-generation sequencing (NGS) to reveal the presence and quantity of RNA in a biological sample at a given moment, analyzing the continuously changing cellular transcriptome. Specifically, RNA-Seq facilitates the ability to look at alternative gene spliced transcripts, post-transcriptional modifications, gene fusion, mutations/SNPs and changes in gene expression over time, or differences in gene expression in different groups or treatments. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries. Recent advances in RNA-Seq include single cell sequencing, in situ sequencing of fixed tissue, and native RNA molecule sequencing with single-molecule real-time sequencing.

The respective HLA status can be determined by routine methods of HLA serotyping and HLA haplotyping, as e.g. disclosed in (Zhang et al. 2014), the content of which is incorporated herein by reference.

A2 is a human leukocyte antigen serotype within the HLA-A serotype group. The serotype is determined by the antibody recognition of the α2 domain of the HLA-A α-chain. For A2, the α chain is encoded by the HLA-A*02 gene and the β chain is encoded by the B2M locus.

HLA-A*02 is one particular class I major histocompatibility complex (MHC) allele group at the HLA-A locus. The A*02 allele group can encode for many proteins; as of December 2013 there were 456 different HLA-A*02 proteins. Serotyping can identify as far as HLA-A*02, which is typically enough to prevent transplant rejection (the original motivation for HLA identification). Genes can further be separated by genetic sequencing and analysis. HLAs can be identified with as many as nine numbers and a letter (ex. HLA-A*02:101:01:02N). HLA-A*02 is globally common, but particular variants of the allele can be separated by geographic prominence.

The term “peptide”, as used herein, shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoroacetate) salts. It has to be noted that the salts of the peptides according to the present description differ substantially from the peptides in their state(s) in vivo, as the peptides are not salts in vivo.

As used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral —NH2 group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

For example, the pharmaceutically acceptable salt is selected from a chloride salt, an acetate salt, a trifluoroacetate salt, a phosphate salt, a nitrate salt, a sulfate salt, a bromide salt, a propionate salt, a glycolate salt, a pyruvate salt, an oxalate salt, a malate salt, a maleate salt, a malonate salt, a succinate salt, a fumarate salt, a tartrate salt, a citrate salt, a benzoate salt, a cinnamate salt, a mandelate salt, a methane sulfonate salt, an ethane sulfonate salt, a p-toluenesulfonate salt, a salicylate salt, a sodium salt, a potassium salt, an ammonium salt, a calcium salt or a trimethylamine salt.

SEQ ID NO: 310 (SLLQHLIGL, alias name: PRAME-004) is a peptide that is related to PRAME, which is a protein encoded by the PRAME gene.

PRAME (Preferentially Expressed Antigen in Melanoma), also known as Opa-interacting protein 4, CT130, and MAPE, is a protein and tumor antigen of the Cancer/Testis antigen group. PRAME has a length of 509 amino acids and a mass of 57,890 Da. PRAME has the Entrez identifier 23532, and the UniProt identifier P78395.

PRAME, which is expressed at a high level in a large proportion of tumors, as well as several types of leukemia. PRAME is the best characterized member of the PRAME family of leucine-rich repeat (LRR) proteins. Mammalian genomes contain multiple members of the PRAME family whereas in other vertebrate genomes only one PRAME-like LRR protein was identified. PRAME is a cancer/testis antigen that is expressed at very low levels in normal adult tissues except testis but at high levels in a variety of cancer cells.

PRAME-004 is a 9 amino acid peptide that is obtained by degradation of PRAME by the ubiquitin-proteasome system (UPS). PRAME-004 is also called PRA425-433, as it comprises AA residues 425-433 of the PRAME protein. PRAME-004 is then presented by major histocompatibility complex (MHC) class I molecules on the cellular surface of the respective cells.

The inventors have found out that PRAME-004 is displayed, with high selectivity, on MHC class I molecules of primary tumors (see, e.g., WO2018172533A2 and US20180273602, the contents which are incorporated by reference in their entireties). As such, the inventors have described that PRAME-004 can be used as a target for entities being capable of binding to PRAME-004, for the treatment of different primary tumors.

However, the inventors have surprisingly found that PRAME-004 is also presented by metastases and metastatic lesions. For these cancer types, only very limited therapeutic options were so far available.

As used herein, the term “metastasization” shall refer to the spread of cancer cells from the place where they first formed (i.e., initial or primary site) to another part of the host's body (i.e., different or secondary site). In metastatic cancer, cancer cells break away from the original (primary) tumor, travel through the blood or lymph system, and form a new (secondary) tumor in the same or in other organs or tissues of the body. These newly formed pathological sites are called metastases or metastatic tumor(s). The new (or secondary) metastatic tumor is of the same type of cancer as the primary tumor. As metastatic cancer cells share some features with the primary cancer, they are commonly referred to by the same designation as the primary cancer. For example, breast cancer that spreads to the lung is commonly called metastatic breast cancer (not lung cancer) and is, thus, treated as breast cancer, not as lung cancer.

In some cases of metastatic cancer, the origin of the cancer cannot be identified (e.g., if the primary tumor cannot be located). This type of cancer is called cancer of unknown primary origin or occult primary cancer.

Cancer that spreads from where it originated to another part of the body is called metastatic cancer. The direct extension and penetration by cancer cells into neighboring tissue is referred to as ‘cancer invasion’, which is the first step in the process of metastasization (see below). For many types of cancer, metastatic cancer is also called advanced cancer or stage IV (4) cancer. However, the terms stage IV (4) cancer and advanced cancer may also refer to a cancer that is large, but has not spread to another body part (e.g., locally advanced cancer).

The process by which cancer cells spread to other parts of the body is called metastasization. The term metastasization refers to the spreading of a pathogenic agent from an initial (primary) site to a different (secondary) site within the host's body. As used herein, the term metastasization shall refer to the spreading of a cancerous cell or tumor from an initial (primary) site to a different (secondary) site within the host's body. Thus, as used herein metastatic cancer is a cancer associated with metastasization, which is the spread of cancer from the primary site (the place where the cancer originated from) to other places in the body.

Also, as used herein, the term metastasization shall mean the development of secondary tumors in parts of the body that are different and/or far away from the original primary cancer (Fares et al. 2020).

Thus, as used herein, metastasization is the dissemination of tumor cells from the primary neoplasm to secondary sites in a multistep process that is often depicted as a simple series of sequential events: escape from the primary tumor and local invasion, intravasation and survival in the circulation and extravasation and metastatic seeding. (Riggio, Varley, and Welm 2021).

Metastasization can be broken down into two major phases; the physical dissemination of cancer cells from the primary tumor to neighboring tissues, and the adaptation of these cells to neighboring tissue microenvironments that result in successful colonization, i.e., the growth of metastases into macroscopic tumors, which includes metastatic lesions. In one embodiment, the terms “metastases” and “metastatic lesion” are used synonymously.

Metastases shall refer to an accumulation of cancer cells, which are of the same type as the primary tumor but locoregionally separated from the site of the primary tumor. This accumulation can be within the same or a different organ or tissue and may lead to tumorous growth. Separation from the primary tumor could for example be confirmed by any of the following invasive or non-invasive methodologies or any combination thereof:
Macroscopic assessment through visual or instrument-guided (e.g., endoscopic) inspection of metastases formation for example during surgical procedures or examinations of the cancer patient.
Histopathological assessment of the tissue collected from surgical procedures including biopsies. For this assessment, a person skilled in the art (e.g., a trained pathologist) might want to additionally make use of different kinds of physical or chemical treatments of the collected tissue (e.g., FFPE preservation), staining with chemical reagents (e.g., including dyes or antibodies binding to molecular or genetic markers) or additional analyses known to the person that might further facilitate the identification of cancer cells to confirm metastases formation.
Medical imaging techniques such as computed tomography (CT), magnetic resonance tomography (MRI), positron-emission tomography (PET), ultrasound, X-ray or any combination of the aforementioned (e.g. PET/MRI).
Biomarker-based assays such as the prostate serum antigen (PSA) screen or other assays that quantify biomolecules indicative of primary and/or metastatic cancer in clinical samples that include but are not limited to blood, urine, stool, and others.

Most spreading cancer cells die at a certain stage during the process of metastasization. However, if conditions are favorable for the cancer cells at every step, some of them are able to form new tumors in other parts of the body. Metastatic cancer cells can also remain inactive at a distant site for many years before they begin to proliferate again, if at all.

Cancer can spread to almost any part of the body, although several types of cancer are more likely to spread to certain areas than others. Certain organ sites (sometimes referred to as “fertile soil” or “metastatic niches”) can be especially permissive for metastatic seeding and colonization by certain types of cancer cell, as a consequence of local properties that are either intrinsic to the normal tissue or induced at a distance by systemic actions of primary tumors. Cancer stem cells may be variably involved in some or all of the different stages or primary tumorigenesis and metastasization (Hanahan and Weinberg 2011).

In a further embodiment, metastatic cancer manifests after a protracted period of undetectable disease following surgery or systemic therapy, owing to relapse or recurrence. In the case of breast cancer, for example, metastatic relapse can occur months to decades after initial diagnosis and treatment.

Thus, metastatic cancer can occur de novo, in which metastases are present at the original diagnosis, the cancer having already spread prior to detection. However, often the de novo occurrence is the result of relapse (recurrence), where metastases manifest after definitive treatment (Riggio, Varley, and Welm 2021).

Representative cancers that are subject to metastasization may include adrenocortical carcinoma, breast carcinoma, lung cancer, melanoma, colon cancer, renal cell carcinoma, prostate cancer, cancer of the cervix, cervical squamous cell carcinoma and endocervical adenocarcinoma, cholangiocarcinoma, bladder cancer, bladder urothelial carcinoma, head and neck squamous cell carcinoma, head and neck adenocarcinoma, rectal cancer, esophageal cancer, esophageal carcinoma, liver cancer, liver hepatocellular carcinoma, mouth and throat cancer, multiple myeloma, ovarian cancer, ovarian serous cystadenocarcinoma, sarcoma, stomach adenocarcinoma, testicular germ cell tumors, thymoma, uterine carcinosarcoma, uterine endometrial carcinoma, and stomach cancer. In some embodiments, the metastases or metastatic lesions may originate from a cancer selected from the group consisting of adrenocortical carcinoma, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, uterine endometrial carcinoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, cholangiocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

(Liu and Cao 2016); the content of which is hereby incorporated by reference in its entirety) shows that primary tumors may create a favorable microenvironment, namely, pre-metastatic niche (PMN), in secondary organs and tissue sites for subsequent metastases. The pre-metastatic niche can be primed and established through a complex interplay among primary tumor-derived factors, tumor-mobilized bone marrow-derived cells, and local stromal components. Liu et al. proposed six characteristics that may define the pre-metastatic niche, which enable tumor cell colonization and promote metastasization, including (1) immunosuppression, (2) inflammation, (3) angiogenesis/vascular permeability, (4) lymphangiogenesis, (5) organotropism, and (6) reprogramming.

For example, primary tumor-derived components, tumor-mobilized bone-marrow-

derived cells (BMDCs), and the local stromal microenvironment of the host (or future metastatic organ components) may be factors crucial for the formation of pre-metastatic niche. Many molecular and cellular components contributing to pre-metastatic niche formation have been identified in different tumor models. These niche-promoting molecular components, in addition to being secreted by tumor cells, can also be produced by myeloid cells and stromal cells. They may work jointly with cellular components to initiate, polarize, and establish premetastatic niche in future metastatic organs.

Representative primary tumor determinants of organ-specific metastasization may be found, for example, in Table 1 of (Liu and Cao 2016), the content of which is incorporated by reference.

Tumor-derived extracellular vesicles (Evs) can travel far from their original site to act as potential mediators for educating the pre-metastatic niche. Evs can be grouped into categories: exosomes (30-100 nm in diameter), microvesicles (100-1,000 nm in diameter), and a newly identified cancer-derived EV population termed “large oncosomes” (1-10 mm in diameter). Exosomes that contain proteins, mRNAs, microRNAs, small RNAs, and/or DNA fragments can facilitate pre-metastatic niche formation by mediating communication between tumor cells with surrounding components or by horizontally transferring their contents into the recipient cells. Tumor-derived microvesicles may mediate crosstalk between tumor cells and host cells in the secondary microenvironment for pre-metastatic niche formation. Tumor-derived large oncosomes contain metalloproteinases, RNA, caveolin-1, and the GTPase ARF6, suggesting that metastatic tumor cells may program the distant sites to be a pre-metastatic niche via secretion of large oncosomes.

Some embodiments of the present disclosure may include methods of inhibiting metastatic lesions in a subject, including selecting a subject having a cancer that presents a peptide consisting of SLLQHLIGL (SEQ ID NO: 310) on the cell surface with increased exosomal levels of one or more markers of metastatic lesions relative to control exosomal levels of the one or more markers of metastatic lesions, wherein the markers of metastatic lesion are at least one selected from the group consisting of the PMN-promoting molecules listed in Table 1 of (Liu and Cao 2016), and administering to the selected subject T cells and/or bispecific molecules of the present disclosure in an amount effective to inhibit metastatic lesion in the subject.

In an embodiment, treatment may be of patients experiencing metastatic cancer. Treatment of the present disclosure may also be administered to patients who have cancer with increased exosomal levels of one or more markers of metastatic lesions, but prior to any identified metastases, in order to prevent metastasization. Similarly, a patient that could develop potentially-malignant neoplasms may be treated by the methods described herein. A subject in need of treatment may be identified by the diagnosis of a potentially-malignant neoplasm. A treatment group may include subjects who are unable to receive conventional cancer treatments, such as surgery, radiation therapy, or chemotherapy. A patient with metastatic cancer or at risk for cancer metastasis may not be able to undergo certain cancer treatments due to other diagnoses, physical conditions, or complications. For example, aged or weakened patients, such as those experiencing cancer cachexia, may not be good candidates for surgery due to a risk of not surviving an invasive procedure. Patients who already have a compromised immune system or a chronic infection may not be able to receive chemotherapy since many chemotherapy drugs may harm the immune system.

Metastases can and will often acquire additional mutations and evolve independently of their original tumor at their metastatic site. As such, information gained from studying primary tumors is not necessarily applicable to their metastases and the independent development of the metastases can lead to several differences between primary tumors and metastases derived thereof that can affect the clinical outcome of the cancer.

Some of these differences can affect the presentation levels of pHLA and may include, but are not limited to:

(c) Differences in the Antigen Peptide Presentation Complex.

An overview of loss of MHC class I antigen presentation in cancer evolution can be found in (Dhatchinamoorthy, Colbert, and Rock 2021). In particular, downregulation of the antigen processing presenting complex in metastases has been shown via reduced expression of TAP1 (Ling et al. 2017), HLA (McGranahan et al. 2017; Watkins et al. 2020) as well as b₂M (Campo et al. 2014).

(d) Downregulation of Specific Genes and Antigens

Apart from the downregulation of MHC presentation pathway in metastases, reduced expression of tumor antigens used in clinical trials like TRPM8 (Fuessel et al. 2006) has also been reported (Yao et al. 2019).

Both mechanisms—the downregulation of the antigen processing pathway and the downregulation of specific antigens—may contribute to the effect seen in FIG. 42, which shows the presentation of the peptide KRT5-004 (STASAITPSV, SEQ ID NO: 312).

KRT5-004 is associated to the parental protein Keratin 5, also known as KRT5, K5, or CK5, which is a protein that is encoded in humans by the KRT5 gene. It dimerizes with keratin 14 and forms the intermediate filaments (IF) that make up the cytoskeleton of basal epithelial cells. This protein is involved in several diseases including epidermolysis bullosa simplex and breast and lung cancers.

The presentation of KRT5-004 is completely lost when comparing HNSCC (Head and neck squamous cell carcinoma) primary tumors with HNSCC metastases: While SEQ ID NO: 312 is detected in nearly 50% of primary HNSCC tumor samples, it is completely absent in the metastatic HNSCC tumor samples analyzed.

Furthermore, when comparing the chemosensitivity of primary and metastatic tumor samples from the same patients, differences in the chemosensitivity to common chemotherapeutic drugs have also been reported (Furukawa et al. 2000).

The skilled person has different routine approaches at his disposal to determine whether or not a cell, or a metastases or metastatic lesion, is PRAME positive. Based on the Entrez identifier 23532, and the UniProt identifier P78395, the skilled person can either use immunohistochemical methods (like ELISA, RIA or the like), in which an antibody or binding agent is used that binds to PRAME protein in a suitable tissue sample. As an alternative, the skilled person can detect presence or absence of PRAME mRNA, by means of RT-PCR or other routine methods.

In a preferred embodiment of the invention, the term metastases or metastatic lesion excludes primary tumors.

According to one embodiment of the invention, said peptide has the ability to bind to an MHC class 1 or class II molecule, and/or said peptide, when bound to said MHC, is capable of being recognized by CD4 or CD8 T cells.

Complexes of peptide and MHC class I are recognized by CD8-positive T cells bearing the appropriate T cell receptor (TCR).

According to one embodiment of the invention, the pharmaceutically acceptable salt is a chloride salt or an acetate salt.

According to further embodiments, the peptide may also have an overall length of from 9 to 30 amino acids. Preferably, it has from 9 to 12 amino acids. In one embodiment said peptide comprises 1 to 4 additional amino acids at the C- and/or N-terminus of SEQ ID NO: 310. See table 1 for further details:

TABLE 1
Combinations of the elongations of peptides of the invention

| C-terminus | N-terminus |
|---|---|
| 4 | 0 |
| 3 | 0 or 1 |
| 2 | 0 or 1 or 2 |
| 1 | 0 or 1 or 2 or 3 |
| 0 | 0 or 1 or 2 or 3 or 4 |
| N-terminus | C-terminus |
| 4 | 0 |
| 3 | 0 or 1 |
| 2 | 0 or 1 or 2 |
| 1 | 0 or 1 or 2 or 3 |
| 0 | 0 or 1 or 2 or 3 or 4 |

In one embodiment, said peptide has a length according to the respective SEQ ID NO: 310. In one embodiment, the peptide consists or consists essentially of the amino acid sequence according to SEQ ID NO: 310.

According to another aspect of the invention, an antibody, or a functional fragment thereof, is provided. The antibody or functional fragment specifically recognizes, or binds to, the peptide according to the above description, or to the peptide according to the above description when bound to an MHC molecule.

The antibody or functional fragment is provided for use in the (manufacture of a medicament for the) treatment of a patient (i) being diagnosed for, (ii) suffering from or (iii) being at risk of developing, metastasis or a metastatic lesion.

Alternatively, or in addition, a method of treating a patient (i) being diagnosed for, (ii) suffering from or (iii) being at risk of developing, metastasis or a metastatic lesion,

is provided.

The method comprises administering to the patient an antibody, or a functional fragment thereof, which specifically recognizes, or binds to, the peptide according to the above description, or to the peptide according to the above description when bound to an MHC molecule, in one or more therapeutically effective doses.

Alternatively, or in addition, a pharmaceutical composition for treating metastasis or a metastatic lesion is provided, comprising an antibody, or a functional fragment thereof, which specifically recognizes, or binds to, the peptide according to the above description, or to the peptide according to the above description when bound to an MHC molecule as an effective ingredient.

In one embodiment, said treatment or composition does not encompass the co-administration (simultaneously or sequentially) with an antibody or functional fragment thereof that binds a peptide that is a fragment of the Prostate specific Membrane antigen (PSMA).

In particular, said treatment does not encompass the co-administration (simultaneously or sequentially) with an antibody or functional fragment thereof that binds to PSMA₂₈₈₋₂₉₇ (GLPSIPVHPI, SEQ ID NO: 376) or PSMA₂₈₈₋₂₉₇ I297V (GLPSIPVHPV, SEQ ID NO: 377).

As used herein, the term “antibody” shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.

As used herein, the term “functional fragment” shall refer to fragments of such antibody retaining target binding capacities, e.g.
a CDR (complementarity determining region)
a hypervariable region,
a variable domain (Fv)
an IgG or IgM heavy chain (consisting of VH, CH1, hinge, CH2 and CH3 regions)
an IgG or IgM light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerized constructs comprising CH3+VL+VH, and antibody conjugates (e.g. antibody or fragments or derivatives linked to a toxin, a cytokine, a radioisotope or a label). These types are well described in the literature and can be used by the skilled person on the basis of the present disclosure, without adding further inventive activity.

Methods for the production of a hybridoma cell are disclosed in (Köhler and Milstein 1975).

Methods for the production and/or selection of chimeric or humanised mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Methods for the production and/or selection of fully human mAbs are known in the art. These can involve the use of a transgenic animal which is immunized with the respective protein or peptide, or the use of a suitable display technique, like yeast display, phage display, B-cell display or ribosome display, where antibodies from a library are screened against human iRhom2 in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis.

IgG, IgM, scFv, Fab, and/or F(ab)₂ are antibody formats well known to the skilled person. Related enabling techniques are available from the respective textbooks.

As used herein, the term “Fab” relates to an IgG/IgM fragment comprising the antigen binding region, said fragment being composed of one constant and one variable domain from each heavy and light chain of the antibody.

As used herein, the term “F(ab)₂” relates to an IgG/IgM fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like. These types are well described in the literature and can be used by the skilled person on the basis of the present disclosure, with adding further inventive activity.

Antibodies capable of binding a peptide bound to an MHC are sometimes called “TCR mimic antibodies” or “TCR like antibodies”. Generally, such antibodies can be generated with the methods described above. Methods how to generate TCR like antibodies are for example disclosed in (He et al. 2019), the content of which is incorporated herein by reference on its entirety.

TCR mimic antibodies binding to HLA restricted peptide derived from PRAME are for example disclosed in (Chang et al. 2017), the content of which is incorporated herein by reference in its entirety. See, also, US 2018/0148503 (T cell receptor-like antibodies specific for a PRAME peptide) (Eureka Therapeutics Inc), the content of which is incorporated herein by reference in its entirety.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

According to another aspect of the invention, a T cell receptor, or a functional fragment thereof, is provided that is reactive with, or binds to, an MHC ligand, wherein said ligand is the peptide according to the above description, or the peptide according to the above description when bound to an MHC molecule. The T cell receptor is provided for use in the (manufacture of a medicament for the) treatment of a patient (i) being diagnosed for, (ii) suffering from, or (iii) being at risk of developing, metastasis or a metastatic lesion.

Alternatively, or in addition, a method of treating a patient (i) being diagnosed for, (ii) suffering from or (iii) being at risk of developing, metastasis or a metastatic lesion, is provided.

The method comprises administering to the patient a T cell receptor, or a functional fragment thereof, that is reactive with, or binds to, an MHC ligand, wherein said ligand is the peptide according to the above description, or the peptide according to the above description when bound to an MHC molecule, in one or more therapeutically effective doses.

Alternatively, or in addition, a pharmaceutical composition for treating metastasis or a metastatic lesion is provided, comprising a T cell receptor, or a functional fragment thereof, that is reactive with, or binds to, an MHC ligand, wherein said ligand is the peptide according to the above description, or the peptide according to the above description when bound to an MHC molecule, as an effective ingredient.

In one embodiment, said treatment does not encompass the co-administration (simultaneously or sequentially) with a T cell receptor or functional fragment thereof that binds a peptide that is a fragment of the Prostate specific Membrane antigen (PSMA), the peptide being bound to an MHC molecule.

In particular, said treatment does not encompass the co-administration (simultaneously or sequentially) with a T cell receptor or functional fragment thereof that binds to PSMA₂₈₈₋₂₉₇ (GLPSIPVHPI, SEQ ID NO: 376) or PSMA₂₈₈₋₂₉₇ I297V (GLPSIPVHPV, SEQ ID NO: 377), the peptide being bound to an MHC molecule.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

According to one embodiment, the T cell receptor is provided as a soluble molecule.

As used herein, a soluble T cell receptor refers to heterodimeric truncated variants of native TCRs, which comprise extracellular portions of the TCR α-chain and β-chain, for example linked by a disulfide bond, but which lack the transmembrane and cytosolic domains of the native protein. The terms “soluble T cell receptor α-chain sequence and soluble T cell receptor R-chain sequence” refer to TCR α-chain and R-chain sequences that lack the transmembrane and cytosolic domains. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Soluble TCR of the invention in any case retain the binding functionality of their parent molecules.

### PRAME-004-Specific TCRs

Complexes of peptide and MHC class I are recognized by CD8-positive T cells bearing the appropriate T cell receptor (TCR), whereas complexes of peptide and MHC class II molecules are recognized by CD4-positive-helper-T cells bearing the appropriate TCR. It is recognized that the TCR, the peptide and the MHC are thereby present in a stoichiometric amount of 1:1:1.

This interaction is highly specific. For example, in the MHC class I dependent immune reaction, peptides not only have to be able to bind to certain MHC class I molecules expressed by tumor cells, they subsequently also have to be recognized by T cells bearing a specific T cell receptor (TCR). Usually, when targeting peptide-MHC complexes by said specific TCRs (e.g., soluble TCRs) and antibodies according to the invention, the presentation is the determining factor for a successful response.

The present invention further relates to T cell receptors (TCRs), in particular soluble TCR (sTCRs) and cloned TCRs engineered into autologous or allogeneic T cells, and methods of making these, as well as NK cells or other cells bearing said TCR or cross-reacting with said TCRs.

Structurally, a subgroup of these T cell receptors (TCRs) comprises an alpha chain and a beta chain (“alpha/beta TCRs”). These TCRs specifically bind to a peptide, e.g., SLLQHLIGL (PRAME-004) (SEQ ID NO: 310), according to the invention when presented by an MHC molecule. The present description also relates to fragments of such TCRs according to the invention that are still capable of specifically binding to a peptide antigen e.g., PRAME-004 (SEQ ID NO: 310), according to the present invention when presented by an HLA molecule. This relates to soluble TCR fragments, for example, TCRs missing the transmembrane parts and/or constant regions, single chain TCRs, and fusions thereof to, for example, with immunoglobulin (Ig). For example, TCRs and fragments thereof of the present disclosure may include those disclosed in U.S. 20180273602, U.S. Ser. No. 10/800,832, and U.S. 20200123221, the contents of which are herein incorporated by reference in their entireties.

The alpha and beta chains of alpha/beta TCRs and the gamma and delta chains of gamma/delta TCRs, structurally have two “domains,” namely variable and constant domains. The variable domain consists of a concatenation of variable region (V) and joining region (J). The variable domain may also include a leader region (L). Beta and delta chains may also include a diversity region (D). The alpha and beta constant domains may also include C-terminal transmembrane (TM) domains that anchor the alpha and beta chains to the cell membrane.

The majority of available TCR structures are αβ TCRs, which are formed of TCRα and TCRβ chains. A small number of TCRs are γδ TCRs, consisting of TCRγ and TCRδ chains. The TCRβ and TCRδ chains are considered to be analogous to antibody heavy chains, while the TCRα and TCRγ chains are considered to be analogous to antibody light chains (Rudolph, Stanfield, and Wilson 2006).

As mentioned above, each TCR chain is characterized by two immunoglobulin domains: a variable domain (V) and a constant (C). Both variable and constant domains have a conserved β-sandwich structure, making it possible to number and compare variable domains from different TCRs (Dunbar and Deane 2016). The IMGT numbering has been used for structural analysis of TCRs (Glanville et al. 2017; Dunbar et al. 2014). On each variable domain, there are three hypervariable loops that have the highest degree of sequence and structural variation, known as the complementary-determining regions (CDR1, CDR2, and CDR3). Flanking the CDRs, the remaining portions of the TCR structure are collectively known as the TCRs “framework.”

The CDRs may comprise one or more “changes,” such as substitutions, additions or deletions from the given sequence, provided that the TCR retains the capacity to bind a peptide:MHC complex. The change may involve substitution of an amino acid for a similar amino acid, e.g., a conservative substitution. A similar amino acid is one which has a side chain moiety with related properties as grouped together, for example, (i) basic side chains: lysine, arginine, histidine, (ii) acidic side chains: aspartic acid and glutamic acid, (iii) uncharged polar side chains: asparagine, glutamine, serine, threonine and tyrosine, and (iv) non-polar side chains: glycine, alanine, valine, leucine, isoleucine, proline, phenylalanine, methionine, tryptophan, and cysteine.

Outside of the variable parts of the TCR, TCR structures are highly conserved, and therefore only a very small part of the chains creates the actual specificity of the TCR repertoire. As mentioned above, TCRs are generated by genomic rearrangement of the germline TCR locus, a process termed V(D)J recombination, that has the potential to generate marked diversity of TCRs (estimated to range from 10¹⁵ to as high as 10⁶¹ possible receptors).

Despite this potential diversity, TCRs from T-cells that recognize the same pMHC epitope often share conserved sequence features. Analyses demonstrate that each epitope-specific repertoire contains a clustered group of receptors that share core sequence similarities, together with a dispersed set of diverse “outlier” sequences. By identifying shared motifs in core sequences, key conserved residues driving essential elements of TCR recognition can be highlighted (Glanville et al. 2017; Dash et al. 2017), both herewith specifically incorporated by reference). These analyses provide insights into the generalizable, underlying features of epitope-specific repertoires and adaptive immune recognition.

Sequence analysis focusing entirely on high probability contact sites in CDR3 seems to provide a means of clustering TCRs by shared specificity, as the majority of these possible contacts are in the CDR3s, and only short, typically linear stretches of amino acids make contact with antigenic peptide residues (IMGT positions 107-116), whereas the stem positions of CDR3 (IMGT positions 104, 105, 106, 117, and 118) are never within 5 Å of the antigen (Glanville et al. 2017). Whereas there is always at least one CDR3β contact, there are multiple cases, in which no CDR3α contact is made, suggesting that the former is required, although typically both are involved. Therefore, now well-established features of TCR repertoire analysis include length, charge, and hydrophobicity of the CDR3 regions, clonal diversity (within individuals), and amino acid sequence sharing (across individuals). Using, for example, the GLIPH algorithm can organize TCR sequences into distinct groups of shared specificity either within an individual or across a group of individuals.

Therefore, the estimated number of specific T cell receptors and thus the repertoire of amino acid sequences of the relevant variable regions is rather small, and the availability of even only one antigen-determining receptor sequence can readily enable the person of skill to create and search for other related T cell receptors sharing the same specificity. Since general methods of making TCRs are known, and the specific interactions between the peptide-MHC and the receptor have been extensively studied, even the knowledge about the peptide-MHC complex should provide the person of skill with sufficient information, to be fully able to produce the herein described specific subset of variable regions for the inventive T cell receptors (or the described specific fragments thereof), without suffering an undue burden, e.g. because of a lack of specific directions regarding the relevant positions of the receptors.

In one aspect, to obtain T cells expressing TCRs of the present description, nucleic acids encoding TCR-alpha and/or TCR-beta chains of the present description are cloned into expression vectors, such as gamma retrovirus, lentivirus, or non-viral vectors, e.g., transposons, nanoplasmids, and CRISPR. The recombinant viruses or vectors are generated and then tested for functionality, such as antigen specificity and functional avidity. An aliquot of the final product is then used to transduce the target T cell population (generally purified from patient PBMCs), which is expanded before infusion into the patient.

In another aspect, to obtain T cells expressing TCRs of the present description, TCR RNAs are synthesized by techniques known in the art, e.g., in vitro transcription systems. The in vitro-synthesized TCR RNAs are then introduced into primary CD8+ T cells obtained from healthy donors by electroporation to re-express tumor specific TCR-alpha and/or TCR-beta chains.

In an embodiment, a TCR of the present description having at least one mutation in the alpha chain and/or having at least one mutation in the beta chain has modified glycosylation compared to the unmutated TCR.

Alpha/beta heterodimeric TCRs of the present description may have an introduced disulfide bond between their constant domains. Preferred TCRs of this type include those which have a TRAC constant domain sequence and a TRBC1 or TRBC2 constant domain sequence except that Thr 48 of TRAC and Ser 57 of TRBC1 or TRBC2 are replaced by cysteine residues, the said cysteines forming a disulfide bond between the TRAC constant domain sequence and the TRBC1 or TRBC2 constant domain sequence of the TCR.

With or without the introduced inter-chain bond mentioned above, alpha/beta hetero-dimeric TCRs of the present description may have a TRAC constant domain sequence and a TRBC1 or TRBC2 constant domain sequence, and the TRAC constant domain sequence and the TRBC1 or TRBC2 constant domain sequence of the TCR may be linked by the native disulfide bond between Cys4 of exon 2 of TRAC and Cys2 of exon 2 of TRBC1 or TRBC2.

Therefore, in one additional or alternative embodiment the antigen recognizing construct of the invention comprises CDR1, CDR2, CDR2bis and CDR3 sequences in a combination as provided in SEQ ID NOs: 12-128, which display the respective variable chain allele together with the CDR3 sequence. Therefore, preferred are antigen recognizing constructs of the invention which comprise at least one, preferably, all four CDR sequences CDR1, CDR2, CDR2bis and CDR3. Preferably, an antigen recognizing construct of the invention comprises the respective CDR1, CDR2bis and CDR3 of one individual herein disclosed TCR variable region of the invention (see SEQ ID NOs: 12-128 and the example section).

In an embodiment, the TCR alpha variable domain has at least one mutation relative to a TCR alpha domain shown in SEQ ID NOs: 12-128, and/or the TCR beta variable domain has at least one mutation relative to a TCR alpha domain shown in SEQ ID NOs: 12-128. In an embodiment, a TCR comprising at least one mutation in the TCR alpha variable domain and/or TCR beta variable domain has a binding affinity for, and/or a binding half-life for, a TAA peptide-HLA molecule complex, which is at least double that of a TCR comprising the unmutated TCR alpha domain and/or unmutated TCR beta variable domain.

The antigen recognizing construct of the invention may comprise a TCR α or γ chain, and/or a TCR β or δ chain, wherein the TCR α or γ chain comprises a CDR3 having at least one, at least two, at least three, at least four, or at least five amino acid substitutions of an amino acid sequence selected from SEQ ID NOs: 14, 26, 38, 50, 62, 74, 86, and 110 and/or wherein the TCR β or δ chain comprises a CDR3 having at least one, at least two, at least three, at least four, or at least five amino acid substitutions of an amino acid sequence selected from SEQ ID NOs: 20, 32, 44, 56, 68, 80, 92, and 116.

Most preferably, in some additional embodiments, wherein the disclosure refers to antigen recognizing constructs comprising any of one, two, three, or all of the CDR1, CDR2, CDR2bis, and CDR3 regions of the herein disclosed TCR chains (see Table 1), such antigen recognizing constructs may be preferred, which comprise the respective CDR sequence of the invention with not more than three, two, and preferably only one, modified amino acid residues. A modified amino acid residue may be selected from an amino acid insertion, deletion, or substitution. Most preferred is that the three, two, preferably only one modified amino acid residue is the first or last amino acid residue of the respective CDR sequence. If the modification is a substitution, then it is preferable in some embodiments that the substitution is a conservative amino acid substitution.

Such conservative substitutions may be, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions are herein defined as exchanges within one of the following five groups: Group 1-small aliphatic, nonpolar or slightly polar residues (Ala, Ser, Thr, Pro, Gly), Group 2-polar, negatively charged residues and their amides (Asp, Asn, Glu, Gln), Group 3-polar, positively charged residues (His, Arg, Lys), Group 4-large, aliphatic, nonpolar residues (Met, Leu, Ile, Val, Cys), and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

If substitutions at more than one position are found to result in an antigen recognizing construct of the invention with substantially equivalent or greater antigen binding activity, then combinations of those substitutions will be tested to determine if the combined substitutions result in additive or synergistic effects on the antigen binding activity. For example, no more than four positions, no more than three positions, no more than two positions, or no more than one position within the CR3 region of an antigen recognizing construct of the invention would be simultaneously substituted.

If the antigen recognizing construct of the invention is composed of at least two amino acid chains, such as a double chain TCR, or antigen binding fragment thereof, the antigen recognizing construct may comprise in a first polypeptide chain the amino acid sequence according to SEQ ID NO: 14, and in a second polypeptide chain the amino acid sequence according to SEQ ID NO: 20, or in a first polypeptide chain the amino acid sequence according to SEQ ID NO: 26, and in a second polypeptide chain the amino acid sequence according to SEQ ID NO: 32, or in a first polypeptide chain the amino acid sequence according to SEQ ID NO: 38, and in a second polypeptide chain the amino acid sequence according to SEQ ID NO: 44, or in a first polypeptide chain the amino acid sequence according to SEQ ID NO: 50, and in a second polypeptide chain the amino acid sequence according to SEQ ID NO: 56, or in a first polypeptide chain the amino acid sequence according to SEQ ID NO: 62, and in a second polypeptide chain the amino acid sequence according to SEQ ID NO: 68, or in a first polypeptide chain the amino acid sequence according to SEQ ID NO: 74, and in a second polypeptide chain the amino acid sequence according to SEQ ID NO: 80, or in a first polypeptide chain the amino acid sequence according to SEQ ID NO: 86, and in a second polypeptide chain the amino acid sequence according to SEQ ID NO: 92, or in a first polypeptide chain the amino acid sequence according to SEQ ID NO: 110, and in a second polypeptide chain the amino acid sequence according to SEQ ID NO: 116.

Any one of the aforementioned double chain TCR, or antigen binding fragments thereof, are preferred TCR of the present invention. In some embodiments, the CDR3 of the double chain TCR of the invention may be mutated. Mutations of the CDR3 sequences as provided above preferably include a substitution, deletion, addition, or insertion of not more than three, preferably two, and most preferably not more than one amino acid residue. In some embodiments, the first polypeptide chain may be a TCR α or γ chain, and the second polypeptide chain may be a TCR β or δ chain. Preferred is the combination of an αβ or γδ TCR.

The TCR, or the antigen binding fragment thereof, is in some embodiments composed of a TCR α and a TCR β chain, or γ and δ chain. Such a double chain TCR comprises within each chain variable regions, and the variable regions each comprise one CDR1, one CDR2, or more preferably one CDR2bis, and one CDR3 sequence. The TCRs comprises the CDR1, CDR2, CDR2bis, and CDR3 sequences as comprised in the variable chain amino acid sequence of SEQ ID NOs: 15 and 21, or 27 and 33, or 39 and 45, or 51 and 57, or 63 and 69, or 75 and 81, or 87 and 93, or 111 and 117.

Some embodiments of the invention pertain to a TCR, or a fragment thereof, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to SEQ ID NOs: 15 and 21, or 27 and 33, or 39 and 45, or 51 and 57, or 63 and 69, or 75 and 81, or 87 and 93, or 111 and 117.

In a particularly preferred embodiment, the present invention provides an improved TCR, designated as R11P3D3_KE, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to SEQ ID NOs: 113 and 119. This TCR showed a surprisingly improved functionality in terms of tumor cell recognition when compared to its parent receptor, designated herein as R11P3D3.

The inventive TCRs may further comprise a constant region derived from any suitable species, such as any mammal, e.g., human, rat, monkey, rabbit, donkey, or mouse. In an embodiment of the invention, the inventive TCRs further comprise a human constant region. In some preferred embodiments, the constant region of the TCR of the invention may be slightly modified, for example, by the introduction of heterologous sequences, preferably mouse sequences, which may increase TCR expression and stability. In some preferred embodiments, the variable region of the TCR of the intervention may be slightly modified, for example, by the introduction of single point mutations to optimize the TCR stability and/or to enhance TCR chain pairing.

Some embodiments of the invention pertain to a TCR, or a fragment thereof, composed of a TCR α and a TCR β chain, wherein said TCR comprises the constant region having at least 50%, 60%, 70%, 80%, 90%, 95%, 98%, 99%, or preferably 100% sequence identity to an amino acid sequence selected from of the α and β chain according to SEQ ID NOs: 16 and 22, or 28 and 34, or 40 and 46, or 52 and 58, or 64 and 70, or 76 and 82, or 88 and 94, or 112 and 118.

The TCR α or γ chain of the invention may further comprise a CDR1 having at least one, at least two, at least three, at least four, or at least five amino acid substitutions of an amino acid sequence selected from SEQ ID NOs: 12, 24, 36, 48, 60, 72, 84 and 108, and/or a CDR2 having at least one, at least two, at least three, at least four, or at least five amino acid substitutions of an amino acid sequence selected from SEQ ID NOs: 13, 25, 37, 49, 61, 73, 85, and 109, and/or more preferably a CDR2bis having at least one, at least two, at least three, at least four, or at least five amino acid substitutions of an amino acid sequence selected from SEQ ID NOs: 120, 121, 122, 123, 124, 125, 126, and 128.

According to the invention the TCR β or δ chain may further comprise a CDR1 having at least one, at least two, at least three, at least four, or at least five amino acid substitutions of an amino acid sequence selected from SEQ ID NOs: 18, 30, 42, 54, 66, 78, 90, and 114, and/or a CDR2 having at least one, at least two, at least three, at least four, or at least five amino acid substitutions of an amino acid sequence selected from SEQ ID NOs: 19, 31, 43, 55, 67, 79, 91, and 115, and/or more preferably a CDR2bis having at least one, at least two, at least three, at least four, or at least five amino acid substitutions of an amino acid sequence selected from SEQ ID NOs: 19, 31, 43, 55, 67, 79, 91, and 115.

The antigen recognizing construct may in a further embodiment comprise a binding fragment of a TCR, and wherein said binding fragment comprises in one chain CDR1, CDR2, CDR2bis and CDR3, optionally selected from the CDR1, CDR2, CDR2bis and CDR3 sequences having the amino acid sequences of SEQ ID NOs: 12, 13, 14, 120, 11, 18, 19, 20, or 24, 25, 26, 121, or 30, 31, 32, or 36, 37, 38, 122, or 42, 43, 44, or 48, 49, 50, 123, or 54, 55, 56, or 60, 61, 62, 124, or 66, 67, 68, or 72, 73, 74, 125, or 78, 79, 80, or 84, 85, 86, 126, or 90, 91, 92, or 108, 109, 110, 128, or 114, 115, 116.

In further embodiments of the invention the antigen recognizing construct as described herein elsewhere is a TCR, or a fragment thereof, composed of at least one TCR α and one TCR β chain sequence, wherein said TCR α chain sequence comprises the CDR1, CDR2, CDR2bis, and CDR3 sequences having the amino acid sequences of SEQ ID NOs: 12 to 14 and 120, and said TCR β chain sequence comprises the CDR1 to CDR3 sequences having the amino acid sequences of SEQ ID NOs: 18 to 20, or wherein said TCR α chain sequence comprises the CDR1, CDR2, CDR2bis, and CDR3 sequences having the amino acid sequences of SEQ ID NOs: 24 to 26 and 121, and said TCR β chain sequence comprises the CDR1 to CDR3 sequences having the amino acid sequences of SEQ ID NOs: 30 to 32, or wherein said TCR α chain sequence comprises the CDR1, CDR2, CDR2bis, and CDR3 sequences having the amino acid sequences of SEQ ID NOs: 36 to 38 and 122 and said TCR β chain sequence comprises the CDR1 to CDR3 sequences having the amino acid sequences of SEQ ID NOs: 42 to 44, or wherein said TCR α chain sequence comprises the CDR1, CDR2, CDR2bis, and CDR3 sequences having the amino acid sequences of SEQ ID NOs: 48 to 50 and 123, and said TCR β chain sequence comprises the CDR1 to CDR3 sequences having the amino acid sequences of SEQ ID NOs: 54 to 56, or wherein said TCR α chain sequence comprises the CDR1, CDR2, CDR2bis, and CDR3 sequences having the amino acid sequences of SEQ ID NOs: 60 to 62 and 124, and said TCR β chain sequence comprises the CDR1 to CDR3 sequences having the amino acid sequences of SEQ ID NOs: 66 to 68, or wherein said TCR α chain sequence comprises the CDR1, CDR2, CDR2bis, and CDR3 sequences having the amino acid sequences of SEQ ID NOs: 72 to 74 and 125, and said TCR β chain sequence comprises the CDR1 to CDR3 sequences having the amino acid sequences of SEQ ID NOs: 78 to 80, or wherein said TCR α chain sequence comprises the CDR1, CDR2, CDR2bis, and CDR3 sequences having the amino acid sequences of SEQ ID NOs: 84 to 86 and 126, and said TCR β chain sequence comprises the CDR1 to CDR3 sequences having the amino acid sequences of SEQ ID NOs: 90 to 92, or wherein said TCR α chain sequence comprises the CDR1, CDR2, CDR2bis, and CDR3 sequences having the amino acid sequences of SEQ ID NOs: 108 to 110 and 128, and said TCR β chain sequence comprises the CDR1 to CDR3 sequences having the amino acid sequences of SEQ ID NOs: 114 to 116.

In further embodiments of the invention the antigen recognizing construct as described herein before is a TCR, or a fragment thereof, comprising at least one TCR α and one TCR β chain sequence, wherein said TCR α chain sequence comprises a variable region sequence having the amino acid sequence of SEQ ID NO: 15, and wherein said TCR β chain sequence comprises a variable region sequence having the amino acid sequence of SEQ ID NO: 21, or wherein said TCR α chain sequence comprises a variable region sequence having the amino acid sequence of SEQ ID NO: 27, and wherein said TCR β chain sequence comprises a variable region sequence having the amino acid sequence of SEQ ID NO: 33, or wherein said TCR α chain sequence comprises a variable region sequence having the amino acid sequence of SEQ ID NO: 39, and wherein said TCR β chain sequence comprises a variable region sequence having the amino acid sequence of SEQ ID NO: 45, or wherein said TCR α chain sequence comprises a variable region sequence having the amino acid sequence of SEQ ID NO: 51, and wherein said TCR β chain sequence comprises a variable region sequence having the amino acid sequence of SEQ ID NO: 57, or wherein said TCR α chain sequence comprises a variable region sequence having the amino acid sequence of SEQ ID NO: 63, and wherein said TCR β chain sequence comprises a variable region sequence having the amino acid sequence of SEQ ID NO: 69, or wherein said TCR α chain sequence comprises a variable region sequence having the amino acid sequence of SEQ ID NO: 75, and wherein said TCR (3 chain sequence comprises a variable region sequence having the amino acid sequence of SEQ ID NO: 81, or wherein said TCR α chain sequence comprises a variable region sequence having the amino acid sequence of SEQ ID NO: 87, and wherein said TCR β chain sequence comprises a variable region sequence having the amino acid sequence of SEQ ID NO: 93, or wherein said TCR α chain sequence comprises a variable region sequence having the amino acid sequence of SEQ ID NO: 111, and wherein said TCR β chain sequence comprises a variable region sequence having the amino acid sequence of SEQ ID NO: 117.

In further embodiments of the invention the antigen recognizing construct as described herein before is a TCR, or a fragment thereof, further comprising a TCR constant region having at least 50%, 60%, 70%, 80%, 90%, 95%, 98%, 99%, or 100% sequence identity to an amino acid sequence selected from SEQ ID NOs: 16, 22, 28, 34, 40, 46, 52, 58, 64, 70, 76, 82, 88, 94, 112, and 118, preferably wherein the TCR is composed of at least one TCR α and one TCR β chain sequence, wherein the TCR α chain sequence comprises a constant region having at least 50%, 60%, 70%, 80%, 90%, 95%, 98%, 99%, or 100% sequence identity to an amino acid sequence selected from SEQ ID NOs: 16, 28, 40, 52, 64, 76, 88, and 112, and wherein the TCR β chain sequence comprises a constant region having at least 50%, 60%, 70%, 80%, 90%, 95%, 98%, 99%, or 100% sequence identity to an amino acid sequence selected from SEQ ID NOs: 22, 34, 46, 58, 70, 82, 94, and 118.

Also disclosed are antigen recognizing constructs as described herein before comprising a first TCR chain having at least 50%, 60%, 70%, 80%, 90%, 95%, 98%, 99%, or 100% sequence identity to the amino acid sequence of SEQ ID NO: 17, and a second TCR chain having at least 50%, 60%, 70%, 80%, 90%, 95%, 98%, 99%, or 100% sequence identity to the amino acid sequence of SEQ ID NO: 23, The invention also provides TCRs comprising a first TCR chain having at least 50%, 60%, 70%, 80%, 90%, 95%, 98%, 99%, or 100% sequence identity to the amino acid sequence of SEQ ID NO: 29, and a second TCR chain having at least 50%, 60%, 70%, 80%, 90%, 95%, 98%, 99%, or 100% sequence identity to the amino acid sequence of SEQ ID NO: 35, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 41, and a second TCR chain having at least 50%, 60%, 70%, 80%, 90%, 95%, 98%, 99%, or 100% sequence identity to the amino acid sequence of SEQ ID NO: 47, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 53, and a second TCR chain having at least 50%, 60%, 70%, 80%, 90%, 95%, 98%, 99%, or 100% sequence identity to the amino acid sequence of SEQ ID NO: 59, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 65, and a second TCR chain having at least 50%, 60%, 70%, 80%, 90%, 95%, 98%, 99%, or 100% sequence identity to the amino acid sequence of SEQ ID NO: 71, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 77, and a second TCR chain having at least 50%, 60%, 70%, 80%, 90%, 95%, 98%, 99%, or 100% sequence identity to the amino acid sequence of SEQ ID NO: 83, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 89, and a second TCR chain having at least 50%, 60%, 70%, 80%, 90%, 95%, 98%, 99%, or 100% sequence identity to the amino acid sequence of SEQ ID NO: 95, In further embodiments, the invention provides antigen recognizing constructs which are TCR and comprise a first TCR chain having at least 50%, 60%, 70%, 80%, 90%, 95%, 98%, 99%, or 100% sequence identity to the amino acid sequence of SEQ ID NO: 113, and a second TCR chain having at least 50%, 60%, 70%, 80%, 90%, 95%, 98%, 99%, or 100% sequence identity to the amino acid sequence of SEQ ID NO: 119.

As used herein, the term “murine” or “human,” when referring to an antigen recognizing construct, or a TCR, or any component of a TCR described herein (e.g., complementarity determining region (CDR), variable region, constant region, αchain, and/or β chain), means a TCR (or component thereof), which is derived from a mouse or a human unrearranged TCR locus, respectively.

In an embodiment of the invention, chimeric TCR are provided, wherein the TCR chains comprise sequences from multiple species. Preferably, a TCR of the invention may comprise an α chain comprising a human variable region of an α chain and, for example, a murine constant region of a murine TCR α chain.

According to another aspect of the invention, a nucleic acid is provided, which encodes for a peptide according to the above description, or for an antibody or fragment thereof according to the above description, or for a T cell receptor or fragment thereof according to the above description.

In different embodiments said nucleic acid is provided in the form of DNA or RNA. In one embodiment said nucleic acid is provided in the form of a vector or a plasmid. In one embodiment, the nucleic acid comprises two or more repeats of the encoding sequence, (concatemer), separated by short nucleotide stretches (“spacers”).

Alternatively, or in addition, a method of treating a patient (i) being diagnosed for, (ii) suffering from or (iii) being at risk of developing, metastasis or a metastatic lesion,

is provided.

The method comprises administering to the patient a nucleic acid which encodes for a peptide according to the above description, or for an antibody or fragment thereof according to the above description, or for a T cell receptor or fragment thereof according to the above description, in one or more therapeutically effective doses.

Alternatively, or in addition, a pharmaceutical composition for treating metastasis or a metastatic lesion is provided, comprising a nucleic acid which encodes for a peptide according to the above description, or for an antibody or fragment thereof according to the above description, or for a T cell receptor or fragment thereof according to the above description, as an effective ingredient.

In one embodiment, said treatment or composition does not encompass the co-administration (simultaneously or sequentially) with a nucleic acid that encodes for a peptide that is a fragment of the Prostate specific Membrane antigen (PSMA), or for an antibody or T cell receptor binding such peptide when bound to an MHC molecule.

In particular, said treatment does not encompass the co-administration (simultaneously or sequentially) with a nucleic acid that encodes for an antibody or T cell receptor or functional fragment thereof that binds to PSMA₂₈₈₋₂₉₇ (GLPSIPVHPI, SEQ ID NO: 376) or PSMA₂₈₈₋₂₉₇ I297V (GLPSIPVHPV, SEQ ID NO: 377), the peptide being bound to an MHC.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

Optionally, said nucleic acid is provided for use in the (manufacture of a medicament for the) treatment of a patient (i) being diagnosed for, (ii) suffering from, or (iii) being at risk of developing, metastasis or a metastatic lesion.

Such nucleic acid can be an mRNA or a DNA. Such nucleic acid can be delivered as a plasmid or a linear molecule. Such nucleic acid can be delivered by a viral vector or encapsulated into a liposome. Such mRNA can comprise modified nucleosides, like pseudouridine or 1-methyl pseudouridine, to reduce immunogenic effects. Such mRNA can be G/C codon optimized to have a decreased uridine content.

According to another aspect of the invention, a recombinant host cell comprising the peptide according to the above description, the antibody or fragment thereof to the above description, the T cell receptor or fragment thereof according to the above description or the nucleic acid according to the above description is provided.

According to another aspect of the invention, a recombinant T lymphocyte is provided which expresses at least one vector encoding a T cell receptor according to the above description.

The T Lymphocyte is provided for use in the (manufacture of a medicament for the) treatment of a patient (i) being diagnosed for, (ii) suffering from, or (iii) being at risk of developing, metastasis or a metastatic lesion.

Alternatively, or in addition, a method of treating a patient (i) being diagnosed for, (ii) suffering from, or (iii) being at risk of developing, metastasis or a metastatic lesion,

is provided.

The method comprises administering to the patient a recombinant T lymphocyte which expresses at least one vector encoding a T cell receptor according to the above description, in one or more therapeutically effective doses.

Alternatively, or in addition, a pharmaceutical composition for treating metastasis or a metastatic lesion is provided, comprising a recombinant T lymphocyte which expresses at least one vector encoding a T cell receptor according to the above description, as an effective ingredient.

In one embodiment, said treatment or composition does not encompass the co-administration (simultaneously or sequentially) with a recombinant T lymphocyte that expresses a vector that encodes for a T cell receptor or functional fragment thereof that binds to a fragment of the Prostate specific Membrane antigen (PSMA), the peptide being bound to an MHC molecule; in particular not to PSMA₂₈₈₋₂₉₇ (GLPSIPVHPI, SEQ ID NO: 376) or PSMA₂₈₈₋₂₉₇ I297V (GLPSIPVHPV, SEQ ID NO: 377), the peptide being bound to an MHC molecule.

In one embodiment, the recombinant T lymphocytes are produced by a method comprising isolating a cell from a subject, transforming the cell with at least one vector encoding the T cell receptor, to produce a recombinant T lymphocyte, and expanding the recombinant T lymphocyte to produce the population of recombinant T lymphocytes.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

In one embodiment, the recombinant T lymphocyte is a CD8+ (CD8 positive) T lymphocyte. A CD8+ T lymphocyte (also called cytotoxic T cell CTL, T-killer cell, cytolytic T cell, or killer T cell) is a T lymphocyte hat kills cancer cells, cells that are infected (particularly with viruses), or cells that are damaged in other ways.

Most cytotoxic T cells express T cell receptors (TCRs) that can recognize a specific antigen. An antigen is a molecule capable of stimulating an immune response and is often produced by cancer cells or viruses. Antigens inside a cell are bound to class I MHC molecules and brought to the surface of the cell by the class I MHC molecule, where they can be recognized by the T cell. If the TCR is specific for that antigen, it binds to the complex of the class I MHC molecule and the antigen, and the T cell destroys the cell.

For the TCR to bind to the class I MHC molecule, the former must be accompanied by a glycoprotein called CD8, which binds to the constant portion of the class I MHC molecule. Therefore, these T cells are called CD8+ T cells.

According to several embodiments, the T cell receptor comprises:
(1) a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
(2) a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
(3) a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
(4) a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
(5) a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
(6) a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
(7) a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92, wherein the T cell receptor is capable of binding to a peptide consisting of the amino acid sequence of SLLQHLIGL (SEQ ID NO: 310) in a complex with HLA-A*02.

According to several embodiments, the T cell receptor comprises:
(1) an α chain variable domain comprising SEQ ID NO: 15, and a β chain variable domain comprising SEQ ID NO: 21, or
(2) an α chain variable domain comprising SEQ ID NO: 27, and a β chain variable domain comprising SEQ ID NO: 33, or
(3) an α chain variable domain comprising SEQ ID NO: 39, and a β chain variable domain comprising SEQ ID NO: 45, or
(4) an α chain variable domain comprising SEQ ID NO: 51, and a β chain variable domain comprising SEQ ID NO: 57, or
(5) an α chain variable domain comprising SEQ ID NO: 63, and a β chain variable domain comprising SEQ ID NO: 69, or
(6) an α chain variable domain comprising SEQ ID NO: 75, and a β chain variable domain comprising SEQ ID NO: 81, or
(7) an α chain variable domain comprising SEQ ID NO: 87, and a β chain variable domain comprising SEQ ID NO: 93, or
(8) an α chain variable domain comprising SEQ ID NO: 111, and a β chain variable domain comprising SEQ ID NO: 117,
   wherein the T cell receptor is capable of binding to a peptide consisting of the amino acid sequence of SLLQHLIGL (SEQ ID NO: 310) in a complex with HLA-A*02.

According to another aspect of the invention, an in vitro method for producing activated T lymphocytes is provided. The method comprises contacting in vitro T cells with antigen-loaded human class I MHC molecules expressed on the surface of a suitable antigen-presenting cell or an artificial construct mimicking an antigen-presenting cell for a period of time sufficient to activate said T lymphocyte in an antigen-specific manner. Said antigen is a peptide according to the above description.

According to another aspect of the invention, an activated T lymphocyte, produced by the method according to the above description is provided, which selectively recognizes a cell which presents a peptide according to the above description.

The T lymphocyte is provided for use in the (manufacture of a medicament for the) treatment of a patient (i) being diagnosed for, (ii) suffering from, or (iii) being at risk of developing, metastasis or a metastatic lesion.

Alternatively, or in addition, a method of treating a patient (i) being diagnosed for, (ii) suffering from, or (iii) being at risk of developing, metastasis or a metastatic lesion,

is provided.

The method comprises administering to the patient an activated T lymphocyte, produced by the method according to the above description, which selectively recognizes a cell which presents a peptide according to the above description, in one or more therapeutically effective doses.

Alternatively, or in addition, a pharmaceutical composition for treating metastasis or a metastatic lesion is provided, comprising an activated T lymphocyte, produced by the method according to the above description, which selectively recognizes a cell which presents a peptide according to the above description, as an effective ingredient.

In one embodiment, said treatment does not encompass the co-administration (simultaneously or sequentially) with an activated T lymphocyte that recognizes a cell which presents a peptide that is a fragment of the Prostate specific Membrane antigen (PSMA), in particular does not present PSMA₂₈₈₋₂₉₇ (GLPSIPVHPI, SEQ ID NO: 376) or PSMA₂₈₈₋₂₉₇ I297V (GLPSIPVHPV, SEQ ID NO: 377).

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

In one embodiment, the activated T lymphocyte is a CD8+(CD8 positive) T lymphocyte.

Adoptive Cellular Therapy: γδ T cell Manufacturing

To isolate γδ T cells, in an aspect, γδ T cells may be isolated from a subject or from a complex sample of a subject. In an aspect, a complex sample may be a peripheral blood sample, a cord blood sample, a tumor, a stem cell precursor, a tumor biopsy, a tissue, a lymph, or from epithelial sites of a subject directly contacting the external milieu or derived from stem precursor cells. γδ T cells may be directly isolated from a complex sample of a subject, for example, by sorting γδ T cells that express one or more cell surface markers with flow cytometry techniques. Wild-type γδ T cells may exhibit numerous antigen recognition, antigen-presentation, co-stimulation, and adhesion molecules that can be associated with a γδ T cells. One or more cell surface markers, such as specific γδ TCRs, antigen recognition, antigen-presentation, ligands, adhesion molecules, or co-stimulatory molecules may be used to isolate wild-type γδ T cells from a complex sample. Various molecules associated with or expressed by γδ T cells may be used to isolate γδ T cells from a complex sample, e.g., isolation of mixed population of Vδ1+, Vδ2+, Vδ3+ cells or any combination thereof.

For example, peripheral blood mononuclear cells can be collected from a subject, for example, with an apheresis machine, including the Ficoll-Paque™ PLUS (GE Healthcare) system, or another suitable device/system. γδ T cell(s), or a desired subpopulation of γδ T cell(s), can be purified from the collected sample with, for example, with flow cytometry techniques. Cord blood cells can also be obtained from cord blood during the birth of a subject.

Positive and/or negative selection of cell surface markers expressed on the collected γδ T cells can be used to directly isolate γδ T cells, or a population of γδ T cells expressing similar cell surface markers from a peripheral blood sample, a cord blood sample, a tumor, a tumor biopsy, a tissue, a lymph, or from an epithelial sample of a subject. For instance, γδ T cells can be isolated from a complex sample based on positive or negative expression of CD2, CD3, CD4, CD8, CD24, CD25, CD44, Kit, TCR α, TCR β, TCR α, TCR δ, NKG2D, CD70, CD27, CD30, CD16, CD337 (NKp30), CD336 (NKp46), OX40, CD46, CCR7, and other suitable cell surface markers.

This process may include collecting or obtaining white blood cells or PBMC from leukapheresis products. Leukapheresis may include collecting whole blood from a donor and separating the components using an apheresis machine. An apheresis machine separates out desired blood components and returns the rest to the donor's circulation. For instance, white blood cells, plasma, and platelets can be collected using apheresis equipment, and the red blood cells and neutrophils are returned to the donor's circulation. Commercially available leukapheresis products may be used in this process. Another way to obtain white blood cells is to obtain them from the buffy coat. To isolate the buffy coat, whole anticoagulated blood is obtained from a donor and centrifuged. After centrifugation, the blood is separated into plasma, red blood cells, and buffy coat. The buffy coat is the layer located between the plasma and red blood cell layers. Leukapheresis collections may result in higher purity and considerably increased mononuclear cell content than that achieved by buffy coat collection. The mononuclear cell content possible with leukapheresis may typically be 20 times higher than that obtained from the buffy coat. In order to enrich for mononuclear cells, the use of a Ficoll gradient may be needed for further separation.

To deplete αβ T cells from PBMC, αβ TCR-expressing cells may be separated from the PBMC by magnetic separation, e.g., using CliniMACS® magnetic beads coated with anti-αβ TCR antibodies, followed by cryopreserving αβ TCR-T cells depleted PBMC. To manufacture “off-the-shelf” T cell products, cryopreserved αβ TCR-T cells depleted PBMC may be thawed and activated in small/mid-scale, e.g., 24 to 4-6 well plates or T75/T175 flasks, or in large scale, e.g., 50 ml-100 liter bags, in the presence of aminobisphosphonate, e.g., zoledronate, and/or isopentenylpyrophosphate (IPP) and/or cytokines, e.g., interleukin 2 (IL-2), interleukin 15 (IL-15), and/or interleukin 18 (IL-18), and/or other activators, e.g., Toll-like receptor 2 (TLR2) ligand, for 1-10 days, e.g., 2-7 days.

### Engineering γδ T Cells Expressing αβ-TCR and CD8αβ

γδ T cells of the disclosure may be engineered for use to treat a subject in need of treatment for a condition. To engineer γδ T cells that express αβ-TCR, e.g., specifically binding to a PRAME-004-MHC complex, αβ-TCR-expressing γ-retrovirus was generated. Because γδ T cells may not express CD8, γδ T cells may need CD8α homodimers or CD8αβ heterodimers in addition to αβ-TCR to recognize PRAME-004/MHC-I complexes presented on cell membrane of target cells, e.g., cancer cells. To that end, αβ-TCR/CD8-expressing γ-retrovirus was generated for transducing isolated γδ T cells using the methods described herein. The sequences of CD8α or the variant thereof and CD8β or the variant thereof may be selected from SEQ ID NO: 1-11.

αβ-TCR-expressing Vγ962 T cells, in which αβ-TCR specifically binds to peptide-MHC complex, were generated by transducing Vγ962 T cells with αβ-TCR retrovirus and CD8αβ retrovirus.

### Autologous T Cell Manufacturing Process

Embodiments of the present disclosure may include an about 7- to about 10-day process leading to the manufacturing of over 10 billion (10×10⁹) cells without the loss of potency. In addition, the concentrations of several raw materials may be optimized to reduce the cost of good by 30%.

T cell manufacturing process of the present disclosure may include thawing PBMC on day 0, followed by resting without cytokines overnight, e.g., 24 hours, followed by activating the rested PBMC with anti-CD3 and anti-CD28 antibodies immobilized on non-tissue culture treated plates. IL-7 is a homeostatic cytokine that promotes survival of T cells by preventing apoptosis. IL-7 may be added to PBMC during resting.

T cell manufacturing process of the present disclosure may include thawing PBMC on day 1, followed by resting in the presence of IL-7 or in the presence of IL-7+IL-15 or without cytokine for 4-6 hours, followed by activating the rested PBMC with anti-CD3 and anti-CD28 antibodies immobilized on non-tissue culture treated plates.

T cell manufacturing process of the present disclosure may include thawing PBMC on day 1 (without resting and without cytokine), followed by activating the thawed PBMC with anti-CD3 and anti-CD28 antibodies immobilized on tissue culture plates. Cells may be harvested and counted on day 8-10, followed by activation panel analysis.

T cell manufacturing process of the present disclosure may include resting PBMC for a period of time of about 4 hours according to one embodiment of the present disclosure. For example, a T cell manufacturing process may include isolation and cryopreservation of PBMC from leukapheresis, in which sterility may be tested; thaw, rest (e.g., about 4 hours) and activate T cells; transduction with a viral vector; expansion with cytokines; split/feed cells, in which cell count and immunophenotyping may be tested; harvest and cryopreservation of drug product cells, in which cell count and mycoplasma may be tested, and post-cryopreservation release, in which viability, sterility, endotoxin, immunophenotyping, copy number of integrated vector, and vesicular stomatitis virus glycoprotein G (VSV-g) may be tested.

T cell manufacturing process of the present disclosure may include resting PBMC overnight (about 16 hours). For example, T cell manufacturing process may include isolation of PBMC, in which PBMC may be used fresh or stored frozen till ready for use, or may be used as starting materials for T cell manufacturing and selection of lymphocyte populations (e.g., CD8, CD4, or both) may also be possible; thaw and rest lymphocytes overnight, e.g., about 16 hours, which may allow apoptotic cells to die off and restore T cell functionality (this step may not be necessary, if fresh materials are used); activation of lymphocytes, which may use anti-CD3 and anti-CD28 antibodies (soluble or surface bound, e.g., magnetic or biodegradable beads); transduction with TCRs or bi-specific molecules, which may use lentiviral or retroviral constructs encoding TCRs or bi-specific molecules or may use non-viral methods; and expansion of lymphocytes, harvest, and cryopreservation, which may be carried out in the presence of cytokine(s), serum (ABS or FBS), and/or cryopreservation media.

Table 2a summarizes characteristics of T cells manufactured with short rest of about 4 hours according to one embodiment of the present disclosure and that with overnight rest of about 16 hours.

TABLE 2a
Characteristics of T cells manufactured with protocols including
4 hours versus 16 hours resting.

| | | | | % Live | | % Dex+ |
|---|---|---|---|---|---|---|
| Resting | Fold | Harvest | Viability | CD3+ | % CD8+ | of CD8+ |
| for | Expansion | Count | ≥70% | ≥80% | of CD3+ | ≥10% |
| 4 hours | 78.7 | 28.0 × 10⁹ | 92.0 | 99.7 | 53.4 | 63.7 |
| 16 hours | 45.0 | 15.7 × 10⁹ | 86.0 | 99.5 | 51.9 | 53.0 |

T cell manufacturing process of the present disclosure may include using fresh PBMCs, which is not obtained by thawing cryopreserved PBMC, thus, minimizing cell loss due to freezing, thawing, and/or resting PBMCs and maximizing cell numbers at the beginning of manufacturing process. For example, T cell manufacturing process may include day 0, isolation of fresh PBMC, activation of fresh lymphocytes using, for example, anti-CD3 and anti-CD28 antibodies (soluble or surface bound, e.g., magnetic or biodegradable beads) in bags, e.g., Saint-Gobain VueLife AC Bags, coated with anti-CD3 and anti-CD28 antibodies; day 1, transduction with TCRs or bi-specific molecules using, for example, lentiviral or retroviral constructs encoding TCRs or bi-specific molecules or non-viral methods, e.g., liposomes; and day 2, expansion of lymphocytes, day 5/6, harvest, and cryopreservation in the presence of cytokine(s), serum (ABS or FBS), and/or cryopreservation media.

### Engineering αβ T Cells Expressing αβ-TCR and CD8αβ

Engineered αβ T cells of the disclosure may be used to treat a subject in need of treatment for a condition. To engineer αβ T cells that express αβ-TCR, e.g., shown below in the sequence listing, specifically binding to a PRAME-004/MHC complex, αβ-TCR-expressing γ-retrovirus was generated. Expression of exogenous CD8α homodimers or CD8αβ heterodimers in CD8+ and/or CD4 T cells may improve αβ-TCR to recognize PRAME-004/MHC-I complexes on cell membrane of target cells, e.g., cancer cells. To that end, up-TCR/CD8-expressing γ-retrovirus was generated for transducing T cells using the methods described herein. The sequences of CD8α or the variant thereof and CD8β or the variant thereof may be selected from SEQ ID NO: 1-11.

### Methods of Treatment

Compositions containing engineered αβ T cells (e.g., CD4+ and CD8+ T cells) and/or γδ T cells that express recombinant TCRs and/or bi-specific molecules binding to PRAME-004 described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. Engineered αβ T cells and/or γδ T cells can also be administered to lessen a likelihood of developing, contracting, or worsening a condition. Effective amounts of a population of engineered αβ T cells and/or γδ T cells for therapeutic use can vary based on the severity and course of the disease or condition, previous therapy, the subject's health status, weight, and/or response to the drugs, and/or the judgment of the treating physician.

The composition of the present disclosure may also include one or more adjuvants. Adjuvants are substances that non-specifically enhance or potentiate the immune response (e.g., immune responses mediated by CD8-positive T cells and helper-T (TH) cells to an antigen and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel 1995). Also, cytokines may be used. Several cytokines have been directly linked to influencing dendritic cell migration to lymphoid tissues (e.g., TNF-), accelerating the maturation of dendritic cells into efficient antigen-presenting cells for T lymphocytes (e.g., GM-CSF, IL-1 and IL-4) (U.S. Pat. No. 5,849,589, incorporated herein by reference in its entirety) and acting as immunoadjuvants (e.g., IL-12, IL-15, IL-23, IL-7, IFN-alpha, IFN-beta).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg 2006). U.S. Pat. No. 6,406,705 B1 describes the combined use of CpG oligonucleotides, non-nucleic acid adjuvants, and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA, mimetics of the bacterial lipopeptide Pam3Cys-Ser-Ser such as Pam3Cys-GDPKHPKSF (XS15). See (Gouttefangeas and Rammensee 2018; Rammensee et al. 2019), the content of which is incorporated herein by reference, for enabling disclosure

Other examples for useful adjuvants include immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Preferred adjuvants are anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), virosomes, and/or interleukin (IL)-1, IL-2, IL-4, IL-7, IL-12, IL-13, IL-15, IL-21, and IL-23.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the invention, the adjuvant is cyclophosphamide, imiquimod, or resiquimod. Even more preferred adjuvants are Montanide IMS 1312, Montanide ISA 206, Montanide ISA 50V, Montanide ISA-51, poly-ICLC (Hiltonol®), and anti-CD40 mAB, or combinations thereof.

Engineered αβ T cells and/or γδ T cells of the present disclosure can be used to treat a subject in need of treatment for a condition, for example, a cancer described herein.

A method of treating a condition (e.g., ailment) in a subject with engineered αβ T cells and/or γδ T cells may include administering to the subject a therapeutically effective amount of engineered αβ T cells and/or γδ T cells. Engineered αβ T cells and/or γδ T cells of the present disclosure may be administered at various regimens (e.g., timing, concentration, dosage, spacing between treatment, and/or formulation). A subject can also be preconditioned with, for example, chemotherapy, radiation, or a combination of both, prior to receiving engineered up T cells and/or γδ T cells of the present disclosure. A population of engineered αβ T cells and/or γδ T cells may also be frozen or cryopreserved prior to being administered to a subject. A population of engineered αβ T cells and/or γδ T cells can include two or more cells that express identical, different, or a combination of identical and different tumor recognition moieties. For instance, a population of engineered αβ T cells and/or γδ T cells can include several distinct engineered αβ T cells and/or γδ T cells that are designed to recognize different antigens, or different epitopes of the same antigen.

In an aspect, engineered αβ T cells and/or γδ T cells of the present disclosure may be used to treat an infectious disease. In another aspect, engineered αβ T cells and/or γδ T cells of the present disclosure may be used to treat an infectious disease, an infectious disease may be caused by a virus. In yet another aspect, engineered αβ T cells and/or γδ T cells of the present disclosure may be used to treat an immune disease, such as an autoimmune disease.

Treatment with αβ T cells and/or γδ T cells of the present disclosure may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered αβ T cells and/or γδ T cells of the present disclosure.

In another aspect, administration of engineered αβ T cells and/or γδ T cells of the present disclosure to a subject may modulate the activity of endogenous lymphocytes in a subject's body. In another aspect, administration of engineered αβ T cells and/or γδ T cells to a subject may provide an antigen to an endogenous T cell and may boost an immune response. In another aspect, the memory T cell may be a CD4+ T cell. In another aspect, the memory T cell may be a CD8+ T cell. In another aspect, administration of engineered αβ T cells and/or γδ T cells of the present disclosure to a subject may activate the cytotoxicity of another immune cell. In another aspect, the other immune cell may be a CD8+ T cell. In another aspect, the other immune cell may be a Natural Killer T cell. In another aspect, administration of engineered αβ T cells and/or γδ T cells of the present disclosure to a subject may suppress a regulatory T cell. In another aspect, the regulatory T cell may be a FOX3+ Treg cell. In another aspect, the regulatory T cell may be a FOX3− Treg cell. Non-limiting examples of cells whose activity can be modulated by engineered αβ T cells and/or γδ T cells of the disclosure may include: hematopoietic stem cells; B cells; CD4; CD8; red blood cells; white blood cells; dendritic cells, including dendritic antigen presenting cells; leukocytes; macrophages; memory B cells; memory T cells; monocytes; natural killer cells; neutrophil granulocytes; T-helper cells; and T-killer cells.

During most bone marrow transplants, a combination of cyclophosphamide with total body irradiation may be conventionally employed to prevent rejection of the hematopoietic stem cells (HSC) in the transplant by the subject's immune system. In an aspect, incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of αβ T cells and/or γδ T cells into humans may require the co-administration of αβ T cells and/or γδ T cells and interleukin-2. However, both low- and high-dosages of IL-2 can have highly toxic side effects. IL-2 toxicity can manifest in multiple organs/systems, most significantly the heart, lungs, kidneys, and central nervous system. In another aspect, the disclosure provides a method for administrating engineered αβ T cells and/or γδ T cells to a subject without the co-administration of a native cytokine or modified versions thereof, such as IL-2, IL-15, IL-12, IL-21. In another aspect, engineered αβ T cells and/or γδ T cells can be administered to a subject without co-administration with IL-2. In another aspect, engineered αβ T cells and/or γδ T cells may be administered to a subject during a procedure, such as a bone marrow transplant without the co-administration of IL-2.

### Methods of Administration

Generally, the therapeutic entities, including vaccines, antibodies, TCRs, bi- or multispecific molecules and T cells can be administered through every feasible mode of administration.

In one embodiment, the therapeutic entities are administered by injection or infusion im (intramuscular), iv (intravenously) or sc (subcutaneous). In one embodiment, the therapeutic entities are not administered intralymphatically. In one embodiment, the therapeutic entities are administered by injection or infusion im (intramuscular), iv (intravenously) or sc (subcutaneous)m, but not intralymphatically.

One or multiple engineered αβ T cells and/or γδ T cells populations may be administered to a subject in any order or simultaneously. If simultaneously, the multiple engineered αβ T cells and/or γδ T cells can be provided in a single, unified form, such as an intravenous injection, or in multiple forms, for example, as multiple intravenous infusions, s.c. injections or pills. Engineered γδ T cells can be packed together or separately, in a single package or in a plurality of packages. One or all of the engineered αβ T cells and/or γδ T cells can be given in multiple doses. If not simultaneous, the timing between the multiple doses may vary to as much as about a week, a month, two months, three months, four months, five months, six months, or about a year. In another aspect, engineered αβ T cells and/or γδ T cells can expand within a subject's body, in vivo, after administration to a subject. Engineered up T cells and/or γδ T cells can be frozen to provide cells for multiple treatments with the same cell preparation. Engineered αβ T cells and/or γδ T cells of the present disclosure, and pharmaceutical compositions comprising the same, can be packaged as a kit. A kit may include instructions (e.g., written instructions) on the use of engineered αβ T cells and/or γδ T cells and compositions comprising the same.

In another aspect, a method of treating a cancer comprises administering to a subject a therapeutically effective amount of engineered αβ T cells and/or γδ T cells, in which the administration treats the cancer. In another embodiment, the therapeutically effective amount of engineered αβ T cells and/or γδ T cells may be administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. In another aspect, the therapeutically effective amount of the engineered αβ T cells and/or γδ T cells may be administered for at least one week. In another aspect, the therapeutically effective amount of engineered αβ T cells and/or γδ T cells may be administered for at least two weeks.

Engineered αβ T cells and/or γδ T cells described herein can be administered before, during, or after the occurrence of a disease or condition, and the timing of administering a pharmaceutical composition containing an engineered αβ T cells and/or γδ T cell can vary. For example, engineered αβ T cells and/or γδ T cells can be used as a prophylactic and can be administered continuously to subjects with a propensity to conditions or diseases in order to lessen the likelihood of occurrence of the disease or condition. Engineered αβ T cells and/or γδ T cells can be administered to a subject during or as soon as possible after the onset of the symptoms. The administration of engineered αβ T cells and/or γδ T cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In another aspect, the administration of engineered αβ T cells and/or γδ T cells of the present disclosure may be an intravenous administration. One or multiple dosages of engineered αβ T cells and/or γδ T cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered αβ T cells and/or γδ T cells can be administered years after onset of the cancer and before or after other treatments. In another aspect, engineered αβ T cells and/or γδ T cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, or at least 5 years. The length of treatment can vary for each subject.

### Preservation

In an aspect, αβ T cells and/or γδ T cells may be formulated in freezing media and placed in cryogenic storage units such as liquid nitrogen freezers (−196° C.) or ultra-low temperature freezers (−65° C., −80° C., −120° C., or −150° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroxyethyl starch (HES) with physiological pH buffering agents to maintain pH between about 6.0 to about 6.5, about 6.5 to about 7.0, about 7.0 to about 7.5, about 7.5 to about 8.0, or about 6.5 to about 7.5. The cryopreserved αβ T cells and/or γδ T cells can be thawed and further processed by stimulation with antibodies, proteins, peptides, and/or cytokines as described herein. The cryopreserved up T cells and/or γδ T cells can be thawed and genetically modified with viral vectors (including retroviral, adeno-associated virus (AAV), and lentiviral vectors) or non-viral means (including RNA, DNA, e.g., transposons, and proteins) as described herein. The modified αβ T cells and/or γδ T cells can be further cryopreserved to generate cell banks in quantities of at least about 1, 5, 10, 100, 150, 200, 500 vials at about at least 101, 102, 103, 104, 105, 106, 107, 108, 109, or at least about 1010 cells per mL in freeze media. The cryopreserved cell banks may retain their functionality and can be thawed and further stimulated and expanded. In another aspect, thawed cells can be stimulated and expanded in suitable closed vessels, such as cell culture bags and/or bioreactors, to generate quantities of cells as allogeneic cell product. Cryopreserved αβ T cells and/or γδ T cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In another aspect, no preservatives may be used in the formulation. Cryopreserved αβ T cells and/or γδ T cells can be thawed and infused into multiple patients as allogeneic off-the-shelf cell product.

In an aspect, engineered αβ T cells and/or γδ T cell described herein may be present in a composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

In an aspect, methods described herein may be used to produce autologous or allogenic products according to an aspect of the disclosure.

According to one embodiment of the invention, the antibody according to the above description or the T cell receptor according to the above description further comprises an effector moiety, selected from the group consisting of
a) toxin, or
b) immune modulator.

Immune modulators are known. They are molecules which induce or stimulate an immune response, through direct or indirect activation of the humoral or cellular arm of the immune system, such as by activation of T cells. Examples include: IL-1, IL-1α, IL-3, IL-4, IL-5, IL-6, IL-7, IL-10, IL-11, IL-12, IL-13, IL-15, IL-21, IL-23, TGF-β, IFN-γ, TNFα, Anti-CD2 antibody, Anti-CD3 antibody, Anti-CD4 antibody, Anti-CD8 antibody, Anti-CD44 antibody, Anti-CD45RA antibody, Anti-CD45RB antibody, Anti-CD45RO antibody, Anti-CD49a antibody, Anti-CD49b antibody, Anti-CD49c antibody, Anti-CD49d antibody, Anti-CD49e antibody, Anti-CD49f antibody, Anti-CD16 antibody, Anti-CD28 antibody, Anti-IL-2R antibodies, viral proteins and peptides, and bacterial proteins or peptides. Where the immune modulator polypeptide is an antibody, it may specifically bind to an antigen presented by a T cell and may be a scFv antibody.

In one embodiment, the immune modulator is an anti CD3 antibody.

In one embodiment, the immune modulator binds to CD3γ, CD3δ, or CD3ε.

In one embodiment, the immune modulator is the anti CD3 antibody OKT3.

In one embodiment, the immune modulator is the anti CD3 antibody UCHT-1, or its humanized variant hUCHT-1.

In one embodiment, the immune modulator is the anti CD3 antibody BMA031.

In one embodiment, the immune modulator is the anti CD3 antibody 12F6.

In several embodiments, fragments, like e.g. the V_{H} and V_{L} domains, of these antibodies can be used. The skilled person is aware of how to derive, from a published antibody, its V_{H} and V_{L} domains.

Humanized antibody hUCHT1 is disclosed in (Zhu and Carter 1995), the content of which is incorporated herein by reference. In particular V_{H} and V_{L} domains derived from the UCHT1 variants UCHT1-V17, UCHT1-V17opt, UCHT1-V21, or UCHT1-V23 can be used, preferably derived from UCHT1-V17. Further preferred embodiments and variants of this antibody are disclosed elsewhere herein.

Antibody BMA031, which targets the TCRα/β CD3 complex, and humanized versions thereof, is disclosed in (Shearman et al. 1991). In particular V_{H} and V_{L} domains derived from BMA031 variants BMA031(V36), or BMA031(V10), preferably derived from BMA031(V36) can be used. Further preferred embodiments and variants of this antibody are disclosed elsewhere herein.

In further embodiments, the immune modulator binds to a cell surface antigen selected from the group consisting of CD4, CD7, CD8, CD10, CD11b, CD11c, CD14, CD16, CD18, CD22, CD25, CD28, CD32a, CD32b, CD33, CD41, CD41b, and/or CD42a.

Toxins to be used to couple with targeting domain are also known. See, e.g., (Storz 2015), the content of which is incorporated herein by reference.

In one embodiment, the toxin is an Auristatin (MMAE, MMAF).

In one embodiment, the toxin is a Maytansinoid,

In one embodiment, the toxin is an Anthracyclin or derivative thereof.

In one embodiment, the toxin is a Calicheamicin.

In one embodiment, the toxin is a Duocarmycin.

In one embodiment, the toxin is a Taxane.

In one embodiment, the toxin is a Pyrrolobenzodiazepine.

In one embodiment, the toxin is a α-Amanitin.

In one embodiment, the toxin is a ribotoxin or RNase.

In one embodiment, the toxin is a Tubulysin.

In one embodiment, the toxin is a Benzodiazepine derivative

According to one embodiment of the invention, a T cell receptor according to the description above is provided for use in the (manufacture of a medicament for the) treatment of a patient (i) being diagnosed for, (ii) suffering from, or (iii) being at risk of developing metastasis or a metastatic lesion.

The T cell receptor comprises a first polypeptide chain and a second polypeptide chain, wherein said first polypeptide chain comprising 95% identity to any one of

SEQ ID NOs 184, 187, 189, 190, 195, 206, 208, 210, 212, 216, 218, 219, 220, 221, 222, 229, 230, 232, 234, 236, 238, 240, 241, 242, 243, 244, 246, 250, 251, 252, 253, 254, 255, 256, 257, 258, 259, 260, 261, 262, 263, 265, 298, 299, 300, 302, or 304 comprises the complementarity determining regions (CDRs) of said sequence;

wherein the second polypeptide chain comprises a second hinge domain and/or a second Fc domain, wherein said second polypeptide comprising 95% identity to any one of

SEQ ID NOs 179, 180, 181, 182, 183, 185, 186, 188, 191, 194, 203, 205, 213, 214, 215, 217, 223, 224, 225, 226, 227, 228, 231, 233, 235, 237, 239, 245, 247, 248, 249, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 301, or 303 comprises the CDRs of said sequence.

Alternatively, or in addition, a method of treating a patient (i) being diagnosed for, (ii) suffering from or (iii) being at risk of developing, metastasis or a metastatic lesion,

is provided.

The method comprises administering to the patient a T cell receptor comprising a first polypeptide chain and a second polypeptide chain, wherein said first polypeptide chain comprising 95% identity to any one of SEQ ID NOs 184, 187, 189, 190, 195, 206, 208, 210, 212, 216, 218, 219, 220, 221, 222, 229, 230, 232, 234, 236, 238, 240, 241, 242, 243, 244, 246, 250, 251, 252, 253, 254, 255, 256, 257, 258, 259, 260, 261, 262, 263, 265, 298, 299, 300, 302, or 304 comprises the complementarity determining regions (CDRs) of said sequence; wherein the second polypeptide chain comprises a second hinge domain and/or a second Fc domain, wherein said second polypeptide comprising 95% identity to any one of SEQ ID NOs 179, 180, 181, 182, 183, 185, 186, 188, 191, 194, 203, 205, 213, 214, 215, 217, 223, 224, 225, 226, 227, 228, 231, 233, 235, 237, 239, 245, 247, 248, 249, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 301, or 303 comprises the CDRs of said sequence.

The said sequences are T cell receptor variable domains. The CDRs of a T cell receptor variable domain can be determined based on (Lefranc et al. 2003), the content of which is incorporated herein by reference. Further disclosure can be found in imgt.org/IMGTScientificChart/Numbering/IMGTIGVLsuperfamily.html

Alternatively, or in addition, a pharmaceutical composition for treating metastasis or a metastatic lesion is provided, comprising such T cell receptor as an effective ingredient.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

In one embodiment, said first polypeptide chain is fused to said second polypeptide chain by covalent and/or non-covalent bonds between the first hinge domain and the second hinge domain, and/or between the first Fc domain and the second Fc domain.

In one embodiment, said first polypeptide chain is fused to said second polypeptide chain by covalent and/or non-covalent bonds between the first hinge domain and the second hinge domain, and/or between the first Fc domain and the second Fc domain

In one embodiment, said first and second Fc domains each comprise at least one Fc effector function silencing mutation.

For example, the Fc domain on one or both, preferably both polypeptide chains can comprise one or more alterations that inhibit Fc gamma receptor (FcyR) binding. Such alterations can include L234A, L235A.

In a further embodiment, the Fc domain on one or both, preferably both polypeptide

chains can comprise a N297Q mutation to remove the N-glycosylation site within the Fc-part. Such a mutation abrogates the Fc-gamma-receptor interaction.

In one embodiment, said first and second Fc domains each comprise a CH3 domain comprising at least one mutation that facilitates the formation of heterodimers.

Accordingly, in some embodiments, the Fc domain of one of the polypeptides, for example Fc1, comprises the amino acid substitutions S354C and T366W (knob) in its CH3 domain and the Fc domain of the other polypeptide, for example Fc2, comprises the amino acid substitution Y349C, T366S, L368A and Y407V (hole) in its CH3 domain, or vice versa. This set of amino acid substitutions can be further extended by inclusion of the amino acid substitutions K409A on one polypeptide and F405K in the other polypeptide as described by (Wei et al. 2017). Accordingly, in some embodiments, the Fc domain of one of the polypeptides, for example Fc1, comprises or further comprises the amino acid substitution K409A in its CH3 domain and the Fc domain of the other polypeptide, for example Fe2, comprises or further the amino acid substitution F405K in its CH3 domain, or vice versa.

Accordingly, in one embodiment, the Fc domain of one of the polypeptides, for example Fc1, comprises or further comprises the charge pair substitutions E356K, E356R, D356R, or D356K and D399K or D399R, and the Fc domain of the other polypeptide, for example Fc2, comprises or further comprises the charge pair substitutions R409D, R409E, K409E, or K409D and N392D, N392E, K392E, or K392D, or vice versa.

In one embodiment, said first and second Fc domains each comprise CH2 and CH3 domains comprising at least two additional cysteine residues.

Such cysteine residues may result into the formation of disulfide bridges, which may improve the stability of the antigen-binding proteins, optimally without interfering with the binding characteristics of the antigen-binding proteins. Such cysteine bridges can further improve heterodimerization. Further amino acid substitutions, such as charged pair substitutions, have been described in the art, for example in EP2970484 to improve the heterodimerization of the resulting proteins.

Some embodiments of the present disclosure may include methods of treating a metastatic lesion that presents a peptide comprising, consisting essentially of, or consisting of SLLQHLIGL (SEQ ID NO: 310), including, for example: identifying a metastatic lesion and administering a T lymphocyte of the present disclosure or activated T lymphocytes produced by methods described herein to the metastatic lesion, wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Some embodiments of the present disclosure may include methods of treating a metastatic lesion that presents a peptide comprising, consisting essentially of, or consisting of SLLQHLIGL (SEQ ID NO: 310), including, for example: identifying a metastatic lesion and treating the metastatic lesion with a population of T lymphocytes that bind to and/or are specific for SLLQHLIGL (SEQ ID NO: 310), wherein the metastasis or metastatic lesion originates from a cancer selected from the group consisting of adrenocortical sarcoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Other embodiments of the present disclosure may include methods of treating a metastatic lesion that presents a peptide comprising, consisting essentially of, or consisting of SLLQHLIGL (SEQ ID NO: 310), including, for example: treating the metastatic lesion with a population of T lymphocytes that bind to and/or are specific for SLLQHLIGL (SEQ ID NO: 310), wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Other embodiments of the present disclosure may include methods of treating a metastatic lesion that presents a peptide comprising, consisting essentially of, or consisting of SLLQHLIGL (SEQ ID NO: 310) on the cell surface, including, for example: selecting a patient having a metastatic lesion and administering to the patient a composition comprising a T lymphocyte of the present disclosure or the activated T lymphocytes produced by methods described herein, wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Some embodiments of the present disclosure may include methods of eliciting an immune response to a metastatic lesion that present a peptide comprising, consisting essentially of, or consisting of SLLQHLIGL (SEQ ID NO: 310), including, for example: identifying a metastatic lesion and administering a T lymphocyte of the present disclosure or activated T lymphocytes produced by methods described herein in the metastatic lesion, wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Some embodiments of the present disclosure may include methods of eliciting an immune response to a metastatic lesion that present a peptide comprising, consisting essentially of, or consisting of SLLQHLIGL (SEQ ID NO: 310), including, for example: identifying a metastatic lesion and treating the metastatic lesion with a population of T lymphocytes that binds to and/or are specific for SLLQHLIGL (SEQ ID NO: 310), wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Other embodiments of the present disclosure may include methods of eliciting an immune response to a metastatic lesion that present a peptide comprising, consisting essentially of, or consisting of SLLQHLIGL (SEQ ID NO: 310) on the cell surface, including, for example: selecting a patient having a metastatic lesion and administering to the patient a composition comprising a T lymphocyte of the present disclosure or the activated T lymphocytes produced by methods described herein, wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, uterine carcinosarcoma, uterine endometrial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Some embodiments of the present disclosure may include administering to a patient at least one adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23).

Some embodiments of the present disclosure may include methods of preparing a T cell population comprising: obtaining the T cell population from PBMCs; activating the obtained T cell population, transducing the activated T cell population with the nucleic acid of the present disclosure, expanding the transduced T cell population, and wherein the activating, transducing, and expanding are performed in the presence of IL-21 with or without a histone deacetylase inhibitor (HDACi).

In one embodiment, the present disclosure provide a method for reprogramming antigen-specific effector T cells (T_{EFF} cells) into central memory T cells (T_{CM} cells), the method may include obtaining a starting population of lymphocytes comprising T_{EFF} cells from a subject; optionally preparing a sample enriched in T_{EFF} cells from the starting population of lymphocytes comprising T_{EFF} cells; and culturing the starting population of lymphocytes comprising T_{EFF} cells or the sample enriched in T_{EFF} cells in the presence of a HDACi and IL-21, each in an amount sufficient to re program the T_{EFF} cells into T_{CM} cells, wherein the re-programming produces a population of lymphocytes enriched for T_{CM} cells as compared to the number of T_{CM} cells in the starting population of lymphocytes comprising T_{EFF} cells obtained from a subject.

In some embodiments, obtaining a starting population of lymphocytes comprising T_{EFF} cells may include taking a sample of tumor infiltrating lymphocytes (TILs) or a sample containing peripheral blood mononuclear cells (PBMCs) from a subject. In some embodiments, the method may further include the step of preparing a sample enriched in T_{EFF} cells from the starting population of lymphocytes comprising T_{EFF} cells. In some embodiments, the step of preparing a sample enriched in T_{EFF} cells from the starting population of lymphocytes comprising T_{EFF} cells may include isolating CD8+ T_{EFF} cells from the starting population of lymphocytes containing T_{EFF} cells.

In some embodiments, IL-21, a HDACi, or combinations thereof may be utilized in the field of cancer treatment, with methods described herein, and/or with ACT processes described herein. In an embodiment, the present disclosure provides methods for re-programming effector T cells to a central memory phenotype comprising culturing the effector T cells with at least one HDACi together with IL-21. Representative HDACi include, for example, trichostatin A, trapoxin B, phenylbutyrate, valproic acid, vorinostat (suberanilohydroxamic acid or SAHA), belinostat, panobinostat, dacinostat, entinostat, tacedinaline, and mocetinostat. In particular aspects, the HDACi may be SAHA. In other aspects, the HDACi may be panobinostat.

### Bi-Specific Molecules Against PRAME-004

The molecules of the present disclosure generally comprise a first polypeptide chain and a second polypeptide chain, wherein the chains jointly provide a variable domain of an antibody specific for an epitope of an immune modulator cell surface antigen, and a variable domain of a TCR that is specific for an MHC-associated peptide epitope, e.g., SLLQHLIGL (PRAME-004) (SEQ ID NO: 310). Antibody and TCR-derived variable domains are stabilized by covalent and non-covalent bonds formed between Fc-parts or portions thereof located on both polypeptide chains. The dual specificity polypeptide molecule is then capable of simultaneously binding the cellular receptor and the MHC-associated peptide epitope.

As discussed, a variable domain of an antibody may specifically bind an epitope of an immune modulator cell surface antigen at least one selected from the group consisting of CD3γ, CD3δ, CD3ε, CD3ζ, CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, FεRI, TCRα/β, TCRγ/δ, and HLA-DR.

In the context of the present invention, variable domains are derived from antibodies capable of recruiting human immune modulator cells by specifically binding to a surface antigen of said effector cells. In one particular embodiment, said antibodies specifically bind to epitopes of the TCR-CD3 complex of human T cells, comprising the peptide chains TCRa, TCRβ, CD3γ, CD3δ, CD3δ, and CD3δ.

In the context of the present invention, the dual affinity polypeptide molecule according to the invention is exemplified by a construct that binds the SLLQHLIGL peptide (SEQ ID NO: 310) when presented as a peptide-MHC complex.

For example, dual affinity polypeptide molecules of the present disclosure may include those disclosed in US20190016801, US20190016802, US20190016803, and US20190016804, the contents of which are herein incorporated by reference in their entireties.

Preferably, the dual specificity polypeptide molecule according to the present invention binds with high specificity to both the immune modulator cell antigen and a specific antigen epitope presented as a peptide-MHC complex, e.g., with a binding affinity (KD) of about 100 nM or less, about 30 nM or less, about 10 nM or less, about 3 nM or less, about 1 nM or less, e.g. measured by Bio-Layer Interferometry or as determined by flow cytometry.

Preferred is a dual specificity polypeptide molecule according to the invention, wherein a knob-into-hole mutation is selected from T366W as knob, and T366'S, L368′A, and Y407′V as hole in the CH3 domain (see, e.g., WO 98/50431). This set of mutations can be further extended by inclusion of the mutations K409A and F405′K as described by (Wei et al. 2017). Another knob can be T366Y and the hole is Y407′T.

Engineering was performed to incorporate knob-into-hole mutations into CH3-domains with and without additional interchain disulfide bond stabilization; to remove an N-glycosylation site in CH2 (e.g. N297Q mutation); to introduce Fc-silencing mutations; to introduce additional disulfide bond stabilization into VL and VH, respectively, according to the methods described by (Reiter et al. 1994). An overview of produced bispecific TCR/mAb diabodies, the variants as well as the corresponding sequences are listed in Table 1.

Preferred is the dual specificity polypeptide molecule according to the invention, wherein said first and second polypeptide chains further comprise at least one hinge domain and/or an Fc domain or portion thereof. In antibodies, the “hinge” or “hinge region” or “hinge domain” refers to the flexible portion of a heavy chain located between the CH1 domain and the CH2 domain. It is approximately 25 amino acids long, and is divided into an “upper hinge,” a “middle hinge” or “core hinge,” and a “lower hinge.” A “hinge subdomain” refers to the upper hinge, middle (or core) hinge or the lower hinge. The amino acids sequence of the hinges of an IgG1 molecule is IgG1: EPKSCDKTHTCPPCPAPELLG (SEQ ID NO: 129), with E being E216 according to EU (imgt.org/IMGTScientificChart/Numbering/Hu_IGHGnber.html) numbering.

Preferred is a dual specificity polypeptide molecule according to the present invention, comprising at least one IgG fragment crystallizable (Fc) domain, i.e., a fragment crystallizable region (Fc region), the tail region of an antibody that interacts with Fc receptors and some proteins of the complement system. Fc regions contain two or three heavy chain constant domains (CH domains 2, 3, and 4) in each polypeptide chain. The Fc regions of IgGs also bear a highly conserved N-glycosylation site. Glycosylation of the Fc fragment is essential for Fc receptor-mediated activity. The small size of bispecific molecule formats such as BiTEs® and DARTs (˜50 kD) can lead to fast clearance and a short half-life. Therefore, for improved pharmacokinetic properties, the TCR variable only regions (scTv)-cellular receptor (e.g., CD3) dual specificity polypeptide molecule can be fused to a (human IgG1) Fc domain, thereby increasing the molecular mass. Several mutations located at the interface between the CH2 and CH3 domains, such as T250Q/M428L and M252Y/S254T/T256E+H433K/N434F, have been shown to increase the binding affinity to neonatal Fc receptor (FcRn) and the half-life of IgG1 in vivo. By this, the serum half-life of an Fc-containing molecule could be further extended.

In the dual specificity polypeptide molecules of the invention, said Fc domain can comprise a CH2 domain comprising at least one Fc effector function silencing mutation. Preferably, these mutations are introduced into the ELLGGP (SEQ ID NO: 130) sequence of human IgG1 (residues 233-238) or corresponding residues of other isotypes) known to be relevant for effector functions. In principle, one or more mutations corresponding to residues derived from IgG2 and/or IgG4 are introduced into IgG1 Fc. Preferred are: E233P, L234V, L235A and no residue or G in position 236. Another mutation is P331S. EP1075496 discloses a recombinant antibody comprising a chimeric domain which is derived from two or more human immunoglobulin heavy chain CH2 domains, which human immunoglobulins are selected from IgG1, IgG2 and IgG4, and wherein the chimeric domain is a human immunoglobulin heavy chain CH2 domain which has the following blocks of amino acids at the stated positions: 233P, 234V, 235A and no residue or G in position 236 and 327G, 330S and 331S in accordance with the EU numbering system, and is at least 98% identical to a CH2 sequence (residues 231-340) from human IgG1, IgG2, or IgG4 having said modified amino acids.

The inventive dual specificity polypeptide molecules according to the present invention are exemplified here by a dual specificity polypeptide molecule comprising a first polypeptide chain comprising SEQ ID NO: 131 and a second polypeptide chain comprising SEQ ID NO: 132, or a dual specificity polypeptide molecule comprising a first polypeptide chain comprising SEQ ID NO: 133 and a second polypeptide chain comprising SEQ ID NO: 134.

In an aspect, the disclosure provides for a polypeptide having at least 50%, at least 60%, at least 70%, at least 80%, at least 90%, at least 95%, at least 96%, at least 97%, at least 98%, or at least 99% sequence identity to the amino acid sequence of SEQ ID NO: 131, 132, 133, or 134.

In another aspect, the polypeptides or dual specific polypeptide molecules as disclosed herein can be modified by the substitution of one or more residues at different, possibly selective, sites within the polypeptide chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions”.

In another aspect of the invention, the above object is solved by providing a nucleic acid(s) encoding for a first polypeptide chain and/or a second polypeptide chain as disclosed herein, or expression vector(s) comprising such nucleic acid.

In another aspect of the invention, the above object is solved by providing a host cell comprising vector(s) as defined herein.

In another aspect of the invention, the above object is solved by providing a method for producing a dual specificity polypeptide molecule according to the present invention, comprising suitable expression of said expression vector(s) comprising the nucleic acid(s) as disclosed in a suitable host cell, and suitable purification of the molecule(s) from the cell and/or the medium thereof.

In another aspect of the invention, the above object is solved by providing a pharmaceutical composition comprising the dual specificity polypeptide molecule according to the invention, the nucleic acid or the expression vector(s) according to the invention, or the cell according to the invention, together with one or more pharmaceutically acceptable carriers or excipients.

In another aspect of the invention, the invention relates to the dual specificity polypeptide molecule according to the invention, the nucleic acid(s) or the expression vector(s) according to the invention, the cell according to the invention, or the pharmaceutical composition according to the invention, for use in medicine.

In another aspect of the invention, the invention relates to the dual specificity polypeptide molecule according to the invention, the nucleic acid or the expression vector(s) according to the invention, the cell according to the invention, or the pharmaceutical composition according to the invention, for use in the treatment of a disease or disorder as disclosed herein, in particular selected from cancer and infectious diseases.

In another aspect of the invention, the invention relates to a method for the treatment of a disease or disorder comprising administering a therapeutically effective amount of the dual specificity polypeptide molecule according to the invention, the nucleic acid or the expression vector(s) according to the invention, the cell according to the invention, or the pharmaceutical composition according to the invention.

In another aspect of the invention, the invention relates to a method of eliciting an immune response in a patient or subject comprising administering a therapeutically effective amount of the dual specificity polypeptide molecule according to the invention or the pharmaceutical composition according to the invention.

In another aspect, the invention relates to a method of killing target cells in a patient or subject comprising administering to the patient an effective amount of the dual specificity polypeptide molecule according to the present invention.

Examples of such dual specificity molecule are given in Table 2b.

TABLE 2b
Exemplary bi-specific molecules according to the invention.

| Molecule | TCR | mAb | SEQ IDs | modifications |
|---|---|---|---|---|
| IA_5 | R16P1C10I | hUCHT1(Var17) | SEQ ID NO: 131 | IgG1 (K/O, KiH-ds) |
| | | | SEQ ID NO: 132 | |
| IA_6 | R16P1C10I#6 | hUCHT1(Var17) | SEQ ID NO: 133 | IgG1 (K/O, KiH-ds) |
| | | | SEQ ID NO: 134 | |

KiH: Knob-into-hole; K/O: Fc-silenced; KiH-ds: Knob-into-hole stabilized with artificial disulfide-bondto connect CH3:CH3′; and VH and VL domains derived from the CD3-specific, humanized antibody hUCHT1(Var17).

In one embodiment, the first variable domain and the second variable domain as herein defined may comprise an amino acid substitution at position 44 according to the IMGT numbering. In a preferred embodiment, said amino acid at position 44 is substituted with another suitable amino acid, in order to improve pairing. In particular embodiments, in which said antigen-binding protein is a TCR, said mutation improves for example the pairing of the chains (i.e. paring of α and β chains or paring of γ and δ). In a preferred embodiment, the amino acid as present at position 44 in the variable domain is substituted by one amino acid selected from the group consisting of Q, R, D, E, K, L, W, and V.

In one embodiment, the first variable domain of the antigen-binding proteins of the invention comprises:
a CDRa1 comprising or consisting of the amino acid sequence selected from the group consisting of the amino acid sequences DRGSQS (SEQ ID NO: 135) and DRGSQL (SEQ ID NO: 136), and/or
a CDRa2 comprising or consisting of the amino acid sequence selected from the group consisting of the amino acid sequences IYSNGD (SEQ ID NO: 137) and IYQEGD (SEQ ID NO: 138) and/or
a CDRa3 comprising or consisting of the amino acid sequence selected from the group consisting of the amino acid sequences CAAVINNPSGGMLTF (SEQ ID NO: 139), CAAVIDNSNGGILTF (SEQ ID NO: 140), CAAVIDNPSGGILTF (SEQ ID NO: 141), CAAVIDNDQGGILTF (SEQ ID NO: 142), CAAVIPNPPGGKLTF (SEQ ID NO: 143), CAAVIPNPGGGALTF (SEQ ID NO: 144), CAAVIPNSAGGRLTF (SEQ ID NO: 145), CAAVIPNLEGGSLTF (SEQ ID NO: 146), CAAVIPNRLGGYLTF (SEQ ID NO: 147), CAAVIPNTDGGRLTF (SEQ ID NO: 148), CAAVIPNQRGGALTF (SEQ ID NO: 149), CAAVIPNVVGGILTF (SEQ ID NO: 150), CAAVITNIAGGSLTF (SEQ ID NO: 151), CAAVIPNNDGGYLTF (SEQ ID NO: 152)), CAAVIPNGRGGLLTF (SEQ ID NO: 153), CAAVIPNTHGGPLTF (SEQ ID NO: 154), CAAVIPNDVGGSLTF (SEQ ID NO: 155), CAAVIENKPGGPLTF (SEQ ID NO: 156), CAAVIDNPVGGPLTF (SEQ ID NO: 157), CAAVIPNNNGGALTF (SEQ ID NO: 158), CAAVIPNDQGGILTF (SEQ ID NO: 159), CAAVIPNVVGGQLTF (SEQ ID NO: 160), CAAVIPNSYGGLLTF (SEQ ID NO: 161), CAAVIPNDDGGLLTF (SEQ ID NO: 162), CAAVIPNAAGGLLTF (SEQ ID NO: 163), CAAVIPNTIGGLLTF (SEQ ID NO: 164) and CAAVIPNTRGGLLTF (SEQ ID NO: 165), and the second variable domain comprises:
a CDRb1 comprising or consisting of the amino acid sequence selected from the group consisting of the amino acid sequences SGHRS (SEQ ID NO: 166) and PGHRA (SEQ ID NO: 167) and/or
a CDRb2 comprising or consisting of the amino acid sequence selected from the group consisting of the amino acid sequences YFSETQ (SEQ ID NO: 169), YVHGEE (SEQ ID NO: 170) and YVHGAE (SEQ ID NO: 171) and/or
a CDRb3 comprising or consisting of the amino acid sequence selected from the group consisting of the amino acid sequences CASSPWDSPNEQYF (SEQ ID NO: 172) and CASSPWDSPNVQYF (SEQ ID NO: 173).

The inventors of the present invention identified in the examples as herein disclosed, the TCR variant “HiAff1” and “LoAff3” of which the CDR amino acid sequences, when used in the antigen-binding proteins of the invention, in particular in bispecific antigen-binding proteins, more particularly in a Fc-containing bispecific TCR/mAb (anti-CD3) diabody format, increase the binding affinity, the stability, and the specificity of the antigen-binding proteins comprising those CDRs, in particular, in comparison to a reference protein.

Such a reference protein may be, for example, an antigen-binding protein comprising the CDRs of the parental/wild type TCR R16P1C10, which is disclosed in WO2018/172533, for instance, a Fc-containing bispecific TCR/mAb (anti-CD3) diabody as herein described comprising the CDRs of said TCR R16P1C10 or the reference protein is an antigen-binding protein comprising the CDRs of said TCR R16P1C10 and is in the same format as the antigen-binding protein with which it is compared. Such a reference protein may also be, for example, an antigen-binding protein comprising the CDRs of “CDR6”, for instance, a Fc-containing bispecific TCR/mAb (anti-CD3) diabody as herein described comprising the CDRs of “CDR6” or the reference protein is an antigen-binding protein comprising the CDRs of “CDR6” and is in the same format as the antigen-binding protein with which it is compared, wherein the CDRs of “CDR6” are disclosed herein above.

The inventors demonstrated furthermore that the antigen-binding proteins of the invention comprising the above described CDRs have an improved stability in comparison to an antigen-binding protein comprising the CDRs of a reference antigen-binding protein called “CDR6”, wherein the antigen-binding protein called “CDR6” comprises the following alpha and beta CDRs:

CDRa1 comprising or consisting of the amino acid sequence DRGSQS (SEQ ID NO: 135), and CDRa2 comprising or consisting of the amino acid sequence IYSNGD (SEQ ID NO: 137), and CDRa3 comprising or consisting of the amino acid sequence CAAVIDNDQGGILTF (SEQ ID NO: 142), and CDRb1 comprising or consisting of the amino acid sequence PGHRA (SEQ ID NO: 167), and CDRb2 comprising or consisting of the amino acid sequence YVHGEE (SEQ ID NO: 170), and CDRb3 comprising or consisting of the amino acid sequence CASSPWDSPNVQYF (SEQ ID NO: 173).

In one particular embodiment the invention refers to antigen-binding proteins comprising the CDRs of the so-called “HiAff1” and “LoAff3” variants and variants thereof. Accordingly, in one preferred embodiment, the antigen-binding protein of the invention comprises
a) a first polypeptide chain comprising a first variable domain comprising three complementary determining regions (CDRs) CDRa1, CDRa2, and CDRa3, wherein
   the CDRa1 comprises or consists of the amino acid sequence DRGSQS (SEQ ID NO: 135) or an amino acid sequence at least 85% identical to SEQ ID NO: 135),
   the CDRa2 comprises or consists of the amino acid sequence IYQEGD (SEQ ID NO: 138) and
   the CDRa3 comprises or consists of the amino acid sequence CAAVIDNDQGGILTF (SEQ ID NO: 142), and
b) a second polypeptide chain comprising a second variable domain comprising three complementary determining regions (CDRs) CDRb1, CDRb2 and CDRb3, wherein
   the CDRb1 comprises or consists of the amino acid sequence PGHRA (SEQ ID NO: 167) or PGHRS (SEQ ID NO: 168), preferably PGHRA (SEQ ID NO: 167), or an amino acid sequence at least 85% identical to SEQ ID NO: 167) or SEQ ID NO: 168), preferably SEQ ID NO: 167);
   the CDRb2 comprises or consists of the amino acid sequence YVHGEE (SEQ ID NO: 170) or an amino acid sequence at least 85% identical to SEQ ID NO: 170), and
   the CDRb3 comprises or consists of the amino acid sequence CASSPWDSPNEQYF (SEQ ID NO: 172) or CASSPWDSPNVQYF (SEQ ID NO: 173), preferably CASSPWDSPNVQYF (SEQ ID NO: 173), or an amino acid sequence at least 85% identical to SEQ ID NO: 172) or SEQ ID NO: 173), preferably CASSPWDSPNVQYF (SEQ ID NO: 173).

TABLE 3
CDR sequences and binding affinities of wild type and maturated TCRs expressed
as scTCR-Fab or diabody-F_{c}

| TCR | | | | | | | |
|---|---|---|---|---|---|---|---|
| variant | CDRa1 | CDRa2 | CDRa3 | CDRb1 | CDRb2 | CDRb3 | KD [M] |
| Wild type | DRGSQS | IYSNGD | CAAVISNFGNEKLTF | SGHRS | YFSETQ | CASSPWDSPNEQYF | Cannot |
| CDRs | (SEQ | (SEQ | (SEQ ID NO: 26) | (SEQ | (SEQ | (SEQ ID NO: | be |
| and | ID NO: | ID NO: | | ID | ID NO: | 172 | expressed |
| framework | 135) | 137) | | NO: | 31) | | in CHO as |
| | | | | 166) | | | scTCR- |
| | | | | | | | Fab or |
| | | | | | | | diabody- |
| | | | | | | | F_{c} |
| Stabilized ¹ | DRGSQS | IYSNGD | CAAVISNFGNEKLTF | PGHRS | YFSETQ | CASSPWDSPNEQYF | 1.2E−06 |
| | (SEQ | (SEQ | (SEQ ID NO: 26) | (SEQ | (SEQ | (SEQ ID NO: | |
| | ID NO: | ID NO: | | ID | ID NO: | 172) | |
| | 135) | 137) | | NO: | 31) | | |
| | | | | 168) | | | |
| Stabilized ² | DRGSQS | IYSNGD | CAAVISNFGNEKLTF | PGHRS | YFSETQ | CASSPWDSPNEQYF | 9.3E−07 |
| | (SEQ | (SEQ | (SEQ ID NO: 26) | (SEQ | (SEQ | (SEQ ID NO: | |
| | ID NO: | ID NO: | | ID | ID NO: | 172) | |
| | 135) | 137) | | NO: | 31) | | |
| | | | | 168) | | | |
| Improved ¹ | DRGSQS | IYSNGD | CAAVIDNSNGGILTF | PGHRS | YVHGAE | CASSPWDSPNEQYF | 1.0E−08 |
| | (SEQ | (SEQ | (SEQ ID NO: 26) | (SEQ | (SEQ | (SEQ ID NO: | |
| | ID NO: | ID NO: | | ID | ID NO: | 172) | |
| | 135) | 137) | | NO: | 171) | | |
| | | | | 168) | | | |
| Improved ² | DRGSQS | IYSNGD | CAAVIDNSNGGILTF | PGHRS | YVHGAE | CASSPWDSPNEQYF | 8.7E−09 |
| | (SEQ | (SEQ | (SEQ ID NO: 26) | (SEQ | (SEQ | (SEQ ID NO: | |
| | ID NO: | ID NO: | | ID | ID NO: | 172) | |
| | 135) | 137) | | NO: | 171) | | |
| | | | | 168) | | | |
| Medium- | DRGSQS | IYQEGD | CAAVIDNDQGGILTF | PGHRS | YVHGEE | CASSPWDSPNEQYF | 1.8E−09 |
| affinity | (SEQ | (SEQ | (SEQ ID NO: 26) | (SEQ | (SEQ | (SEQ ID NO: | |
| LoAff3 ² | ID NO: | ID NO: | | ID | ID NO: | 172) | |
| | 135) | 138) | | NO: | 170) | | |
| | | | | 168) | | | |
| High- | DRGSQS | IYSNGD | CAAVIDNDQGGILTF | PGHRA | YVHGEE | CASSPWDSPNVQYF | 3.9E−10 |
| affinity | (SEQ | (SEQ | (SEQ ID NO: 26) | (SEQ | (SEQ | (SEQ ID NO: | |
| CDR6 ² | ID NO: | ID NO: | | ID | ID NO: | 173) | |
| | 135) | 137) | | NO: | 170) | | |
| | | | | 167 | | | |
| High- | DRGSQS | IYQEGD | CAAVIDNDQGGILTF | PGHRA | YVHGEE | CASSPWDSPNVQYF | 3.8E−10 |
| affinity | (SEQ | (SEQ | (SEQ ID NO: 26) | (SEQ | (SEQ | (SEQ ID NO: | |
| HiAff1 ² | ID NO: | ID NO: | | ID | ID NO: | 173) | |
| | 135) | 138) | | NO: | 170) | | |
| | | | | 167 | | | |

¹ expressed as scTCR-Fab
² expressed as diabody-F_{c}

All positions and CDR definitions are according to Kabat numbering scheme. TCRs consisting of Vapha and Vbeta domains were designed, produced, and tested in a single-chain (scTCR) format coupled to a Fab-fragment of a humanized UCHT1-antibody (Table 4). Vectors for the expression of recombinant proteins were designed as mono-cistronic, controlled by HCMV-derived promoter elements, pUC19-derivatives. Plasmid DNA was amplified in E. coli according to standard culture methods and subsequently purified using commercial-available kits (Macherey & Nagel). Purified plasmid DNA was used for transient transfection of CHO cells. Transfected CHO-cells were cultured for 10-11 days at 32° C. to 37° C.

TABLE 4
Bispecific molecules

| ID | α-chain | β-chain |
|---|---|---|
| TPP-70 | 178 | 179 |
| TPP-71 | 178 | 180 |
| TPP-72 | 178 | 181 |
| TPP-73 | 178 | 182 |
| TPP-74 | 178 | 183 |
| TPP-93 | 184 | 185 |
| TPP-79 | 187 | 186 |
| TPP-105 | 189 | 188 |
| TPP-106 | 190 | 191 |
| TPP-108 | 190 | 185 |
| TPP-109 | 195 | 194 |
| TPP-110 | 195 | 186 |
| TPP-111 | 187 | 194 |
| TPP-112 | 184 | 191 |
| TPP-113 | 184 | 203 |
| TPP-114 | 184 | 205 |
| TPP-115 | 206 | 205 |
| TPP-116 | 208 | 205 |
| TPP-117 | 210 | 205 |
| TPP-118 | 212 | 205 |
| TPP-119 | 184 | 213 |
| TPP-120 | 184 | 214 |
| TPP-121 | 206 | 214 |
| TPP-122 | 208 | 214 |
| TPP-123 | 210 | 214 |
| TPP-124 | 212 | 214 |
| TPP-125 | 184 | 215 |
| TPP-126 | 206 | 215 |
| TPP-127 | 208 | 215 |
| TPP-128 | 210 | 215 |
| TPP-129 | 212 | 215 |
| TPP-207 | 187 | 217 |
| TPP-208 | 218 | 217 |
| TPP-209 | 220 | 217 |
| TPP-210 | 222 | 217 |
| TPP-211 | 187 | 223 |
| TPP-212 | 218 | 225 |
| TPP-213 | 220 | 225 |
| TPP-214 | 230 | 223 |
| TPP-215 | 232 | 231 |
| TPP-216 | 234 | 231 |
| TPP-217 | 236 | 231 |
| TPP-218 | 230 | 231 |
| TPP-219 | 240 | 239 |
| TPP-220 | 242 | 239 |
| TPP-221 | 244 | 239 |
| TPP-222 | 246 | 239 |
| TPP-226 | 222 | 247 |
| TPP-227 | 189 | 249 |
| TPP-228 | 250 | 249 |
| TPP-229 | 251 | 249 |
| TPP-230 | 344 | 349 |
| TPP-235 | 253 | 223 |
| TPP-236 | 254 | 223 |
| TPP-237 | 255 | 223 |
| TPP-238 | 256 | 223 |
| TPP-239 | 257 | 223 |
| TPP-240 | 258 | 223 |
| TPP-241 | 259 | 223 |
| TPP-242 | 260 | 223 |
| TPP-243 | 261 | 223 |
| TPP-244 | 262 | 223 |
| TPP-245 | 263 | 223 |
| TPP-246 | 265 | 264 |
| TPP-247 | 265 | 266 |
| TPP-248 | 265 | 267 |
| TPP-249 | 265 | 268 |
| TPP-250 | 265 | 269 |
| TPP-252 | 265 | 270 |
| TPP-253 | 265 | 271 |
| TPP-254 | 265 | 272 |
| TPP-255 | 265 | 273 |
| TPP-256 | 265 | 274 |
| TPP-257 | 265 | 275 |
| TPP-258 | 265 | 276 |
| TPP-259 | 265 | 277 |
| TPP-260 | 265 | 278 |
| TPP-261 | 265 | 279 |
| TPP-262 | 265 | 280 |
| TPP-263 | 265 | 281 |
| TPP-264 | 265 | 282 |
| TPP-265 | 265 | 283 |
| TPP-266 | 265 | 284 |
| TPP-267 | 265 | 285 |
| TPP-268 | 265 | 286 |
| TPP-269 | 265 | 287 |
| TPP-270 | 265 | 288 |
| TPP-271 | 265 | 289 |
| TPP-272 | 218 | 290 |
| TPP-273 | 250 | 291 |
| TPP-274 | 250 | 292 |
| TPP-275 | 250 | 293 |
| TPP-276 | 250 | 294 |
| TPP-277 | 250 | 295 |
| TPP-279 | 250 | 296 |
| TPP-666 | 298 | 297 |
| TPP-669 | 354 | 359 |
| TPP-871 | 300 | 249 |
| TPP-872 | 300 | 301 |
| TPP-876 | 302 | 225 |
| TPP-879 | 298 | 303 |
| TPP-891 | 304 | 225 |
| TPP-892 | 304 | 297 |
| TPP-894 | 299 | 303 |
| TPP-1292 | 216 | 297 |
| TPP-1293 | 219 | 225 |
| TPP-1294 | 221 | 297 |
| TPP-1295 | 324 | 329 |
| TPP-1296 | 304 | 224 |
| TPP-1297 | 304 | 226 |
| TPP-1298 | 334 | 339 |
| TPP-1300 | 299 | 228 |
| TPP-1301 | 229 | 303 |
| TPP-1302 | 299 | 233 |
| TPP-1303 | 299 | 235 |
| TPP-1304 | 299 | 237 |
| TPP-1305 | 229 | 233 |
| TPP-1306 | 229 | 235 |
| TPP-1307 | 229 | 237 |
| TPP-1308 | 299 | 245 |
| TPP-1309 | 299 | 248 |
| TPP-1332 | 238 | 249 |
| TPP-1333 | 364 | 369 |
| TPP-1334 | 243 | 249 |

In this table, except for TPP-70, TPP-71, TPP-72, TPP-73 and TPP74, the term “μ-chain” refers to a polypeptide chain comprising a V_{α}, i.e. a variable domain derived from a TCR α-chain. The term “β-chain” refers to a polypeptide chain comprising a V_{β}, i.e. a variable domain derived from a TCR β-chain. For TPP-70, TPP-71, TPP-72, TPP-73 and TPP74, the “α-chain” does not comprise any TCR derived variable domains, but the “β-chain” comprises two TCR-derived variable domains, one derived from a TCR α-chain and one derived from a TCR β-chain.

The present disclosure provides an antigen-binding protein for use in the (manufacture of a medicament for the) treatment of metastasis or a metastatic lesion, which antigen-binding protein is selected from the group consisting of TPP-1295, TPP-1298, TPP-230, TPP-669, or TPP-1333.

Alternatively, or in addition, a method of treating a patient (i) being diagnosed for, (ii) suffering from, or (iii) being at risk of developing metastasis or a metastatic lesion, is provided, the method comprising administration of an antigen-binding protein selected from the group consisting of TPP-1298, TPP-1295, TPP-230, TPP-669, or TPP-1333, in one or more therapeutically effective doses.

According to one embodiment, the antigen-binding protein is TPP-1295, with the following set of sequences:

| TPP-1295 | SEQ ID NO: |
|---|---|
| CDRa1 | 320 |
| CDRa2 | 321 |
| CDRa3 | 322 |
| CDRb1 | 325 |
| CDRb2 | 326 |
| CDRb3 | 327 |
| V alpha | 323 |
| V beta | 328 |
| alpha chain | 324 |
| beta chain | 329 |

According to one embodiment, the antigen-binding protein comprises a first polypeptide chain and a second polypeptide chain linked together forming a first antigen binding domain and a second antigen binding domain,
wherein the first antigen binding domain comprises
   a T cell receptor (TCR) α variable domain comprising
      a complementary determining region (CDR)a1 comprising the amino acid sequence of SEQ ID NO: 320,
      optionally, a CDRa2 comprising the amino acid sequence of SEQ ID NO: 321, and
      a CDRa3 comprising the amino acid sequence of SEQ ID NO: 322, and
   a TCR β variable domain comprising
      a CDRb1 comprising the amino acid sequence of SEQ ID NO: 325,
      optionally, a CDRb2 comprising the amino acid sequence of SEQ ID NO: 326, and
      a CDRb3 comprising the amino acid sequence of SEQ ID NO: 327.

The first antigen binding domain of the antigen-binding protein binds to a peptide comprising or consisting of the amino acid sequence of SLLQHLIGL in a complex with an MHC molecule, suitably HLA-A*02.

The antigen-binding protein may have a TCR α variable domain comprising SEQ ID NO: 323 and a TCR β variable domain comprising SEQ ID NO: 328.

The antigen-binding protein may have a first polypeptide chain comprising SEQ ID NO: 324 and a second polypeptide chain comprising SEQ ID NO: 329.

According to one embodiment, the antigen-binding protein is TPP-1298, with the following set of sequences:

| TPP-1298 | SEQ ID NO: |
|---|---|
| CDRa1 | 330 |
| CDRa2 | 331 |
| CDRa3 | 332 |
| CDRb1 | 335 |
| CDRb2 | 336 |
| CDRb3 | 337 |
| V alpha | 333 |
| V beta | 338 |
| alpha chain | 334 |
| beta chain | 339 |

According to one embodiment, the antigen-binding protein comprises a first polypeptide chain and a second polypeptide chain linked together forming a first antigen binding domain and a second antigen binding domain,
wherein the first antigen binding domain comprises
   a TCR α variable domain comprising
      a CDRa1 comprising the amino acid sequence of SEQ ID NO: 330,
      optionally, a CDRa2 comprising the amino acid sequence of SEQ ID NO: 331, and
      a CDRa3 comprising the amino acid sequence of SEQ ID NO: 332, and
   a TCR β variable domain comprising
      a CDRb1 comprising the amino acid sequence of SEQ ID NO: 335,
      optionally, a CDRb2 comprising the amino acid sequence of SEQ ID NO: 336, and
      a CDRb3 comprising the amino acid sequence of SEQ ID NO: 337.

The first antigen binding domain of the antigen-binding protein binds to a peptide comprising or consisting of the amino acid sequence of SLLQHLIGL in a complex with an MHC molecule, suitably HLA-A*02.

The antigen-binding protein may have a TCR α variable domain comprising SEQ ID NO: 333 and a TCR β variable domain comprising SEQ ID NO: 338.

The antigen-binding protein may have a first polypeptide chain comprising SEQ ID NO: 334 and a second polypeptide chain comprising SEQ ID NO: 339.

According to one embodiment, the antigen-binding protein is TPP-230, with the following set of sequences:

| TPP-230 | SEQ ID NO: |
|---|---|
| CDRa1 | 340 |
| CDRa2 | 341 |
| CDRa3 | 342 |
| CDRb1 | 345 |
| CDRb2 | 346 |
| CDRb3 | 347 |
| V alpha | 343 |
| V beta | 348 |
| alpha chain | 344 |
| beta chain | 349 |

According to one embodiment, the antigen-binding protein comprises a first polypeptide chain and a second polypeptide chain linked together forming a first antigen binding domain and a second antigen binding domain,
wherein the first antigen binding domain comprises
   a TCR α variable domain comprising
      a CDRa1 comprising the amino acid sequence of SEQ ID NO: 340,
      optionally, a CDRa2 comprising the amino acid sequence of SEQ ID NO: 341, and
      a CDRa3 comprising the amino acid sequence of SEQ ID NO: 342, and
   a TCR β variable domain comprising
      a CDRb1 comprising the amino acid sequence of SEQ ID NO: 345,
      optionally, a CDRb2 comprising the amino acid sequence of SEQ ID NO: 346, and
      a CDRb3 comprising the amino acid sequence of SEQ ID NO: 347.

The first antigen binding domain of the antigen-binding protein binds to a peptide comprising or consisting of the amino acid sequence of SLLQHLIGL in a complex with an MHC molecule, suitably HLA-A*02.

The antigen-binding protein may have a TCR α variable domain comprising SEQ ID NO: 343 and a TCR β variable domain comprising SEQ ID NO: 348.

The antigen-binding protein may have a first polypeptide chain comprising SEQ ID NO: 344 and a second polypeptide chain comprising SEQ ID NO: 349.

According to one embodiment, the antigen-binding protein is TPP-669, with the following set of sequences:

| TPP-669 | SEQ ID NO: |
|---|---|
| CDRa1 | 350 |
| CDRa2 | 351 |
| CDRa3 | 352 |
| CDRb1 | 355 |
| CDRb2 | 356 |
| CDRb3 | 357 |
| V alpha | 353 |
| V beta | 358 |
| alpha chain | 354 |
| beta chain | 359 |

According to one embodiment, the antigen-binding protein comprises a first polypeptide chain and a second polypeptide chain linked together forming a first antigen binding domain and a second antigen binding domain,
wherein the first antigen binding domain comprises
   a TCR α variable domain comprising
      a CDRa1 comprising the amino acid sequence of SEQ ID NO: 350,
      optionally, a CDRa2 comprising the amino acid sequence of SEQ ID NO: 351, and
      a CDRa3 comprising the amino acid sequence of SEQ ID NO: 352, and
   a TCR β variable domain comprising
      a CDRb1 comprising the amino acid sequence of SEQ ID NO: 355,
      optionally, a CDRb2 comprising the amino acid sequence of SEQ ID NO: 356, and
      a CDRb3 comprising the amino acid sequence of SEQ ID NO: 357.

The first antigen binding domain of the antigen-binding protein binds to a peptide comprising or consisting of the amino acid sequence of SLLQHLIGL in a complex with an MHC molecule, suitably HLA-A*02.

The antigen-binding protein may have a TCR α variable domain comprising SEQ ID NO: 353 and a TCR β variable domain comprising SEQ ID NO: 358.

The antigen-binding protein may have a first polypeptide chain comprising SEQ ID NO: 354 and a second polypeptide chain comprising SEQ ID NO: 359.

According to one embodiment, the antigen-binding protein is TPP-1333, with the following set of sequences:

| TPP-1333 | SEQ ID NO: |
|---|---|
| CDRa1 | 360 |
| CDRa2 | 361 |
| CDRa3 | 362 |
| CDRb1 | 365 |
| CDRb2 | 366 |
| CDRb3 | 367 |
| V alpha | 363 |
| V beta | 368 |
| alpha chain | 364 |
| beta chain | 369 |

According to one embodiment, the antigen-binding protein comprises a first polypeptide chain and a second polypeptide chain linked together forming a first antigen binding domain and a second antigen binding domain,
wherein the first antigen binding domain comprises
   a TCR α variable domain comprising
      a CDRa1 comprising the amino acid sequence of SEQ ID NO: 360,
      optionally, a CDRa2 comprising the amino acid sequence of SEQ ID NO: 361, and
      a CDRa3 comprising the amino acid sequence of SEQ ID NO: 362, and
   a TCR β variable domain comprising
      a CDRb1 comprising the amino acid sequence of SEQ ID NO: 365,
      optionally, a CDRb2 comprising the amino acid sequence of SEQ ID NO: 366, and
      a CDRb3 comprising the amino acid sequence of SEQ ID NO: 367.

The first antigen binding domain of the antigen-binding protein binds to a peptide comprising or consisting of the amino acid sequence of SLLQHLIGL in a complex with an MHC molecule, suitably HLA-A*02.

The antigen-binding protein may have a TCR α variable domain comprising SEQ ID NO: 363 and a TCR β variable domain comprising SEQ ID NO: 368.

The antigen-binding protein may have a first polypeptide chain comprising SEQ ID NO: 364 and a second polypeptide chain comprising SEQ ID NO: 369.

Purification and quality control of the antigen-binding proteins provided herein may be performed as exemplified below.

According to several embodiments, the metastasis or metastatic lesion is at least one selected from the group consisting of
ACC metastasis
BLCA metastasis
BRCA metastasis
TNBC metastasis
CRC metastasis
HNSCC metastasis
HNAC metastasis
MEL metastasis
SKCM metastasis
UVM metastasis
LC metastasis
NSCLC metastasis
NSCLCadeno metastasis
NSCLCsquam metastasis
NSCLCother metastasis
SCLC metastasis
CHOL metastasis
ESCA metastasis
CESC metastasis
OC metastasis
OV metastasis
LIHC metastasis
RCC metastasis
KIRC metastasis
KIRP metastasis
SARC metastasis
FS metastasis
LPS metastasis
MPNST metastasis
SS metastasis
STAD metastasis
TGCT metastasis
THYM metastasis
UCS metastasis
UCEC metastasis, and/or
UEC metastasis

According to several embodiments, the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, uterine carcinoma, uterine carcinosarcoma, head and neck squamous cell carcinomas, head and neck adenocarcinoma, colon cancer, gastro-intestinal cancer, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

Conditioned cell supernatant was cleared by filtration (0.22 μm) utilizing Sartoclear Dynamics® Lab Filter Aid (Sartorius). Bispecific molecules were purified using an Äkta Pure 25 L FPLC system (GE Lifesciences) equipped to perform affinity and size-exclusion chromatography in line. Affinity chromatography was performed on protein L columns (GE Lifesciences) following standard affinity chromatographic protocols. Size exclusion chromatography was performed directly after elution (pH 2.8) from the affinity column to obtain highly pure monomeric protein using Superdex 200 μg 16/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/mL at temperatures of 2-8° C. Final product yield was calculated after completed purification and formulation.

Quality of purified bispecific molecules was determined by HPLC-SEC on MabPac SEC-1 columns (5 μm, 4×300 mm) running in 50 mM sodium-phosphate pH 6.8 containing 300 mM NaCl within a Vanquish uHPLC-System.

Stress stability testing was performed by incubation of the molecules formulated in PBS for up to two weeks at 40° C. Integrity, aggregate-content as well as monomer-recovery was analyzed by HPLC-SEC analyses.

The inventors demonstrate that the antigen-binding proteins, in particular TCER® molecules, cause cytolysis in T2 cells loaded with target peptide PRAME-004 by LDH release assay (Table 5). The inventors further demonstrate that the antigen-binding proteins, in particular TCER® molecules, cause cytolysis in a PRAME-positive tumor cell line by LDH release assay while a PRAME-negative tumor cell line was not affected by co-incubation with the TCER® molecules (FIGS. 35-37). These in vitro experiments further evidence the safety of the antigen-binding proteins of the invention and document that the cytotoxic effect is highly selective for PRAME-positive tumor tissue. The molecules of the present invention therefore show beneficial safety profiles.

TCER® Slot III variants TPP-214, -222, -230, -666, -669, -871, -872, -876, -879, -891, -894 were additionally characterized for their ability to kill T2 cells loaded with varying levels of target peptide. After loading of the T2 cells with the respective concentrations of PRAME-004 for 2 h, peptide-loaded T2 cells were co-cultured with human PBMCs at an E:T ratio of 5:1 in the presence of increasing concentrations of TCER® variants for 48 h. Levels of LDH released into the supernatant were quantified using CytoTox 96 Non-Radioactive Cytotoxicity Assay Kit (Promega). All TCER® variants showed potent killing of PRAME-004-loaded T2 cells with subpicomolar EC₅₀ values at a peptide loading concentration of 10 nM (FIG. 38, Table 5). EC₅₀ values increased for decreasing PRAME-004 loading levels. However, even at a very low PRAME-004 loading concentration of 10 pM, killing was induced by all TCER® variants, except for TPP-214.

TABLE 5
In vitro cytotoxicity of TCER ® Slot III variants on PRAME-004-loaded T2 cells. T2 cells were
co-cultured with human PBMCs at an E:T ratio of 5:1 for 48 h. PRAME-004 loading concentrations
are indicated. Ec₅₀ values and cytotoxicity levels in the plateau (Top) were calculated using
non-linear 4-point curve fitting.

| | | | 10 nM | | 1 nM | | 100 pM | | 10 pM | |
|---|---|---|---|---|---|---|---|---|---|---|
| | | Va, Vb | PRAME-004 | | PRAME-004 | | PRAME-004 | | PRAME-004 | |
| TCER ® | | (SEQ ID | EC₅₀ | | EC₅₀ | | EC₅₀ | | EC₅₀ | |
| variant | Recruiter | NO:) | [pM] | Top | [pM] | Top | [pM] | Top | [pM] | Top |
| TPP-871 | H2C | 309, 307 | 0.13 | 109 | 1.6 | 143 | 76.5¹ | 90 | 361 | 76 |
| TPP-222 | H2C | 305, 306 | complete | 109 | complete | 78 | 2.8¹ | 127 | 58 | 90 |
| | | | killing | | killing | | | | | |
| TPP-872 | H2C | 309, 306 | complete | 109 | complete | 151 | 4.3¹ | 84 | 49 | 74 |
| | | | killing | | killing | | | | | |
| TPP-876 | BMA031 | 309, 306 | 0.16 | 111 | 2.0 | 113 | 24.4 | 100 | 539 | 40 |
| | (V36)A02 | | | | | | | | | |
| TPP-666 | BMA031 | 305, 308 | 0.15 | 113 | 2.4 | 113 | 39.8 | 100 | 182 | 35 |
| | (V36)A02 | | | | | | | | | |
| TPP-879 | BMA031 | 305, 307 | 0.54 | 106 | 6.2 | 109 | 94.4 | 117 | 1070 | 39 |
| | (V36)A02 | | | | | | | | | |
| TPP-214 | BMA031 | 305, 306 | 0.22 | 108 | 5.0 | 109 | 92.8 | 102 | no killing | 20 |
| | (V36) | | | | | | | | | |
| TPP-891 | BMA031 | 309, 306 | 0.19 | 120 | 2.2 | 112 | 54.0 | 125 | 611 | 45 |
| | (V36)D01 | | | | | | | | | |
| TPP-894 | BMA031 | 305, 307 | 0.87 | 108 | 9.9 | 115 | 226.0 | 129 | 1084 | 44 |
| | (V36)D01 | | | | | | | | | |
| TPP-214 | BMA031 | 305, 306 | 0.26 | 121 | 5.4 | 111 | 105.4 | 99 | no killing | 23 |
| | (V36) | | | | | | | | | |

¹High variability within replicates do not allow for reliable EC₅₀ calculation.

According to yet another aspect of the invention, a pharmaceutical composition comprising at least one active agent is provided, the agent selected from the group consisting of at least one of
the peptide according to the above description
the antibody or fragment thereof according to the above description
the T cell receptor or fragment thereof according to the above description
the nucleic acid or the expression vector according to the above description
the host cell according to the above description,
the recombinant T lymphocyte according to the above description, and/or
the activated T lymphocyte according to the above description
   and a pharmaceutically acceptable carrier. The composition is for use in the (manufacture of a medicament for the) treatment of a patient (i) being diagnosed for, (ii) suffering from, or (iii) being at risk of developing metastasis or a metastatic lesion.

Alternatively, or in addition, a method of treating a patient (i) being diagnosed for, (ii) suffering from, or (iii) being at risk of developing metastasis or a metastatic lesion, is provided.

The method comprises administering to the patient at least one active ingredient selected from the group consisting of at least one of
the peptide according to the above description
the antibody or fragment thereof according to the above description
the T cell receptor or fragment thereof according to the above description
the nucleic acid or the expression vector according to the above description
the host cell according to the above description,
the recombinant T lymphocyte according to the above description, and/or
the activated T lymphocyte according to the above description
   and a pharmaceutically acceptable carrier, in one or more therapeutically effective doses.

Alternatively, or in addition, a pharmaceutical composition for treating metastasis or a metastatic lesion is provided, comprising such active ingredient as an effective ingredient.

In one embodiment, the metastases or metastatic lesion is PRAME positive. In one embodiment, the metastases or metastatic lesion displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03, HLA-A*02:05, HLA-A*02:06, HLA-A*02:07, and HLA-A*02:11. In one embodiment, the patient is positive for HLA-A*02:01.

In different embodiments of the present invention, the metastases or metastatic lesion is at least one selected from the group consisting of at least one of:
ACC (Adrenocortical Carcinoma) metastasis
BLCA (Bladder Urothelial Carcinoma) metastasis
BRCA (Breast Cancer) metastasis
TNBC (Triple-Negative Breast Cancer) metastasis
CRC (Colorectal Cancer) metastasis
HNSCC (Head and Neck Squamous Cell Carcinoma) metastasis
HNAC (Head and Neck Adenocarcinoma) metastasis
MEL (Melanoma) metastasis
SKCM (Skin Cutaneous Melanoma) metastasis
UVM (Uveal Melanoma) metastasis
LC (Lung Cancer) metastasis
NSCLC (Non-small Cell Lung Cancer) metastasis
NSCLCsquam (Non-small Cell Lung Squamous Cell Carcinoma) metastasis
NSCLCadeno (Non-small Cell Lung Adenocarcinoma) metastasis
NSCLCother (metastasis of NSCLC samples that could not unambiguously be assigned to NSCLCadeno or NSCLCsquam) metastasis
SCLC (Small Cell Lung Cancer) metastasis
CHOL (Cholangiocarcinoma) metastasis
ESCA (Esophageal Carcinoma) metastasis
CESC (Cervical Squamous Cell Carcinoma and Endocervical Adenocarcinoma) metastasis
OC (Ovarian Carcinoma) metastasis
OV (Ovarian Serous Cystadenocarcinoma) metastasis
LIHC (Liver Hepatocellular Carcinoma) metastasis
RCC (Renal Cell Carcinoma) metastasis
KIRC (Kidney Renal Clear Cell Carcinoma) metastasis
KIRP (Kidney Renal Papillary Cell Carcinoma) metastasis
SARC (Sarcoma) metastasis
FS (Fibrosarcoma) metastasis
LPS (Liposarcoma) metastasis
MPNST (Malignant Peripheral Nerve Sheath Tumors) metastasis
SS (Synovial Sarcoma) metastasis
STAD (Stomach Adenocarcinoma) metastasis
TGCT (Testicular Germ Cell Tumors) metastasis
THYM (Thymoma) metastasis
UCS (Uterine Carcinosarcoma) metastasis and/or
UEC (Uterine Endometrial Carcinoma) metastasis.

According to further embodiments, the following is provided:

1. An in vitro method for producing activated T lymphocytes specific for use in the (manufacture of a medicament for the) treatment of a patient (i) being diagnosed for, (ii) suffering from, or (iii) being at risk of developing metastasis or a metastatic lesion, the method comprising the steps of providing a synthetic or recombinant peptide consisting in the amino acid sequence of SEQ ID NO: 310, contacting in vitro T cells with antigen-loaded human class I major histocompatibility complex (MHC) molecules expressed on the surface of a suitable antigen-presenting cell or an artificial construct mimicking an antigen-presenting cell for a period of time sufficient to activate said T cells in an antigen-specific manner, wherein said antigen is a peptide consisting in the amino acid sequence of SEQ ID NO: 310.

2. A cell line of activated T lymphocytes produced by the method according to item 1, characterized in that said cell line is capable of selectively recognizing metastatic cells which present a peptide consisting of the amino acid sequence of SEQ ID NO: 310.

3. An in vitro method for producing a soluble T cell receptor, characterized in that the method comprises the steps of:
(i) selecting a specific T cell clone that expresses a T cell receptor which binds to an HLA ligand that consists of a synthetic or recombinant peptide consisting of the amino acid sequence of SEQ ID NO: 310, optionally wherein said peptide is bound to an MHC, optionally wherein said T cell clone been created by immunizing a genetically engineered non-human mammal which is transgenic for the entire human TCR gene loci with a peptide comprising the amino acid sequence of SEQ ID NO: 310, or with a peptide-MHC complex comprising such peptide, optionally selecting, for example form a library of TCRs or CDR mutants by yeast, phage, or T cell display, a specific T cell receptor that binds to a synthetic or recombinant peptide comprising the amino acid sequence of SEQ ID NO: 310, optionally when bound to an MHC; or
(ii) selecting a specific T cell receptor that binds to an HLA ligand that consists of a synthetic or recombinant peptide consisting of the amino acid sequence of SEQ ID NO: 310, optionally wherein said peptide is bound to an MHC from a phage display system,
wherein said T cell receptor by virtue of binding to a peptide-MHC complex that comprises a peptide comprising SEQ ID NO: 310 bound to an MHC molecule is capable of reacting with an HLA ligand consisting of a peptide of SEQ ID NO: 310, which is presented metastatic cells.

4. An in vitro method for producing a recombinant antibody specifically binding to a human major histocompatibility complex (MHC) class I being complexed with a peptide of amino acid sequence of SEQ ID NO: 310, characterized in that the method comprises the steps of
immunizing a genetically engineered non-human mammal which is transgenic for the entire human immunoglobulin gene loci with a peptide comprising the amino acid sequence of SEQ ID NO: 310, or with a peptide-MHC complex comprising such peptide;
isolating mRNA molecules from antibody producing cells of said non-human mammal;
producing a phage display library displaying protein molecules encoded by said mRNA molecules; and
(iv) isolating at least one phage from said phage display library, in which the at least one phage contains said antibody that specifically binds to the peptide comprising SEQ ID NO: 310 bound to an MHC class I molecule;
wherein said antibody by virtue of binding to a peptide-MHC complex that comprises a peptide comprising SEQ ID NO: 310 bound to an MHC class I molecule is capable of specifically recognizing said peptide of SEQ ID NO: 310 when complexed with said MHC molecule,
wherein said peptide of SEQ ID NO: 310 is expressed in the surface of metastatic cells.

5. A pharmaceutically acceptable salt of the peptide consisting of the amino acid sequence of SEQ ID NO: 310, characterized in that the salt is an acetate, a trifluoro acetate, or a chloride.

6. A pharmaceutical composition comprising the cell line produced according to the method of item 2, the TCR produced according to the in vitro method of item 3, or the antibody produced according to the in vitro method of item 4 and a pharmaceutically acceptable carrier.

According to a further aspect of the invention, a nucleic acid is provided comprising at least one coding sequence encoding at least one antigenic peptide consisting of SLLQHLIGL (SEQ ID NO: 310).

In one embodiment, the nucleic acid comprises two or more encoding repeats (“concatemer”), separated by short nucleotide stretches (“spacers”).

Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a b-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) and/or chimeras and/or combinations thereof.

According to one embodiment the nucleic acid is an mRNA.

According to one embodiment, the mRNA comprises a 5′ untranslated region (UTR) and/or a 3′ UTR.

In several embodiments, the 3′-UTR comprises or consists of a nucleic acid sequence derived from a 3′-UTR of a gene selected from PSMB3, ALB7, alpha-globin, CASP1, COX6B1, GNAS, NDUFA1, and RPS9 or from a homolog, a fragment, or a variant of any one of these genes.

In several embodiments, the 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B, and UBQLN2 or from a homolog, a fragment, or variant of any one of these genes.

In several embodiments, the 5′-UTR and the heterologous 3′ UTR is selected from UTR design a-1 (HSD17B4/PSMB3), a-3 (SLC7A3/PSMB3), e-2 (RPL31/RPS9), and i-3 (−/muag), wherein UTR design a-1 (HSD17B4/PSMB3) and i-3 (−/muag).

According to one embodiment, the mRNA comprises a modified nucleoside in place of uridine.

According to one embodiment, the modified nucleoside is selected from pseudouridine (ψ), N 1-methyl-pseudouridine (m 1Ψ), and 5-methyl-uridine (m5U).

According to one embodiment, the nucleic acid comprises a coding sequence which is codon-optimized and/or in which the G/C content is increased and the uridine content is decreased compared to wild type coding sequence, wherein the codon-optimization and/or the increase in the G/C content preferably does not change the sequence of the encoded amino acid sequence.

The generation of a G/C content optimized nucleic acid sequence (RNA or DNA) may be carried out using a method according to WO2002/098443. In this context, the disclosure of WO2002/098443 is included in its full scope in the present invention.

In preferred embodiments, the nucleic acid may be modified, wherein the codons in the at least one coding sequence may be adapted to human codon usage (herein referred to as “human codon usage adapted coding sequence”).

Codons encoding the same amino acid occur at different frequencies in humans. Accordingly, the coding sequence of the nucleic acid is preferably modified such that the frequency of the codons encoding the same amino acid corresponds to the naturally occurring frequency of that codon according to the human codon usage. For example, in the case of the amino acid alanine, the wild type or reference coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. Accordingly, such a procedure (as exemplified for alanine) is applied for each amino acid encoded by the coding sequence of the nucleic acid to obtain sequences adapted to human codon usage.

According to several embodiments, the nucleic acid is at least one selected from the group consisting of SEQ ID NO:
314 (PRAME mRNA)
315 (PRAME mRNA GC enriched)
316 (PRAME cDNA)
317 (PRAME 004 mRNA)
318 (PRAME 004 mRNA GC enriched)
319 (PRAME 004 cDNA)

According to another aspect of the invention, a composition or medical preparation comprising the nucleic acid according to the above description is provided.

In one embodiment, said composition does not comprise a nucleic acid that encodes for a peptide that is a fragment of the Prostate specific Membrane antigen (PSMA), in particular not for PSMA₂₈₈₋₂₉₇ (GLPSIPVHPI, SEQ ID NO: 376) or PSMA₂₈₈₋₂₉₇ I297V (GLPSIPVHPV, SEQ ID NO: 377).

According to one embodiment, the composition comprises mRNA with an RNA integrity of 70% or more.

The term “RNA integrity” generally describes whether the complete RNA sequence is present in the liquid composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH, or other factors (e.g. nucleophilic attacks, hydrolysis etc.), which may reduce the RNA integrity and, consequently, the functionality of the RNA.

According to one embodiment, the composition comprises mRNA with a capping degree of 70% or more, preferably wherein at least 70%, 80%, or 90% of the mRNA species comprise a Cap1 structure.

5′-capping of polynucleotides may be completed concomitantly during the in vitro transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-0 methyl-transferase to generate: m7G(5′)ppp(5′)G-2 ′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source.

According to several embodiments, the at least one nucleic acid is complexed or associated with one or more lipids or lipid-based carriers, thereby forming liposomes, lipid nanoparticles (LNP), lipoplexes, and/or nanoliposomes, preferably encapsulating the at least one nucleic acid.

According to one embodiment, the LNP comprises
(i) at least one cationic lipid;
(ii) at least one neutral lipid;
(iii) at least one steroid or steroid analogue; and
(iv) at least one polymer conjugated lipid, preferably a PEG-lipid.

According to one embodiment, (i) to (iv) are in a molar ratio of about 20-60% cationic lipid, 5-25% neutral lipid, 25-55% sterol, and 0.5-15% PEG-lipid.

According to several embodiments, the cationic lipid is at least one selected from the group consisting of
a) SM-102 (Heptadecan-9-yl-8-{(2-hydroxyethyl) [6-oxo-6-(undecyloxy)hexyl]amino}-octanoat)

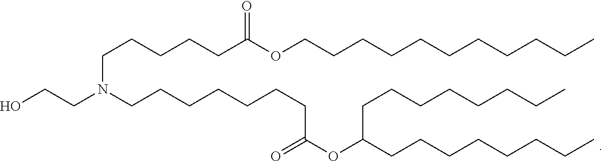 [SMILES: CCCCCCCCCCCOC(=O)CCCCCN(CCO)CCCCCCCC(=O)OC(CCCCCCCC)CCCCCCCC]

b) ALC-0315 ([(4-Hydroxybutyl)azandiyl]bis(hex an-6,1-diyl)bis(2-hexyldecanoat)

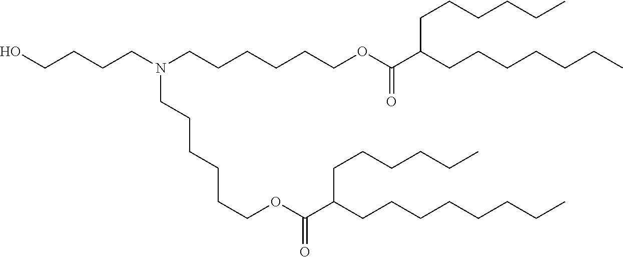 [SMILES: CCCCCCCCC(CCCCCC)C(=O)OCCCCCCN(CCCCO)CCCCCCOC(=O)C(CCCCCC)CCCCCCC]

According to several embodiments, the polymer conjugated lipid is at least one selected from the group consisting of:
a)

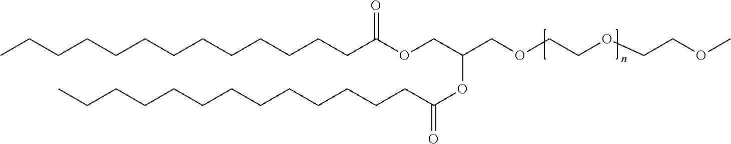

wherein n has a mean value ranging from ≥30 to ≤60, preferably wherein n has a mean value of 44 or 45,
b)

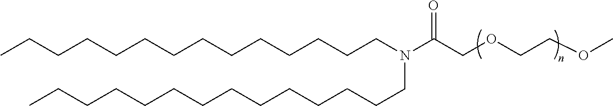

preferably 1,2-Dimyristoyl-rac-glycero-3-methoxypolyethylene glycol-2000 (PEG2000 DMG)
   wherein n has a mean value ranging from ≥30 to ≤60, preferably wherein n has a mean value of 49 or 45, preferably 2-[(polyethylene glycol)-2000]-N,N-ditetradecylacetamide (ALC-0159)

According to one embodiment, the neutral lipid is 1,2-distearoyl-sn-glycero-3-phosphocholine (DSPC).

According to one embodiment, the steroid or steroid analogue is cholesterol.

According to one embodiment, the composition or medical preparation is a vaccine.

According to another aspect of the invention, a method is provided of eliciting an immune response to a tumor or a metastatic lesion that presents a peptide comprising SLLQHLIGL (SEQ ID NO: 310) on a cell surface, which method comprises administering to a patient the composition according to the above description.

According to another aspect of the invention, a composition according to the above description is provided for use in the (manufacture of a medicament for the) treatment of a patient (i) being diagnosed for, (ii) suffering from, or (iii) being at risk of developing a tumor or a metastatic lesion that presents a peptide comprising SLLQHLIGL (SEQ ID NO: 310) on a cell surface.

According to several embodiments thereof, the tumor is selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, uterine carcinoma, uterine carcinosarcoma, head and neck squamous cell carcinomas, head and neck adenocarcinoma, colon cancer, gastro-intestinal cancer, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

According to several embodiments thereof, the metastatic lesion is at least one selected from the group consisting of
ACC metastasis
BLCA metastasis
BRCA metastasis
TNBC metastasis
CRC metastasis
HNSCC metastasis
HNAC metastasis
MEL metastasis
SKCM metastasis
UVM metastasis
LC metastasis
NSCLC metastasis
NSCLCadeno metastasis
NSCLCsquam metastasis
NSCLCother metastasis
SCLC metastasis
CHOL metastasis
ESCA metastasis
CESC metastasis
OC metastasis
OV metastasis
LIHC metastasis
RCC metastasis
KIRC metastasis
KIRP metastasis
SARC metastasis
FS metastasis
LPS metastasis
MPNST metastasis
SS metastasis
STAD metastasis
TGCT metastasis
THYM metastasis
UCS metastasis
UCEC metastasis, and/or
UEC metastasis.

According to several embodiments thereof, the metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, uterine carcinoma, uterine carcinosarcoma, head and neck squamous cell carcinomas, head and neck adenocarcinoma, colon cancer, gastro-intestinal cancer, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

### BRIEF DESCRIPTION OF THE FIGURES

FIG. 1 shows γδ T cell expansion using Zoledronate (Zometa) in defined medium, which contains IL-2, IL-15, and Amphotericin B. Fold increase in absolute number of γδ T cells is 3,350-fold, 11,060-fold, and 31,666-fold for donor 20 from day 0 to day 17, from day 0 to day 22, and from day 0 to day 29, respectively. Similarly, fold increase in absolute number of γδ T cells is 4,633-fold, 12,320-fold, and 32,833-fold for donor 21 from day 0 to day 17, from day 0 to day 22, and from day 0 to day 29, respectively. In contrast, as noted above, classic Vγ962 T cell expansion protocol, at best, could yield only a 100-fold increase in total Vγ962 T cells within 14 days, thereafter, the expansion rate decreases, which may be caused by an increase of cell death. In an aspect, using the afore-mentioned methods, fold increase in absolute number of γδ T cells after expansion on day 29 as compared with that of day 0 may be from about 1000-fold to about 40,000-fold, from about 3000-fold to about 35,000-fold, from about 5000-fold to about 35,000-fold, from about 6000-fold to about 35,000-fold, from about 7000-fold to about 35,000-fold, from about 8000-fold to 30,000-fold, from about 10,000-fold to about 35,000-fold, from about 15,000-fold to about 35,000-fold, from about 20,000-fold to about 35,000-fold, from about 25,000-fold to about 35,000-fold, from about 30,000-fold to about 35,000-fold, more than about 10,000 fold, more than about 15,000 fold, more than about 20,000 fold, more than about 25,000 fold, more than about 30,000 fold, more than about 40,000 fold, or more than about 40,000 fold.

FIG. 2A shows, as compared with Vγ962 T cells without viral transduction (Mock), 34.9% of Vγ962 T cells transducing with αβ-TCR retrovirus and CD84a retrovirus αβ-TCR+CD8) stained positive by peptide-MHC-dextramer (TAA/MHC-dex) and anti-CD8 antibody (CD8), indicating the generation of Vγ962 T cells expressing both αβ-TCR and CD84a on cell surface (αβ-TCR+CD8αβ engineered Vg9d2 T cells).

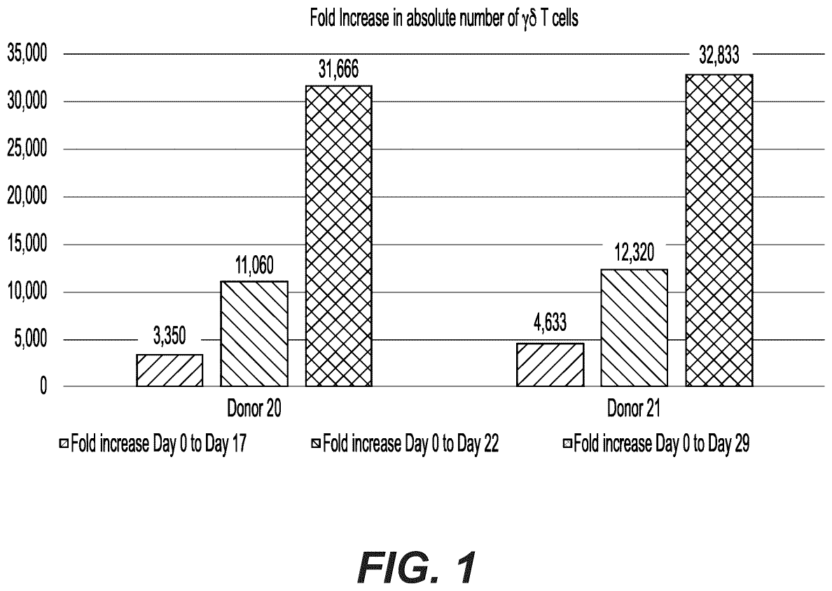

The principle of CD107a degranulation assay is based on killing of target cells via a granule-dependent pathway that utilizes pre-formed lytic granules located within the cytoplasm of cytotoxic cells. The lipid bilayer surrounding these granules contains lysosomal associated membrane glycoproteins (LAMPs), including CD107a (LAMP-1). Rapidly upon recognition of target cells via the T cell receptor complex, apoptosis-inducing proteins like granzymes and perforin are released into the immunological synapse, a process referred to as degranulation. Thereby, the transmembrane protein CD107a is exposed to the cell surface and can be stained by specific monoclonal antibodies.

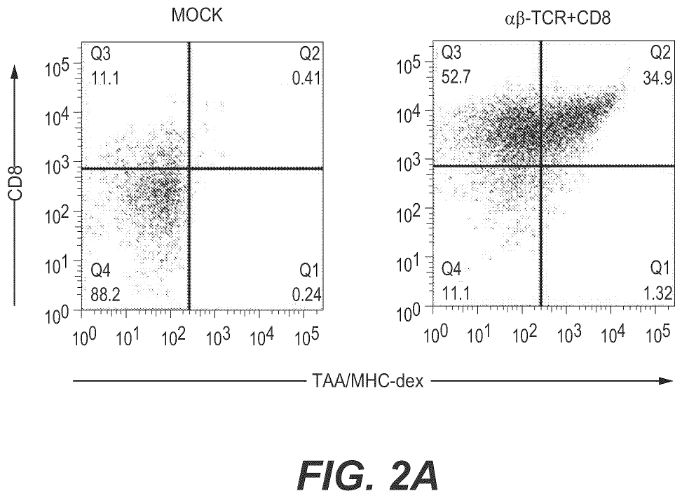

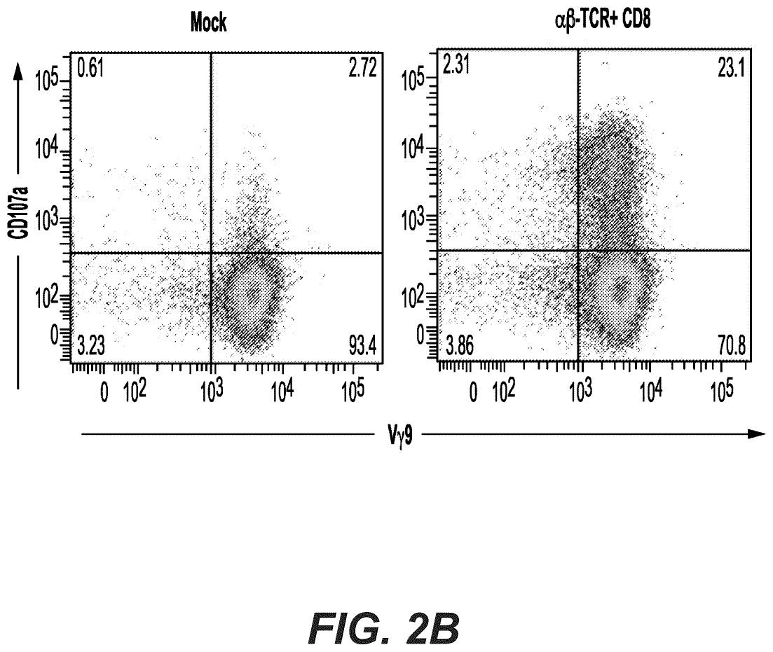

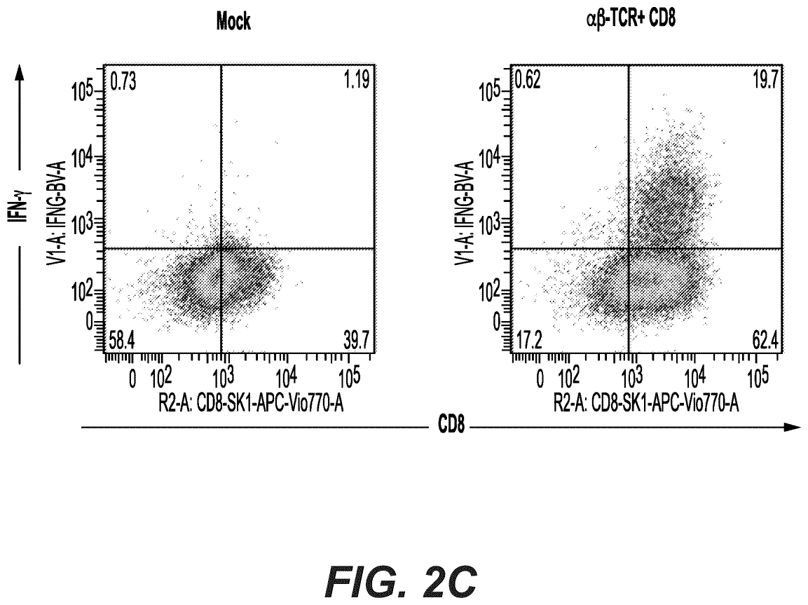

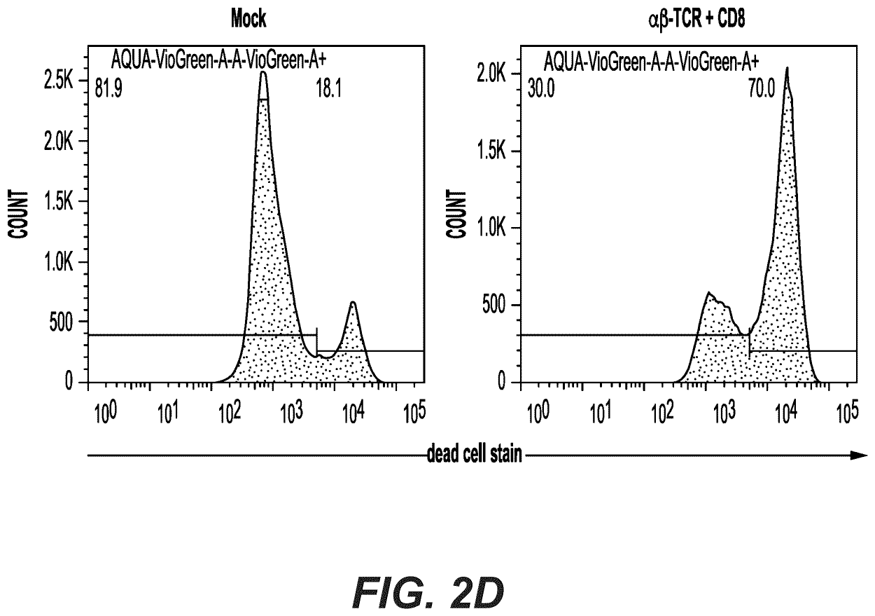

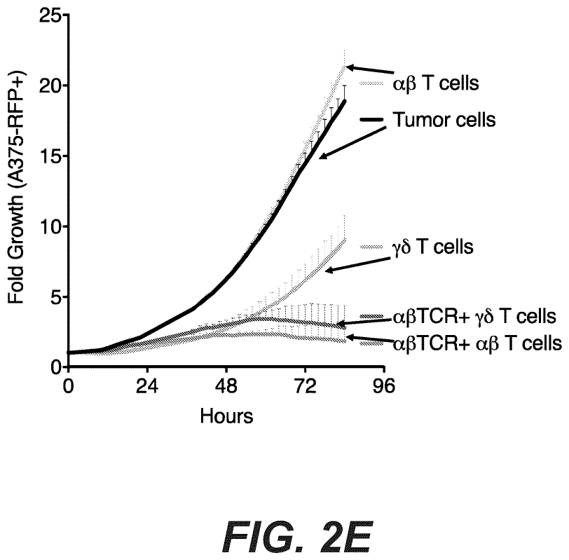

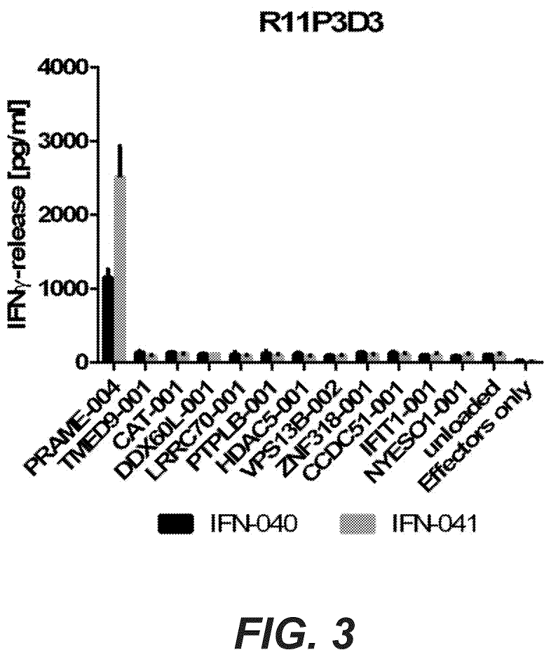

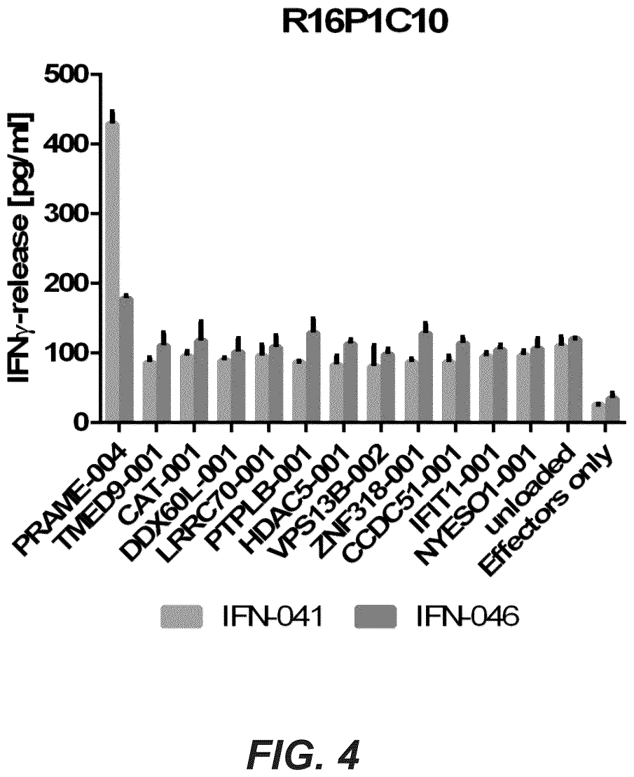

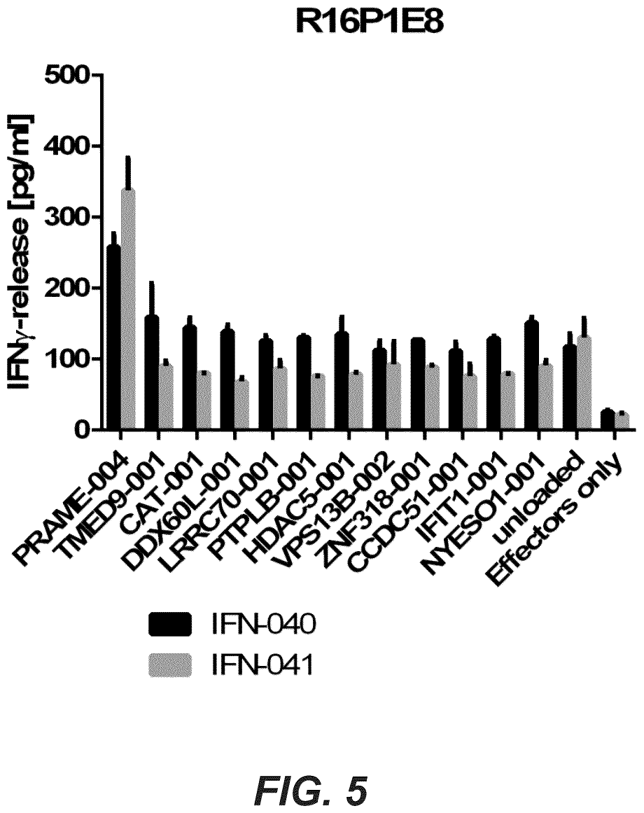

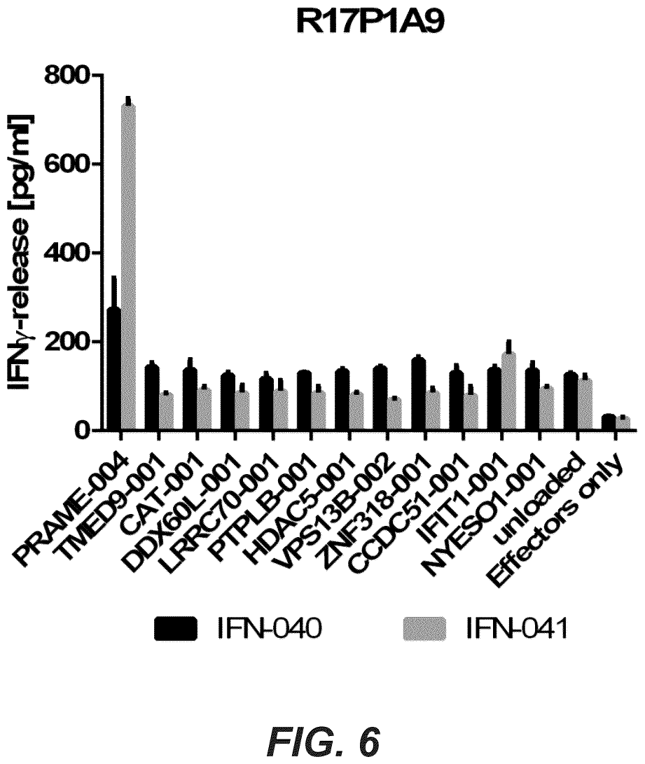

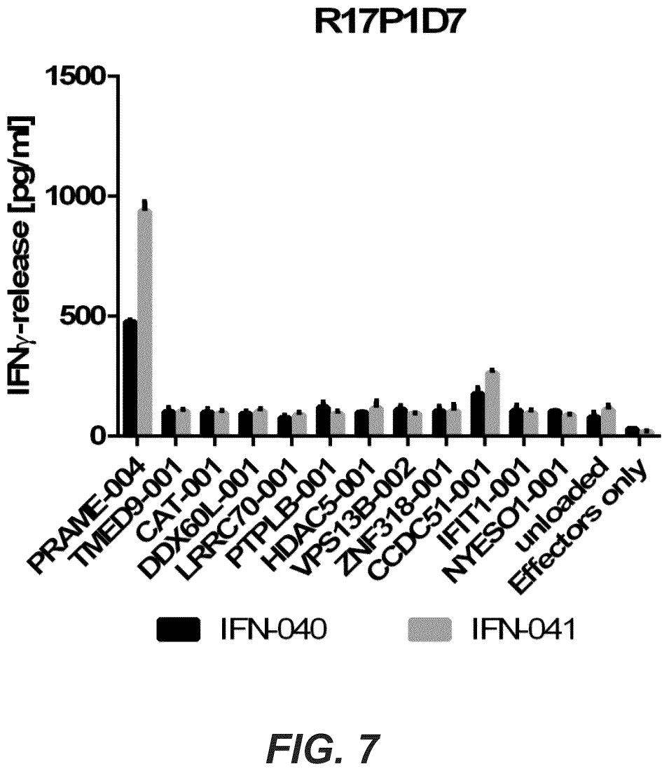

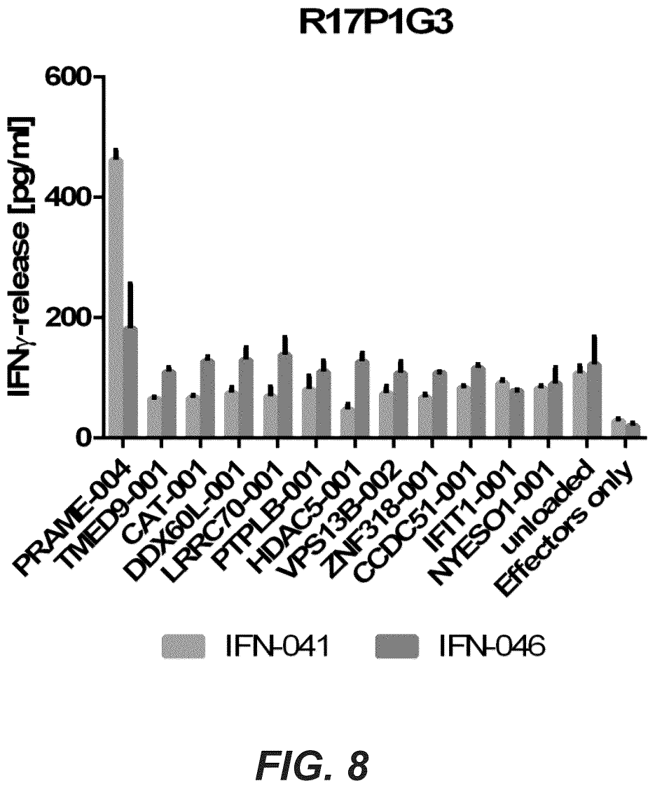

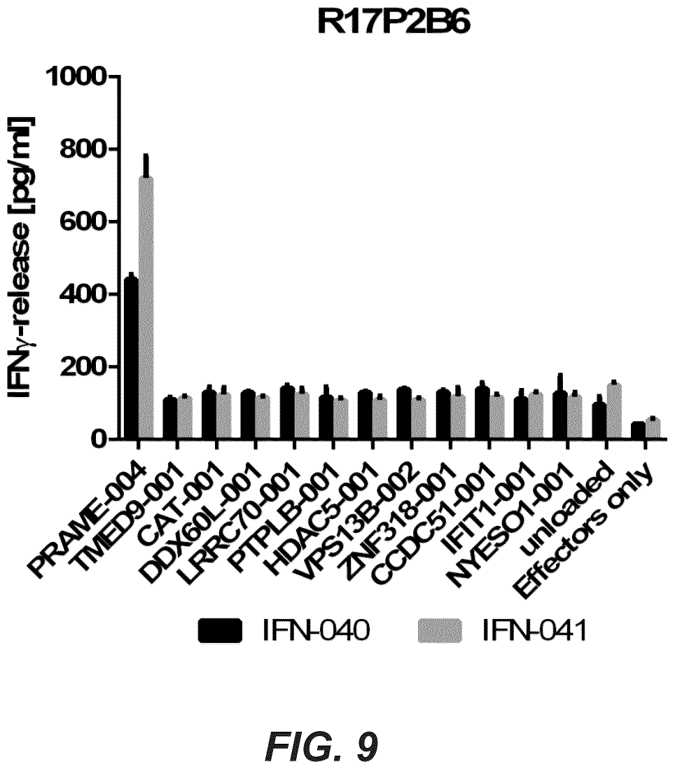

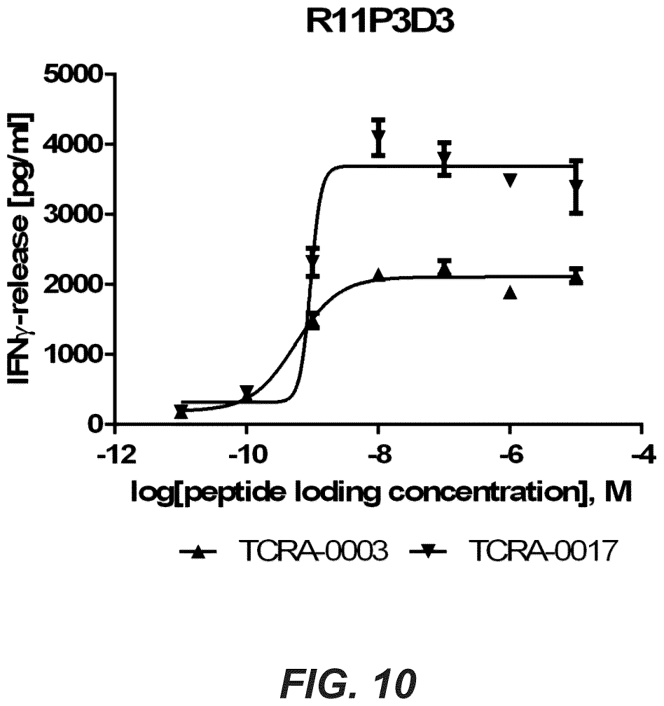

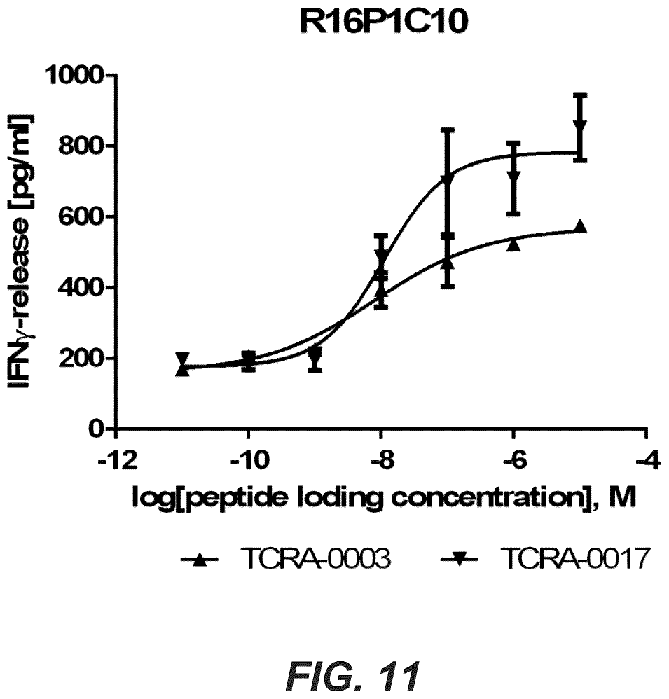

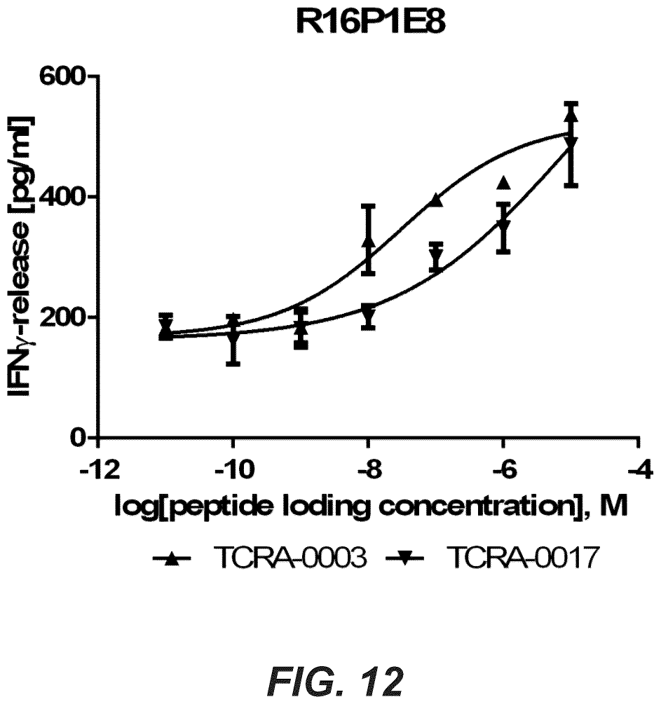

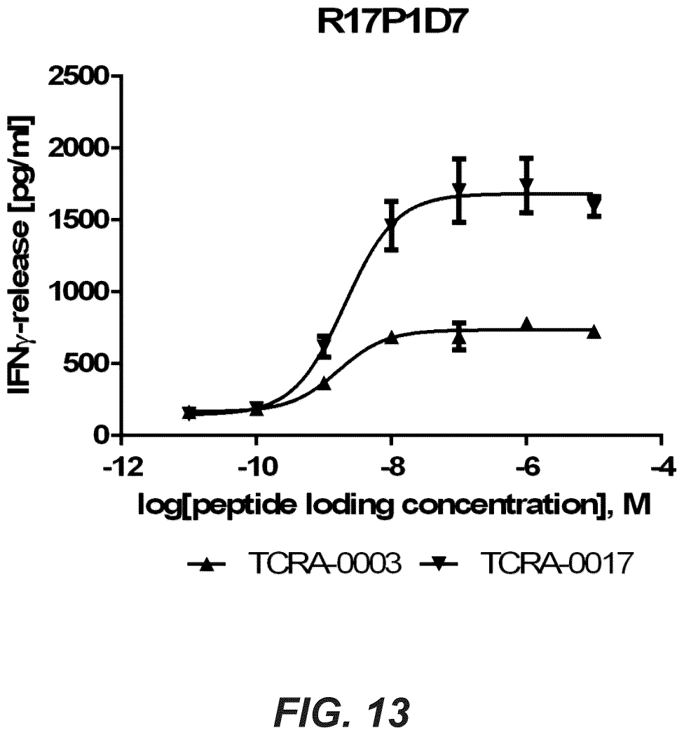

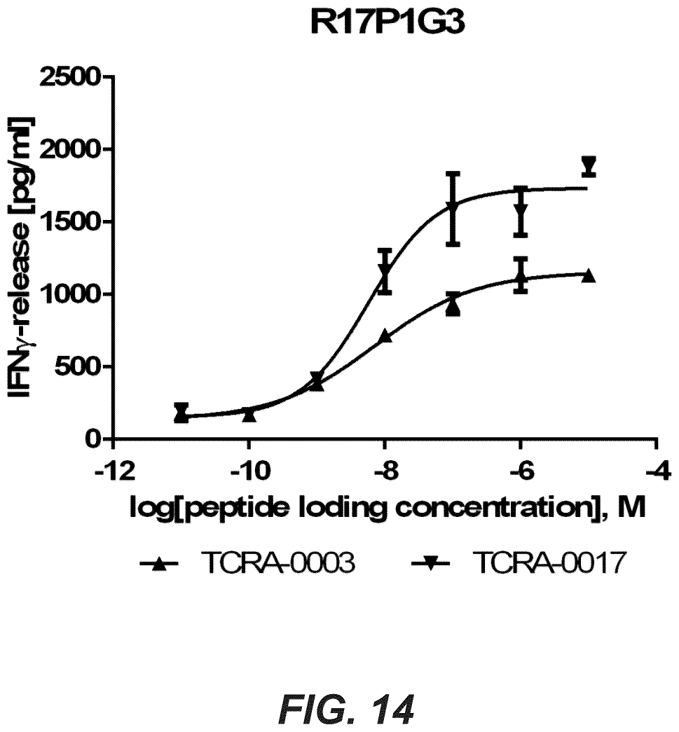

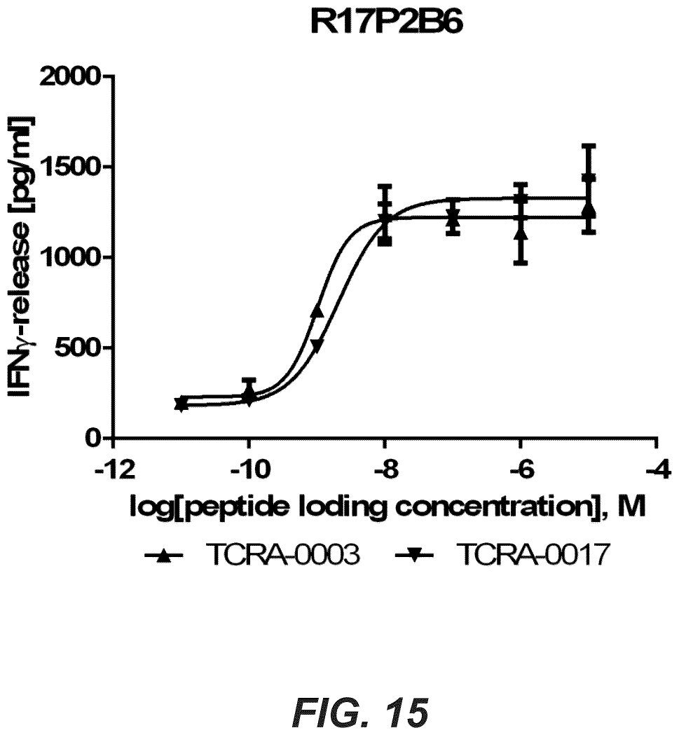

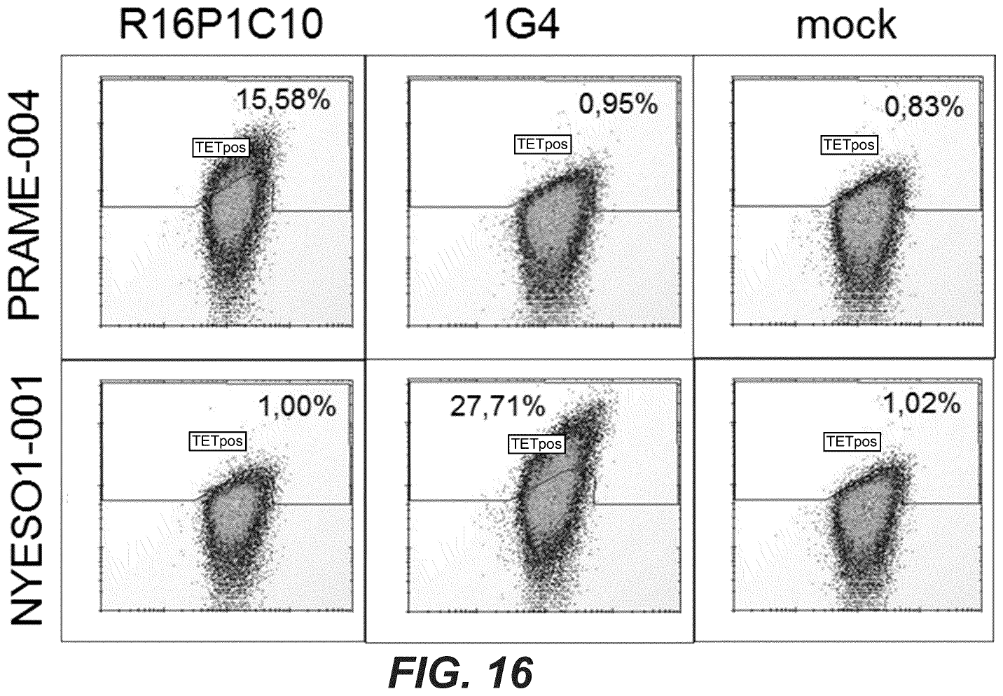

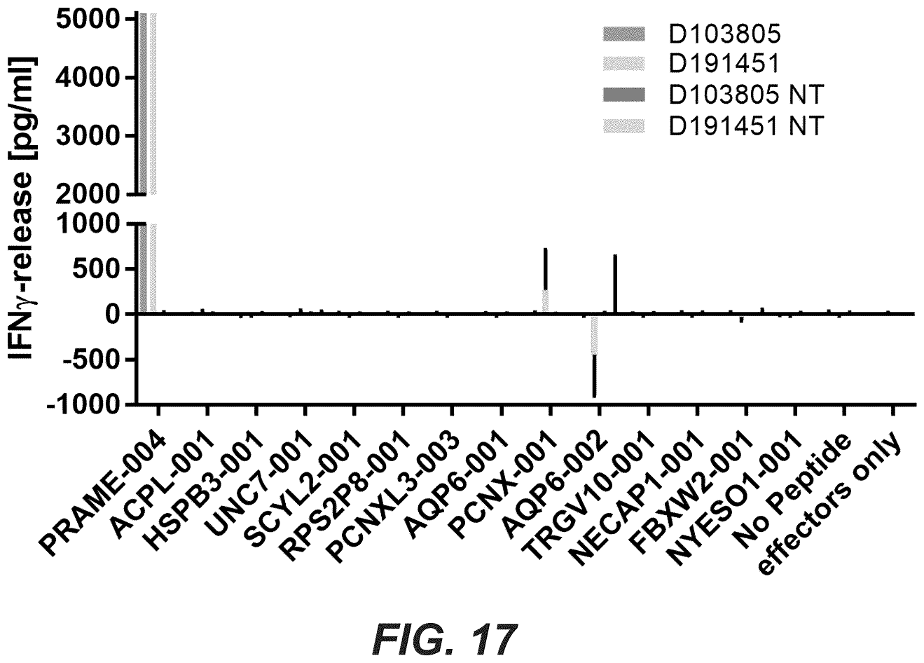

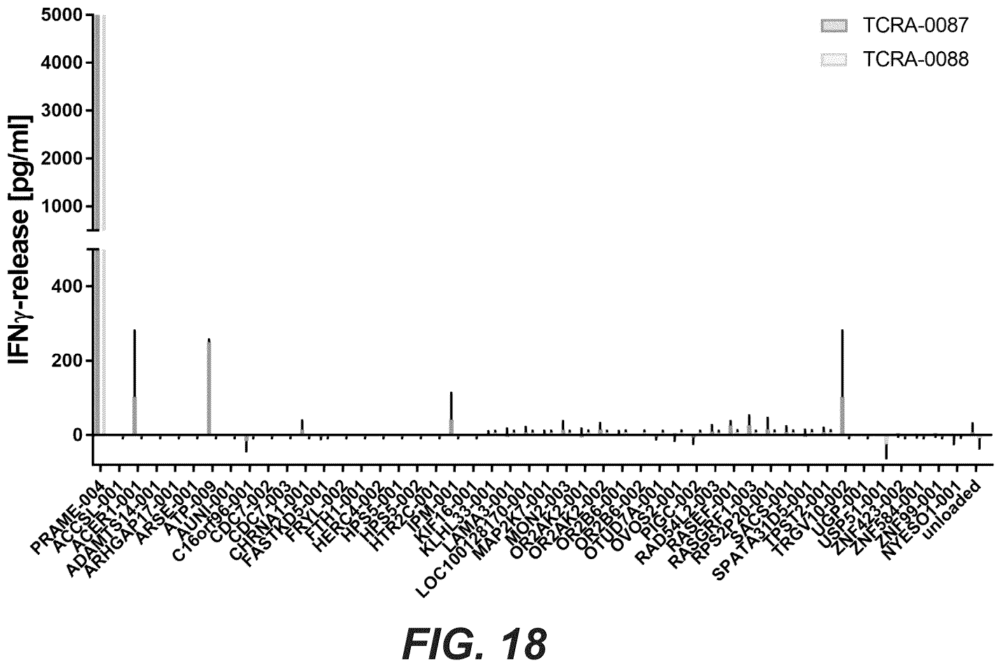

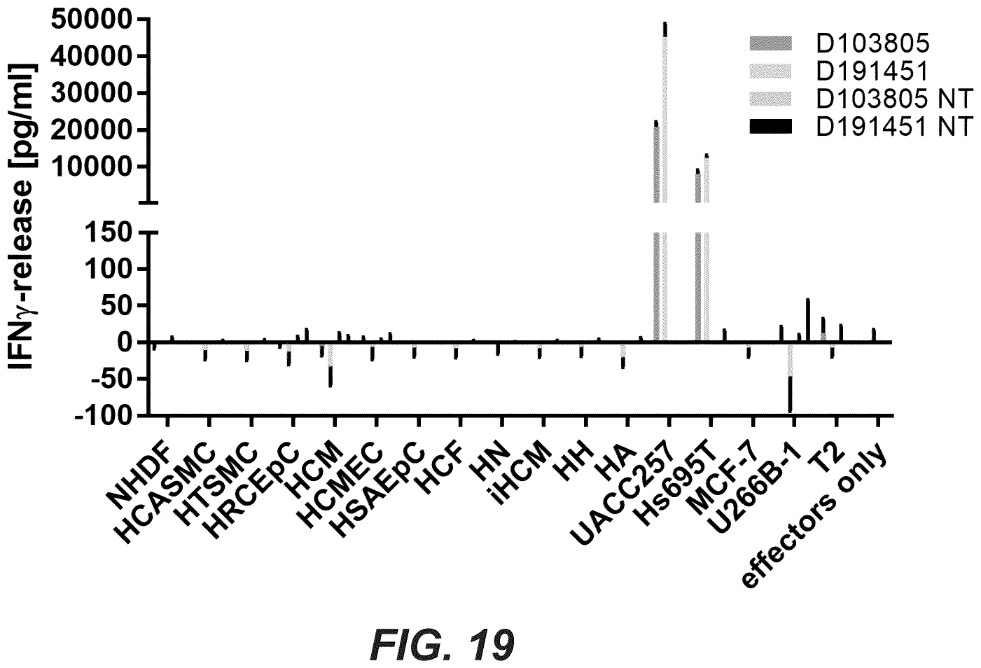

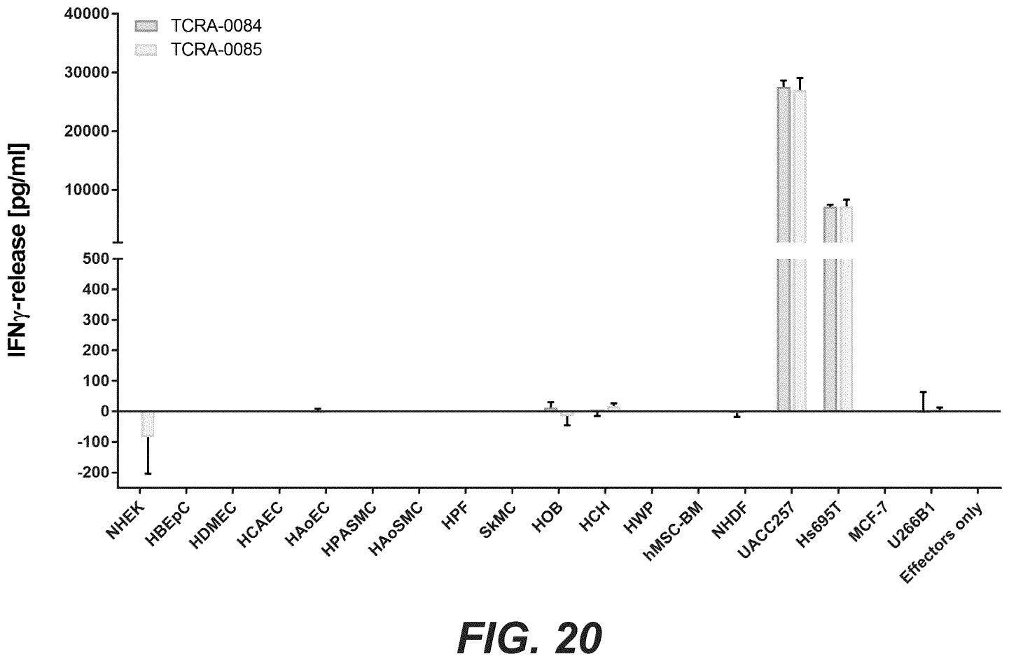

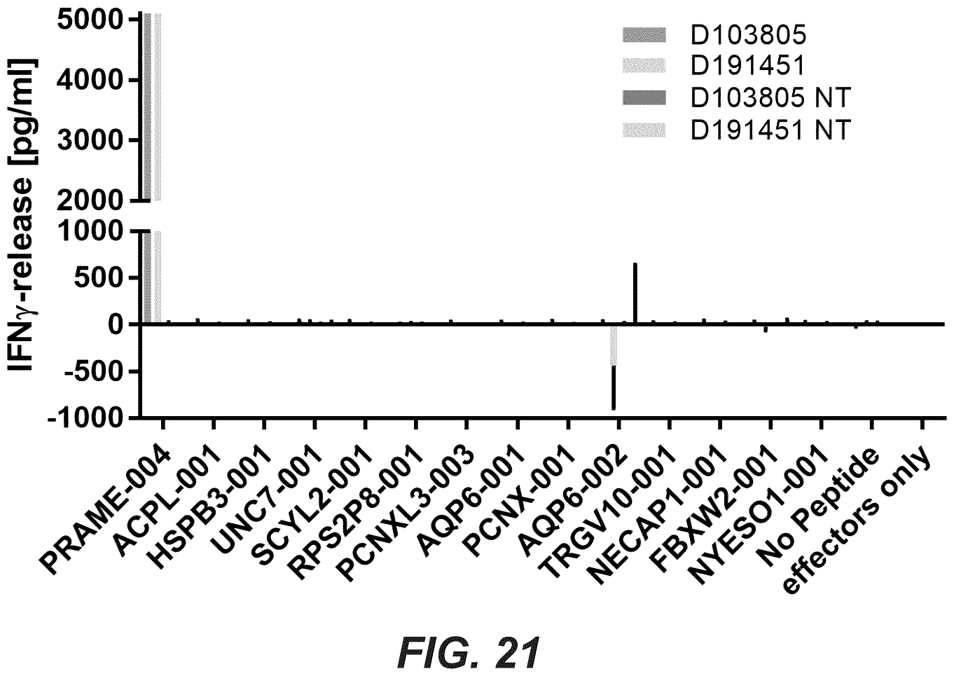

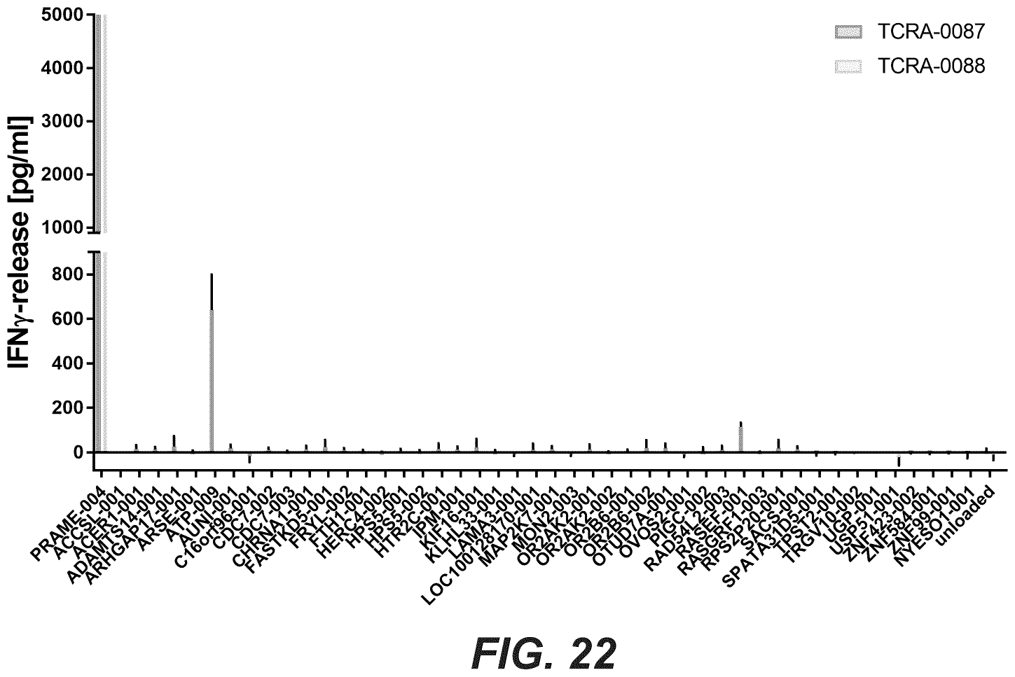

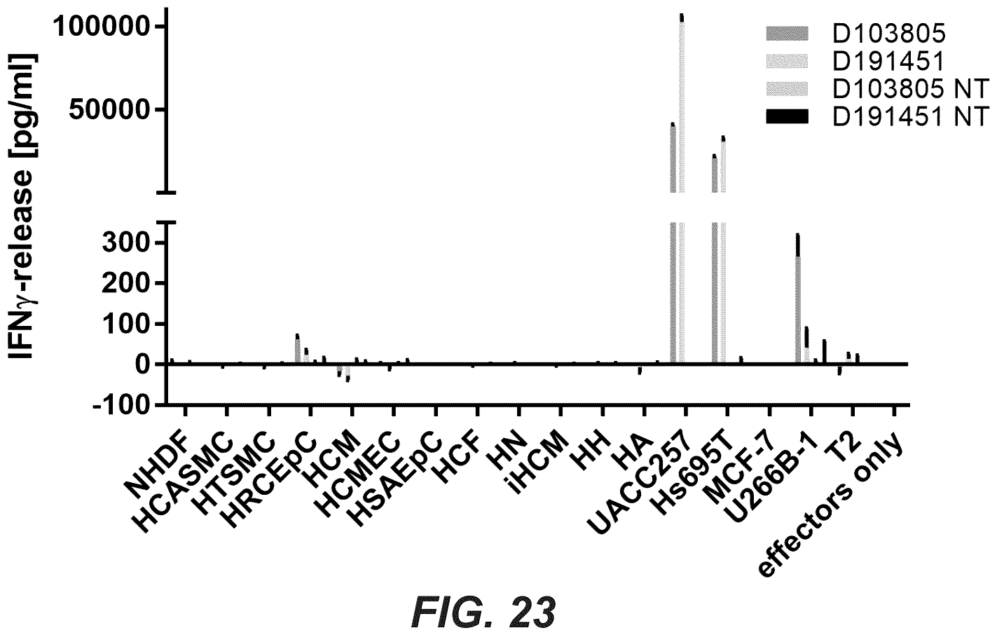

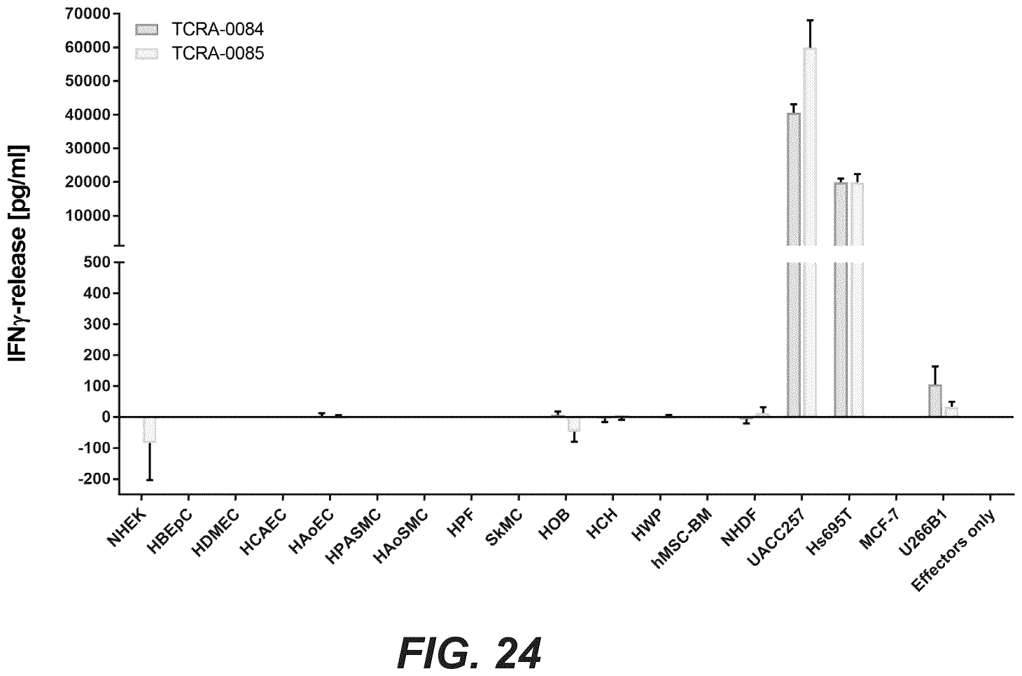

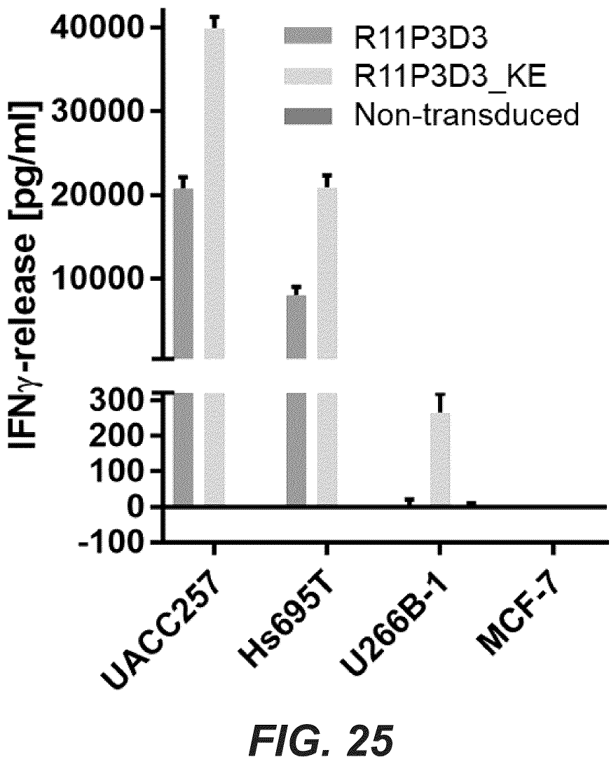

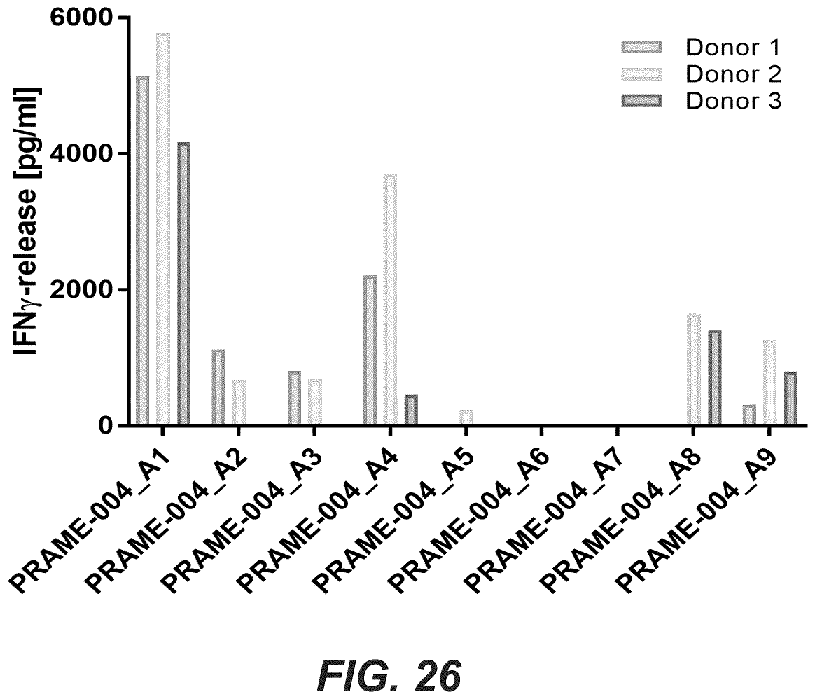

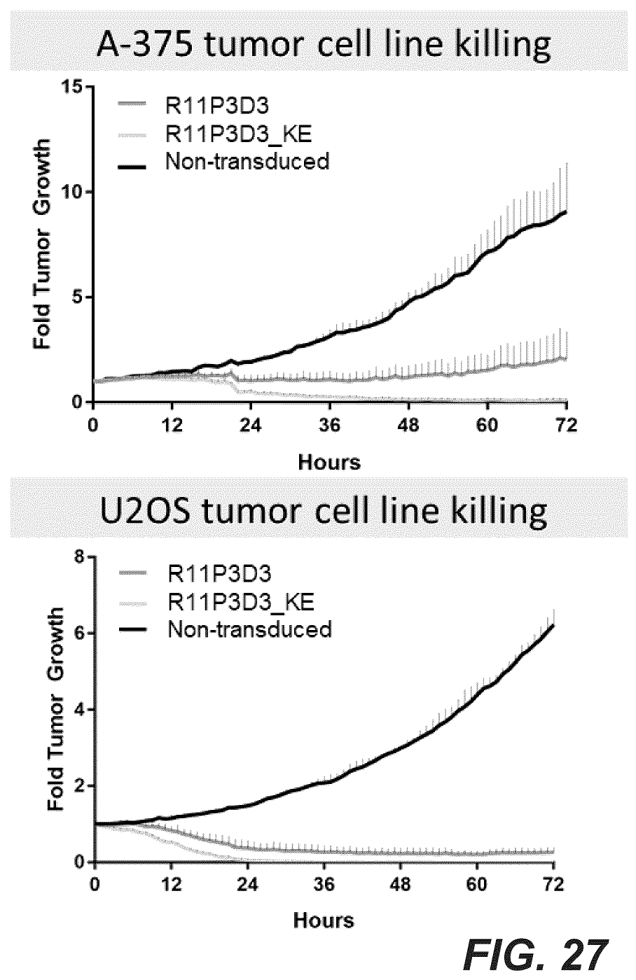

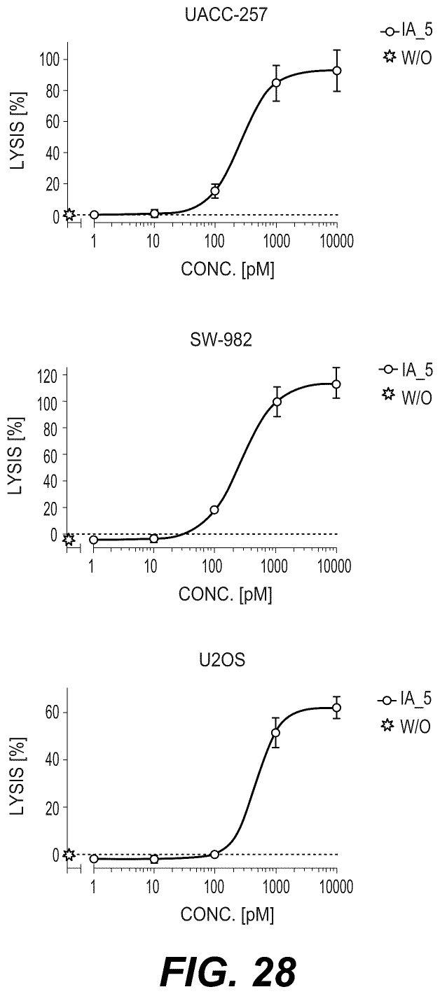

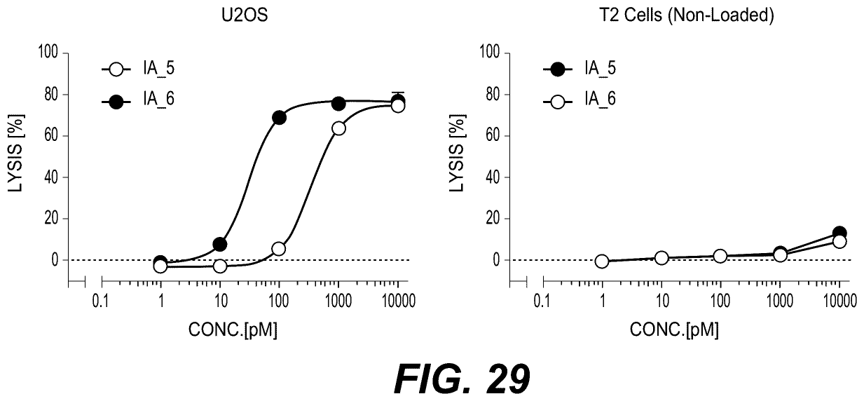

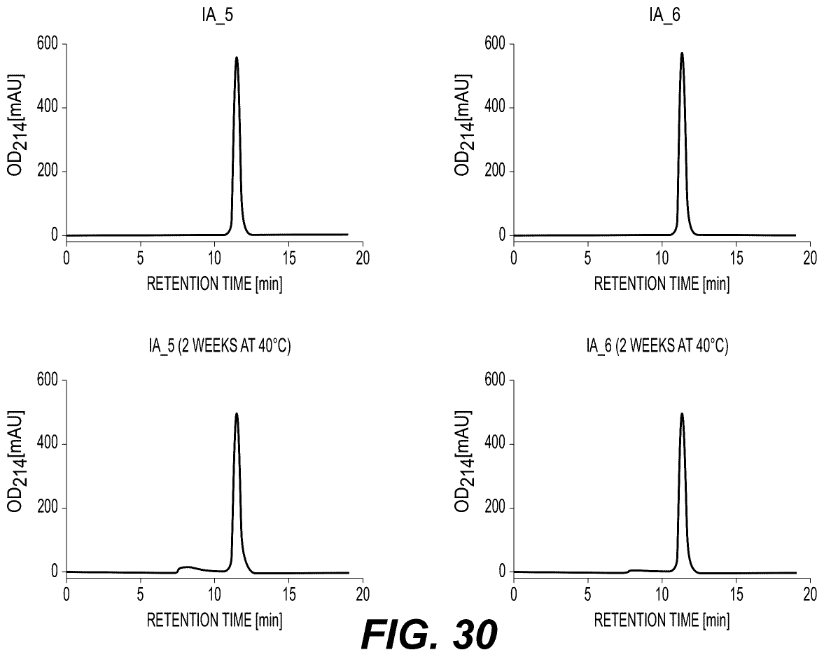

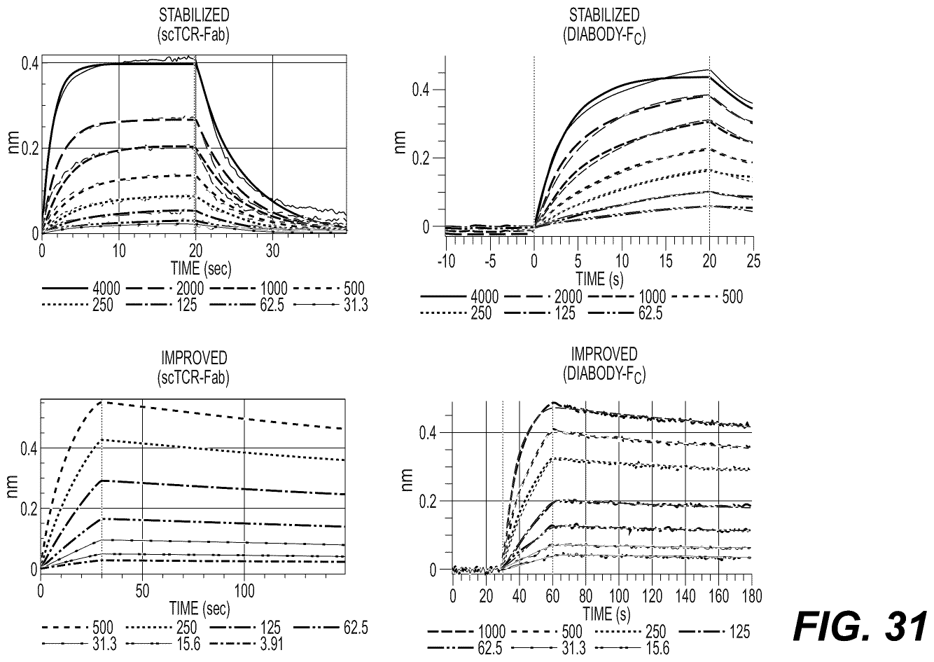

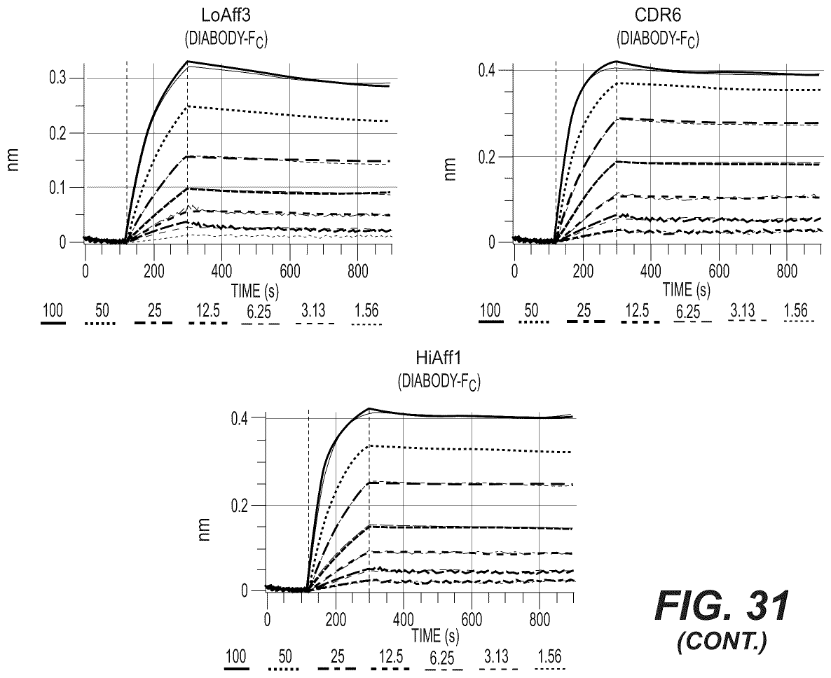

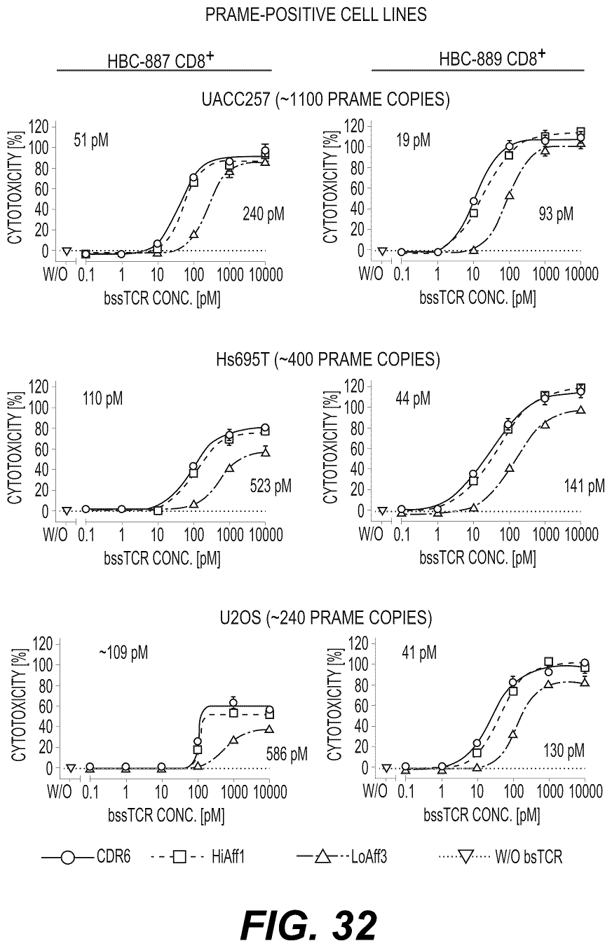

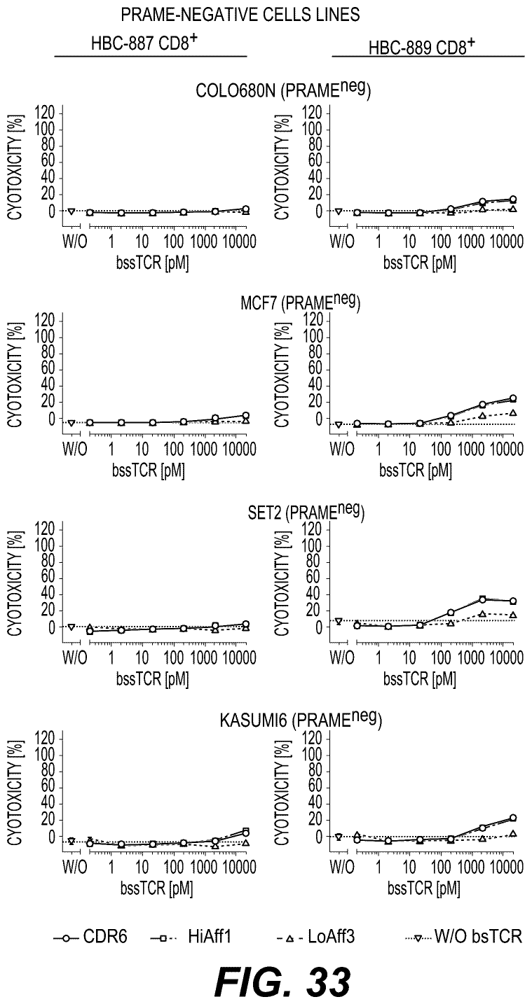

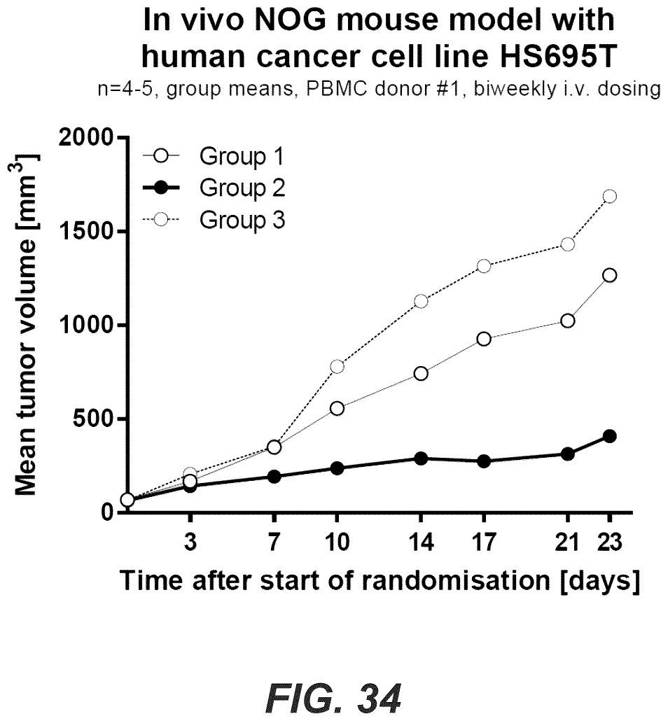

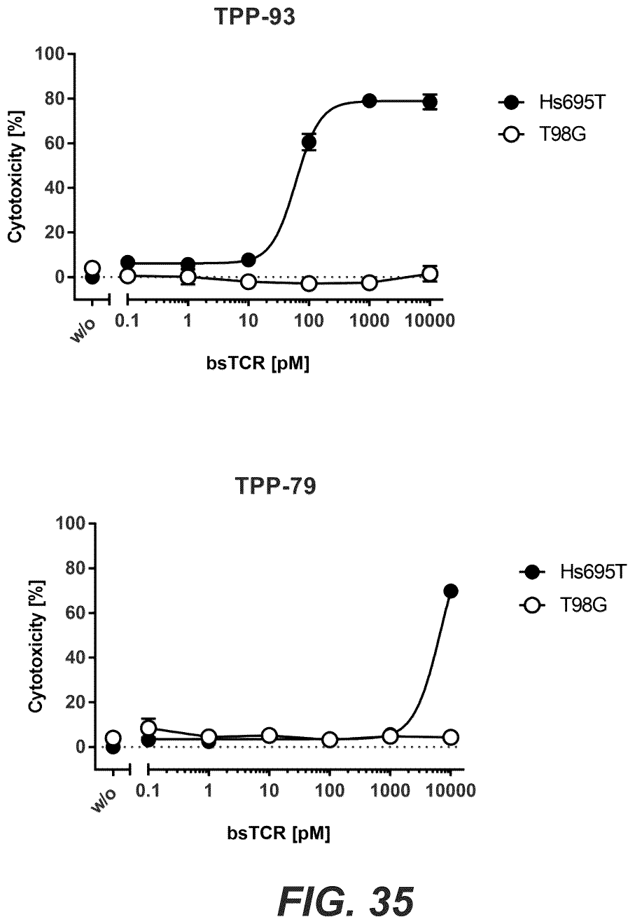

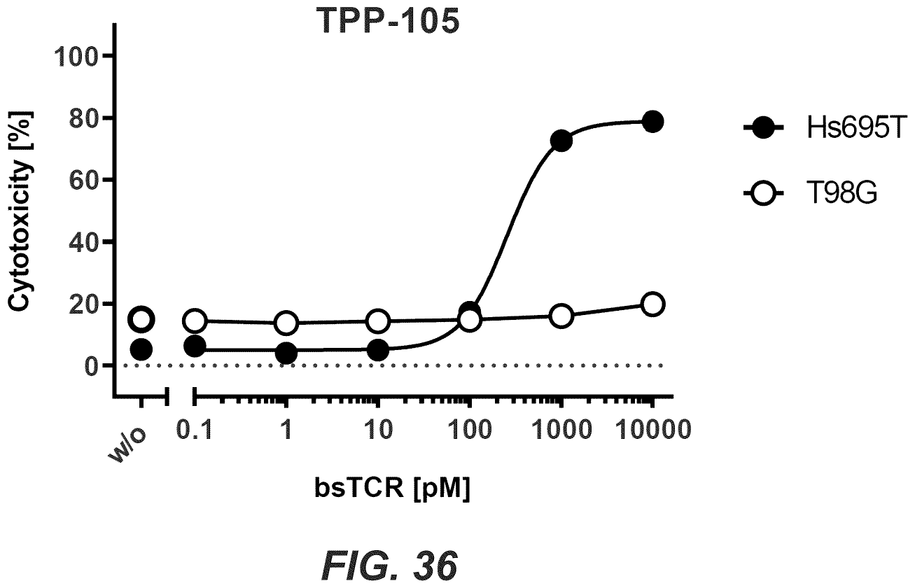

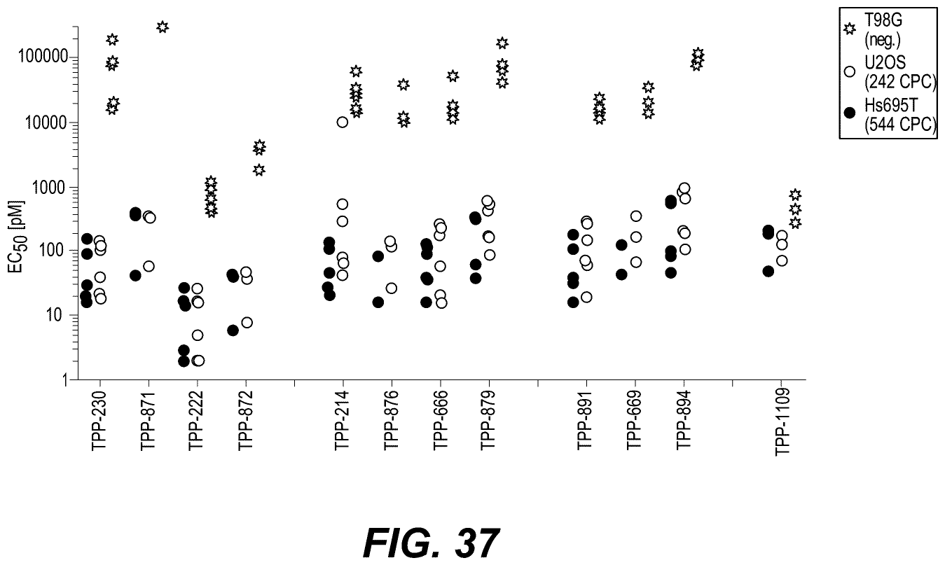

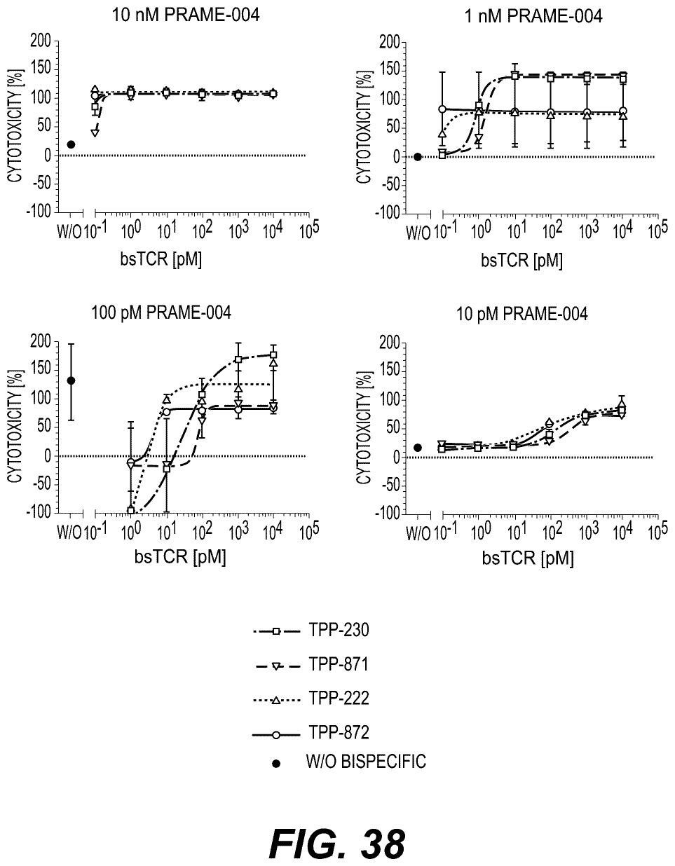

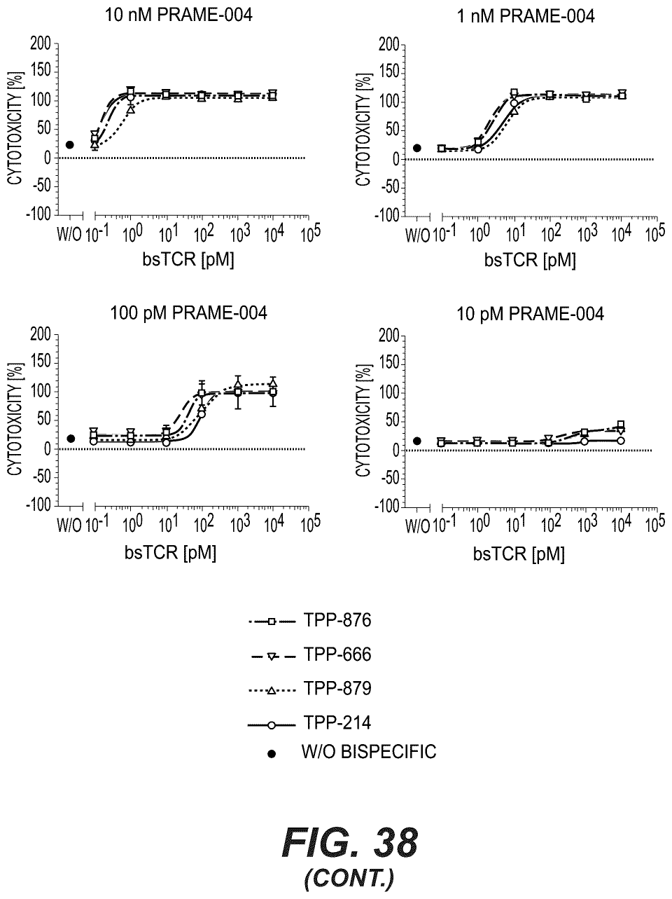

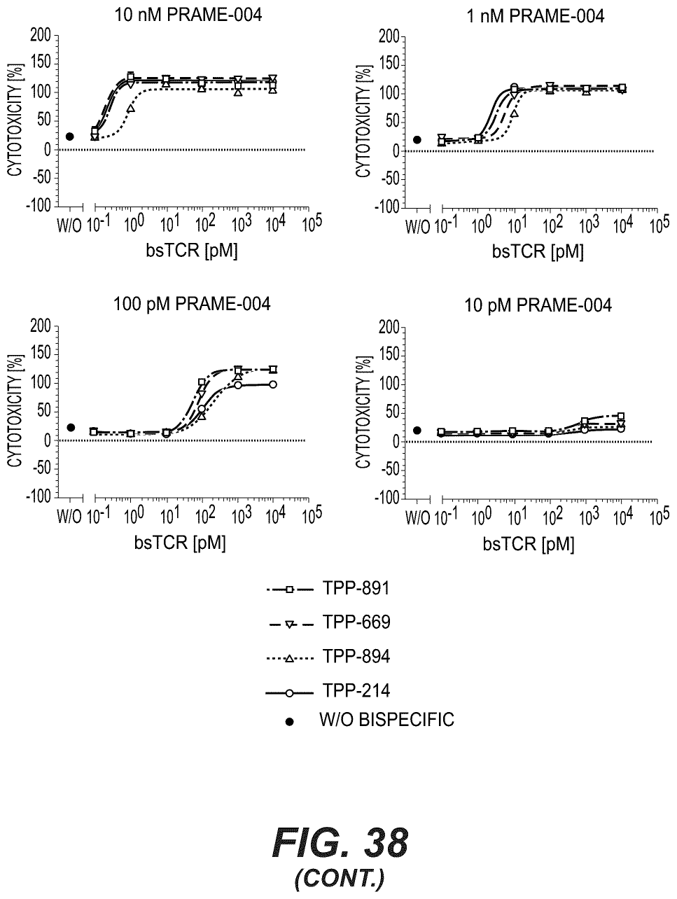

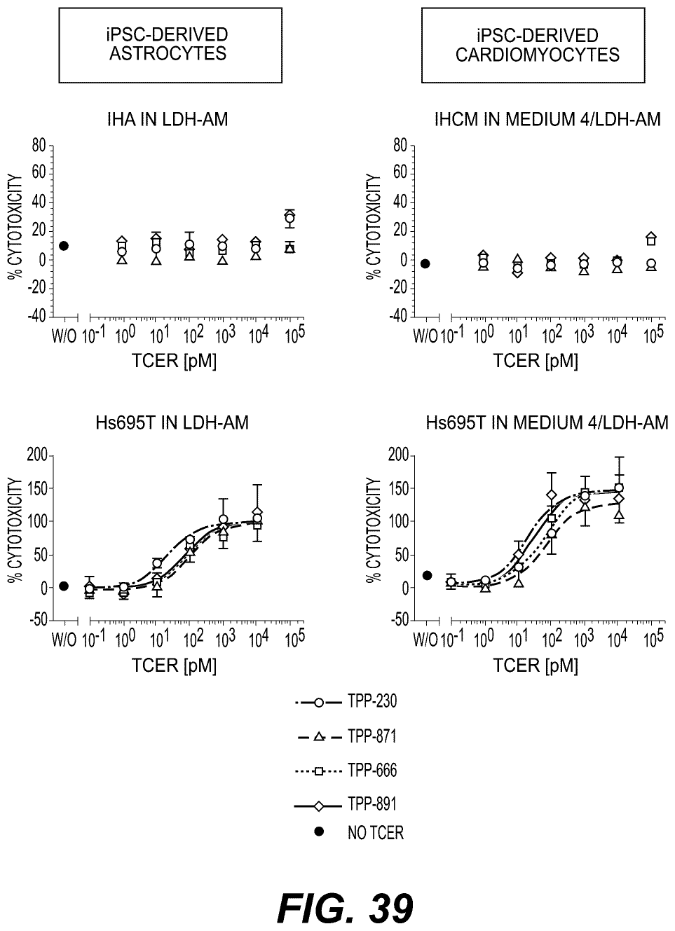

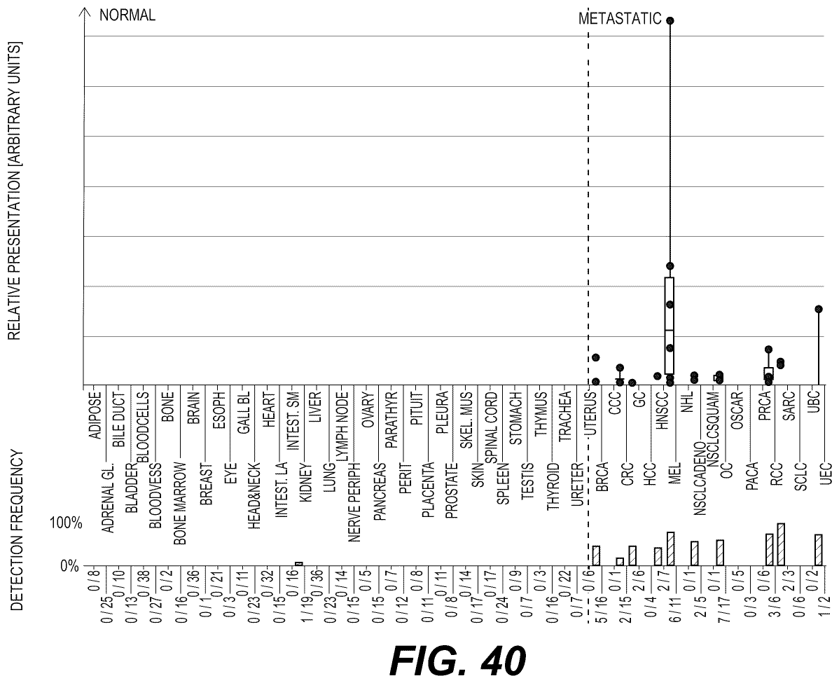

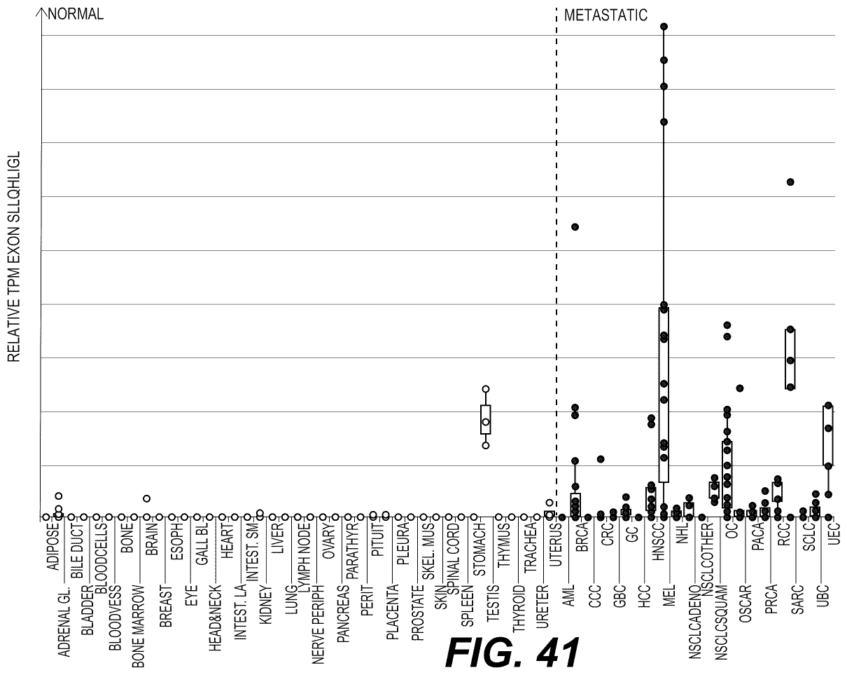

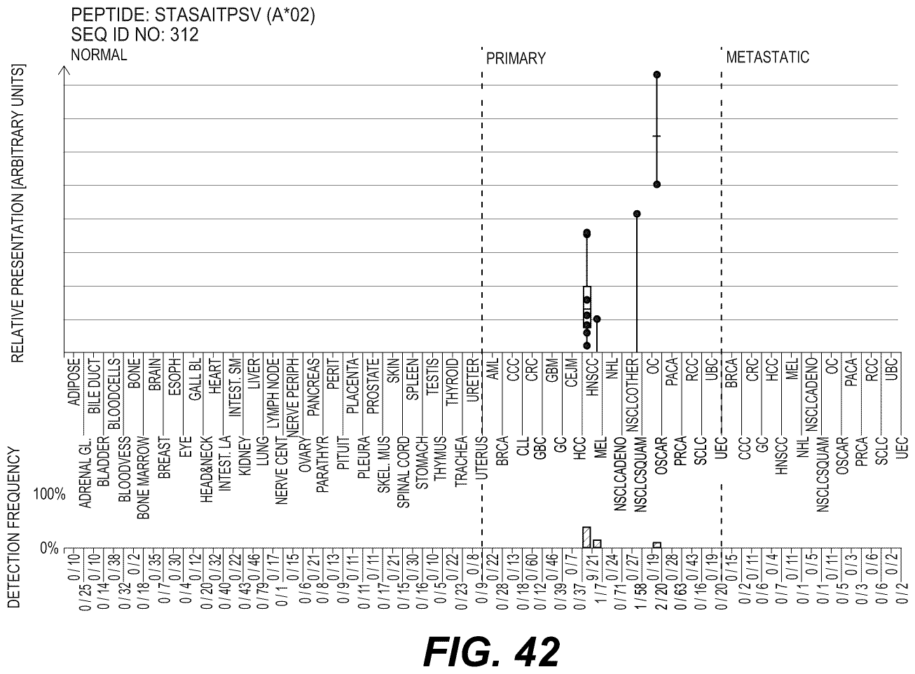

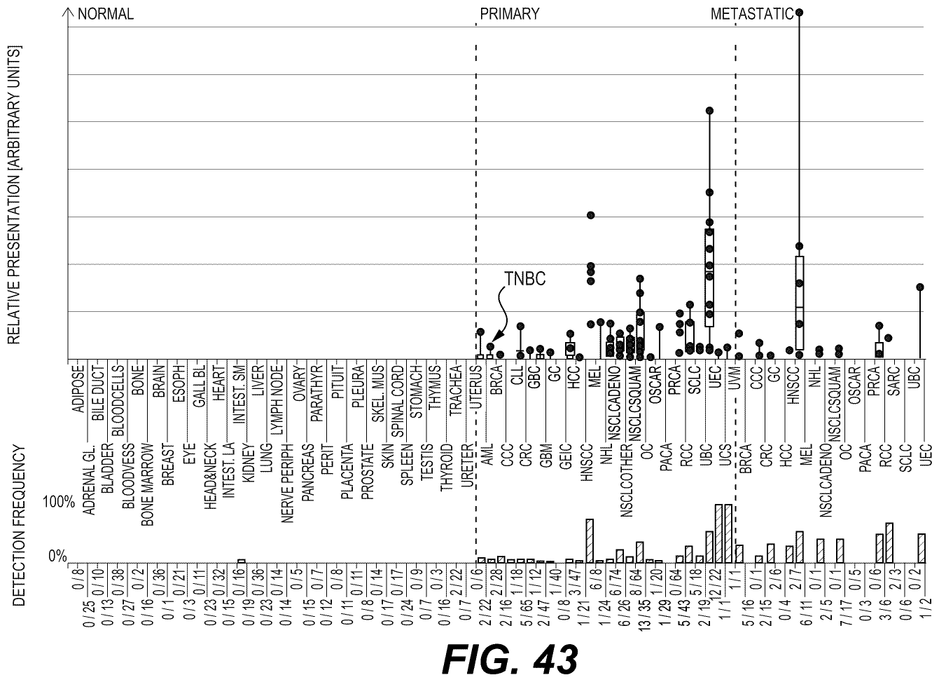

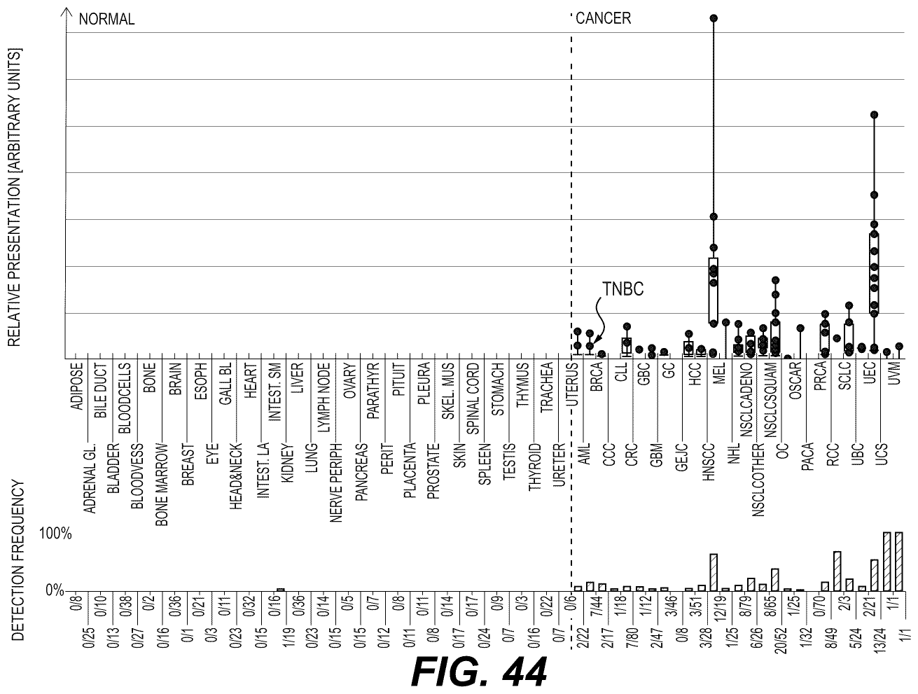

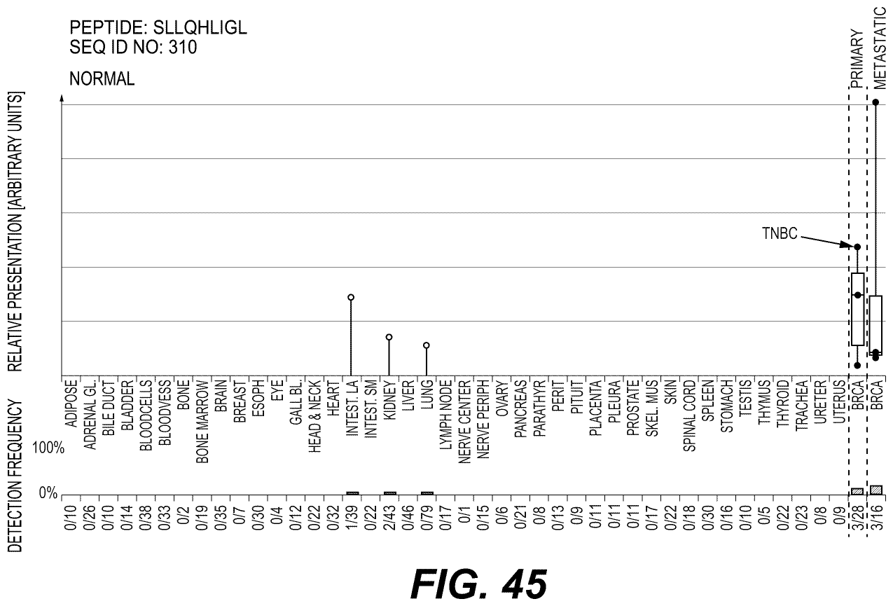

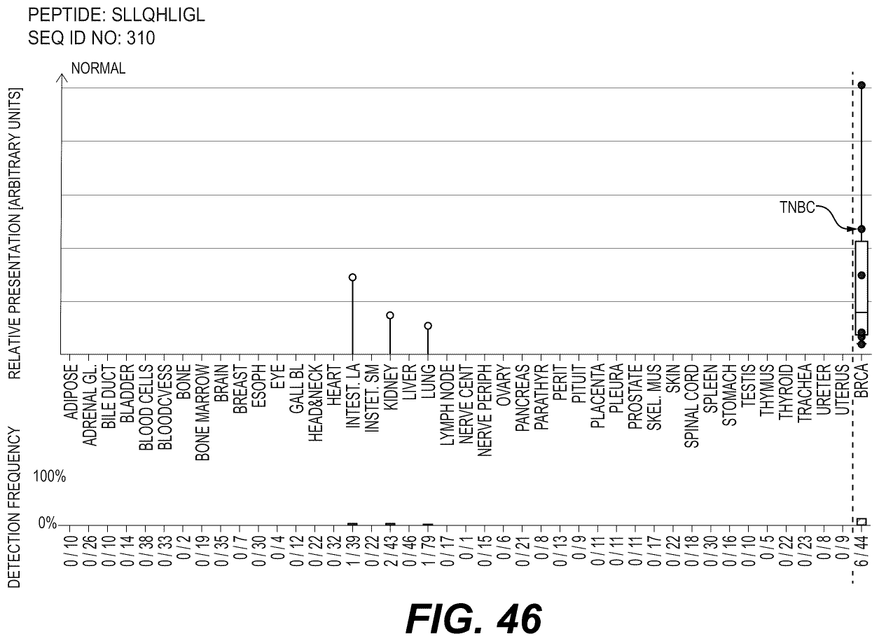

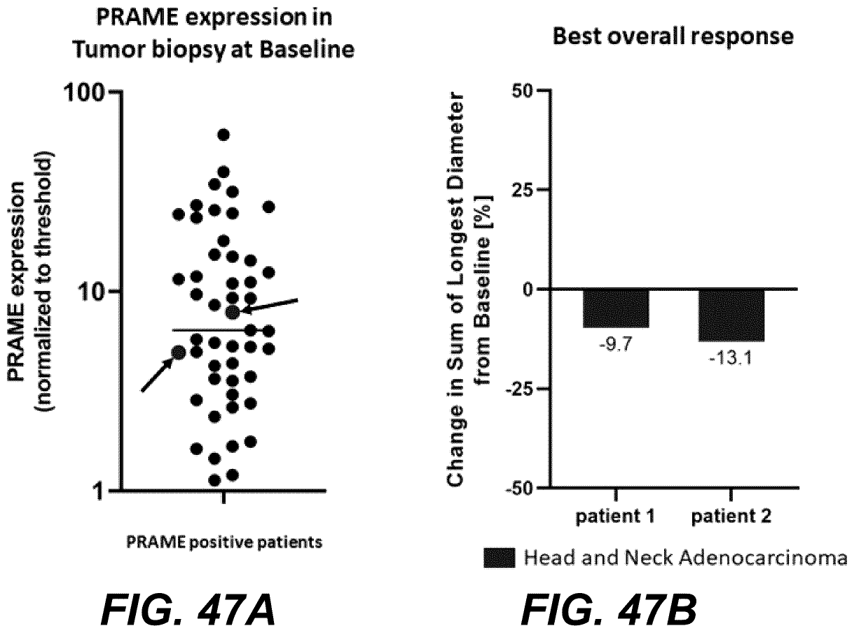

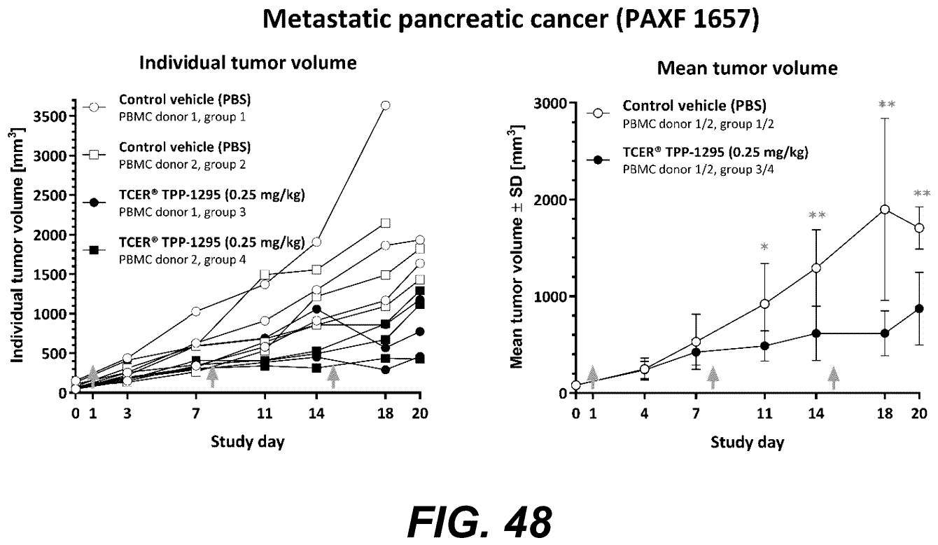

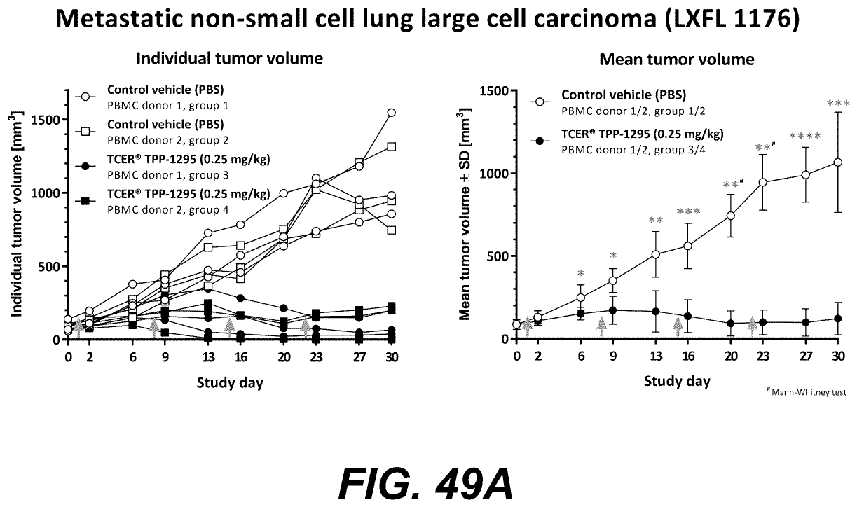

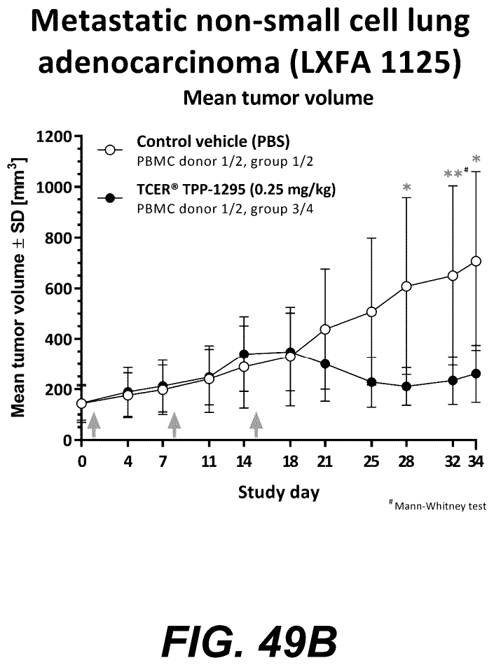

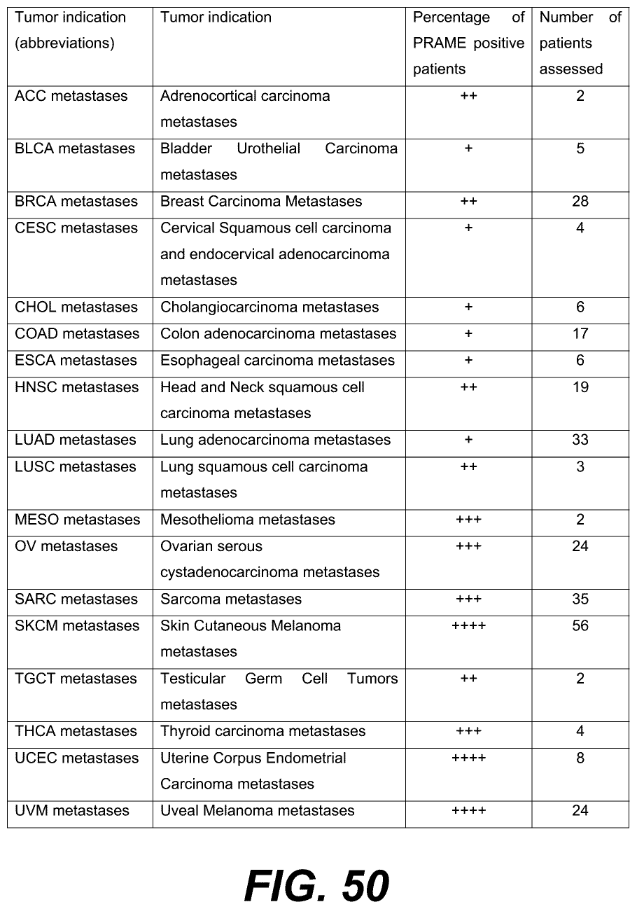

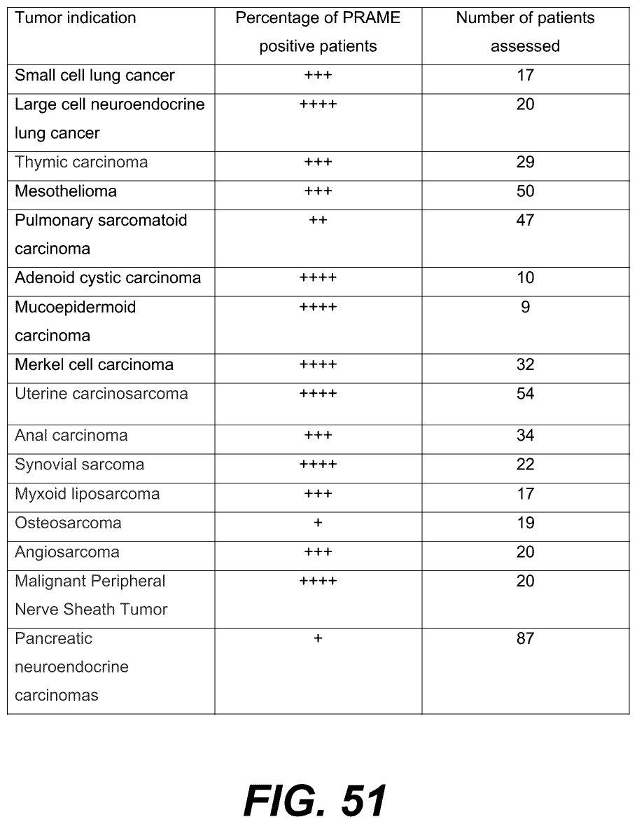

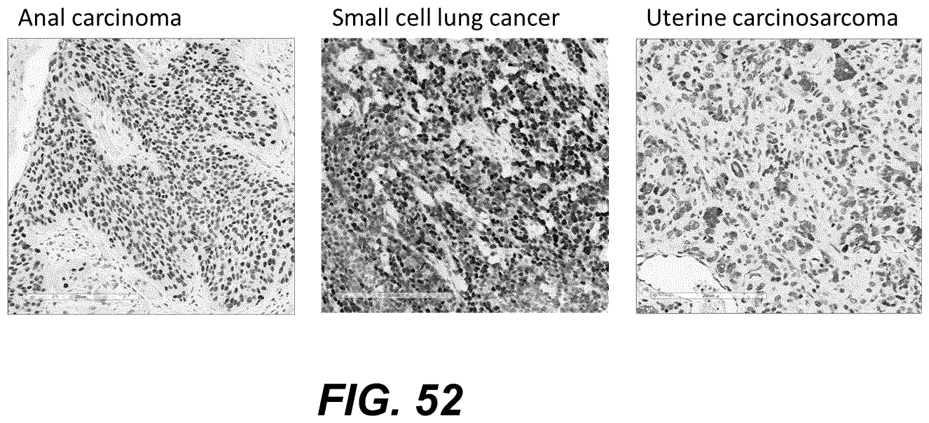

FIG. 2B shows, as compared with Vγ982 T cells without viral transduction (Mock), 23.1% of Vγ982 T cells transduced with αβ-TCR retrovirus and CD8αβ retrovirus (αβ-TCR+CD8) incubated with target cells, e.g., A375 cells, stained positive by anti-CD107a antibody, indicating that αβ-TCR+CD8αβ engineered Vg9d2 T cells are cytolytic by carrying out degranulation, when exposed to A375 cells. IFN-γ release assays measure the cell mediated response to antigen-presenting cells, e.g., A375 cells, through the levels of IFN-γ released, when TCR of T cells specifically binds to peptide-MHC complex of antigen-presenting cells on cell surface.

FIG. 2C shows, as compared with Vγ982 T cells without viral transduction (Mock), 19.7% of Vγ982 T cells transduced with αβ-TCR retrovirus and CD8αβ retrovirus (αβ-TCR+CD8) stained positive by anti-IFN-γ antibody, indicating that αβTCR+CD8αβ engineered Vγ982 T cells are cytolytic by releasing IFN-γ, when exposed to A375 cells.

Cytolytic activity were evaluated at 24 hours post-exposure to A375 cells by gating on apoptosis of non-CD3 T cells, i.e., A375 cells. Apoptosis was assessed by staining the harvested culture with live/dead dye.

FIG. 2D shows, as compared with Vγ982 T cells without viral transduction (Mock), αβTCR+CD8αβ engineered Vγ982 T cells (αβ-TCR+CD8) induced apoptosis in 70% of A375 cells, indicating that αβ-TCR+CD8αβ engineered Vγ982 T cells are cytolytic by killing A375 cells. Cytolytic activity was also evaluated in real-time during an 84-hour co-culture assay. Non-transduced and αβTCR+CD8αβ transduced γδ T cells were co-culture with target positive A375-RFP tumor cells at an effector to target ratio of 3:1. Lysis of target positive A375-RFP tumor cells was assessed in real time by IncuCyte® live cell analysis system (Essen BioScience). Tumor cells alone and non-transduced and αβTCR transduced αβ T cells were used as negative and positive controls, respectively.

As shown in FIG. 2E, while non-transduced γδ T cells showed cytotoxic potential due to intrinsic anti-tumor properties of γδ T cells, αβTCR+CD8αβ transduced γδ T cells showed similar cytotoxic potential as compared to αβTCR transduced αβ T cells, indicating that αβTCR+CD8αβ transduced γδ T cells can be engineered to target and kill tumor cells. These data indicate engineered Vγ982 T cells produced by the methods of the present disclosure are functional and can be used to kill target cells, e.g., cancer cells, in a peptide-specific manner.

FIG. 3: IFNγ release from CD8+ T cells electroporated with alpha and beta chain RNA of TCR R11P3D3 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-040 and IFN-041.

FIG. 4: IFNγ release from CD8+ T cells electroporated with alpha and beta chain RNA of TCR R16P1C10 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-046 and IFN-041.

FIG. 5: IFNγ release from CD8+ T cells electroporated with alpha and beta chain RNA of TCR R16P1E8 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-040 and IFN-041.

FIG. 6: IFNγ release from CD8+ T cells electroporated with alpha and beta chain RNA of TCR R17P1A9 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-040 and IFN-041.

FIG. 7: IFNγ release from CD8+ T cells electroporated with alpha and beta chain RNA of TCR R17P1D7 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-040 and IFN-041.

FIG. 8: IFNγ release from CD8+ T cells electroporated with alpha and beta chain RNA of TCR R17P1G3 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-046 and IFN-041.

FIG. 9: IFNγ release from CD8+ T cells electroporated with alpha and beta chain RNA of TCR R17P2B6 after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T cells derived from two different healthy donors. RNA electroporated CD8+ T cells alone or in co-incubation with unloaded target cells served as controls. Different donors were analyzed, IFN-040 and IFN-041.

FIG. 10: IFNγ release from CD8+ T cells electroporated with alpha and beta chain RNA of TCR R11P3D3 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 11: IFNγ release from CD8+ T cells electroporated with alpha and beta chain RNA of TCR R16P1C10 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 12: IFNγ release from CD8+ T cells electroporated with alpha and beta chain RNA of TCR R16P1E8 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 13: IFNγ release from CD8+ T cells electroporated with alpha and beta chain RNA of TCR R17P1D7 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 14: IFNγ release from CD8+ T cells electroporated with alpha and beta chain RNA of TCR R17P1G3 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 15: IFNγ release from CD8+ T cells electroporated with alpha and beta chain RNA of TCR R17P2B6 after co-incubation with T2 target cells loaded with PRAME-004 peptide (SEQ ID NO: 310) in various peptide loading concentrations from 10 μM to 10 pM. IFNγ release data were obtained with CD8+ T cells derived from two different healthy donors. Different donors were analyzed, TCRA-0003 and TCRA-0017.

FIG. 16: HLA-A*02/PRAME-004 (SEQ ID NO: 310) tetramer or HLA-A*02/NYESO1-001 (SEQ ID NO: 311) tetramer staining, respectively, of CD8+ T cells electroporated with alpha and beta chain RNA of TCR R16P1C10. CD8+ T cells electroporated with RNA of 1G4 TCR (SEQ ID: 85-96) that specifically binds to the HLA-A*02/NYESO1-001 (SEQ ID NO: 311) complex and mock electroporated CD8+ T cells served as controls.

FIG. 17: IFNγ release from CD8+ T cells lentivirally transduced with TCR R11P3D3 (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6, and 7) but unrelated peptides ACPL-001, HSPB3-001, UNC7-001, SCYL2-001, RPS2P8-001, PCNXL3-003, AQP6-001, PCNX-001, AQP6-002 TRGV10-001, NECAP1-001, FBXW2-001, or control peptide NYESO1-001 (SEQ ID NO: 311). IFNγ release data were obtained with CD8+ T cells derived from two different healthy donors, D103805 and D191451.

FIG. 18: IFNγ release from CD8+ T cells lentivirally transduced with TCR R11P3D3 after co-incubation with T2 target cells loaded with 100 nM PRAME-004 peptide (SEQ ID NO: 310) or similar (identical to PRAME-004 in positions 3, 5, 6, and 7) but unrelated peptides or control peptide NYESO1-001 (SEQ ID NO: 311). IFNγ release data were obtained with CD8+ T cells derived from two different healthy donors, TCRA-0087 and TCRA-0088.

FIG. 19: IFNγ release from CD8+ T cells lentivirally transduced with TCR R11P3D3 (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (derived from primary melanoma, PRAME-004 high), Hs695T (PRAME-004 medium), U266B1 (derived from peripheral blood of myeloma patient, PRAME-004 very low) and MCF-7 (no PRAME-004) present different amounts of PRAME-004 per cell. T cells alone served as controls. IFNγ release data were obtained with CD8+ T cells derived from two different healthy donors, D103805 and D191451.

FIG. 20: IFNγ release from CD8+ T cells lentivirally transduced with TCR R11P3D3 after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copy numbers of PRAME-004 per cell. T cells alone served as controls. IFNγ release data were obtained with CD8+ T cells derived from two different healthy donors, TCRA-0084 and TCRA-0085.

FIG. 21: IFNγ release from CD8+ T cells lentivirally transduced with enhanced TCR R11P3D3_KE (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6, and 7) but unrelated peptide ACPL-001, HSPB3-001, UNC7-001, SCYL2-001, RPS2P8-001, PCNXL3-003, AQP6-001, PCNX-001, AQP6-002, TRGV10-001, NECAP1-001, FBXW2-001, or control peptide NYESO1-001 (SEQ ID NO: 311). IFNγ release data were obtained with CD8+ T cells derived from two different healthy donors, D103805 and D191451.

FIG. 22: IFNγ release from CD8+ T cells lentivirally transduced with enhanced TCR R11P3D3_KE after co-incubation with T2 target cells loaded with 100 nM PRAME-004 peptide (SEQ ID NO: 310) or similar (identical to PRAME-004 in positions 3, 5, 6 and 7) but unrelated peptides or control peptide NYESO1-001 (SEQ ID NO: 311). IFNγ release data were obtained with CD8+ T cells derived from two different healthy donors, TCRA-0087 and TCRA-0088.

FIG. 23: IFNγ release from CD8+ T cells lentivirally transduced with enhanced TCR R11P3D3_KE (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (PRAME-004 high), Hs695T (PRAME-004 medium), U266B1 (PRAME-004 very low) and MCF-7 (no PRAME-004) present different copy numbers of PRAME-004 per cell. T cells alone served as controls. IFNγ release data were obtained with CD8+ T cells derived from two different healthy donors, D103805 and D191451.

FIG. 24: IFNγ release from CD8+ T cells lentivirally transduced with enhanced TCR R11P3D3_KE after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copy numbers of PRAME-004 per cell. T cells alone served as controls. IFNγ release data were obtained with CD8+ T cells derived from two different healthy donors, TCRA-0084 and TCRA-0085.

FIG. 25: IFNγ release from CD8+ T cells lentivirally transduced with TCR R11P3D3 or enhanced TCR R11P3D3_KE or non-transduced cells after co-incubation with tumor cell lines UACC-257 (PRAME-004 high), Hs695T (PRAME-004 medium), U266B1 (PRAME-004 very low) and MCF-7 (no PRAME-004) present different amounts of PRAME-004 per cells. T cells alone served as controls. IFNγ release of both TCRs correlates with PRAME-004 presentation and R11P3D3_KE induces higher responses compared to R11P3D3.

FIG. 26: Potency assay evaluating cytolytic activity of lentivirally transduced T cells expressing TCR R11P3D3 or enhanced TCR R11P3D3_KE against PRAME-004-positive tumor cells. Cytotoxic response of R11P3D3 and R11P3D3_KE transduced and non-transduced (NT) T cells measured against A-375 (primary skin cancer cell line, PRAME-004 low) or U2OS (Primary Osteosarcoma, PRAME-004 medium) tumor cells. The assays were performed in a 72-hour fluorescence microscopy-based cytotoxicity assay. Results are shown as fold tumor growth over time.

FIG. 27: Potency assay evaluating cytolytic activity of lentivirally transduced T cells expressing TCR R11P3D3 or enhanced TCR R11P3D3_KE against PRAME-004-positive tumor cells. Cytotoxic response of R11P3D3 and R11P3D3_KE transduced and non-transduced (NT) T cells measured against A-375 (PRAME-004 low) or U2OS (PRAME-004 medium) tumor cells. The assays were performed in a 72-hour fluorescence microscopy-based cytotoxicity assay. Results are shown as fold tumor growth over time.

FIG. 28 shows the results of an LDH-release assay with the bispecific TCR/mAb diabody construct IA_5 targeting tumor-associated peptide PRAME-004 (SEQ ID NO: 310) presented on HLA-A*02. CD8-positive T cells isolated from a healthy donor were co-incubated with cancer cell lines UACC-257, SW982 (Primary Synovial Sarcoma cell line) and U2OS presenting differing amounts of PRAME-004:HLA-A*02-1 complexes on the cell surface (approx. 1100, approx. 780 and approx. 240 copies per cell, respectively, as determined by targeted MS analysis) at an effector:target ratio of 5:1 in the presence of increasing concentrations of TCR/mAb diabody molecules. After 48 hours of co-culture target cell lysis was quantified utilizing LDH-release assays according to the manufacturer's instructions (Promega).

FIG. 29 shows the results of an LDH-release assay with the bispecific TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. CD8-positive T cells isolated from a healthy donor were co-incubated with the cancer cell line U2OS presenting approx. 240 copies per cell of PRAME-004:HLA-A*02:1 complexes or non-loaded PRAME-004-negative T2 cells (effector:target ratio of 5:1) in the presence of increasing concentrations of TCR/mAb diabody molecules. After 48 hours of coculture target cell lysis was quantified utilizing LDH-release assays according to the manufacturer's instructions (Promega).

FIG. 30 shows the results of a heat-stress stability study of the TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. For this, the proteins were formulated in PBS at a concentration of 1 mg/mL and subsequently stored at 40° C. for two weeks. Protein integrity and recovery was assessed utilizing HPLC-SEC. Thereby the amount of high-molecular weight species was determined according to percentage of peak area eluting before the main peak. Recovery of monomeric protein was calculated by comparing main peak areas of unstressed and stressed samples.

FIG. 31: Binding kinetics of bispecific molecules comprising different R16P1C10 variants. FAB2G sensors were used for the scTCR-Fab format (20 μg/ml loaded for 120 s), AHC sensors for the diabody-Fc formats (10 μg/ml loaded for 120 s for improved variant; 5 μg/ml loaded for 120 s for stabilized variant, LoAff3, CDR6, HiAff1). Analyzed concentrations of HLA-A*02/PRAME-004 are represented in nM. Graphs show curves of measured data and calculated fits.

FIG. 32: Lysis of PRAME-positive tumor cell lines induced by bispecific molecules containing CDR6, HiAff1 or LoAff3 TCR variants, respectively, in presence of CD8+ T cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 33: Lysis of PRAME-negative tumor cell lines induced by bispecific molecules containing CDR6, HiAff1 or LoAff3 TCR variants, respectively, in presence of CD8+ T cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 34: In vivo efficacy. NOG mice bearing Hs695T tumors of approximately 50 mm³ were transplanted with human PBMCs and treated with PBS (group 1), 0.5 mg/kg body weight HiAff1/antiCD3 diabody-Fc (group 2) or 0.5 mg/kg antiHIV/antiCD3 diabody-Fc (group 3) i.v. twice a week. Tumor volumes were measured with a caliper and calculated by length×width²/2.

FIG. 35: In vitro cytotoxicity of TCER® molecules on target-positive and target-negative tumor cell lines. PBMC from a healthy HLA-A*02-positive donor were incubated with either target-positive tumor cell line Hs695T (●) or target-negative, but HLA-A*02-positive tumor cell line T98G (Glioblastoma cell line (negative control) (∘), respectively, at a ratio of 1:10 in the presence of increasing TCER© concentrations. TCER®-induced cytotoxicity was quantified after 48 hours of co-culture by measurement of released LDH. Results for experiments assessing TPP-93 and TPP-79 are shown in the upper and lower panel, respectively.

FIG. 36: In vitro cytotoxicity of TCER® molecule TPP-105 on target-positive and target-negative tumor cell lines. PBMC from a healthy HLA-A*02-positive donor were incubated with either target-positive tumor cell line Hs695T (●) or target-negative, but HLA-A*02-positive tumor cell line T98G (∘), respectively, at a ratio of 1:10 in the presence of increasing concentrations of TPP-105. TCER®-induced cytotoxicity was quantified after 48 hours of co-culture by measurement of released LDH.

FIG. 37: Summary of cytotoxicity data of TCER® Slot III molecules. EC₅₀ values of dose-response curves obtained in LDH-release assays were calculated utilizing non-linear 4-point curve fitting. For each assessed TCER®-molecule calculated EC₅₀ values on target-positive tumor cell lines Hs695T (●), U2OS (∘), and target-negative but HLA-A*02-positive tumor cell line T98G (*) are depicted. Thereby, each symbol represents one assay utilizing PBMC derived from various HLA-A*02-positive donors. For TPP-871/T98G, the EC₅₀ is estimated, as T98G was not recognized by TPP-871.

FIG. 38: In vitro cytotoxicity of TCER® Slot III variants on T2 cells loaded with different concentrations of target peptide. Cytotoxicity was determined by quantifying LDH released into the supernatants. Human PBMC were used as effector cells at an E:T ratio of 5:1. Read-out was performed after 48 h.

FIG. 39: Normal tissue cell safety analysis for selected TCER® Slot III variants.

TCER®-mediated cytotoxicity against 5 different normal tissue cell types expressing HLA-A*02 was assessed in comparison to cytotoxicity directed against PRAME-004-positive Hs695T tumor cells. PBMCs from a healthy HLA-A*02+ donor were co-cultured at a ratio of 10:1 with the normal tissue cells or Hs695T tumor cells (in triplicates) in a 1:1 mixture of the respective normal tissue cell medium (4, 10a or 13a) and T cell medium (LDH-AM) or in T cell medium alone. After 48 hours, lysis of normal tissue cells and Hs695T cells was assessed by measuring LDH release (LDH-Glo™ Kit, Promega).

FIG. 40: Over-presentation of SEQ ID NO: 310 in different tumor metastases

This Figure shows the over-presentation of SEQ ID NO: 310 in different tumor metastases compared to normal tissues. Upper part: Median MS signal intensities from technical replicate measurements are plotted as dots for single normal (grey dots, left part of Figure) and metastatic samples (black dots, right part of Figure) of the SEQ ID NO: 310 identifications on HLA-A*02. Boxes display median, 25th and 75th percentile of normalized signal intensities, while whiskers extend to the lowest data point still within 1.5 interquartile range (IQR) of the lower quartile, and the highest data point still within 1.5 IQR of the upper quartile. Lower part: The relative peptide detection frequency in every organ is shown as spine plot. Numbers below the panel indicate number of samples on which the peptide was detected out of the total number of samples analyzed for each organ (N=762) or metastatic indication (N=102 for HLA-A*02 positive metastatic samples).

If the peptide has been detected on a sample but could not be quantified for technical reasons, the sample is included in this representation of detection frequency, but no dot is shown in the upper part of the Figure. Tissues (from left to right):

Normal samples: adipose (adipose tissue); adrenal gl (adrenal gland); bile duct; bladder; bloodcells; bloodvess (blood vessels); bone marrow; brain; breast; esoph (esophagus); eye; gall bl (gallbladder); nead&neck; heart; intest. la (large intestine); intest. sm (small intestine); kidney; liver; lung; lymph nodes; nerve cent (central nerve); nerve periph (peripheral nerve); ovary; pancreas; parathyr (parathyroid gland); perit (peritoneum); pituit (pituitary); placenta; pleura; prostate; skel. mus (skeletal muscle); skin; spinal cord; spleen; stomach; testis; thymus; thyroid; trachea; ureter; uterus.

Metastatic samples: BRCA (breast cancer metastasis); CCC (cholangiocellular carcinoma metastasis); CRC (colorectal cancer metastasis); GC (gastric cancer metastasis); HCC (hepatocellular carcinoma metastasis); HNSCC (head and neck squamous cell carcinoma metastasis); MEL (melanoma metastasis); NHL (non-Hodgkin lymphoma metastasis); NSCLCadeno (non-small cell lung cancer adenocarcinoma metastasis); NSCLCsquam (squamous cell non-small cell lung cancer metastasis); OC (ovarian cancer metastasis); OSCAR (esophageal cancer metastasis); PACA (pancreatic cancer metastasis); PRCA (prostate cancer metastasis); RCC (renal cell carcinoma metastasis); SARC (sarcoma metastasis); SCLC (small cell lung cancer metastasis); UBC (urinary bladder carcinoma metastasis); UEC (uterine endometrial cancer metastasis).

FIG. 41: Expression profile of PRAME

Tumor (black dots) and normal (grey dots) samples are grouped according to organ of origin. Box-and-whisker plots represent median value, 25th and 75th percentile (box) plus whiskers that extend to the lowest data point still within 1.5 interquartile range (IQR) of the lower quartile and the highest data point still within 1.5 IQR of the upper quartile. Tissues (from left to right):

Normal samples: adipose (adipose tissue); adrenal gl (adrenal gland); bile duct; bladder; bloodcells; bloodvess (blood vessels); bone marrow; brain; breast; esoph (esophagus); eye; gall bl (gallbladder); nead&neck; heart; intest. la (large intestine); intest. sm (small intestine); kidney; liver; lung; lymph nodes; nerve periph (peripheral nerve); ovary; pancreas; parathyr (parathyroid gland); perit (peritoneum); pituit (pituitary); placenta; pleura; prostate; skel. mus (skeletal muscle); skin; spinal cord; spleen; stomach; testis; thymus; thyroid; trachea; ureter; uterus.

Metastatic samples: AML (acute myeloid leukemia metastasis); BRCA (breast cancer metastasis); CCC (cholangiocellular carcinoma metastasis); CRC (colorectal cancer metastasis); GBC (gallbladder cancer metastasis); GC (gastric cancer metastasis); HCC (hepatocellular carcinoma metastasis); HNSCC (head and neck squamous cell carcinoma metastasis); MEL (melanoma metastasis); NHL (non-Hodgkin lymphoma metastasis); NSCLCadeno (non-small cell lung cancer adenocarcinoma metastasis); NSCLCother (metastasis of NSCLC samples that could not unambiguously be assigned to NSCLCadeno or NSCLCsquam); NSCLCsquam (squamous cell non-small cell lung cancer metastasis); OC (ovarian cancer metastasis); OSCAR (esophageal cancer metastasis); PACA (pancreatic cancer metastasis); PRCA (prostate cancer metastasis); RCC (renal cell carcinoma metastasis); SCLC (small cell lung cancer metastasis); UBC (urinary bladder carcinoma metastasis); UEC (uterine endometrial cancer metastasis).

FIG. 42: Presentation of KRT5-004 (SEQ ID NO: 312) on primary tumors and metastases.

It can be seen that the presentation of SEQ ID NO: 312 is completely lost when comparing HNSCC primary tumors with HNSCC metastases: While SEQ ID NO: 312 is detected in nearly 50% of primary HNSCC tumor samples, it is completely absent in the metastatic HNSCC tumor samples analyzed.

FIG. 43: Presentation of PRAME-004 (SLLQHLIGL) (SEQ ID NO: 310) on normal tissues, primary tumors, and metastatic cancerous tissues.

Metastatic samples: BRCA (breast cancer metastasis); CCC (cholangiocellular carcinoma metastasis); CRC (colorectal cancer metastasis); GC (gastric cancer metastasis); HCC (hepatocellular carcinoma metastasis); HNSCC (head and neck squamous cell carcinoma metastasis); MEL (melanoma metastasis); NHL (non-Hodgkin lymphoma metastasis); NSCLCadeno (non-small cell lung cancer adenocarcinoma metastasis); NSCLCsquam (squamous cell non-small cell lung cancer metastasis); OC (ovarian cancer metastasis); OSCAR (esophageal cancer metastasis metastasis); PACA (pancreatic cancer metastasis); PRCA (prostate cancer metastasis); RCC (renal cell carcinoma metastasis); SARC (sarcoma metastasis); SCLC (small cell lung cancer metastasis); UBC (urinary bladder carcinoma metastasis); UEC (uterine endometrial cancer metastasis).

FIG. 44: Presentation of PRAME-004 (SLLQHLIGL) (SEQ ID NO: 310) on normal tissues and cancerous tissues, which combine primary and metastatic cancerous tissues.

FIG. 45: Presentation of PRAME-004 (SLLQHLIGL) (SEQ ID NO: 310) on normal tissues, primary triple-negative breast cancer (TNBC), and metastases being qualified as TNBC.

FIG. 46: Presentation of PRAME-004 (SLLQHLIGL) (SEQ ID NO: 310) on normal tissues and TNBC, which combine primary TNBC and metastases being qualified as TNBC.

FIG. 47A: Baseline PRAME expression in tumor biopsies obtained from PRAME-positive patients

Patients were involved in a clinical trial, and were treated with engineered T cells expressing PRAME-004-specific TCR. The arrows indicate the PRAME expression of patient 1 and patient 2 who had head and neck adenocarcinomas with best overall response in the trial (see FIG. 47B).

FIG. 47B: Preliminary results of the clinical trial

Patient 1 and patient 2 who had head and neck adenocarcinomas treated with engineered T cells expressing PRAME-004-specific TCR in the trial exhibited 9.7% and 13.1% tumor reduction, respectively, as compared with that at baseline.

FIG. 48: In vivo efficacy in a metastatic pancreatic cancer patient-derived xenograft (PDX) model.

Female NOG mice bearing PAXF 1657 (lung metastasis of pancreatic cancer) tumors of approximately 80 mm³ were transplanted with human PBMCs and treated with 5 mL/kg body weight PBS (group 1, 2) or 0.25 mg/kg body weight TCER© TPP-1295 (group 3, 4) on days 1, 8, and 15. Tumor volumes were measured with a caliper and calculated by (length×width²)/2, length>width.

FIG. 49A: In vivo efficacy in a metastatic non-small cell lung carcinoma patient-derived xenograft (PDX) model.

Female NOG mice bearing LXFL 1176 (lymph node metastasis of non-small cell lung large cell carcinoma) tumors of approximately 80 mm³ were transplanted with human PBMCs and treated with 5 mL/kg body weight PBS (group 1, 2) or 0.25 mg/kg body weight TCER® TPP-1295 (group 3, 4) on days 1, 8, 15, and 22. Tumor volumes were measured with a caliper and calculated by (length×width²)/2, length>width.

FIG. 49B: In vivo efficacy in a metastatic non-small cell lung adenocarcinoma patient-derived xenograft (PDX) model.

Female NOG mice bearing LXFA 1125 (ovary metastasis of non-small cell lung adenocarcinoma) tumors of approximately 80 mm³ were transplanted with human PBMCs and treated with 5 mL/kg body weight PBS (group 1, 2) or 0.25 mg/kg body weight TCER® TPP-1295 (group 3, 4) on days 1, 8, and 15. Tumor volumes were measured with a caliper and calculated by (length×width²)/2, length>width.

FIG. 50: PRAME-004 prevalences in metastatic cancer patients with different tumor indications.

Tumor positivity is determined from tumor biopsy samples of metastatic cancer patients using a dedicated targeted PRAME-004 qPCR assay (IMADetect*). The threshold for PRAME-004 positivity is determined using paired PRAME-004 immunopeptidomics mass spectrometry and exon expression data (Fritsche et al. 2018).

The table in FIG. 50 lists the results of PRAME positivity in patient-derived metastatic tumor samples

| | |
|---|---|
| ≥1-<25% = | + |
| ≥25 = | ++ |
| ≥50 = | +++ |
| ≥75 = | ++++ |

The number of assessed patent-derived metastatic tumor samples is indicated.

PRAME-004 positivity could also be established for the following tumor indications. The number of samples with PRAME positivity is indicated: squamous cell anal carcinoma (5), gastric cancer (2), tonsil cancer (1), bronchial carcinoma (2), mucosal melanoma (1), esophageal melanoma (1), anal melanoma (1), rectal cancer (1), pancreatic neuroendocrine tumor (1), tongue carcinoma (1), malign peripheral nerve sheath tumor (1).

FIG. 51: PRAME-004 prevalences in cancer patients with different tumor indications.

Tumor positivity is determined from tumor biopsy samples of cancer patients analyzed immunohistochemistry staining for PRAME. Tumor samples with a P score ≥1(%) were considered PRAME-positive.

The table in FIG. 51 lists the results of PRAME positivity in patient-derived metastatic tumor samples as assessed by immunohistochemistry

| | |
|---|---|
| ≥1-<25% = | + |
| ≥25 = | ++ |
| ≥50 = | +++ |
| ≥75 = | ++++ |

The number of assessed patent-derived tumor samples is indicated.

FIG. 52 Immunohistochemistry staining of PRAME-positive cancers

Exemplary PRAME-positive tissue sections of anal carcinoma (left image), small cell lung cancer (middle image) and uterine carcinosarcoma (right image).

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1: T Cell Receptor R11P3D3

TCR R11P3D3 (SEQ ID NO: 12-23 and 120) is restricted towards HLA-A*02-presented PRAME-004 (SEQ ID NO: 310) (see FIG. 3).

R11P3D3 specifically recognizes PRAME-004, as human primary CD8+ T cells re-expressing this TCR release IFNγ upon co-incubation with HLA-A*02+ target cells, loaded with PRAME-004 peptide or different peptides showing high degree of sequence similarity to PRAME-004 (FIG. 3). NYESO1-001 (SEQ ID NO: 311) peptide is used as negative control. TCR R11P3D3 has an EC₅₀ of 0.74 nM (FIG. 10) and a binding affinity (K_{D}) of 18-26 μM towards HLA-A*02-presented PRAME-004 (SEQ ID NO: 310).

Re-expression of R11P3D3 in human primary CD8+ T cells leads to selective recognition and killing of HLA-A*02/PRAME-004-presenting tumor cell lines (FIGS. 19, 20, 25, and 27). TCR R11P3D3 does not respond to any of the 25 tested healthy, primary or iPSC-derived cell types (FIGS. 19 and 20) and was tested for cross-reactivity towards further 67 similar peptides (of which 57 were identical to PRAME-004 in positions 3, 5, 6, and 7) but unrelated peptides in the context of HLA-A*02 (FIGS. 3, 17, and 18).

### Example 2: T Cell Receptor R16P1C10

TCR R16P1C10 (SEQ ID NOs: 24-35 and 121) is restricted towards HLA-A*02-presented PRAME-004 (SEQ ID NO: 310) (see FIG. 4).

R16P1C10 specifically recognizes PRAME-004, as human primary CD8+ T cells re-expressing this TCR release IFNγ upon co-incubation with HLA-A*02+ target cells and bind HLA-A*02 tetramers (FIG. 16), respectively, loaded either with PRAME-004 peptide or different peptides showing high degree of sequence similarity to PRAME-004 (FIG. 4). NYESO1-001 (SEQ ID NO: 311) peptide is used as negative control. TCR R16P1C10 has an EC₅₀ of 9.6 nM (FIG. 11).

### Example 3: T Cell Receptor R16P1E8

TCR R16P1E8 (SEQ ID NOs: 36-47 and 122) is restricted towards HLA-A*02-presented PRAME-004 (SEQ ID NO: 310) (see FIG. 5).

R16P1E8 specifically recognizes PRAME-004, as human primary CD8+ T cells re-expressing this TCR release IFNγ upon co-incubation with HLA-A*02+ target cells, loaded either with PRAME-004 peptide or alanine or different peptides showing high degree of sequence similarity to PRAME-004 (FIG. 5). NYESO1-001 (SEQ ID NO: 311) peptide (SLLMWITQV, SEQ ID NO: 311) is used as negative control. TCR R16P1E8 has an EC₅₀ of ˜1 μM (FIG. 12).

### Example 4: T Cell Receptor R17P1A9

TCR R17P1A9 (SEQ ID NOs: 48-59 and 123) is restricted towards HLA-A*02-presented PRAME-004 (SEQ ID NO: 310) (see FIG. 6).

R17P1A9 specifically recognizes PRAME-004, as human primary CD8+ T cells re-expressing this TCR release IFNγ upon co-incubation with HLA-A*02+ target cells, loaded either with PRAME-004 peptide or different peptides showing high degree of sequence similarity to PRAME-004 (FIG. 6). NYESO1-001 (SEQ ID NO: 311) peptide is used as negative control.

### Example 5: T Cell Receptor R17P1D7

TCR R17P1D7 (SEQ ID NOs: 60-71 and 124) is restricted towards HLA-A*02-presented PRAME-004 (SEQ ID NO: 310) (see FIG. 7).

R17P1D7 specifically recognizes PRAME-004, as human primary CD8+ T cells re-expressing this TCR release IFNγ upon co-incubation with HLA-A*02+ target cells, loaded either with PRAME-004 peptide or alanine or different peptides showing high degree of sequence similarity to PRAME-004 (FIG. 7). NYESO1-001 (SEQ ID NO: 311) peptide is used as negative control. TCR R17P1D7 has an EC₅₀ of 1.83 nM (FIG. 13).

### Example 6: T Cell Receptor R17P1G3

TCR R17P1G3 (SEQ ID NOS: 72-83 and 125) is restricted towards HLA-A*02-presented PRAME-004 (SEQ ID NO: 310) (see FIG. 8).

R17P1G3 specifically recognizes PRAME-004, as human primary CD8+ T cells re-expressing this TCR release IFNγ upon co-incubation with HLA-A*02+ target cells, loaded either with PRAME-004 peptide or different peptides showing high degree of sequence similarity to PRAME-004 (FIG. 8). NYESO1-001 (SEQ ID NO: 311) peptide is used as negative control. TCR R17P1G3 has an EC₅₀ of 8.63 nM (FIG. 14).

### Example 7: T Cell Receptor R17P2B6

TCR R17P2B6 (SEQ ID NOS: 84-95 and 126) is restricted towards HLA-A*02-presented PRAME-004 (SEQ ID NO: 310) (see FIG. 9).

R17P2B6 specifically recognizes PRAME-004, as human primary CD8+ T cells re-expressing this TCR release IFNγ upon co-incubation with HLA-A*02+ target cells, loaded either with PRAME-004 peptide or alanine or different peptides showing high degree of sequence similarity to PRAME-004 (FIG. 9). NYESO1-001 (SEQ ID NO: 311) peptide is used as negative control. TCR R17P2B6 has an EC₅₀ of 2.11 nM (FIG. 15) and a binding affinity (K_{D}) of 13 μM towards HLA-A*02-presented PRAME-004.

### Example 8: Enhanced T Cell Receptor R11P3D3_KE

The mutated “enhanced pairing” TCR R11P3D3_KE is introduced as a variant of R11P3D3, where α and β variable domains, naturally bearing αW44/βQ44, have been mutated to αK44/βE44. The double mutation is selected among the list present in PCT/EP2017/081745, herewith specifically incorporated by reference. It is specifically designed to restore an optimal interaction and shape complementarity to the TCR scaffold.

Compared with the parental TCR R11P3D3 the enhanced TCR R11P3D3_KE shows superior sensitivity of PRAME-004 recognition. The response towards PRAME-004-presenting tumor cell lines are stronger with the enhanced TCR R11P3D3_KE compared to the parental TCR R11P3D3 (FIG. 25). Furthermore, the cytolytic activity of R11P3D3_KE is stronger compared to R11P3D3 (FIG. 27). The observed improved functional response of the enhanced TCR R11P3D3_KE is well in line with an increased binding affinity towards PRAME-004, as described in Example 1 (R11P3D3, K_{D}=18-26 μM) and Example 8 (R11P3D3_KE, K_{D}=5.3 μM).

### Example 9: Generation of Cancer-Targeting Bispecific TCR/mAb Diabody Molecules

To further validate the platform capabilities of bispecific TCR/mAb diabody constructs, the TCR-derived variable domains were exchanged with variable domains of a TCR, which was stability/affinity maturated by yeast display according to a method described previously (Smith, Harris, and Kranz 2015). The TCR variable domains specifically bind to the tumor-associated peptide PRAME-004 (SEQ ID NO: 310) bound to HLA-A*02. Furthermore, the variable domains of hUCHT1(Var17), a humanized version of the UCHT1 antibody, was used to generate the PRAME-004-targeting TCR/mAb diabody molecule IA_5 (comprising SEQ ID NO: 131 and SEQ ID NO: 132). Expression, purification, and characterization of this molecule was performed. Purity and integrity of final preparation exceeded 96% according to HPLC-SEC analysis.

Binding affinities of bispecific TCR/mAb diabody constructs towards PRAME-004:HLA-A*02 were determined by biolayer interferometry. Measurements were done on an Octet RED384 system using settings recommended by the manufacturer. Briefly, purified bispecific TCR/mAb diabody molecules were loaded onto biosensors (AHC) prior to analyzing serial dilutions of HLA-A*02/PRAME-004.

The activity of this PRAME-004-targeting TCR/mAb diabody construct with respect to the induction of tumor cell lysis was evaluated by assessing human CD8-positive T cell-mediated lysis of the human cancer cell lines UACC-257, SW982, and U2OS presenting different copy numbers of PRAME-004 peptide in the context of HLA-A*02 on the tumor cell surface (UACC-257-about 1100, SW982-about 780, U2OS-about 240 PRAME-004 copies per cell, as determined by quantitative MS analysis) as determined by LDH-release assay.

As depicted in FIG. 28, the PRAME-004-targeting TCR/mAb diabody construct IA_5 induced a concentration-dependent lysis of PRAME-004 positive tumor cell lines. Even tumor cells U2OS expressing as little as 240 PRAME-004 copy numbers per tumor cell were efficiently lysed by this TCR/mAb diabody molecule. These results further demonstrate that TCR/mAb diabody format is applicable as molecular platform allowing to introduce variable domains of different TCRs as well as variable domains of different T cell recruiting antibodies.

### Example 10: Engineerability of TCR/mAb Diabody Constructs

The variable TCR domains utilized in construct IA_5 were further enhanced regarding affinity towards PRAME-004 and TCR stability, and used for engineering into TCR/mAb diabody scaffold resulting in construct IA_6 (comprising SEQ ID NO: 133 and SEQ ID NO: 134). Expression, purification and characterization of TCR/mAb diabody molecules IA_5 and IA_6 were performed. Purity and integrity of final preparations exceeded 97% according to HPLC-SEC analysis.

Potency of the stability and affinity enhanced TCR/mAb diabody variant IA_6 against PRAME-004 was assessed in cytotoxicity experiments with the tumor cell line U2OS presenting low amounts of PRAME-004:HLA-A*02 or non-loaded T2 cells as target cells and human CD8-positive T cells as effector cells.

As depicted in FIG. 29, the inventors observed an increased cytotoxic potency of the TCR/Ab diabody molecule IA_6 comprising the variable domains of the stability/affinity enhanced TCR variant when compared to the precursor construct IA_5. For both constructs, IA_5 and IA_6, the PRAME-004-dependent lysis could be confirmed as no cytolysis of target-negative T2 cells was detected.

The protein constructs were further subjected to heat-stress at 40° C. for up to two weeks to analyze stability of the PRAME-004-specific TCR/mAb diabody variants IA_5 and IA_6. HPLC-SEC analyses after heat-stress revealed a significantly improved stability of the variant IA_6 when compared to the precursor construct IA_5 (see FIG. 30). The temperature-induced increase of high-molecular species (i.e., eluting before the main peak) of the constructs was less pronounced for IA_6 than for IA_5. In line with this result, the recovery of intact, monomeric protein after heat-stress was 87% and 92% for IA_5 and IA_6, respectively.

These exemplary engineering data demonstrate that the highly potent and stable TCR/mAB diabody constructs can further be improved by incorporating stability/affinity enhanced TCR variable domains resulting in therapeutic proteins with superior characteristics.

### Example 11: Binding Affinities of Maturated TCR Variants

Maturated R16P1C10 TCR variants expressed as soluble bispecific molecules (stabilized, improved: scTCR/antiCD3 Fab format; stabilized, improved, CDR6, HiAff1 and LoAff3: TCR/antiCD3 diabody-Fc format) were analyzed for their binding affinity towards HLA-A*02/PRAME-004 monomers via biolayer interferometry. Measurements were performed on an Octet RED384 system using settings recommended by the manufacturer. Briefly, binding kinetics were measured at 30° C. and 1000 rpm shake speed using PBS, 0.05% Tween-20, 0.1% BSA as buffer. Bispecific molecules were loaded onto biosensors (FAB2G or AHC) prior to analyzing serial dilutions of HLA-A*02/PRAME-004. While a stabilized version of R16P1C10 showed an affinity of approximately 1 μM (1.2 μM as scTCR-Fab, 930 nM as diabody-F_{c}), considerably lower K_{D} values were determined for all variants containing maturated CDRs (Table 5, FIG. 31). To further validate that the affinity of a TCR variant is influenced by the format only to a minor extent, K_{D} values of an affinity-maturated TCR variant were measured as scTCR-Fab or diabody-Fc format. The scTCR-Fab and diabody-Fc formats showed K_{D} values of 10 nM and 8.7 nM, respectively, further highlighting good comparability between the different formats (Table 5, FIG. 31).

### Example 12: Killing of Target-Positive and Target-Negative Tumor Cell Lines

Maturated R16P1C10 TCR variants were expressed as soluble bispecific molecules employing a TCR/antiCD3 diabody-Fc format. The cytotoxic activity of the bispecific molecules against PRAME-positive and PRAME-negative tumor cell lines, respectively was analyzed by LDH-release assay. Therefore, tumor cell lines presenting variable amounts of HLA-A*02/PRAME-004 on the cell surface were co-incubated with CD8+ T cells isolated from two healthy donors in presence of increasing concentrations of bispecific molecules. After 48 hours, lysis of target cell lines was measured utilizing CytoTox 96 Non-Radioactive Cytotoxicity Assay Kits (PROMEGA). As shown in FIG. 32, for all tested PRAME-positive cell lines, highly efficient induction of lysis was detectable and clearly depending on concentration of bispecific molecules. In similar experiments utilizing cell lines expressing HLA-A*02 but not presenting the peptide PRAME-004 at detectable levels, FIG. 33 shows no or only marginal lysis of targets was induced by the bispecific molecules indicating the specificity of the TCR domains.

### Example 13: In Vivo Efficacy

Maturated R16P1C10 TCR variant HiAff1 and a HIV-specific high affinity control TCR were expressed as soluble bispecific molecules employing a TCR/antiCD3 diabody-Fc format. A pharmacodynamic study designed to test the ability of the bispecific TCR molecules in recruiting and directing the activity of human cytotoxic CD3+ T cells against a PRAME-positive tumor cell line Hs695T was performed in the hyper immune-deficient NOG mouse strain. The NOG mouse strain hosted the subcutaneously injected human tumor cell line Hs695T and intravenously injected human peripheral blood mononuclear cell xenografts. Human peripheral blood mononuclear cells (5×10⁶ cells/mouse, intravenous injection) were transplanted within 24 hours when individual tumor volume reached 50 mm³. Treatment was initiated within one hour after transplantation of human blood cells. Four to five female mice per group received intravenous bolus injections (5 mL/kg body weight, twice weekly dosing, up to seven doses, starting one day after randomization) into the tail vein. The injected dose of the PRAME-targeting bispecific TCR molecule was 0.5 mg/kg body weight per injection (group 2), PBS was used in the vehicle control group (group 1) and the HIV-targeting control TCR bispecific molecule (0.5 mg/kg body weight per injection) in the negative control substance group (group 3). At the indicated time points, mean tumor volumes were calculated for every group based on the individual tumor volumes that were measured with a caliper and calculated as length×width²/2. Treatment with PRAME-targeting bispecific TCR molecule inhibited tumor growth as indicated by reduced increase of tumor volume from basal levels (start of randomization) of 65 to 409 mm³ in comparison to the increase observed in the vehicle control group from basal levels of 69 to 1266 mm³ and the negative control substance group from basal levels of 66 to 1686 mm³ at day 23 (FIG. 34).

### Example 14: Production and Characterization of Soluble scTCR-Fab Molecules

The variable domains of TCR that bind the PRAME-004:MHC complex may be selected from the following:
V_{A} comprises or consists of the amino acid sequence of SEQ ID NO: 305; and V_{B}
comprises or consists of the amino acid sequence of SEQ ID NO: 306;
V_{A} comprises or consists of the amino acid sequence of SEQ ID NO: 305; and V_{B} comprises or consists of the amino acid sequence of SEQ ID NO: 307;
V_{A} comprises or consists of the amino acid sequence of SEQ ID NO: 305; and V_{B} comprises or consists of the amino acid sequence of SEQ ID NO: 308;
V_{A} comprises or consists of the amino acid sequence of SEQ ID NO: 309; and V_{B} comprises or consists of the amino acid sequence of SEQ ID NO: 306;
V_{A} comprises or consists of the amino acid sequence of SEQ ID NO: 309; and V_{B} comprises or consists of the amino acid sequence of SEQ ID NO: 307; or
V_{A} comprises or consists of the amino acid sequence of SEQ ID NO: 309; and V_{B} comprises or consists of the amino acid sequence of SEQ ID NO: 306.

Most preferably, V_{A} comprises or consists of the amino acid sequence of SEQ ID NO: 305; and V_{B} comprises or consists of the amino acid sequence of SEQ ID NO: 306.

For targeting of the TCR-CD3 complex, V_{H} and V_{L} domains derived from the CD3-specific, humanized antibody hUCHT1 (Zhu and Carter 1995) can be used, in particular V_{H} and V_{L} domains derived from the UCHT1 variants UCHT1-V17, UCHT1-V17opt, UCHT1-V21, or UCHT1-V23, preferably derived from UCHT1-V17, more preferably a V_{H} comprising or consisting of SEQ ID NO: 193; and a V_{L} comprising or consisting of SEQ ID NO: 192; Alternatively, V_{H} and V_{L} domains derived from the antibody BMA031, which targets the TCRα/β CD3 complex, and humanized versions thereof (Shearman et al. 1991) may be used, in particular V_{H} and V_{L} domains derived from BMA031 variants BMA031(V36) or BMA031(V10), preferably derived from BMA031(V36), more preferably a V_{H} Comprising or consisting of SEQ ID NO: 196; or SEQ ID NO: 198; (A02) or SEQ ID NO: 199; (DO1), or SEQ ID NO: 200; (A02_H90Y) or SEQ ID NO: 201; (DO1_H90Y), and a V_{L} Comprising or consisting of SEQ ID NO: 197; As another alternative, V_{H} and V_{L} domains derived from the CD3E-specific antibody H2C (described in EP 2155783) may be used, in particular a V_{H} comprising or consisting of SEQ ID NO: 202; or SEQ ID NO: 207; (N100D) or SEQ ID NO: 209; (N100E) or SEQ ID NO: 211; (S101A) and a V_{L} comprising or consisting of SEQ ID NO: 204.

### Example 15: Identification and Quantitation of Tumor Associated Peptides Presented on the Cell Surface

### Tissue Samples

Patients' tissues were obtained from: BioIVT (Detroit, MI, USA & Royston, Herts, UK); Bio-Options Inc. (Brea, CA, USA); BioServe (Beltsville, MD, USA); Capital BioScience Inc. (Rockville, MD, USA); Conversant Bio (Huntsville, AL, USA); Cureline Inc. (Brisbane, CA, USA); DxBiosamples (San Diego, CA, USA); Geneticist Inc. (Glendale, CA, USA); Indivumed GmbH (Hamburg, Germany); Kyoto Prefectural University of Medicine (KPUM) (Kyoto, Japan); Osaka City University (OCU) (Osaka, Japan); ProteoGenex Inc. (Culver City, CA, USA); Tissue Solutions Ltd (Glasgow, UK); Universitat Bonn (Bonn, Germany); Asklepios Clinic St. Georg (Hamburg, Germany); Val d'Hebron University Hospital (Barcelona, Spain); Center for cancer immune therapy (CCIT), Herlev Hospital (Herlev, Denmark); Leiden University Medical Center (LUMC) (Leiden, Netherlands); Istituto Nazionale Tumori “Pascale”, Molecular Biology and Viral Oncology Unit (Naples, Italy); Stanford Cancer Center (Palo Alto, CA, USA); University Hospital Geneva (Geneva, Switzerland); University Hospital Heidelberg (Heidelberg, Germany); University Hospital Munich (Munich, Germany); University Hospital Tuebingen (Tuebingen, Germany).

Written informed consents of all patients had been given before surgery or autopsy. Tissues were shock-frozen immediately after excision and stored until isolation of TUMAPs at −70° C. or below.

Isolation of HLA Peptides from Tissue Samples

HLA peptide pools from shock-frozen tissue samples were obtained by immune precipitation from solid tissues according to a slightly modified protocol (Falk et al. 1991; Seeger et al. 1999) using the HLA-A*02-specific antibody BB7.2, the HLA-A, -B, -C-specific antibody w6/32, the HLA-DR-specific antibody L243 and the HLA-DP-specific antibody B7/21, CNBr-activated sepharose, acid treatment, and ultrafiltration.

### Mass Spectrometry Analyses

The HLA peptide pools as obtained were separated according to their hydrophobicity by reversed-phase chromatography (nanoAcquity UPLC system, Waters) and the eluting peptides were analyzed in LTQ Velos and Fusion hybrid mass spectrometers (Thermo) equipped with an ESI source. Peptide pools were loaded directly onto the analytical fused-silica micro-capillary column (75 μm i.d.×250 mm) packed with 1.7 μm C18 reversed-phase material (Waters) applying a flow rate of 400 nL per minute. Subsequently, the peptides were separated using a two-step 180 minute-binary gradient from 10% to 33% B at a flow rate of 300 nL per minute. The gradient was composed of Solvent A (0.1% formic acid in water) and solvent B (0.1% formic acid in acetonitrile). A gold coated glass capillary (PicoTip, New Objective) was used for introduction into the nanoESI source. The LTQ-Orbitrap mass spectrometers were operated in the data-dependent mode using a TOP5 strategy. In brief, a scan cycle was initiated with a full scan of high mass accuracy in the orbitrap (R=30000), which was followed by MS/MS scans also in the orbitrap (R=7500) on the 5 most abundant precursor ions with dynamic exclusion of previously selected ions. Tandem mass spectra were interpreted by SEQUEST at a fixed false discovery rate (q<0.05) and additional manual control. In cases where the identified peptide sequence was uncertain it was additionally validated by comparison of the generated natural peptide fragmentation pattern with the fragmentation pattern of a synthetic sequence-identical reference peptide.

Label-free relative LC-MS quantitation was performed by ion counting i.e., by extraction and analysis of LC-MS features (Mueller et al. 2007). The method assumes that the peptide's LC-MS signal area correlates with its abundance in the sample. Extracted features were further processed by charge state deconvolution and retention time alignment (Mueller et al., 2008; Sturm et al., 2008). Finally, all LC-MS features were cross-referenced with the sequence identification results to combine quantitative data of different samples and tissues to peptide presentation profiles. The quantitative data were normalized in a two-tier fashion according to central tendency to account for variation within technical and biological replicates. Thus, each identified peptide can be associated with quantitative data allowing relative quantification between samples and tissues. In addition, all quantitative data acquired for peptide candidates was inspected manually to assure data consistency and to verify the accuracy of the automated analysis. A presentation profile was calculated showing the mean sample presentation as well as replicate variations. The profiles juxtapose BRCA (breast cancer metastases); CCC (cholangiocellular carcinoma metastases); CRC (colorectal cancer metastases); GC (gastric cancer metastases); HCC (hepatocellular carcinoma metastases); HNSCC (head and neck squamous cell carcinoma metastases); MEL (melanoma metastases); NHL (non-Hodgkin lymphoma metastases); NSCLCadeno (non-small cell lung cancer adenocarcinoma metastases); NSCLCsquam (squamous cell non-small cell lung cancer metastases); OC (ovarian cancer metastases); OSCAR (esophageal cancer metastases); PACA (pancreatic cancer metastases); PRCA (prostate cancer metastases); RCC (renal cell carcinoma metastases); SCLC (small cell lung cancer metastases); UBC (urinary bladder carcinoma metastases); UEC (uterine endometrial cancer metastases) samples to a baseline of normal tissue samples. The presentation profile of SEQ ID NO: 310 is shown in FIG. 40. The plot shows only those identifications of peptides as dots which were made on tissue samples positive for the respective HLA allotype which were processed using HLA-specific antibodies.

Peptide presentation on the various indications for SEQ ID NO: 310 are shown in Table 6. This table lists all indication on which the respective peptide was identified at least once, independent of the HLA typing of the sample or the antibody used to process said sample.

### Example 16: Absolute Quantitation of Tumor-Associated Peptides Presented on Cell Surface

The generation of binders, such as antibodies and/or TCRs, is a laborious process, which may be conducted only for a number of selected targets. In the case of tumor-associated and -specific peptides, selection criteria include, but are not restricted to, exclusiveness of presentation and the density of peptide presented on the cell surface. In addition to the isolation and relative quantitation of peptides as described in the examples, the inventors analyzed absolute peptide copies per cell as described in WO 2016/107740. The quantitation of TUMAP copies per cell in solid tumor samples requires the absolute quantitation of the isolated TUMAP, the efficiency of the TUMAP isolation process, and the cell count of the tissue sample analyzed.

### Peptide Quantitation by Nano LC-MS/MS

For an accurate quantitation of peptides by mass spectrometry, a calibration curve was generated for SEQ ID NO: 310/PRAME-004, using two different isotope labeled peptide variants (one or two isotope-labeled amino acids are included during TUMAP synthesis). These isotope-labeled variants differ from the tumor-associated peptide only in their mass but show no difference in other physicochemical properties (Anderson et al., 2012). For the peptide calibration curve, a series of nano LC-MS/MS measurements was performed to determine the ration of MS/MS signals of titrated (singly isotope-labeled peptide) to constant (doubly isotope labeled peptide) isotope-labeled peptides.

The doubly isotope-labeled peptide, also called internal standard, was further spiked to each MS sample and all MS signals were normalized to the MS signal of the internal standard to level out potential technical variances between MS experiments.

The calibration curves were prepared in at least three different matrices, i.e., HLA peptide eluates from natural samples similar to the routine MS samples, and each preparation was measured in duplicate MS runs. For evaluation, MS signals were normalized to the signal of the internal standard and a calibration curve was calculated by logistic regression.

For the quantitation of tumor-associated peptides from tissue samples, the respective samples were also spiked with the internal standard; the MS signals were normalized to the internal standard and quantified using the peptide calibration curve.

### Efficiency of Peptide-MHC Isolation

As for any protein purification process, the isolation of proteins from tissue samples is associated with a certain loss of the protein of interest. To determine the efficiency of TUMAP isolation, peptide-MHC complexes were generated for all TUMAPs selected for absolute quantitation. To be able to discriminate the spiked from the natural peptide-MHC complexes, single-isotope-labelled versions of the TUMAPs were used, i.e., one isotope-labelled amino acid was included in TUMAP synthesis. These complexes were spiked into the freshly prepared tissue lysates, i.e., at the earliest possible point of the TUMAP isolation procedure, and then captured like the natural peptide-MHC complexes in the following affinity purification. Measuring the recovery of the single-labelled TUMAPs therefore allows conclusions regarding the efficiency of isolation of individual natural TUMAPs.

The efficiency of isolation was analyzed in a small set of samples and was comparable among these tissue samples. In contrast, the isolation efficiency differs between individual peptides. This suggests that the isolation efficiency, although determined in only a limited number of tissue samples, may be extrapolated to any other tissue preparation. However, it is necessary to analyze each TUMAP individually as the isolation efficiency may not be extrapolated from one peptide to others.

### Determination of the Cell Count in Solid, Frozen Tissue

In order to determine the cell count of the tissue samples subjected to absolute peptide quantitation, the inventors applied DNA content analysis. This method is applicable to a wide range of samples of different origin and, most importantly, frozen samples (Alcoser et al., 2011; Forsey and Chaudhuri, 2009; Silva et al., 2013). During the peptide isolation protocol, a tissue sample is processed to a homogenous lysate, from which a small lysate aliquot is taken. The aliquot is divided in three parts, from which DNA is isolated (QiaAmp DNA Mini Kit, Qiagen, Hilden, Germany). The total DNA content from each DNA isolation is quantified using a fluorescence-based DNA quantitation assay (Qubit dsDNA HS Assay Kit, Life Technologies, Darmstadt, Germany) in at least two replicates.

In order to calculate the cell number, a DNA standard curve from aliquots of isolated healthy blood cells from several donors, with a range of defined cell numbers, has been generated. The standard curve is used to calculate the total cell content from the total DNA content from each DNA isolation. The mean total cell count of the tissue sample used for peptide isolation is then extrapolated considering the known volume of the lysate aliquots and the total lysate volume.

### Peptide Copies Per Cell

With data of the aforementioned experiments, the inventors calculated the number of TUMAP copies per cell by dividing the total peptide amount by the total cell count of the sample, followed by division through isolation efficiency. Copy cell number for SEQ ID NO: 310 is shown in Table 7.

TABLE 7
Copy cell number for SEQ ID NO: 310 in different metastases

| Entity | Copies per cell (median) | Number of samples |
|---|---|---|
| Metastases | ++ | 15 |
| BRCA met. | +++ | 2 |
| HNSCC met. | +++ | 5 |
| MEL met. | + | 1 |
| NSCLCadeno met. | +++ | 1 |
| OC met. | ++ | 4 |
| OSCAR met. | + | 2 |
| PRCA met. | + | 1 |

BRCA met. = Breast Cancer metastasis
HNSCC met. = Head and Neck Squamous-Cell Carcinoma metastasis
MEL met. = Melanoma metastasis
NSCLCadeno met. = Non-small cell lung adenocarcinoma metastasis
OC met. = Ovarian Cancer metastasis
OSCAR met. = Esophageal Squamous cell Carcinoma metastasis
PRCA met. = Prostate cancer metastasis

Absolute Copy Numbers:

The table lists the results of absolute peptide quantitation in metastatic samples.

| | |
|---|---|
| ≥1-<25 = | + |
| ≥25 = | ++ |
| ≥50 = | +++ |
| ≥75 = | ++++ |

The number of samples, in which evaluable, high quality MS data are available, is indicated.

A more elaborate disclosure of the method to absolutely quantify the peptides is disclosed in international patent publication WO2016107740A1 and U.S. patent application Ser. No. 14/969,423, the contents of both of which is incorporated herein by reference.

### Example 17: Expression Profiling of Genes Encoding the Peptides of the Invention

Over-presentation or specific presentation of a peptide on tumor cells compared to normal cells is sufficient for its usefulness in immunotherapy, and some peptides are tumor-specific despite their source protein occurring also in normal tissues. Still, mRNA expression profiling adds an additional level of safety in selection of peptide targets for immunotherapies. Especially for therapeutic options with high safety risks, such as affinity-matured TCRs, the ideal target peptide will be derived from a protein that is unique to the tumor and not found on normal tissues.

### RNA Sources and Preparation

Surgically removed tissue specimens were provided as indicated above (see Example 1) after written informed consent had been obtained from each patient. Tumor tissue specimens were snap-frozen immediately after surgery and later homogenized with mortar and pestle under liquid nitrogen. Total RNA was prepared from these samples using TRI Reagent (Ambion, Darmstadt, Germany) followed by a cleanup with RNeasy (QIAGEN, Hilden, Germany); both methods were performed according to the manufacturer's protocol.

Total RNA from healthy human tissues for RNASeq experiments was obtained from: Asterand (Detroit, MI, USA & Royston, Herts, UK); Bio-Options Inc. (Brea, CA, USA); Geneticist Inc. (Glendale, CA, USA); ProteoGenex Inc. (Culver City, CA, USA); Tissue Solutions Ltd (Glasgow, UK).

Total RNA from tumor tissues for RNASeq experiments was obtained from: Asterand (Detroit, MI, USA & Royston, Herts, UK); BioCat GmbH (Heidelberg, Germany); BioServe (Beltsville, MD, USA); Geneticist Inc. (Glendale, CA, USA); Istituto Nazionale Tumori “Pascale” (Naples, Italy); ProteoGenex Inc. (Culver City, CA, USA); University Hospital Heidelberg (Heidelberg, Germany).

Quality and quantity of all RNA samples were assessed on an Agilent 2100 Bioanalyzer (Agilent, Waldbronn, Germany) using the RNA 6000 Pico LabChip Kit (Agilent).

### RNAseq Experiments

Gene expression analysis of tumor and normal tissue RNA samples was performed by next-generation sequencing (RNAseq) by GENEWIZ Germany GmbH (Leipzig, Germany). Briefly, sequencing libraries were prepared from total RNA using the NEBNext® Ultra™ II Directional RNA Library Prep Kit for Illumina according to the manufacturer's instructions (New England Biolabs, Ipswich, MA, USA), which includes mRNA selection, RNA fragmentation, cDNA conversion and addition of sequencing adaptors. For sequencing, libraries were multiplexed and loaded onto the Illumina NovaSeq 6000 sequencer (Illumina Inc., San Diego, CA, USA) according to the manufacturer's instructions, generating a minimum of 80 million 150 bp paired-end raw reads per sample. After quality control, adapter trimming and mapping to the reference genome, RNA reads supporting the peptide were counted and are shown as exemplary expression profiles of peptides of the present invention that are highly overexpressed or exclusively expressed in AML (acute myeloid leukemia metastases); BRCA (breast cancer metastases); CCC (cholangiocellular carcinoma metastases); CRC (colorectal cancer metastases); GBC (gallbladder cancer metastases); GC (gastric cancer metastases); HCC (hepatocellular carcinoma metastases); HNSCC (head and neck squamous cell carcinoma metastases); MEL (melanoma metastases); NHL (non-Hodgkin lymphoma metastases); NSCLCadeno (non-small cell lung cancer adenocarcinoma metastases); NSCLCother (NSCLC samples that could not unambiguously be assigned to NSCLCadeno or NSCLCsquam metastases); NSCLCsquam (squamous cell non-small cell lung cancer metastases); OC (ovarian cancer metastases); OSCAR (esophageal cancer metastases); PACA (pancreatic cancer metastases); PRCA (prostate cancer metastases); RCC (renal cell carcinoma metastases); SCLC (small cell lung cancer metastases); UBC (urinary bladder carcinoma metastases); UEC (uterine endometrial cancer metastases) (FIG. 41).

### Example 18: In Vivo Efficacy in Metastatic Patient-Derived Xenograft Models

TCER® TPP-1295 was subjected to a pharmacodynamic study designed to test the ability of the bispecific TCR molecules in recruiting and directing the activity of human cytotoxic CD3+ T cells against PRAME-positive tumors. Most importantly, these metastases/metastatic tumors were patient-derived xenografts (PDX) offering the opportunity for efficacy testing in a preclinical model with tumor biology as close as possible to the in vivo situation in patients. Main genetic and histological properties of the patient's tumor remain unchanged over a certain period of time (passages in mice) making PDX models superior in comparison to cell line-derived xenografts (CDX) e.g. with regard to the predictive value of patient response (Hidalgo et al. 2014; Johnson et al. 2001; Gillet et al. 2011).

The pharmacodynamic assessment of TCER® TPP-1295 was performed in the hyper immune-deficient NOG mouse strain and for three different metastatic PDX models: PAXF 1657 (lung metastasis of pancreatic cancer), LXFL 1176 (lymph node metastasis of non-small cell lung large cell carcinoma), and LXFA 1125 (ovary metastasis of non-small cell lung adenocarcinoma). Human tumor pieces were implanted subcutaneously (and unilaterally) into the right dorsal flank and tumor volumes were measured with a caliper and calculated by (length×width²)/2. Once individual tumor volumes reached approximately 80 mm³, mice were randomized and humanized with human peripheral blood mononuclear cells (PBMCs) (1×10⁷ cells/mouse, intravenously). To address donor-to-donor variability, PBMCs from two different healthy random donors were used (PBMC donor 1: group 1 and 3; PBMC donor 2: group 2 and 4). Treatment was initiated within 24 hours of randomization and three female mice per group (1-4 for each PDX model) received intravenous bolus injections (5 mL/kg body weight) into the tail vein with weekly dosing (PAXF 1657: days 1, 8, and 15; LXFL 1176: days 1, 8, 15, and 22; LXFA 1125: days 1, 8, and 15). The injected dose of the PRAME-targeting bispecific TCER® molecule TPP-1295 molecule was 0.25 mg/kg body weight per injection (groups 3 and 4), while PBS was used as control vehicle (groups 1 and 2). Individual tumor volumes were measured twice weekly (indicated time points see FIGS. 48, 49A, and 49B). Based on individual tumor volumes, mean tumor volumes were calculated for every group as well as for treatment groups (control vehicle [PBS]: group 1 and 2; TCER® TPP-1295 0.25 mg/kg body weight: group 3 and 4). Treatment with PRAME-targeting bispecific TCER® molecule inhibited tumor growth as indicated by reduced increase of tumor volume from basal levels (start of randomization). In the metastatic pancreatic cancer PDX model PAXF 1657 treated with 0.25 mg/kg TCER® TPP-1295 (group 3 and 4), mean basal tumor volume changed from 81 mm³ (day 0) to 873 mm³ (day 20) in comparison to the increase observed in the vehicle control (PBS; group 1 and 2) from 80 mm³ (basal level on day 0) to 1705 mm³ (day 20) (FIG. 48). In the metastatic non-small cell lung large cell carcinoma PDX model LXFL 1176 treated with 0.25 mg/kg TCER® TPP-1295 (group 3 and 4), mean basal tumor volume changed from 83 mm³ (day 0) to 122 mm³ (day 30) compared with the growth observed in the vehicle control (PBS; group 1 and 2) from 86 mm³ (day 0) to 1065 mm³ (day 30) (FIG. 49A). In the metastatic non-small cell lung adenocarcinoma PDX model LXFA 1125 treated with 0.25 mg/kg TCER® TPP-1295 (group 3 and 4), mean basal tumor volume changed from 145 mm³ (day 0) to 261 mm³ (day 34) compared with the growth observed in the vehicle control (PBS; group 1 and 2) from 144 mm³ (day 0) to 707 mm³ (day 34) (FIG. 49B).

These data plausibly suggest that treatment of metastasis or a metastatic lesion, which are PRAME positive, with the pharmaceutical agents as disclosed herein, is a promising option.

### Example 19: Immunohistochemical (IHC) Staining of PRAME

Staining was done following the manufacturer's instructions on an automated IHC staining system (Leica Bond Max). Staining of FFPE tissue samples was done using the following protocol:
bake at 60° C.
dewax, 3× at 60° C.
alcohol rinse, 3×
bond wash, 3× for 5 minutes each
epitope retrieval, 20 minutes at 100° C.
bond wash, 4× for 3 minutes at 35° C.
peroxide block, 1× for 5 minutes
bond wash, 3× for 5 minutes each
PRAME staining PRAME clone EPR20330, abcam), 15 minutes
bond wash, 3×
post primary (poly-HRP anti-mouse), 8 minutes
bond wash, 3× for 2 minutes
polymer (poly-HRP anti-rabbit IgG), 8 minutes
bond wash, 2× for 2 minutes
deionized water, 1×
DAB define, 10 minutes
Deionized water, 3×
Hemotoxylin, 8 minutes
Deionized water, 1×
Bond wash, 1×
Deionized water, 1×
Dehydration of slides and cover slip with cytoseal
   Results are shown in FIGS. 51 and 52.

### Example 20—TCER® Variants (Slot III)

### Productivity and Stress Stability

DNA constructs coding for selected TCER® variants and the reference TCER® TPP-1109 (SEQ ID NOs: 374 and 375) were used for transfection of CHO-S cells by electroporation (MaxCyte) for transient expression and production of TCER® variants. Productivity and stress stability data were then obtained for the respective TCER® variants. Conditioned cell supernatant was cleared by filtration (0.22 μm) utilizing Sartoclear Dynamics® Lab Filter Aid (Sartorius). Bispecific molecules were purified using an Akta Pure 25 L FPLC system (GE Lifesciences) equipped to perform affinity and size-exclusion chromatography in line. Affinity chromatography was performed on protein L columns (GE Lifesciences) following standard affinity chromatographic protocols. Size exclusion chromatography was performed directly after elution (pH 2.8) from the affinity column to obtain highly pure monomeric protein using Superdex 200 μg 16/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/mL at temperatures of 2-8° C. Final product yield was calculated after completed purification and formulation. Quality of purified bispecific molecules was determined by HPLC-SEC on MabPac SEC-1 columns (5 μm, 4×300 mm) running in 50 mM sodium-phosphate pH 6.8 containing 300 mM NaCl within a Vanquish uHPLC-System. Stress stability testing was performed by incubation of the molecules formulated in PBS for up to two weeks at 40° C. Integrity, aggregate-content as well as monomer-recovery was analyzed by HPLC-SEC analyses as described above. Results are shown in Table 8.

TABLE 8
Summary of productivity and stress stability data obtained
for TCER ® molecules of slot III.

| | | Final | | Monomer |
|---|---|---|---|---|
| | | product | | (%) after |
| TCER ® | | yield | Monomer | 14 days at |
| variant | Recruiter | (mg/L) | (%) | 40° C. |
| TPP-230 | ID4 | 73.8 | 98.83 | 95.13 |
| TPP-669 | BMA31(V36)D01 | 72.9 | 97.83 | 94.66 |
| TPP-1109 | UCHT1-V17 | 13.6 | 98.10 | 92.62 |

### Affinity, Specificity and Potency

Potency of TCER® molecules with respect to killing of HLA-A*02-positive tumor cell lines presenting different levels of PRAME-004 target peptide on their cell surface, was assessed in LDH-release assays. In addition, an HLA-A*02-positive but PRAME-004-negative tumor cell line (e.g. T98G) was assessed to characterize unspecific or off-target activity of the TCER® variants. Tumor cell lines were co-incubated with PBMC effectors derived from healthy HLA-A*02-positive donors at a ratio of 1:10 and in the presence of increasing TCER® concentrations. TCER®-induced cytotoxicity was quantified after 48 hours of co-culture by measurement of released LDH. EC₅₀ values of dose-response curves were calculated utilizing non-linear 4-point curve fitting. EC₅₀ values for two PRAME-004-positive tumor cell lines (Hs695T and U2OS) and a PRAME-004-negative tumor cell line (T98G) were determined in different experiments with different HLA-A*02-positive PBMC donors. The EC₅₀ values for T98G were about 100× increased compared to that of Hs695T and U2OS.

TCER® Slot III variants TPP-230 and TPP-669 were analyzed for their binding affinity to the target peptide-HLA complex (HLA-A*02/PRAME-004) via bio-layer interferometry. Measurements were performed on an Octet HTX system at 30° C. Assays were run at a sensor offset of 3 mm and an acquisition rate of 5 Hz on HIS1K biosensors in 16-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (300 s, twofold serial dilution of TCER® ranging from 100 nM to 1.56 nM or 50 nM to 0.78 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). Strong binding affinities were found (Table 9). Furthermore, binding affinities were determined for four previously identified potential off-target peptides: SMARCD1-001 (SEQ ID NO: 370), VIM-009 (SEQ ID NO: 371), FARSA-001 (SEQ ID NO: 372) and GIMAP8-001 (SEQ ID NO: 373). K_{D} windows were calculated compared to binding of the target peptide-HLA. Measurements were performed on an Octet RED384 or HTX system at 30° C. Assays were run at a sensor offset of 3 mm and an acquisition rate of 5 Hz on HIS1K biosensors in 16-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (300 s, twofold serial dilution of TCER® ranging from 500 nM to 7.81 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with the respective peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). Overall, considerable weaker binding to the potential off-target peptides compared to target peptide was found for all variants showing windows of at least 60-fold to even no binding at all. For VIM-009, the smallest measured K_{D} windows were >100-fold (Table 9). Thus, binding to VIM-009 is not relevant and affinity determination of NOMAP-3-1408 binding was not considered necessary based on its binding signals comparable to VIM-009. For one interaction, a K_{D} window of 50-fold was calculated. However, for this interaction and also several others, the Rₘₐₓ value calculated by the fitting algorithm was too low, so that the interaction is assumed to be weaker than calculated and thus the window larger. Respective interactions are indicated in Table 9. To further analyze specificity of the different variants, binding motifs were determined by measuring the affinities for the target peptide-HLA complex as well as for the alanine-substituted variants for positions 1, 3, 4, 5, 6, 7, 8. Measurements were performed on an Octet HTX system at 30° C. Assays were run at a sensor offset of 3 mm and an acquisition rate of 5 Hz on HIS1K biosensors in 16- or 8-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (150 s, twofold serial dilution of TCER® ranging from 400 nM to 6.25 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with the respective peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). A position was considered part of the binding motif for an at least 2-fold reduction in affinity or binding signal (measured for the highest concentration analyzed). All tested TCER® variants showed broad binding motifs recognizing at least four and up to all analyzed peptide positions (Table 10). Positive effects on the binding motif were observed for bA84, aN114L and bA110S/bT115A, which is in accordance with previous data. For comparison, the binding motif of an alternative PRAME-004-targeting TCER® reference molecule (TPP-1109, SEQ ID NOs: 374 and 375) was analyzed. This TCER® recognized positions 5-8 of the peptide and thus binding is limited to this peptide stretch, while positions recognized by TCER® Slot III variants are more evenly distributed throughout the whole peptide.

TCER® Slot III variants TPP-230 and TPP-669 were additionally characterized for their ability to kill T2 cells loaded with varying levels of target peptide. After loading of the T2 cells with the respective concentrations of PRAME-004 for 2 h, peptide-loaded T2 cells were co-cultured with human PBMCs at an E:T ratio of 5:1 in the presence of increasing concentrations of TCER® variants for 48 h. Levels of LDH released into the supernatant were quantified using CytoTox 96 Non-Radioactive Cytotoxicity Assay Kit (Promega). All TCER® variants showed potent killing of PRAME-004-loaded T2 cells with subpicomolar EC₅₀ values at a peptide loading concentration of 10 nM (Table 11). EC₅₀ values increased for decreasing PRAME-004 loading levels. However, even at a very low PRAME-004 loading concentration of 10 pM, killing was induced by TCER® variants TPP-230 and TPP-669.

TABLE 9
K_{D} values for binding to HLA-A*02/PRAME-004 and K_{D} windows of four selected off-target peptides
measured via bio-layer interferometry for TCER ® Slot III variants.

| TCER ® | | PRAME-004 | K_{D} FARSA-001/ | K_{D} GIMAP8-001/ | K_{D} SMARCD1-001/ | K_{D} VIM-009/ |
|---|---|---|---|---|---|---|
| variant | Recruiter | K_{D} (M) | K_{D} PRAME-004 | K_{D} PRAME-004 | K_{D} PRAME-004 | K_{D} PRAMR-004 |
| TPP-230 | ID4 | 3.05E−09 | — | 120¹ | 130¹ | — |
| TPP-669 | BMA031 | 3.65E−09 | 83¹ | 50¹ | 84 | 165 |
| | (V36)D01 | | | | | |

¹K_{D} windows are expected to be higher than the values given in the table (calculated Rₘₐₓ values for these interactions are too low due to overall low binding signals).

TABLE 10
K_{D} values for binding to HLA-A*02/PRAME-004 and K_{D} windows of Ala-substituted
peptide variants for binding motif determination measured via bio-layer interferometry
for TCER ® Slot III variants. For position 5, a threshold of 100 is given for the K_{D}
window. Recognition of this position is at least 100-fold.

| TCER ® | | PRAME-004 | | K_{D} Ala/target | | | | | | |
|---|---|---|---|---|---|---|---|---|---|---|
| variant | Recruiter | K_{D,motif} (M) | Binding motif | A1 | A3 | A4 | A5 | A6 | A7 | A8 |
| TPP-230 | ID4 | 3.03E−09 | -x3-5678x | 1.2 | 12.2 | 1.7 | 100.0 | 3.9 | 25.5 | 3.0 |
| TPP-669 | BMA031 | 3.28E−09 | -x3-5678x | 1.1 | 9.1 | 1.2 | 100.0 | 2.5 | 11.0 | 2.4 |
| | (V36)D01 | | | | | | | | | |
| TPP-1109 | UCHT1- | 2.47E−09 | -x-5678x | 0.9 | 0.8 | 1.2 | 49.0 | 7.9 | 55.7 | 4.1 |
| | V17 | | | | | | | | | |

TABLE 11
In vitro cytotoxicity of TCER ® Slot III variants on PRAME-004-loaded T2 cells.
T2 cells were co-cultured with human PBMCs at an E:T ratio of 5:1 for 48 h.
PRAME-004 loading concentrations are indicated. EC₅₀ values and cytotoxicity
levels in the plateau (Top) were calculated using non-linear 4-point curve fitting.

| | | 10 nM | | 1 nM | | 100 pM | | 10 pM | |
|---|---|---|---|---|---|---|---|---|---|
| | | PRAME-004 | | PRAME-004 | | PRAME-004 | | PRAME-004 | |
| TCER ® | | EC₅₀ | | EC₅₀ | | EC₅₀ | | EC₅₀ | |
| variant | Recruiter | [pM] | Top | [pM] | Top | [pM] | Top | [pM] | Top |
| TPP-230 | ID4 | 0.09 | 109 | 0.9 | 139 | 23.2¹ | 179 | 145 | 80 |
| TPP-669 | BMA031 | 0.22 | 124 | 3.2 | 108 | 84.0 | 126 | 246 | 31 |
| | (V36)D01 | | | | | | | | |

¹High variability within replicates do not allow for reliable EC₅₀ calculation.

### Safety Assessment

The safety profile of the TCER® molecule TPP-230 was assessed in killing experiments with astrocytes and cardiomyocytes (derived from induced pluripotent stem cells) as well as aortic endothelial cells, mesenchymal stem cells and tracheal smooth muscle cells. Co-cultures of the above normal cell types (all expressing HLA-A*02) with PBMC effector cells from a healthy HLA-A*02+ donor were performed at a ratio of 1:10 (target cells:effector cells) in presence of increasing TCER® concentrations. The cells were co-cultured in a 1:1 mixture of the respective normal tissue cell medium and T cell medium or in T cell medium alone (LDH-AM). After 48 h of co-culture, supernatants were harvested and TCER®-induced normal tissue cell lysis was assessed by measuring lactate dehydrogenase (LDH) release with the LDH-Glo™ Kit (Promega). To determine a safety window, the TCER® molecules were co-incubated in an identical setup with the PRAME-004-positive tumor cell line Hs695T in the respective 1:1 mixture of normal tissue cell medium and T cell medium followed by the assessment of LDH release.

No cytotoxicity against normal tissue cells was observed with TPP-230 even at the highest TCER® concentration of 100 nM. When compared to Hs695T tumor cells that showed pronounced lysis at 100 pM for the tested TCER® molecule and even lysis at 10 pM concentration, the normal tissue cell lysis at 100 nM concentration indicates a safety window of more than 1,000-fold for TPP-230.

### Example 21—TCER® Variants (Slot IV)

### Productivity and Stress Stability

DNA constructs coding for selected TCER® variants were used for transfection of CHO-S cells by electroporation (MaxCyte) for transient expression and production of TCER® variants. Productivity and stress stability data were then obtained for the respective TCER® variants. Purification, formulation and initial characterization of molecules (productivity and stress stability) was performed as outlined above in example 20. Results are shown in Table 12.

TABLE 12
Summary of productivity and stress stability data obtained
for TCER ® molecules of slot IV.

| | | Final | | Monomer |
|---|---|---|---|---|
| | | product | | (%) after |
| TCER ® | | yield | Monomer | 14 days at |
| variant | Recruiter | (mg/L) | (%) | 40° C. |
| TPP-1295 | BMA031(V36)D01_H90Y | 56.5 | 94.89 | 91.49 |
| TPP-1298 | BMA031(V36)D01 | 68.1 | 94.41 | 89.7 |
| TPP-1333 | ID4 variant | 61.1 | 98.52 | 95.51 |

### Affinity, Specificity and Potency

Potency of TCER® molecules with respect to killing of HLA-A*02-positive tumor cell lines presenting different levels of PRAME-004 target peptide on their cell surface, was assessed in LDH-release assays. In addition, an HLA-A*02-positive but PRAME-004-negative tumor cell line (e.g. T98G) was assessed to characterize unspecific or off-target activity of the TCER® variants. Tumor cell lines were co-incubated with PBMC effectors derived from healthy HLA-A*02-positive donors at a ratio of 1:10 and in the presence of increasing TCER® concentrations. TCER®-induced cytotoxicity was quantified after 48 hours of co-culture by measurement of released LDH. EC₅₀ values of dose-response curves were calculated utilizing non-linear 4-point curve fitting. EC₅₀ values for a PRAME-004-positive tumor cell lines U2OS and a PRAME-004-negative tumor cell line (T98G) were determined in different experiments with different PBMC donors and are summarized in table 13.

TABLE 13
Summary of LDH-release assay data obtained for TCER ® molecules of slot IV.

| TCER ® | EC₅₀ [pM] for HBC- | EC₅₀ [pM] for HBC- | EC₅₀ [pM] for HBC- | EC₅₀ [pM] for HBC- |
|---|---|---|---|---|
| variant | 1005 vs U2OS | 1005 vs T98G | 848 vs U2OS | 848 vs T98G |
| TPP-1295 | 150 | >100,000 | 663 | >100,000 |
| TPP-1298 | 48 | 37,953 | 249 | >100,000 |
| TPP-1333 | 226 | >100,000 | 719 | >100,000 |

TCER® Slot IV variants TPP-1295, TPP-1298 and TPP-1333 were analyzed for their binding affinity to the target peptide-HLA complex (HLA-A*02/PRAME-004) via bio-layer interferometry. Measurements were performed on an Octet HTX system at 30° C. Assays were run at a sensor offset of 3 mm and an acquisition rate of 5 Hz on HIS1K biosensors in 16-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (300 s, twofold serial dilution of TCER® ranging from 100 nM to 1.56 nM or 50 nM to 0.78 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). Strong binding affinities were found (Table 14). Furthermore, binding affinities were determined for two previously identified potential off-target peptides: IFIT-001 and MCMB-002. K_{D} windows were calculated compared to binding of the target peptide-HLA. Measurements were performed on an Octet RED384 or HTX system at 30° C. Assays were run at a sensor offset of 3 mm and an acquisition rate of 5 Hz on HIS1K biosensors in 16-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (300 s, twofold serial dilution of TCER® ranging from 500 nM to 7.81 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with the respective peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). Overall, considerable weaker binding to the potential off-target peptides compared to target peptide was found for all variants showing windows of at least 10-fold to even no binding at all. Respective interactions are indicated in Table 14. To further analyze specificity of the variants TPP-1295, TPP-1298 and TPP-1333, binding motifs were determined by measuring the affinities for the target peptide-HLA complex as well as for the alanine-substituted variants for positions 1, 3, 4, 5, 6, 7, 8. Measurements were performed on an Octet HTX system at 30° C. Assays were run at a sensor offset of 3 mm and an acquisition rate of 5 Hz on HIS1K biosensors in 16- or 8-channel mode using PBS, 0.05% Tween-20, 0.1% BSA as assay buffer. The following assay step sequence was repeated to measure all binding affinities: regeneration (5 s, 10 mM glycine pH 1.5)/neutralization (5 s, assay buffer; one regeneration cycle consists of four repeats of regeneration/neutralization), baseline (60 s, assay buffer), loading (120 s, 10 μg/ml peptide-HLA), baseline (120 s, assay buffer), association (150 s, twofold serial dilution of TCER® ranging from 400 nM to 6.25 nM, assay buffer as reference), dissociation (300 s, assay buffer). Data evaluation was done using Octet Data Analysis HT Software. Reference sensor subtraction was performed to subtract potential dissociation of peptide-HLA loaded onto the biosensor (via a biosensor loaded with the respective peptide-HLA measured in buffer). Data traces were aligned to baseline (average of the last 5 s), inter-step correction was done to the dissociation step, Savitzky-Golay filtering was applied and curves were fitted globally using a 1:1 binding model (with Rₘₐₓ unlinked by sensor). A position was considered part of the binding motif for an at least 2-fold reduction in affinity or binding signal (measured for the highest concentration analyzed). All tested TCER® variants showed broad binding motifs recognizing at least five and up to all analyzed peptide positions (Table 15).

TABLE 14
K_{D} values for binding to HLA-A*02/PRAME-004 and K_{D} windows
of two selected off-target peptides measured via bio-
layer interferometry for TCER ® Slot IV variants.

| TCER ® | PRAME-004 | K_{D} IFIT-001/ | K_{D} MCMB-002/ |
|---|---|---|---|
| variant | K_{D} (M) | K_{D} PRAME-004 | K_{D} PRAME-004 |
| TPP-1295 | 3.39E−09 | 45.2 | 28.6 |
| TPP-1298 | 2.47E−09 | 24.1 | 17.2 |
| TPP-1333 | 2.94E−09 | 27.3 | 16.0 |

TABLE 15
K_{D} values for binding to HLA-A*02/PRAME-004 and K_{D} windows of Ala-substituted
peptide variants for binding motif determination measured via bio-layer interferometry
for TCER ® Slot IV variants. For position 5, a threshold of 100 is given for the K_{D}
window. Recognition of this position is at least 100-fold.

| TCER ® | PRAME-004 | Binding | K_{D} Ala/target | | | | | | |
|---|---|---|---|---|---|---|---|---|---|
| variant | K_{D,motif} (M) | motif | A1 | A3 | A4 | A5 | A6 | A7 | A8 |
| TPP-1295 | 3.87E−09 | 1x345678x | 2.2 | 21.8 | 2.8 | 20.7 | 5.2 | 35.3 | 5.0 |
| TPP-1298 | 2.87E−09 | -x3-5678x | 1.4 | 10.3 | 1.6 | 100.0 | 2.9 | 9.6 | 2.8 |
| TPP-1333 | 2.60E−09 | -x3-5678x | 1.4 | 12.8 | 2.0 | 100.0 | 3.9 | 21.0 | 3.7 |

### Safety Assessment

The safety profile of the TCER® molecules TPP-1295, TPP-1298 and TPP-1333 was assessed in killing experiments with astrocytes, GABAergic neurons and cardiomyocytes (derived from induced pluripotent stem cells; iHA, iHN and iHCM, respectively) as well as pulmonary fibroblasts (HPF), cardiac microvascular endothelial cells (HCMEC), dermal microvascular endothelial cells (HDMEC), aortic endothelial cells (HAoEC), coronary artery smooth muscle cells (HCASMC), renal cortical epithelial cells (HRCEpC) and tracheal smooth muscle cells (HTSMC). Furthermore, TPP-669 from slot III was tested. Co-cultures of the above normal cell types (all expressing HLA-A*02) with PBMC effector cells from a healthy HLA-A*02+ donor were perfomed at a ratio of 1:10 (target cells:effector cells) in presence of increasing TCER® concentrations. The cells were co-cultured in a 1:1 mixture of the respective normal tissue cell medium and T cell medium or in T cell medium alone (LDH-AM). After 48 h of co-culture, supernatants were harvested and TCER®-induced normal tissue cell lysis was assessed by measuring LDH release with the LDH-Glo™ Kit (Promega). To determine a safety window, the TCER® molecules were co-incubated in an identical setup with the PRAME-004-positive tumor cell line Hs695T in the respective 1:1 mixture of normal tissue cell medium and T cell medium followed by the assessment of LDH release.

No cytotoxicity against normal tissue cells was observed for any of the tested molecules until a concentration of 10 nM TCER®. When compared to Hs695T tumor cells that showed pronounced lysis at 100 pM for all tested TCER® molecules and for some molecules even lysis at 10 pM concentration, the normal tissue cell lysis at 100 nM concentration indicates a safety window of more than 1,000-fold (TPP-1295, TPP-1298).

### REFERENCES

Allison, James P., and Matthew F. Krummel. 1995. “The Yin and Yang of T Cell Costimulation.” Science 270 (5238): 932-932.
Brossart, Peter, and Michael J Bevan. 1997. “Presentation of Exogenous Protein Antigens on Major Histocompatability Complex Class I Molecules by Dendritic Cells: Pathway of Presentation and Regulation by Cytokines.” Blood 90 (4): 1594-99.
Campo, Ana B. del, Jon Amund Kyte, Javier Carretero, Svitlana Zinchencko, Rosa Méndez, Gloria Gonzalez-Aseguinolaza, Francisco Ruiz-Cabello, et al. 2014. “Immune Escape of Cancer Cells with Beta2-microglobulin Loss over the Course of Metastatic Melanoma.” International Journal of Cancer 134 (1): 102-13.
Chang, A. Y., T. Dao, R. S. Gejman, C. A. Jarvis, A. Scott, L. Dubrovsky, M. D. Mathias, et al. 2017. “A Therapeutic T Cell Receptor Mimic Antibody Targets Tumor-Associated PRAME Peptide/HLA-I Antigens.” J Clin Invest 127 (7): 2705-18.
Dash, P., A. J. Fiore-Gartland, T. Hertz, G. C. Wang, S. Sharma, A. Souquette, J. C. Crawford, et al. 2017. “Quantifiable Predictive Features Define Epitope-Specific T Cell Receptor Repertoires.” Nature 547 (7661): 89-93.
Dhatchinamoorthy, Karthik, Jeff D. Colbert, and Kenneth L. Rock. 2021. “Cancer Immune Evasion Through Loss of MHC Class I Antigen Presentation.” Frontiers in Immunology 12: 636568.
Dunbar, James, and Charlotte M. Deane. 2016. “ANARCI: Antigen Receptor Numbering and Receptor Classification.” Bioinformatics 32 (2): 298-300.
Dunbar, James, Bernhard Knapp, Angelika Fuchs, Jiye Shi, and Charlotte M. Deane. 2014. “Examining Variable Domain Orientations in Antigen Receptors Gives Insight into TCR-Like Antibody Design.” PLoS Computational Biology 10 (9): e1003852.
Falk, K., O. Rotzschke, S. Stevanovic, G. Jung, and H. G. Rammensee. 1991. “Allele-Specific Motifs Revealed by Sequencing of Self-Peptides Eluted from MHC Molecules.” Nature 351 (6324): 290-96.
Fares, Jawad, Mohamad Y. Fares, Hussein H. Khachfe, Hamza A. Salhab, and Youssef Fares. 2020. “Molecular Principles of Metastasis: A Hallmark of Cancer Revisited.” Signal Transduction and Targeted Therapy 5 (1): 28.
Fritsche, J., B. Rakitsch, F. Hoffgaard, M. Romer, H. Schuster, D. J. Kowalewski, M. Priemer, et al. 2018. “Translating Immunopeptidomics to Immunotherapy-Decision-Making for Patient and Personalized Target Selection.” Proteomics 18 (12): e1700284.
Fuessel, S., A. Meye, M. Schmitz, S. Zastrow, C. Linne, K. Richter, B. Lobel, et al. 2006. “Vaccination of Hormone-Refractory Prostate Cancer Patients with Peptide Cocktail-Loaded Dendritic Cells: Results of a Phase I Clinical Trial.” Prostate 66 (8): 811-21.
Furukawa, T, T Kubota, H Tanino, S Oura, S Yuasa, H Murate, K Morita, K Kozakai, T Yano, and R M Hoffman. 2000. “Chemosensitivity of Breast Cancer Lymph Node Metastasis Compared to the Primary Tumor from Individual Patients Tested in the Histoculture Drug Response Assay.” Anticancer Research 20 (5C): 3657-58.
Gillet, Jean-Pierre, Anna Maria Calcagno, Sudhir Varma, Miguel Marino, Lisa J. Green, Meena I. Vora, Chirayu Patel, et al. 2011. “Redefining the Relevance of Established Cancer Cell Lines to the Study of Mechanisms of Clinical Anti-Cancer Drug Resistance.” Proceedings of the National Academy of Sciences 108 (46): 18708-13.
Glanville, J., H. Huang, A. Nau, O. Hatton, L. E. Wagar, F. Rubelt, X. Ji, et al. 2017. “Identifying Specificity Groups in the T Cell Receptor Repertoire.” Nature 547 (7661): 94-98.
Gouttefangeas, C., and H. G. Rammensee. 2018. “Personalized Cancer Vaccines: Adjuvants Are Important, Too.” Cancer Immunol Immunother 67 (12): 1911-18.
Hanahan, D., and R. A. Weinberg. 2000. “The Hallmarks of Cancer.” Cell 100: 57-70.
Hanahan, D., and R. A. Weinberg. 2011. “Hallmarks of Cancer: The next Generation.” Cell 144 (5): 646-74.
He, Q., Z. Liu, Z. Liu, Y. Lai, X. Zhou, and J. Weng. 2019. “TCR-like Antibodies in Cancer Immunotherapy.” J Hematol Oncol 12 (1): 99.
Hidalgo, Manuel, Frederic Amant, Andrew V. Biankin, Eva Budinská, Annette T. Byrne, Carlos Caldas, Robert B. Clarke, et al. 2014. “Patient-Derived Xenograft Models: An Emerging Platform for Translational Cancer Research.” Cancer Discovery 4 (9): 998-1013.
Høydahl, Lene Stskken, Rahel Frick, Inger Sandlie, and Geir Åge Løset. 2019. “Targeting the MHC Ligandome by Use of TCR-Like Antibodies.” Antibodies 8 (2): 32.
Johnson, J I, S Decker, D Zaharevitz, L V Rubinstein, J M Venditti, S Schepartz, S Kalyandrug, et al. 2001. “Relationships between Drug Activity in NCI Preclinical in Vitro and in Vivo Models and Early Clinical Trials.” British Journal of Cancer 84 (10): 1424-31.
Köhler, G., and C. Milstein. 1975. “Continuous Cultures of Fused Cells Secreting Antibody of Predefined Specificity.” Nature 256 (5517): 495-97.
Krieg, A. M. 2006. “Therapeutic Potential of Toll-like Receptor 9 Activation.” Nat Rev Drug Discov 5 (6): 471-84.
Lefranc, Marie-Paule, Christelle Pommié, Manuel Ruiz, Véronique Giudicelli, Elodie Foulquier, Lisa Truong, Valérie Thouvenin-Contet, and Gérard Lefranc. 2003. “IMGT Unique Numbering for Immunoglobulin and T Cell Receptor Variable Domains and Ig Superfamily V-like Domains.” Developmental & Comparative Immunology 27 (1): 55-77.
Ling, Agnes, Anna Lö\ddot{f}gren-Burstram, Pär Larsson, Xingru Li, Maria L. Wikberg, Åke Öberg, Roger Stenling, Sofia Edin, and Richard Palmqvist. 2017. “TAP1 Down-Regulation Elicits Immune Escape and Poor Prognosis in Colorectal Cancer.” Oncolmmunology 6 (11): 00-00.
Liu, Yang, and Xuetao Cao. 2016. “Characteristics and Significance of the Pre-Metastatic Niche.” Cancer Cell 30 (5): 668-81.
McGranahan, Nicholas, Rachel Rosenthal, Crispin T. Hiley, Andrew J. Rowan, Thomas B. K. Watkins, Gareth A. Wilson, Nicolai J. Birkbak, et al. 2017. “Allele-Specific HLA Loss and Immune Escape in Lung Cancer Evolution.” Cell 171 (6): 1259-1271.e11.
Moon, Jeong, Jaewoo Lim, Seoyoung Lee, Hye Young Son, Hyun Wook Rho, Hongki Kim, Hyunju Kang, et al. 2020. “Urinary Exosomal MRNA Detection Using Novel Isothermal Gene Amplification Method Based on Three-Way Junction.” Biosensors and Bioelectronics 167: 112474.
Mueller, L. N., O. Rinner, A. Schmidt, S. Letarte, B. Bodenmiller, M. Y. Brusniak, O. Vitek, R. Aebersold, and M. Muller. 2007. “SuperHirn—a Novel Tool for High Resolution LC-MS-Based Peptide/Protein Profiling.” Proteomics 7 (19): 3470-80.
Rammensee, Hans-Georg, Karl-Heinz Wiesmuller, P. Anoop Chandran, Henning Zelba, Elisa Rusch, Cécile Gouttefangeas, Daniel J. Kowalewski, et al. 2019. “A New Synthetic Toll-like Receptor 1/2 Ligand Is an Efficient Adjuvant for Peptide Vaccination in a Human Volunteer.” Journal for Immunotherapy of Cancer 7 (1): 307.
Reiter, Yoram, Ulrich Brinkmann, Robert J Kreitman, Sun-Hee Jung, Byungkook Lee, and Ira Pastan. 1994. “Stabilization of the Fv Fragments in Recombinant Immunotoxins by Disulfide Bonds Engineered into Conserved Framework Regions.” Biochemistry 33 (18): 5451-59.
Riggio, Alessandra I., Katherine E. Varley, and Alana L. Welm. 2021. “The Lingering Mysteries of Metastatic Recurrence in Breast Cancer.” British Journal of Cancer 124 (1): 13-26.
Rock, K. L., S. Gamble, and L. Rothstein. 1990. “Presentation of Exogenous Antigen with Class I Major Histocompatibility Complex Molecules.” Science 249: 918-21.
Rudolph, Markus G., Robyn L. Stanfield, and Ian A. Wilson. 2006. “HOW TCRS BIND MHCS, PEPTIDES, AND CORECEPTORS.” Annual Review of Immunology 24 (1): 419-66.
Seeger, F. H., M. Schirle, J. Gatfield, D. Arnold, W. Keilholz, P. Nickolaus, H. G. Rammensee, and S. Stevanovic. 1999. “The HLA-A*6601 Peptide Motif: Prediction by Pocket Structure and Verification by Peptide Analysis.” Immunogenetics 49: 571-76.
Shearman, C W, D Pollock, G White, K Hehir, G P Moore, E J Kanzy, and R Kurrle. 1991. “Construction, Expression and Characterization of Humanized Antibodies Directed against the Human Alpha/Beta T Cell Receptor.” Journal of Immunology (Baltimore, Md.: 1950) 147 (12): 4366-73.
Smith, S. N., D. T. Harris, and D. M. Kranz. 2015. “T Cell Receptor Engineering and Analysis Using the Yeast Display Platform.” Methods Mol. Biol 1319: 95-141.
Storz, Ulrich. 2015. “Antibody-Drug Conjugates: Intellectual Property Considerations.” MAbs 7 (6): 989-1009.
Watkins, Thomas B. K., Emilia L. Lim, Marina Petkovic, Sergi Elizalde, Nicolai J. Birkbak, Gareth A. Wilson, David A. Moore, et al. 2020. “Pervasive Chromosomal Instability and Karyotype Order in Tumour Evolution.” Nature 587 (7832): 126-32.
Wei, Hudie, Haiyan Cai, Yuhao Jin, Pilin Wang, Qingqing Zhang, Yihui Lin, Weixiao Wang, et al. 2017. “Structural Basis of a Novel Heterodimeric Fc for Bispecific Antibody Production.” Oncotarget 5 (0): 51037-49.
Wong, M. L., and J. F. Medrano. 2005. “Real-Time PCR for MRNA Quantitation.” Biotechniques 39: 75-85.
Yao, J., Y. Chen, D. T. Nguyen, Z. J. Thompson, A. M. Eroshkin, N. Nerlakanti, A. K.
Patel, et al. 2019. “The Homeobox Gene, HOXB13, Regulates a Mitotic Protein-Kinase Interaction Network in Metastatic Prostate Cancers.” Sci Rep 9 (1): 9715.
Zhang, Guang Lan, Derin B. Keskin, Hsin-Nan Lin, Hong Huang Lin, David S.
DeLuca, Scott Leppanen, Edgar L. Milford, Ellis L. Reinherz, and Vladimir Brusic. 2014. “Human Leukocyte Antigen Typing Using a Knowledge Base Coupled with a High-Throughput Oligonucleotide Probe Array Analysis.” Frontiers in Immunology 5: 597.
Zhu, Z, and P Carter. 1995. “Identification of Heavy Chain Residues in a Humanized Anti-CD3 Antibody Important for Efficient Antigen Binding and T Cell Activation.” Journal of Immunology (Baltimore, Md.: 1950) 155 (4): 1903-10.

### SEQUENCES

The following sequences form pant of the disclosure of the present application. A WIPO ST26 compatible electronic sequence listing is provided with this application, too. For the avoidance of doubt, if discrepancies exist between the sequences in the following table and the electronic sequence listing, the sequences in this table shall be deemed to be the correct ones.

In some cases, the signal peptides may be encompassed in the reproduced sequences. In such case, the sequences shall be deemed disclosed with and without signal peptides. A readily available tool to identify signal peptides in a given protein sequence is SignalP-6.0 provided by Dansk Technical University under services.healthtech.dtu.dk/service.php?SignalP

TABLE 16
Sequences

| SEQ | | |
|---|---|---|
| ID | Identifier | Sequence |
| 1 | CD8α1 | MALPVTALLLPLALLLHAARPSQFRVSPLDRTWNLGETVELKCQVLLSNPTSGCSWLFQP |
| | | RGAAASPTFLLYLSQNKPKAAEGLDTQRFSGKRLGDTFVLTLSDFRRENEGYYFCSALSN |
| | | SIMYFSHFVPVFLPAKPTTTPAPRPPTPAPTIASQPLSLRPEACRPAAGGAVHTRGLDFA |
| | | CDIYIWAPLAGTCGVLLLSLVITLYCNHRNRRRVCKCPRPVVKSGDKPSLSARYV |
| 2 | CD8α2 | MALPVTALLLPLALLLHAARPSQFRVSPLDRTWNLGETVELKCQVLLSNPTSGCSWLFQP |
| | | RGAAASPTFLLYLSQNKPKAAEGLDTQRFSGKRLGDTFVLTLSDFRRENEGCYFCSALSN |
| | | SIMYFSHFVPVFLPAKPTTTPAPRPPTPAPTIASQPLSLRPEACRPAAGGAVHTRGLDFA |
| | | CDIYIWAPLAGTCGVLLLSLVITLYCNHRNRRRVCKCPRPVVKSGDKPSLSARYV |
| 3 | m1CD8α | MALPVTALLLPLALLLHAARPSQFRVSPLDRTWNLGETVELKCQVLLSNPTSGCSWLFQP |
| | | RGAAASPTFLLYLSQNKPKAAEGLDTQRFSGKRLGDTFVLTLSDFRRENEGYYFCSALSN |
| | | SIMYFSHFVPVFLPASVVDFLPTTAQPTKKSTLKKRVCRLPRPETQKGPLCSPIYIWAPL |
| | | AGTCGVLLLSLVITLYCNHRNRRRVCKCPRPVVKSGDKPSLSARYV |
| 4 | m2CD8α | MALPVTALLLPLALLLHAARPSQFRVSPLDRTWNLGETVELKCQVLLSNPTSGCSWLFQP |
| | | RGAAASPTFLLYLSQNKPKAAEGLDTQRFSGKRLGDTFVLTLSDFRRENEGCYFCSALSN |
| | | SIMYFSHFVPVFLPASVVDFLPTTAQPTKKSTLKKRVCRLPRPETQKGPLCSPIYIWAPL |
| | | AGTCGVLLLSLVITLYCNHRNRRRVCKCPRPVVKSGDKPSLSARYV |
| 5 | CD8β1 | MRPRLWLLLAAQLTVLHGNSVLQQTPAYIKVQTNKMVMLSCEAKISLSNMRIYWLRQRQA |
| | | PSSDSHHEFLALWDSAKGTIHGEEVEQEKIAVFRDASRFILNLTSVKPEDSGIYFCMIVG |
| | | SPELTFGKGTQLSVVDFLPTTAQPTKKSTLKKRVCRLPRPETQKGPLCSPITLGLLVAGV |
| | | LVLLVSLGVAIHLCCRRRRARLRFMKQPQGEGISGTFVPQCLHGYYSNTTTSQKLLNPWI |
| | | LKT |
| 6 | CD8β2 | MRPRLWLLLAAQLTVLHGNSVLQQTPAYIKVQTNKMVMLSCEAKISLSNMRIYWLRQRQA |
| | | PSSDSHHEFLALWDSAKGTIHGEEVEQEKIAVFRDASRFILNLTSVKPEDSGIYFCMIVG |
| | | SPELTFGKGTQLSVVDFLPTTAQPTKKSTLKKRVCRLPRPETQKGLKGKVYQEPLSPNAC |
| | | MDTTAILQPHRSCLTHGS |
| 7 | CD8β3 | LQQTPAYIKVQTNKMVMLSCEAKISLSNMRIYWLRQRQAPSSDSHHEFLALWDSAKGTIH |
| | | GEEVEQEKIAVFRDASRFILNLTSVKPEDSGIYFCMIVGSPELTFGKGTQLSVVDFLPTT |
| | | AQPTKKSTLKKRVCRLPRPETQKGPLCSPITLGLLVAGVLVLLVSLGVAIHLCCRRRRAR |
| | | LRFMKQFYK |
| 8 | CD8β4 | LQQTPAYIKVQTNKMVMLSCEAKISLSNMRIYWLRQRQAPSSDSHHEFLALWDSAKGTIH |
| | | GEEVEQEKIAVFRDASRFILNLTSVKPEDSGIYFCMIVGSPELTFGKGTQLSVVDFLPTT |
| | | AQPTKKSTLKKRVCRLPRPETQKGPLCSPITLGLLVAGVLVLLVSLGVAIHLCCRRRRAR |
| | | LRFMKQLRLHPLEKCSRMDY |
| 9 | CD8β5 | LQQTPAYIKVQTNKMVMLSCEAKISLSNMRIYWLRQRQAPSSDSHHEFLALWDSAKGTIH |
| | | GEEVEQEKIAVFRDASRFILNLTSVKPEDSGIYFCMIVGSPELTFGKGTQLSVVDFLPTT |
| | | AQPTKKSTLKKRVCRLPRPETQKGPLCSPITLGLLVAGVLVLLVSLGVAIHLCCRRRRAR |
| | | LRFMKQKFNIVCLKISGFTTCCCFQILQISREYGFGVLLQKDIGQ |
| 10 | CD8β6 | LQQTPAYIKVQTNKMVMLSCEAKISLSNMRIYWLRQRQAPSSDSHHEFLALWDSAKGTIH |
| | | GEEVEQEKIAVFRDASRFILNLTSVKPEDSGIYFCMIVGSPELTFGKGTQLSVVDFLPTT |
| | | AQPTKKSTLKKRVCRLPRPETQKGPLCSPITLGLLVAGVLVLLVSLGVAIHLCCRRRRAR |
| | | LRFMKQKFNIVCLKISGFTTCCCFQILQISREYGFGVLLQKDIGQ |
| 11 | CD8β7 | LQQTPAYIKVQTNKMVMLSCEAKISLSNMRIYWLRQRQAPSSDSHHEFLALWDSAKGTIH |
| | | GEEVEQEKIAVFRDASRFILNLTSVKPEDSGIYFCMIVGSPELTFGKGTQLSVVDFLPTT |
| | | AQPTKKSTLKKRVCRLPRPETQKGPLCSPITLGLLVAGVLVLLVSLGVAIHLCCRRRRAR |
| | | LRFMKQPQGEGISGTFVPQCLHGYYSNTTTSQKLLNPWILKT |
| 12 | R11P3D3 alpha | SSNFYA |
| | CDR1 | |
| 13 | R11P3D3 alpha | MTL |
| | CDR2 | |
| 14 | R11P3D3 alpha | CALYNNNDMRF |
| | CDR3 | |
| 15 | R11P3D3 alpha | MEKNPLAAPLLILWFHLDCVSSILNVEQSPQSLHVQEGDSTNFTCSFPSSNFYALHWYRW |
| | variable domain | ETAKSPEALFVMTLNGDEKKKGRISATLNTKEGYSYLYIKGSQPEDSATYLCALYNNNDM |
| | | RFGAGTRLTVKP |
| 16 | R11P3D3 alpha | NIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 17 | R11P3D3 alpha | MEKNPLAAPLLILWFHLDCVSSILNVEQSPQSLHVQEGDSTNFTCSFPSSNFYALHWYRW |
| | full-length | ETAKSPEALFVMTLNGDEKKKGRISATLNTKEGYSYLYIKGSQPEDSATYLCALYNNNDM |
| | | RFGAGTRLTVKPNIQNPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESSCDVKLVEKSFETDT |
| | | NLNFQNLSVIGFRILLLKVAGFNLLMTLRLWSS |
| 18 | R11P3D3 beta | SGHNS |
| | CDR1 | |
| 19 | R11P3D3 beta | FNNNVP |
| | CDR2 | |
| 20 | R11P3D3 beta | CASSPGSTDTQYF |
| | CDR3 | |
| 21 | R11P3D3 beta | MDSWTFCCVSLCILVAKHTDAGVIQSPRHEVTEMGQEVTLRCKPISGHNSLFWYRQTMMR |
| | variable domain | GLELLIYFNNNVPIDDSGMPEDRFSAKMPNASFSTLKIQPSEPRDSAVYFCASSPGSTDT |
| | | QYFGPGTRLTVL |
| 22 | R11P3D3 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 23 | R11P3D3 beta | MDSWTFCCVSLCILVAKHTDAGVIQSPRHEVTEMGQEVTLRCKPISGHNSLFWYRQTMMR |
| | full-length | GLELLIYFNNNVPIDDSGMPEDRFSAKMPNASFSTLKIQPSEPRDSAVYFCASSPGSTDT |
| | | QYFGPGTRLTVLEDLKNVFPPEVAVFEPSEAEISHTQKATLVCLATGFYPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATLYAVLVSALVL |
| | | MAMVKRKDSRG |
| 24 | R16P1C10 alpha | DRGSQS |
| | CDR1 | |
| 25 | R16P1C10 alpha | IY |
| | CDR2 | |
| 26 | R16P1C10 alpha | CAAVISNFGNEKLTF |
| | CDR3 | |
| 27 | R16P1C10 alpha | MKSLRVLLVILWLQLSWVWSQQKEVEQNSGPLSVPEGAIASLNCTYSDRGSQSFFWYRQY |
| | variable domain | SGKSPELIMFIYSNGDKEDGRFTAQLNKASQYVSLLIRDSQPSDSATYLCAAVISNFGNE |
| | | KLTFGTGTRLTIIP |
| 28 | R16P1C10 alpha | NIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 29 | R16P1C10 alpha | MKSLRVLLVILWLQLSWVWSQQKEVEQNSGPLSVPEGAIASLNCTYSDRGSQSFFWYRQY |
| | full-length | SGKSPELIMFIYSNGDKEDGRFTAQLNKASQYVSLLIRDSQPSDSATYLCAAVISNFGNE |
| | | KLTFGTGTRLTIIPNIQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSPESSCDVKLVEKSFET |
| | | DTNLNFQNLSVIGFRILLLKVAGFNLLMTLRLWSS |
| 30 | R16P1C10 beta | SGHRS |
| | CDR1 | |
| 31 | R16P1C10 beta | YFSETQ |
| | CDR2 | |
| 32 | R16P1C10 beta | CASSPWDSPNEQYF |
| | CDR3 | |
| 33 | R16P1C10 beta | MGSRLLCWVLLCLLGAGPVKAGVTQTPRYLIKTRGQQVTLSCSPISGHRSVSWYQQTPGQ |
| | variable domain | GLQFLFEYFSETQRNKGNFPGRFSGRQFSNSRSEMNVSTLELGDSALYLCASSPWDSPNE |
| | | QYFGPGTRLTVT |
| 34 | R16P1C10 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 35 | R16P1C10 beta | MGSRLLCWVLLCLLGAGPVKAGVTQTPRYLIKTRGQQVTLSCSPISGHRSVSWYQQTPGQ |
| | full-length | GLQFLFEYFSETQRNKGNFPGRFSGRQFSNSRSEMNVSTLELGDSALYLCASSPWDSPNE |
| | | QYFGPGTRLTVTEDLKNVFPPEVAVFEPSEAEISHTQKATLVCLATGFYPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATLYAVLVSALVL |
| | | MAMVKRKDSRG |
| 36 | R16P1E8 alpha | NSAFQY |
| | CDR1 | |
| 37 | R16P1E8 alpha | TY |
| | CDR2 | |
| 38 | R16P1E8 alpha | CAMSEAAGNKLTF |
| | CDR3 | |
| 39 | R16P1E8 alpha | MMKSLRVLLVILWLQLSWVWSQQKEVEQDPGPLSVPEGAIVSLNCTYSNSAFQYFMWYRQ |
| | variable domain | YSRKGPELLMYTYSSGNKEDGRFTAQVDKSSKYISLFIRDSQPSDSATYLCAMSEAAGNK |
| | | LTFGGGTRVLVKP |
| 40 | R16P1E8 alpha | NIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 41 | R16P1E8 alpha | MMKSLRVLLVILWLQLSWVWSQQKEVEQDPGPLSVPEGAIVSLNCTYSNSAFQYFMWYRQ |
| | full-length | YSRKGPELLMYTYSSGNKEDGRFTAQVDKSSKYISLFIRDSQPSDSATYLCAMSEAAGNK |
| | | LTFGGGTRVLVKPNIQNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSCDVKLVEKSFETD |
| | | TNLNFQNLSVIGFRILLLKVAGFNLLMTLRLWSS |
| 42 | R16P1E8 beta | SGHAT |
| | CDR1 | |
| 43 | R16P1E8 beta | FQNNGV |
| | CDR2 | |
| 44 | R16P1E8 beta | CASSYTNQGEAFF |
| | CDR3 | |
| 45 | R16P1E8 beta | MGTRLLCWAALCLLGAELTEAGVAQSPRYKIIEKRQSVAFWCNPISGHATLYWYQQILGQ |
| | variable domain | GPKLLIQFQNNGVVDDSQLPKDRFSAERLKGVDSTLKIQPAKLEDSAVYLCASSYTNQGE |
| | | AFFGQGTRLTVV |
| 46 | R16P1E8 beta | EDLNKVFPPEVAVFEPSEAEISHTQKATLVCLATGFFPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSVSYQQGVLSATILYEILLGKATLYAVLVSALVLMAMVKRKDF |
| 47 | R16P1E8 beta | MGTRLLCWAALCLLGAELTEAGVAQSPRYKIIEKRQSVAFWCNPISGHATLYWYQQILGQ |
| | full-length | GPKLLIQFQNNGVVDDSQLPKDRFSAERLKGVDSTLKIQPAKLEDSAVYLCASSYTNQGE |
| | | AFFGQGTRLTVVEDLNKVFPPEVAVFEPSEAEISHTQKATLVCLATGFFPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSVSYQQGVLSATILYEILLGKATLYAVLVSALVL |
| | | MAMVKRKDF |
| 48 | R17P1A9 alpha | DRGSQS |
| | CDR1 | |
| 49 | R17P1A9 alpha | IY |
| | CDR2 | |
| 50 | R17P1A9 alpha | CAVLNQAGTALIF |
| | CDR3 | |
| 51 | R17P1A9 alpha | MKSLRVLLVILWLQLSWVWSQQKEVEQNSGPLSVPEGAIASLNCTYSDRGSQSFFWYRQY |
| | variable domain | SGKSPELIMSIYSNGDKEDGRFTAQLNKASQYVSLLIRDSQPSDSATYLCAVLNQAGTAL |
| | | IFGKGTTLSVSS |
| 52 | R17P1A9 alpha | NIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 53 | R17P1A9 alpha | MKSLRVLLVILWLQLSWVWSQQKEVEQNSGPLSVPEGAIASLNCTYSDRGSQSFFWYRQY |
| | full-length | SGKSPELIMSIYSNGDKEDGRFTAQLNKASQYVSLLIRDSQPSDSATYLCAVLNQAGTAL |
| | | IFGKGTTLSVSSNIQNPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESSCDVKLVEKSFETDT |
| | | NLNFQNLSVIGFRILLLKVAGFNLLMTLRLWSS |
| 54 | R17P1A9 beta | SGDLS |
| | CDR1 | |
| 55 | R17P1A9 beta | YYNGEE |
| | CDR2 | |
| 56 | R17P1A9 beta | CASSAETGPWLGNEQFF |
| | CDR3 | |
| 57 | R17P1A9 beta | MGFRLLCCVAFCLLGAGPVDSGVTQTPKHLITATGQRVTLRCSPRSGDLSVYWYQQSLDQ |
| | variable domain | GLQFLIQYYNGEERAKGNILERFSAQQFPDLHSELNLSSLELGDSALYFCASSAETGPWL |
| | | GNEQFFGPGTRLTVL |
| 58 | R17P1A9 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 59 | R17P1A9 beta | MGFRLLCCVAFCLLGAGPVDSGVTQTPKHLITATGQRVTLRCSPRSGDLSVYWYQQSLDQ |
| | full-length | GLQFLIQYYNGEERAKGNILERFSAQQFPDLHSELNLSSLELGDSALYFCASSAETGPWL |
| | | GNEQFFGPGTRLTVLEDLKNVFPPEVAVFEPSEAEISHTQKATLVCLATGFYPDHVELSW |
| | | WVNGKEVHSGVSTDPQPLKEQPALNDSRYCLSSRLRVSATFWQNPRNHFRCQVQFYGLSE |
| | | NDEWTQDRAKPVTQIVSAEAWGRADCGFTSESYQQGVLSATILYEILLGKATLYAVLVSA |
| | | LVLMAMVKRKDSRG |
| 60 | R17P1D7 alpha | TSESDYY |
| | CDR1 | |
| 61 | R17P1D7 alpha | QEAY |
| | CDR2 | |
| 62 | R17P1D7 alpha | CAYRWAQGGSEKLVF |
| | CDR3 | |
| 63 | R17P1D7 alpha | MACPGFLWALVISTCLEFSMAQTVTQSQPEMSVQEAETVTLSCTYDTSESDYYLFWYKQP |
| | variable domain | PSRQMILVIRQEAYKQQNATENRFSVNFQKAAKSFSLKISDSQLGDAAMYFCAYRWAQGG |
| | | SEKLVFGKGTKLTVNP |
| 64 | R17P1D7 alpha | YIQKPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 65 | R17P1D7 alpha | MACPGFLWALVISTCLEFSMAQTVTQSQPEMSVQEAETVTLSCTYDTSESDYYLFWYKQP |
| | full-length | PSRQMILVIRQEAYKQQNATENRFSVNFQKAAKSFSLKISDSQLGDAAMYFCAYRWAQGG |
| | | SEKLVFGKGTKLTVNPYIQKPDPAVYQLRDSKSSDKSVCLFTDFDSQTNVSQSKDSDVYI |
| | | TDKTVLDMRSMDFKSNSAVAWSNKSDFACANAFNNSIIPEDTFFPSPESSCDVKLVEKSF |
| | | ETDTNLNFQNLSVIGFRILLLKVAGFNLLMTLRLWSS |
| 66 | R17P1D7 beta | MGHDK |
| | CDR1 | |
| 67 | R17P1D7 beta | SYGVNS |
| | CDR2 | |
| 68 | R17P1D7 beta | CATELWSSGGTGELFF |
| | CDR3 | |
| 69 | R17P1D7 beta | MTIRLLCYMGFYFLGAGLMEADIYQTPRYLVIGTGKKITLECSQTMGHDKMYWYQQDPGM |
| | variable domain | ELHLIHYSYGVNSTEKGDLSSESTVSRIRTEHFPLTLESARPSHTSQYLCATELWSSGGT |
| | | GELFFGEGSRLTVL |
| 70 | R17P1D7 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 71 | R17P1D7 beta | MTIRLLCYMGFYFLGAGLMEADIYQTPRYLVIGTGKKITLECSQTMGHDKMYWYQQDPGM |
| | full-length | ELHLIHYSYGVNSTEKGDLSSESTVSRIRTEHFPLTLESARPSHTSQYLCATELWSSGGT |
| | | GELFFGEGSRLTVLEDLKNVFPPEVAVFEPSEAEISHTQKATLVCLATGFYPDHVELSWW |
| | | VNGKEVHSGVSTDPQPLKEQPALNDSRYCLSSRLRVSATFWQNPRNHFRCQVQFYGLSEN |
| | | DEWTQDRAKPVTQIVSAEAWGRADCGFTSESYQQGVLSATILYEILLGKATLYAVLVSAL |
| | | VLMAMVKRKDSRG |
| 72 | R17P1G3 alpha | DRGSQS |
| | CDR1 | |
| 73 | R17P1G3 alpha | IY |
| | CDR2 | |
| 74 | R17P1G3 alpha | CAVGPSGTYKYIF |
| | CDR3 | |
| 75 | R17P1G3 alpha | MKSLRVLLVILWLQLSWVWSQQKEVEQNSGPLSVPEGAIASLNCTYSDRGSQSFFWYRQY |
| | variable domain | SGKSPELIMSIYSNGDKEDGRFTAQLNKASQYVSLLIRDSQPSDSATYLCAVGPSGTYKY |
| | | IFGTGTRLKVLA |
| 76 | R17P1G3 alpha | NIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 77 | R17P1G3 alpha | MKSLRVLLVILWLQLSWVWSQQKEVEQNSGPLSVPEGAIASLNCTYSDRGSQSFFWYRQY |
| | full-length | SGKSPELIMSIYSNGDKEDGRFTAQLNKASQYVSLLIRDSQPSDSATYLCAVGPSGTYKY |
| | | IFGTGTRLKVLANIQNPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESSCDVKLVEKSFETDT |
| | | NLNFQNLSVIGFRILLLKVAGFNLLMTLRLWSS |
| 78 | R17P1G3 beta | MNHEY |
| | CDR1 | |
| 79 | R17P1G3 beta | SMNVEV |
| | CDR2 | |
| 80 | R17P1G3 beta | CASSPGGSGNEQFF |
| | CDR3 | |
| 81 | R17P1G3 beta | MGPQLLGYVVLCLLGAGPLEAQVTQNPRYLITVTGKKLTVTCSQNMNHEYMSWYRQDPGL |
| | variable domain | GLRQIYYSMNVEVTDKGDVPEGYKVSRKEKRNFPLILESPSPNQTSLYFCASSPGGSGNE |
| | | QFFGPGTRLTVL |
| 82 | R17P1G3 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 83 | R17P1G3 beta | MGPQLLGYVVLCLLGAGPLEAQVTQNPRYLITVTGKKLTVTCSQNMNHEYMSWYRQDPGL |
| | full-length | GLRQIYYSMNVEVTDKGDVPEGYKVSRKEKRNFPLILESPSPNQTSLYFCASSPGGSGNE |
| | | QFFGPGTRLTVLEDLKNVFPPEVAVFEPSEAEISHTQKATLVCLATGFYPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATLYAVLVSALVL |
| | | MAMVKRKDSRG |
| 84 | R17P2B6 alpha | DRGSQS |
| | CDR1 | |
| 85 | R17P2B6 alpha | IY |
| | CDR2 | |
| 86 | R17P2B6 alpha | CAVVSGGGADGLTF |
| | CDR3 | |
| 87 | R17P2B6 alpha | MKSLRVLLVILWLQLSWVWSQQKEVEQNSGPLSVPEGAIASLNCTYSDRGSQSFFWYRQY |
| | variable domain | SGKSPELIMFIYSNGDKEDGRFTAQLNKASQYVSLLIRDSQPSDSATYLCAVVSGGGADG |
| | | LTFGKGTHLIIQP |
| 88 | R17P2B6 alpha | YIQKPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 89 | R17P2B6 alpha | MKSLRVLLVILWLQLSWVWSQQKEVEQNSGPLSVPEGAIASLNCTYSDRGSQSFFWYRQY |
| | full-length | SGKSPELIMFIYSNGDKEDGRFTAQLNKASQYVSLLIRDSQPSDSATYLCAVVSGGGADG |
| | | LTFGKGTHLIIQPYIQKPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSCDVKLVEKSFETD |
| | | TNLNFQNLSVIGFRILLLKVAGFNLLMTLRLWSS |
| 90 | R17P2B6 beta | PRHDT |
| | CDR1 | |
| 91 | R17P2B6 beta | FYEKMQ |
| | CDR2 | |
| 92 | R17P2B6 beta | CASSLGRGGQPQHF |
| | CDR3 | |
| 93 | R17P2B6 beta | MLSPDLPDSAWNTRLLCHVMLCLLGAVSVAAGVIQSPRHLIKEKRETATLKCYPIPRHDT |
| | variable domain | VYWYQQGPGQDPQFLISFYEKMQSDKGSIPDRFSAQQFSDYHSELNMSSLELGDSALYFC |
| | | ASSLGRGGQPQHFGDGTRLSIL |
| 94 | R17P2B6 beta | EDLNKVFPPEVAVFEPSEAEISHTQKATLVCLATGFFPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSVSYQQGVLSATILYEILLGKATLYAVLVSALVLMAMVKRKDF |
| 95 | R17P2B6 beta | MLSPDLPDSAWNTRLLCHVMLCLLGAVSVAAGVIQSPRHLIKEKRETATLKCYPIPRHDT |
| | full-length | VYWYQQGPGQDPQFLISFYEKMQSDKGSIPDRFSAQQFSDYHSELNMSSLELGDSALYFC |
| | | ASSLGRGGQPQHFGDGTRLSILEDLNKVFPPEVAVFEPSEAEISHTQKATLVCLATGFFP |
| | | DHVELSWWVNGKEVHSGVSTDPQPLKEQPALNDSRYCLSSRLRVSATFWQNPRNHFRCQV |
| | | QFYGLSENDEWTQDRAKPVTQIVSAEAWGRADCGFTSVSYQQGVLSATILYEILLGKATL |
| | | YAVLVSALVLMAMVKRKDF |
| 96 | 1G4 alpha CDR1 | DSAIYN |
| 97 | 1G4 alpha CDR2 | IQS |
| 98 | 1G4 alpha CDR3 | CAVRPTSGGSYIPTF |
| 99 | 1G4 alpha | METLLGLLILWLQLQWVSSKQEVTQIPAALSVPEGENLVLNCSFTDSAIYNLQWFRQDPG |
| | variable domain | KGLTSLLLIQSSQREQTSGRLNASLDKSSGRSTLYIAASQPGDSATYLCAVRPTSGGSYI |
| | | PTFGRGTSLIVHP |
| 100 | 1G4 alpha | YIQNPDPAVYQLRDSKSSDKSVCLFTDFDSQTNVSQSKDSDVYITDKTVLDMRSMDFKSN |
| | constant domain | SAVAWSNKSDFACANAFNNSIIPEDTFFPSPESSCDVKLVEKSFETDTNLNFQNLSVIGF |
| | | RILLLKVAGFNLLMTLRLWSS |
| 101 | 1G4 alpha | METLLGLLILWLQLQWVSSKQEVTQIPAALSVPEGENLVLNCSFTDSAIYNLQWFRQDPG |
| | full-length | KGLTSLLLIQSSQREQTSGRLNASLDKSSGRSTLYIAASQPGDSATYLCAVRPTSGGSYI |
| | | PTFGRGTSLIVHPYIQNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSCDVKLVEKSFETD |
| | | TNLNFQNLSVIGFRILLLKVAGFNLLMTLRLWSS |
| 102 | 1G4 beta CDR1 | MNHEY |
| 103 | 1G4 beta CDR2 | SVGAGI |
| 104 | 1G4 beta CDR3 | CASSYVGNTGELFF |
| 105 | 1G4 beta | MSIGLLCCAALSLLWAGPVNAGVTQTPKFQVLKTGQSMTLQCAQDMNHEYMSWYRQDPGM |
| | variable domain | GLRLIHYSVGAGITDQGEVPNGYNVSRSTTEDFPLRLLSAAPSQTSVYFCASSYVGNTGE |
| | | LFFGEGSRLTVL |
| 106 | 1G4 beta | EDLKNVFPPEVAVFEPSEAEISHTQKATLVCLATGFYPDHVELSWWVNGKEVHSGVSTDP |
| | constant domain | QPLKEQPALNDSRYCLSSRLRVSATFWQNPRNHFRCQVQFYGLSENDEWTQDRAKPVTQI |
| | | VSAEAWGRADCGFTSESYQQGVLSATILYEILLGKATLYAVLVSALVLMAMVKRKDSRG |
| 107 | 1G4 beta | MSIGLLCCAALSLLWAGPVNAGVTQTPKFQVLKTGQSMTLQCAQDMNHEYMSWYRQDPGM |
| | full-length | GLRLIHYSVGAGITDQGEVPNGYNVSRSTTEDFPLRLLSAAPSQTSVYFCASSYVGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATLYAVLVSALVL |
| | | MAMVKRKDSRG |
| 108 | R11P3D3_KE | SSNFYA |
| | alpha CDR1 | |
| 109 | R11P3D3_KE | MTL |
| | alpha CDR2 | |
| 110 | R11P3D3_KE | CALYNNNDMRF |
| | alpha CDR3 | |
| 111 | R11P3D3_KE | MEKNPLAAPLLILWFHLDCVSSILNVEQSPQSLHVQEGDSTNFTCSFPSSNFYALHWYRK |
| | alpha | ETAKSPEALFVMTLNGDEKKKGRISATLNTKEGYSYLYIKGSQPEDSATYLCALYNNNDM |
| | variable domain | RFGAGTRLTVKP |
| 112 | R11P3D3_KE | NIQNPDPAVYQLRDSKSSDKSVCLFTDFDSQTNVSQSKDSDVYITDKTVLDMRSMDFKSN |
| | alpha | SAVAWSNKSDFACANAFNNSIIPEDTFFPSPESSCDVKLVEKSFETDTNLNFQNLSVIGF |
| | constant domain | RILLLKVAGFNLLMTLRLWSS |
| 113 | R11P3D3_KE | MEKNPLAAPLLILWFHLDCVSSILNVEQSPQSLHVQEGDSTNFTCSFPSSNFYALHWYRK |
| | alpha | ETAKSPEALFVMTLNGDEKKKGRISATLNTKEGYSYLYIKGSQPEDSATYLCALYNNNDM |
| | full-length | RFGAGTRLTVKPNIQNPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESSCDVKLVEKSFETDT |
| | | NLNFQNLSVIGFRILLLKVAGFNLLMTLRLWSS |
| 114 | R11P3D3_KE | SGHNS |
| | beta CDR1 | |
| 115 | R11P3D3_KE | FNNNVP |
| | beta CDR2 | |
| 116 | R11P3D3_KE | CASSPGSTDTQYF |
| | beta CDR3 | |
| 117 | R11P3D3_KE | MDSWTFCCVSLCILVAKHTDAGVIQSPRHEVTEMGQEVTLRCKPISGHNSLFWYRETMMR |
| | beta | GLELLIYFNNNVPIDDSGMPEDRFSAKMPNASFSTLKIQPSEPRDSAVYFCASSPGSTDT |
| | variable domain | QYFGPGTRLTVL |
| 118 | R11P3D3_KE | EDLKNVFPPEVAVFEPSEAEISHTQKATLVCLATGFYPDHVELSWWVNGKEVHSGVSTDP |
| | beta | QPLKEQPALNDSRYCLSSRLRVSATFWQNPRNHFRCQVQFYGLSENDEWTQDRAKPVTQI |
| | constant domain | VSAEAWGRADCGFTSESYQQGVLSATILYEILLGKATLYAVLVSALVLMAMVKRKDSRG |
| 119 | R11P3D3_KE | MDSWTFCCVSLCILVAKHTDAGVIQSPRHEVTEMGQEVTLRCKPISGHNSLFWYRETMMR |
| | beta | GLELLIYFNNNVPIDDSGMPEDRFSAKMPNASFSTLKIQPSEPRDSAVYFCASSPGSTDT |
| | full-length | QYFGPGTRLTVLEDLKNVFPPEVAVFEPSEAEISHTQKATLVCLATGFYPDHVELSWWVN |
| | | GKEVHSGVSTDPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATLYAVLVSALVL |
| | | MAMVKRKDSRG |
| 120 | R11P3D3 alpha | MTLNGDE |
| | CDR2bis | |
| 121 | R16P1C10 alpha | IYSNGD |
| | CDR2bis | |
| 122 | R16P1E8 alpha | TYSSGN |
| | CDR2bis | |
| 123 | R17P1A9 alpha | IYSNGD |
| | CDR2bis | |
| 124 | R17P1D7 alpha | QEAYKQQ |
| | CDR2bis | |
| 125 | R17P1G3 alpha | IYSNGD |
| | CDR2bis | |
| 126 | R17P2B6 alpha | IYSNGD |
| | CDR2bis | |
| 127 | 1G4 alpha | IQSSQRE |
| | CDR2bis | |
| 128 | R11P3D3_KE | MTLNGDE |
| | alpha | |
| | CDR2bis | |
| 129 | hinges of an IgG1 | EPKSCDKTHTCPPCPAPELLG |
| | molecule is (EU | |
| | numbering | |
| | indicated), | |
| | staring with E216 | |
| 130 | Fc domain can | ELLGGP |
| | comprise a CH2 | |
| | domain | |
| | comprising | |
| | at least one | |
| | effector | |
| | function silencing | |
| | mutation | |
| 131 | IA_5R16P1C10I | QKEVEQNSGPLSVPEGAIASLNCTYSDRGSQSFFWYRQYSGKSPELIMSIYSNGDKEDGR |
| | hUCHT1(Var17) | FTAQLNKASQYFSLLIRDSQPSDSATYLCAAVIDNSNGGILTFGTGTRLTIIPNIQNGGG |
| | | SGGGGDIQMTQSPSSLSASVGDRVTITCRASQDIRNYLNWYQQKPGKAPKLLIYYTSRLH |
| | | SGVPSRFSGSGSGTDYTLTISSLQPEDIATYFCQQGQTLPWTFGQGTKVEIKEPKSSDKT |
| | | HTCPPCPAPPVAGPSVFLFPPKPKDTLMISRTPEVTCVVVDVSHEDPEVKFNWYVDGVEV |
| | | HNAKTKPREEQYQSTYRVVSVLTVLHQDWLNGKEYKCKVSNKALPASIEKTISKAKGQPR |
| | | EPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSP |
| 132 | IA_5R16P1C10I | EVQLVQSGAEVKKPGASVKVSCKASGYSFTGYTMNWVRQAPGQGLEWMGLINPYKGVSTY |
| | hUCHT1(Var17) | AQKFQDRVTLTVDKSTSTAYMELSSLRSEDTAVYYCARSGYYGDSDWYFDVWGQGTLVTV |
| | | SSGGGSGGGGKAGVTQTPRYLIKTRGQQVTLSCSPIPGHRSVSWYQQTPGQGLQFLFEYV |
| | | HGAERNKGNFPGRFSGRQFSNSSSEMNISNLELGDSALYLCASSPWDSPNEQYFGPGTRL |
| | | TVTEDLKNEPKSSDKTHTCPPCPAPPVAGPSVFLFPPKPKDTLMISRTPEVTCVVVDVSH |
| | | EDPEVKFNWYVDGVEVHNAKTKPREEQYQSTYRVVSVLTVLHQDWLNGKEYKCKVSNKAL |
| | | PASIEKTISKAKGQPREPQVCTLPPSRDELTKNQVSLSCAVKGFYPSDIAVEWESNGQPE |
| | | NNYKTTPPVLDSDGSFFLVSKLTVDKSRWQQGNVFSCSVMHEALHNHYTQKSLSLSP |
| 133 | IA_6R16P1C10I#6 | QKEVEQNSGPLSVPEGAIASLNCTYSDRGSQSFFWYRQYSGKSPELIMSIYSNGDKEDGR |
| | hUCHT1(Var17) | FTAQLNKASQYVSLLIRDSQPSDSATYLCAAVIDNDQGGILTFGTGTRLTIIPNIQNGGG |
| | | SGGGGDIQMTQSPSSLSASVGDRVTITCRASQDIRNYLNWYQQKPGKAPKLLIYYTSRLH |
| | | SGVPSRFSGSGSGTDYTLTISSLQPEDIATYFCQQGQTLPWTFGQGTKVEIKEPKSSDKT |
| | | HTCPPCPAPPVAGPSVFLFPPKPKDTLMISRTPEVTCVVVDVSHEDPEVKFNWYVDGVEV |
| | | HNAKTKPREEQYQSTYRVVSVLTVLHQDWLNGKEYKCKVSNKALPASIEKTISKAKGQPR |
| | | EPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 134 | IA_6R16P1C10I# | EVQLVQSGAEVKKPGASVKVSCKASGYSFTGYTMNWVRQAPGQGLEWMGLINPYKGVSTY |
| | 6hUCHT1(Var17) | AQKFQDRVTLTVDKSTSTAYMELSSLRSEDTAVYYCARSGYYGDSDWYFDVWGQGTLVTV |
| | | SSGGGSGGGGKAGVTQTPRYLIKTRGQQVTLSCSPIPGHRAVSWYQQTPGQGLQFLFEYV |
| | | HGEERNKGNFPGRFSGRQFSNSSSEMNISNLELGDSALYLCASSPWDSPNVQYFGPGTRL |
| | | TVTEDLKNEPKSSDKTHTCPPCPAPPVAGPSVFLFPPKPKDTLMISRTPEVTCVVVDVSH |
| | | EDPEVKFNWYVDGVEVHNAKTKPREEQYQSTYRVVSVLTVLHQDWLNGKEYKCKVSNKAL |
| | | PASIEKTISKAKGQPREPQVCTLPPSRDELTKNQVSLSCAVKGFYPSDIAVEWESNGQPE |
| | | NNYKTTPPVLDSDGSFFLVSKLTVDKSRWQQGNVFSCSVMHEALHNHYTQKSLSLSPGK |
| 135 | alpha CDRa1 | DRGSQS |
| 136 | alpha CDRa1 | DRGSQL |
| 137 | alpha CDRa2 | IYSNGD |
| 138 | alpha CDRa2 | IYQEGD |
| 139 | alpha CDRa3 | CAAVINNPSGGMLTF |
| 140 | alpha CDRa3 | CAAVIDNSNGGILTF |
| 141 | alpha CDRa3 | CAAVIDNPSGGILTF |
| 142 | alpha CDRa3 | CAAVIDNDQGGILTF |
| 143 | alpha CDRa3 | CAAVIPNPPGGKLTF |
| 144 | alpha CDRa3 | CAAVIPNPGGGALTF |
| 145 | alpha CDRa3 | CAAVIPNSAGGRLTF |
| 146 | alpha CDRa3 | CAAVIPNLEGGSLTF |
| 147 | alpha CDRa3 | CAAVIPNRLGGYLTF |
| 148 | alpha CDRa3 | CAAVIPNTDGGRLTF |
| 149 | alpha CDRa3 | CAAVIPNQRGGALTF |
| 150 | alpha CDRa3 | CAAVIPNVVGGILTF |
| 151 | alpha CDRa3 | CAAVITNIAGGSLTF |
| 152 | alpha CDRa3 | CAAVIPNNDGGYLTF |
| 153 | alpha CDRa3 | CAAVIPNGRGGLLTF |
| 154 | alpha CDRa3 | CAAVIPNTHGGPLTF |
| 155 | alpha CDRa3 | CAAVIPNDVGGSLTF |
| 156 | alpha CDRa3 | CAAVIENKPGGPLTF |
| 157 | alpha CDRa3 | CAAVIDNPVGGPLTF |
| 158 | alpha CDRa3 | CAAVIPNNNGGALTF |
| 159 | alpha CDRa3 | CAAVIPNDQGGILTF |
| 160 | alpha CDRa3 | CAAVIPNVVGGQLTF |
| 161 | alpha CDRa3 | CAAVIPNSYGGLLTF |
| 162 | alpha CDRa3 | CAAVIPNDDGGLLTF |
| 163 | alpha CDRa3 | CAAVIPNAAGGLLTF |
| 164 | alpha CDRa3 | CAAVIPNTIGGLLTF |
| 165 | alpha CDRa3 | CAAVIPNTRGGLLTF |
| 166 | beta CDRb1 | SGHRS |
| 167 | beta CDRb1 | PGHRA |
| 168 | beta CDRb1 | PGHRS |
| 169 | beta CDRb2 | YFSETQ |
| 170 | beta CDRb2 | YVHGEE |
| 171 | beta CDRb2 | YVHGAE |
| 172 | beta CDRb3 | CASSPWDSPNEQYF |
| 173 | beta CDRb3 | CASSPWDSPNVQYF |
| 174 | scTCR-Fab | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GQKEVEQNSGPLSVPEGAIASLNCTYSDRGSQSFFWYRQYSGKSPELIMSIYQEGDKEDG |
| | | RFTAQLNKASQYVSLLIRDSQPSDSATYLCAAVIDNDQGGILTFGTGTRLTIIPNIQNGG |
| | | GGSGGGGSGGGGSGGGGSGGGGSGSKAGVTQTPRYLIKTRGQQVTLSCSPIPGHRAVSWY |
| | | QQTPGQGLQFLFEYVHGEERNKGNFPGRFSGRQFSNSSSEMNISNLELGDSALYLCASSP |
| | | WDSPNVQYFGPGTRLTVTEDLKN |
| 175 | scTCR-Fab | DIQMTQSPSSLSASVGDRVTITCRASQDIRNYLNWYQQKPGKAPKLLIYYTSRLHSGVPS |
| | | RFSGSGSGTDYTLTISSLQPEDIATYFCQQGQTLPWTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 176 | diabody-Fc | QKEVEQNSGPLSVPEGAIASLNCTYSDRGSQSFFWYRQYSGKSPELIMSIYQEGDKEDGR |
| | | FTAQLNKASQYVSLLIRDSQPSDSATYLCAAVIDNDQGGILTFGTGTRLTIIPNIQNGGG |
| | | SGGGGDIQMTQSPSSLSASVGDRVTITCRASQDIRNYLNWYQQKPGKAPKLLIYYTSRLH |
| | | SGVPSRFSGSGSGTDYTLTISSLQPEDIATYFCQQGQTLPWTFGQGTKVEIKEPKSSDKT |
| | | HTCPPCPAPPVAGPSVFLFPPKPKDTLMISRTPEVTCVVVDVSHEDPEVKFNWYVDGVEV |
| | | HNAKTKPREEQYQSTYRVVSVLTVLHQDWLNGKEYKCKVSNKALPASIEKTISKAKGQPR |
| | | EPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSP |
| 177 | diabody-Fc | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGKAGVTQTPRYLIKTRGQQVTLSCSPIPGHRAVSWYQQTPGQGLQFLFEYV |
| | | HGEERNKGNFPGRFSGRQFSNSSSEMNISNLELGDSALYLCASSPWDSPNVQYFGPGTRL |
| | | TVTEDLKNEPKSSDKTHTCPPCPAPPVAGPSVFLFPPKPKDTLMISRTPEVTCVVVDVSH |
| | | EDPEVKFNWYVDGVEVHNAKTKPREEQYQSTYRVVSVLTVLHQDWLNGKEYKCKVSNKAL |
| | | PASIEKTISKAKGQPREPQVCTLPPSRDELTKNQVSLSCAVKGFYPSDIAVEWESNGQPE |
| | | NNYKTTPPVLDSDGSFFLVSKLTVDKSRWQQGNVFSCSVMHEALHNHYTQKSLSLSP |
| 178 | | DIQMTQSPSSLSASVGDRVTITCRASQDIRNYLNWYQQKPGKAPKLLIYYTSRLHSGVPS |
| | | RFSGSGSGTDYTLTISSLQPEDIATYFCQQGQTLPWTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 179 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GILNVEQSPQSLHVQEGDSTNFTCSFPTREFQDLHWYRKETAKSPEFLFYFGPYGVEKKK |
| | | GRISATLNTKEGYSYLYITDSQPEDSATYLCALYNNNDMRFGAGTRLTVKPGGGGSGGGG |
| | | SGGGGSGGGGSGGGGSGVIQSPRHLVTEMGQEVTLRCKPISGHNSLFWYRETPMQGLELL |
| | | IYFQNTAVIDDSGMPEDRFSAKMPNASFSTLKIQPSEPRDSAVYFCASSPGSTDTQYFGP |
| | | GTRLTVL |
| 180 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GILNVEQSPQSLHVQEGDSTNFTCSFPTKEFQDLHWYRKETAKSPEFLFYFGPYGREKKK |
| | | GRISATLNTKEGYSYLYITDSQPEDSATYLCALYNNNDMRFGAGTRLTVKPGGGGSGGGG |
| | | SGGGGSGGGGSGGGGSGVIQSPRHLVTEMGQEVTLRCKPISGHNSLFWYRETPMQGLELL |
| | | IYFQNTAVIDDSGMPEDRFSAKMPNASFSTLKIQPSEPRDSAVYFCASSPGATDTQYFGP |
| | | GTRLTVL |
| 181 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GILNVEQSPQSLHVQEGDSTNFTCSFPSSNFYNLHWYRKETAKSPEFLFYFGPYGVEKKK |
| | | GRISATLNTKEGYSYLYITDSQPEDSATYLCALYNNNDMRFGAGTRLTVKPGGGGSGGGG |
| | | SGGGGSGGGGSGGGGSGVIQSPRHLVTEMGQEVTLRCKPISGHNSLFWYRETPMQGLELL |
| | | IYFNSETVIDDSGMPEDRFSAKMPNASFSTLKIQPSEPRDSAVYFCASSPGATDTQYFGP |
| | | GTRLTVL |
| 182 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GILNVEQSPQSLHVQEGDSTNFTCSFPNKEFQDLHWYRKETAKSPEFLFYFGPYGTEKKK |
| | | GRISATLNTKEGYSYLYITDSQPEDSATYLCALYNNNDMRFGAGTRLTVKPGGGGSGGGG |
| | | SGGGGSGGGGSGGGGSGVIQSPRHLVTEMGQEVTLRCKPISGHNSLFWYRETPMQGLELL |
| | | IYFQNTAVIDDSGMPEDRFSAKMPNASFSTLKIQPSEPRDSAVYFCASSPGSTDTQYFGP |
| | | GTRLTVL |
| 183 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTSPPSPAPPVA |
| | | GILNVEQSPQSLHVQEGDSTNFTCSFPVKEFQDLHWYRKETAKSPEFLFYFGPYGKEKKK |
| | | GRISATLNTKEGYSYLYITDSQPEDSATYLCALYNNNDMRFGAGTRLTVKPGGGGSGGGG |
| | | SGGGGSGGGGSGGGGSGVIQSPRHLVTEMGQEVTLRCKPISGHNSLFWYRETPMQGLELL |
| | | IYFQNTAVIDDSGMPEDRFSAKMPNASFSTLKIQPSEPRDSAVYFCASSPGATDTQYFGP |
| | | GTRLTVL |
| 184 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 185 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHLVTEMGQEVTLRCKPISGHNSLFWYRETPMQGLELLIYFQNT |
| | | AVIDDSGMPEDRFSAKMPNASFSTLKIQPSEPRDSAVYFCASSPGATDTQYFGPGTRLTV |
| | | LEPKSSDKTHTCPPCPAPPVAGPSVFLFPPKPKDTLMISRTPEVTCVVVDVSHEDPEVKF |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 186 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHLVTEMGQEVTLRCKPISGHNSLFWYRETPMQGLELLIYFQNTAVIDDSGMPEDRFSAK |
| | | MPNASFSTLKIQPSEPRDSAVYFCASSPGATDTQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 187 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 188 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHLVTEMGQEVTLRCKPISGHNSLFWYRETPMQGLELLIYFQNTAVIDDSGMPEDRF |
| | | SAKMPNASFSTLKIQPSEPRDSAVYFCASSPGATDTQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 189 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 190 | | IMNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 191 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHLVTEMGQEVTLRCKPISGHNSLFWYRETPMQGLELLIYFQNT |
| | | AVIDDSGMPEDRFSAKMPNDSFSTLKIQPSEPRDSAVYFCASSPGATDTQYFGPGTRLTV |
| | | LEPKSSDKTHTCPPCPAPPVAGPSVFLFPPKPKDTLMISRTPEVTCVVVDVSHEDPEVKF |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 192 | | DIQMTQSPSSLSASVGDRVTITCRASQDIRNYLNWYQQKPGKAPKLLIYYTSRLHSGVPS |
| | | RFSGSGSGTDYTLTISSLQPEDIATYFCQQGQTLPWTFGQGTKVEIK |
| 193 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SS |
| 194 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHLVTEMGQEVTLRCKPISGHNSLFWYRETPMQGLELLIYFQNTAVIDDSGMPEDRFSAK |
| | | MPNDSFSTLKIQPSEPRDSAVYFCASSPGATDTQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 195 | | IMNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 196 | | EVQLVQSGAEVKKPGASVKVSCKASGYKFTSYVMHWVRQAPGQGLEWMGYINPYNDVTKY |
| | | AEKFQGRVTLTSDTSTSTAYMELSSLRSEDTAVHYCARGSYYDYEGFVYWGQGTLVTVSS |
| 197 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIK |
| 198 | | EVQLVQSGAEVKKPGASVKVSCKASGYKFTRYVMHWVRQAPGQGLEWMGYINPYNDVTKY |
| | | AEKFQGRVTLTSDTSTSTAYMELSSLRSEDTAVHYCARGSYYDYEGFVYWGQGTLVTVSS |
| 199 | | EVQLVQSGAEVKKPGASVKVSCKASGYKFTSYVMHWVRQAPGQGLEWMGYINPRNDVTKY |
| | | AEKFQGRVTLTSDTSTSTAYMELSSLRSEDTAVHYCARGSYYDYEGFVYWGQGTLVTVSS |
| 200 | | EVQLVQSGAEVKKPGASVKVSCKASGYKFTRYVMHWVRQAPGQGLEWMGYINPYNDVTKY |
| | | AEKFQGRVTLTSDTSTSTAYMELSSLRSEDTAVYYCARGSYYDYEGFVYWGQGTLVTVSS |
| 201 | | EVQLVQSGAEVKKPGASVKVSCKASGYKFTSYVMHWVRQAPGQGLEWMGYINPRNDVTKY |
| | | AEKFQGRVTLTSDTSTSTAYMELSSLRSEDTAVYYCARGSYYDYEGFVYWGQGTLVTVSS |
| 202 | | EVQLVESGGGLVQPGGSLKLSCAASGFTFNKYAMNWVRQAPGKGLEWVARIRSKYNNYAT |
| | | YYADSVKDRFTISRDDSKNTAYLQMNNLKTEDTAVYYCVRHGNFGNSYISYWAYWGQGTL |
| | | VTVSS |
| 203 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHLVTEMGQEVTLRCKPISGHNSLFWYRETPMQGLELLIYFQNT |
| | | AVIDDSGMPEDRFSAKMPNDSFSTLKIQPSEPRDSAVYFCASSPGATDLQYFGPGTRLTV |
| | | LEPKSSDKTHTCPPCPAPPVAGPSVFLFPPKPKDTLMISRTPEVTCVVVDVSHEDPEVKF |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 204 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVL |
| 205 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHEVTEMGQEVTLRCKPISGHNSLFWYRETPMQGLELLIYFQNT |
| | | AVIDDSGMPEDRFSAKMPNDSFSTLKIQPSEPRDSAVYFCASSPGATDLQYFGPGTRLTV |
| | | LEPKSSDKTHTCPPCPAPPVAGPSVFLFPPKPKDTLMISRTPEVTCVVVDVSHEDPEVKF |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 206 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 207 | | EVQLVESGGGLVQPGGSLKLSCAASGFTFNKYAMNWVRQAPGKGLEWVARIRSKYNNYAT |
| | | YYADSVKDRFTISRDDSKNTAYLQMNNLKTEDTAVYYCVRHGNFGDSYISYWAYWGQGTL |
| | | VTVSS |
| 208 | | IMNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 209 | | EVQLVESGGGLVQPGGSLKLSCAASGFTFNKYAMNWVRQAPGKGLEWVARIRSKYNNYAT |
| | | YYADSVKDRFTISRDDSKNTAYLQMNNLKTEDTAVYYCVRHGNFGESYISYWAYWGQGTL |
| | | VTVSS |
| 210 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 211 | | EVQLVESGGGLVQPGGSLKLSCAASGFTFNKYAMNWVRQAPGKGLEWVARIRSKYNNYAT |
| | | YYADSVKDRFTISRDDSKNTAYLQMNNLKTEDTAVYYCVRHGNFGNAYISYWAYWGQGTL |
| | | VTVSS |
| 212 | | IMNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGDI |
| | | QMTQSPSSLSASVGDRVTITCRASQDIRNYLNWYQQKPGKAPKLLIYYTSRLHSGVPSRF |
| | | SGSGSGTDYTLTISSLQPEDIATYFCQQGQTLPWTFGQGTKVEIKEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 213 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHEVTEMGQEVTLRCKPISGHNSLFWYRETPMQGLELLIYFQNT |
| | | AVIDDSGMPEDRFSAKMPNDSFSTLKIQPSEPRDSAVYFCASSPGATDKQYFGPGTRLTV |
| | | LEPKSSDKTHTCPPCPAPPVAGPSVFLFPPKPKDTLMISRTPEVTCVVVDVSHEDPEVKE |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 214 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHEVTEMGQEVTLRCKPISGHNSLFWYRETMMQGLELLIYFQNT |
| | | AVIDDSGMPEDRFSAKMPNDSFSTLKIQPSEPRDSAVYFCASSPGATDLQYFGPGTRLTV |
| | | LEPKSSDKTHTCPPCPAPPVAGPSVFLFPPKPKDTLMISRTPEVTCVVVDVSHEDPEVKE |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 215 | | EVQLVQSGAEVKKPGASVKVSCKASGYSFTGYTMNWVRQAPGQGLEWMGLINPYKGVSTY |
| | | AQKFQDRVTLTVDKSTSTAYMELSSLRSEDTAVYYCARSGYYGDSDWYFDVWGQGTLVTV |
| | | SSGGGSGGGGGVIQSPRHEVTEMGQEVTLRCKPISGHNSLFWYRETMMRGLELLIYFQNT |
| | | AVIDDSGMPEDRFSAKMPNDSFSTLKIQPSEPRDSAVYFCASSPGATDLQYFGPGTRLTV |
| | | LEPKSSDKTHTCPPCPAPPVAGPSVFLFPPKPKDTLMISRTPEVTCVVVDVSHEDPEVKE |
| | | NWYVDGVEVHNAKTKPREEQYQSTYRVVSVLTVLHQDWLNGKEYKCKVSNKALPASIEKT |
| | | ISKAKGQPREPQVCTLPPSRDELTKNQVSLSCAVKGFYPSDIAVEWESNGQPENNYKTTP |
| | | PVLDSDGSFFLVSKLTVDKSRWQQGNVFSCSVMHEALHNHYTQKSLSLSP |
| 216 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTRYVMHWVRQAPGQGLEWMGYINPYNDVTKYAE |
| | | KFQGRVTLTSDTSTSTAYMELSSLRSEDTAVYYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 217 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDLQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 218 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 219 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNLDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVYYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 220 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 221 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVYYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 222 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 223 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 224 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSAGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 225 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 226 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGAIDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 227 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSAGSTDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 228 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSIDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 229 | | ILNVEQSPQSLHVQEGDSTKFTCSFPVKEFQDIHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 230 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 231 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETMMRGLELLIYFQNTAVIDDSGMPEDRFSAK |
| | | MPNDSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 232 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 233 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSAGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 234 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 235 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSTGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 236 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 237 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSPGAIDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRWSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 238 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGDSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 239 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNDSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 240 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 241 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGESYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 242 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 243 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNAYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 244 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 245 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSAGAIDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 246 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 247 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 248 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSTGAIDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 249 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNASFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 250 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 251 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNNDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 252 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 253 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNADMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 254 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNDDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 255 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNEDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 256 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNFDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 257 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNHDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 258 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNIDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 259 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNLDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 260 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNKDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 261 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNQDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 262 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNRDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 263 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNVDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 264 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNESFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 265 | | ILNVEQSPQSLHVQEGDSTNFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 266 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNRSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRWSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 267 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNKSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 268 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNQSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 269 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNNSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 270 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNSSFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 271 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDRQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 272 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDHQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 273 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDEQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 274 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 275 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDQQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 276 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDNQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 277 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDFQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 278 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDYQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 279 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDIQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 280 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGATDVQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 281 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDRQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 282 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDHQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 283 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDEQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 284 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 285 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDQQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 286 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDNQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 287 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDFQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 288 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDYQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 289 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDIQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 290 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDVQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 291 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNESFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 292 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNRSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 293 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNKSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 294 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNQSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 295 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNNSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 296 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNSSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 297 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 298 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTRYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 299 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 300 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNLDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 301 | | QTVVTQEPSLTVSPGGTVTLTCGSSTGAVTSGYYPNWVQQKPGQAPRGLIGGTKFLAPGT |
| | | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNDSFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 302 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNLDMRFGAGTRLTVKPGGGSGGGGEV |
| | | QLVQSGAEVKKPGASVKVSCKASGYKFTRYVMHWVRQAPGQGLEWMGYINPYNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 303 | | QIQMTQSPSSLSASVGDRVTITCSATSSVSYMHWYQQKPGKAPKRWIYDTSKLASGVPSR |
| | | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 304 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNLDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 305 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNYDMRFGAGTRLTVKP |
| 306 | | GVIQSPRHEVTEMGQEVTLRCKPISGHNSLFWYRETPMQGLELLIYFQNTAVIDDSGMPE |
| | | DRFSAKMPNDSFSTLKIQPSEPRDSAVYFCASSPGATDKQYFGPGTRLTVL |
| 307 | | GVIQSPRHEVTEMGQEVTLRCKPISGHNSLFWYRETPMQGLELLIYFQNTAVIDDSGMPE |
| | | DRFSAKMPNASFSTLKIQPSEPRDSAVYFCASSPGATDKQYFGPGTRLTVL |
| 308 | | GVIQSPRHEVTEMGQEVTLRCKPISGHNSLFWYRETPMQGLELLIYFQNTAVIDDSGMPE |
| | | DRFSAKMPNDSFSTLKIQPSEPRDSAVYFCASSPGSTDAQYFGPGTRLTVL |
| 309 | | ILNVEQSPQSLHVQEGDSTKFTCSFPVKEFQDLHWYRKETAKSPEFLFYFGPYGKEKKKG |
| | | RISATLNTKEGYSYLYITDSQPEDSATYLCALYNNLDMRFGAGTRLTVKP |
| 310 | PRAME−004 | SLLQHLIGL |
| 311 | NY-ESO1-001 | SLLMWITQV |
| 312 | KRT5-004 | STASAITPSV |
| 313 | PRAME (UniProt | MERRRLWGSIQSRYISMSVWTSPRRLVELAGQSLLKDEALAIAALELLPRELFPPLFMAA |
| | P78395) | FDGRHSQTLKAMVQAWPFTCLPLGVLMKGQHLHLETFKAVLDGLDVLLAQEVRPRRWKLQ |
| | | VLDLRKNSHQDFWTVWSGNRASLYSFPEPEAAQPMTKKRKVDGLSTEAEQPFIPVEVLVD |
| | | LFLKEGACDELFSYLIEKVKRKKNVLRLCCKKLKIFAMPMQDIKMILKMVQLDSIEDLEV |
| | | TCTWKLPTLAKFSPYLGQMINLRRLLLSHIHASSYISPEKEEQYIAQFTSQFLSLQCLQA |
| | | LYVDSLFFLRGRLDQLLRHVMNPLETLSITNCRLSEGDVMHLSQSPSVSQLSVLSLSGVM |
| | | LTDVSPEPLQALLERASATLQDLVFDECGITDDQLLALLPSLSHCSQLTTLSFYGNSISI |
| | | SALQSLLQHLIGLSNLTHVLYPVPLESYEDIHGTLHLERLAYLHARLRELLCELGRPSMV |
| | | WLSANPCPHCGDRTFYDPEPILCPCFMPN |
| 314 | PRAME mRNA | AUGGAACGAAGGCGUUUGUGGGGUUCCAUUCAGAGCCGAUACAUCAGCAUGAGUGUGUGG |
| | (1527 | ACAAGCCCACGGAGACUUGUGGAGCUGGCAGGGCAGAGCCUGCUGAAGGAUGAGGCCCUG |
| | nucleotides out | GCCAUUGCCGCCCUGGAGUUGCUGCCCAGGGAGCUCUUCCCGCCACUCUUCAUGGCAGCC |
| | of which 370 U) | UUUGACGGGAGACACAGCCAGACCCUGAAGGCAAUGGUGCAGGCCUGGCCCUUCACCUGC |
| | | CUCCCUCUGGGAGUGCUGAUGAAGGGACAACAUCUUCACCUGGAGACCUUCAAAGCUGUG |
| | | CUUGAUGGACUUGAUGUGCUCCUUGCCCAGGAGGUUCGCCCCAGGAGGUGGAAACUUCAA |
| | | GUGCUGGAUUUACGGAAGAACUCUCAUCAGGACUUCUGGACUGUAUGGUCUGGAAACAGG |
| | | GCCAGUCUGUACUCAUUUCCAGAGCCAGAAGCAGCUCAGCCCAUGACAAAGAAGCGAAAA |
| | | GUAGAUGGUUUGAGCACAGAGGCAGAGCAGCCCUUCAUUCCAGUAGAGGUGCUCGUAGAC |
| | | CUGUUCCUCAAGGAAGGUGCCUGUGAUGAAUUGUUCUCCUACCUCAUUGAGAAAGUGAAG |
| | | CGAAAGAAAAAUGUACUACGCCUGUGCUGUAAGAAGCUGAAGAUUUUUGCAAUGCCCAUG |
| | | CAGGAUAUCAAGAUGAUCCUGAAAAUGGUGCAGCUGGACUCUAUUGAAGAUUUGGAAGUG |
| | | ACUUGUACCUGGAAGCUACCCACCUUGGCGAAAUUUUCUCCUUACCUGGGCCAGAUGAUU |
| | | AAUCUGCGUAGACUCCUCCUCUCCCACAUCCAUGCAUCUUCCUACAUUUCCCCGGAGAAG |
| | | GAAGAGCAGUAUAUCGCCCAGUUCACCUCUCAGUUCCUCAGUCUGCAGUGCCUGCAGGCU |
| | | CUCUAUGUGGACUCUUUAUUUUUCCUUAGAGGCCGCCUGGAUCAGUUGCUCAGGCACGUG |
| | | AUGAACCCCUUGGAAACCCUCUCAAUAACUAACUGCCGGCUUUCGGAAGGGGAUGUGAUG |
| | | CAUCUGUCCCAGAGUCCCAGCGUCAGUCAGCUAAGUGUCCUGAGUCUAAGUGGGGUCAUG |
| | | CUGACCGAUGUAAGUCCCGAGCCCCUCCAAGCUCUGCUGGAGAGAGCCUCUGCCACCCUC |
| | | CAGGACCUGGUCUUUGAUGAGUGUGGGAUCACGGAUGAUCAGCUCCUUGCCCUCCUGCCU |
| | | UCCCUGAGCCACUGCUCCCAGCUUACAACCUUAAGCUUCUACGGGAAUUCCAUCUCCAUA |
| | | UCUGCCUUGCAGAGUCUCCUGCAGCACCUCAUCGGGCUGAGCAAUCUGACCCACGUGCUG |
| | | UAUCCUGUCCCCCUGGAGAGUUAUGAGGACAUCCAUGGUACCCUCCACCUGGAGAGGCUU |
| | | GCCUAUCUGCAUGCCAGGCUCAGGGAGUUGCUGUGUGAGUUGGGGCGGCCCAGCAUGGUC |
| | | UGGCUUAGUGCCAACCCCUGUCCUCACUGUGGGGACAGAACCUUCUAUGACCCGGAGCCC |
| | | AUCCUGUGCCCCUGUUUCAUGCCUAAC |
| 315 | GC enriched | AUGGAACGAAGGCGCUUGUGGGGCUCCAUCCAGAGCCGAUACAUCAGCAUGAGCGUGUGG |
| | PRAME mRNA | ACAAGCCCACGGAGACUCGUGGAGCUGGCAGGGCAGAGCCUGCUGAAGGACGAGGCCCUG |
| | (1527 | GCCAUCGCCGCCCUGGAGUUGCUGCCCAGGGAGCUCUUCCCGCCACUCUUCAUGGCAGCC |
| | nucleotides out | UUCGACGGGAGACACAGCCAGACCCUGAAGGCAAUGGUGCAGGCCUGGCCCUUCACCUGC |
| | of which 265 U) | CUCCCCCUGGGAGUGCUGAUGAAGGGACAACACCUCCACCUGGAGACCUUCAAAGCCGUG |
| | | CUCGACGGACUCGACGUGCUCCUCGCCCAGGAGGUCCGCCCCAGGAGGUGGAAACUCCAA |
| | | GUGCUGGACUUACGGAAGAACUCCCACCAGGACUUCUGGACCGUAUGGUCCGGAAACAGG |
| | | GCCAGCCUGUACUCAUUCCCAGAGCCAGAAGCAGCCCAGCCCAUGACAAAGAAGCGAAAA |
| | | GUAGACGGCUUGAGCACAGAGGCAGAGCAGCCCUUCAUCCCAGUAGAGGUGCUCGUAGAC |
| | | CUGUUCCUCAAGGAAGGCGCCUGCGACGAAUUGUUCUCCUACCUCAUCGAGAAAGUGAAG |
| | | CGAAAGAAAAACGUACUACGCCUGUGCUGCAAGAAGCUGAAGAUCUUCGCAAUGCCCAUG |
| | | CAGGACAUCAAGAUGAUCCUGAAAAUGGUGCAGCUGGACUCCAUCGAAGACUUGGAAGUG |
| | | ACCUGCACCUGGAAGCUACCCACCUUGGCGAAAUUCUCCCCCUACCUGGGCCAGAUGAUC |
| | | AACCUGCGCAGACUCCUCCUCUCCCACAUCCACGCAUCCUCCUACAUCUCCCCGGAGAAG |
| | | GAAGAGCAGUACAUCGCCCAGUUCACCUCCCAGUUCCUCAGCCUGCAGUGCCUGCAGGCC |
| | | CUCUACGUGGACUCCUUAUUCUUCCUCAGAGGCCGCCUGGACCAGUUGCUCAGGCACGUG |
| | | AUGAACCCCUUGGAAACCCUCUCAAUAACCAACUGCCGGCUCUCGGAAGGGGACGUGAUG |
| | | CACCUGUCCCAGAGCCCCAGCGUCAGCCAGCUAAGCGUCCUGAGCCUAAGCGGGGUCAUG |
| | | CUGACCGACGUAAGCCCCGAGCCCCUCCAAGCCCUGCUGGAGAGAGCCUCCGCCACCCUC |
| | | CAGGACCUGGUCUUCGACGAGUGCGGGAUCACGGACGACCAGCUCCUCGCCCUCCUGCCC |
| | | UCCCUGAGCCACUGCUCCCAGCUCACAACCUUAAGCUUCUACGGGAACUCCAUCUCCAUA |
| | | UCCGCCUUGCAGAGCCUCCUGCAGCACCUCAUCGGGCUGAGCAACCUGACCCACGUGCUG |
| | | UACCCCGUCCCCCUGGAGAGCUACGAGGACAUCCACGGCACCCUCCACCUGGAGAGGCUC |
| | | GCCUACCUGCACGCCAGGCUCAGGGAGUUGCUGUGCGAGUUGGGGCGGCCCAGCAUGGUC |
| | | UGGCUCAGCGCCAACCCCUGCCCCCACUGCGGGGACAGAACCUUCUACGACCCGGAGCCC |
| | | AUCCUGUGCCCCUGCUUCAUGCCCAAC |
| 316 | PRAME cDNA | ATGGAACGAAGGCGTTTGTGGGGTTCCATTCAGAGCCGATACATCAGCATGAGTGTGTGG |
| | | ACAAGCCCACGGAGACTTGTGGAGCTGGCAGGGCAGAGCCTGCTGAAGGATGAGGCCCTG |
| | | GCCATTGCCGCCCTGGAGTTGCTGCCCAGGGAGCTCTTCCCGCCACTCTTCATGGCAGCC |
| | | TTTGACGGGAGACACAGCCAGACCCTGAAGGCAATGGTGCAGGCCTGGCCCTTCACCTGC |
| | | CTCCCTCTGGGAGTGCTGATGAAGGGACAACATCTTCACCTGGAGACCTTCAAAGCTGTG |
| | | CTTGATGGACTTGATGTGCTCCTTGCCCAGGAGGTTCGCCCCAGGAGGTGGAAACTTCAA |
| | | GTGCTGGATTTACGGAAGAACTCTCATCAGGACTTCTGGACTGTATGGTCTGGAAACAGG |
| | | GCCAGTCTGTACTCATTTCCAGAGCCAGAAGCAGCTCAGCCCATGACAAAGAAGCGAAAA |
| | | GTAGATGGTTTGAGCACAGAGGCAGAGCAGCCCTTCATTCCAGTAGAGGTGCTCGTAGAC |
| | | CTGTTCCTCAAGGAAGGTGCCTGTGATGAATTGTTCTCCTACCTCATTGAGAAAGTGAAG |
| | | CGAAAGAAAAATGTACTACGCCTGTGCTGTAAGAAGCTGAAGATTTTTGCAATGCCCATG |
| | | CAGGATATCAAGATGATCCTGAAAATGGTGCAGCTGGACTCTATTGAAGATTTGGAAGTG |
| | | ACTTGTACCTGGAAGCTACCCACCTTGGCGAAATTTTCTCCTTACCTGGGCCAGATGATT |
| | | AATCTGCGTAGACTCCTCCTCTCCCACATCCATGCATCTTCCTACATTTCCCCGGAGAAG |
| | | GAAGAGCAGTATATCGCCCAGTTCACCTCTCAGTTCCTCAGTCTGCAGTGCCTGCAGGCT |
| | | CTCTATGTGGACTCTTTATTTTTCCTTAGAGGCCGCCTGGATCAGTTGCTCAGGCACGTG |
| | | ATGAACCCCTTGGAAACCCTCTCAATAACTAACTGCCGGCTTTCGGAAGGGGATGTGATG |
| | | CATCTGTCCCAGAGTCCCAGCGTCAGTCAGCTAAGTGTCCTGAGTCTAAGTGGGGTCATG |
| | | CTGACCGATGTAAGTCCCGAGCCCCTCCAAGCTCTGCTGGAGAGAGCCTCTGCCACCCTC |
| | | CAGGACCTGGTCTTTGATGAGTGTGGGATCACGGATGATCAGCTCCTTGCCCTCCTGCCT |
| | | TCCCTGAGCCACTGCTCCCAGCTTACAACCTTAAGCTTCTACGGGAATTCCATCTCCATA |
| | | TCTGCCTTGCAGAGTCTCCTGCAGCACCTCATCGGGCTGAGCAATCTGACCCACGTGCTG |
| | | TATCCTGTCCCCCTGGAGAGTTATGAGGACATCCATGGTACCCTCCACCTGGAGAGGCTT |
| | | GCCTATCTGCATGCCAGGCTCAGGGAGTTGCTGTGTGAGTTGGGGCGGCCCAGCATGGTC |
| | | TGGCTTAGTGCCAACCCCTGTCCTCACTGTGGGGACAGAACCTTCTATGACCCGGAGCCC |
| | | ATCCTGTGCCCCTGTTTCATGCCTAAC |
| 317 | PRAME 004 | AGUCUCCUGCAGCACCUCAUCGGGCUG |
| | mRNA | |
| 318 | GC enriched | AGCCUCCUGCAGCACCUCAUCGGGCUG |
| | PRAME 004 | |
| | mRNA | |
| 319 | PRAME 004 cDNA | AGTCTCCTGCAGCACCTCATCGGGCTG |
| 320 | TPP-1295 alpha | VKEFQD |
| | CDR1 | |
| 321 | TPP-1295 alpha | FGPYGKE |
| | CDR2 | |
| 322 | TPP-1295 alpha | ALYNNYDMR |
| | CDR3 | |
| 323 | TPP-1295 alpha | ILNVEQSPQSLHVQEGDSTKFTCSFPVKEFQDLHWYRKETAKSPEFLFYFGPYGKEKKKG |
| | variable domain | RISATLNTKEGYSYLYITDSQPEDSATYLCALYNNYDMRFGAGTRLTVKP |
| 324 | TPP-1295 alpha | ILNVEQSPQSLHVQEGDSTKFTCSFPVKEFQDLHWYRKETAKSPEFLFYFGPYGKEKKKG |
| | full-length | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVYYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 325 | TPP-1295 beta | SGHNS |
| | CDR1 | |
| 326 | TPP-1295 beta | FQNTAV |
| | CDR2 | |
| 327 | TPP-1295 beta | ASSPGATDKQY |
| | CDR3 | |
| 328 | TPP-1295 beta | GVIQSPRHEVTEMGQEVTLRCKPISGHNSLFWYRETPMQGLELLIYFQNTAVIDDSGMPE |
| | variable domain | DRFSAKMPNASFSTLKIQPSEPRDSAVYFCASSPGATDKQYFGPGTRLTVL |
| 329 | TPP-1295 beta | QIQMTQSPSSLSASVGDRVTITCSATSSVSYMHWYQQKPGKAPKRWIYDTSKLASGVPSR |
| | full-length | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNASFSTLKIQPSEPRDSAVYFCASSPGATDKQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 330 | TPP-1298 alpha | VKEFQD |
| | CDR1 | |
| 331 | TPP-1298 alpha | FGPYGKE |
| | CDR2 | |
| 332 | TPP-1298 alpha | ALYNNYDMR |
| | CDR3 | |
| 333 | TPP-1298 alpha | ILNVEQSPQSLHVQEGDSTKFTCSFPVKEFQDLHWYRKETAKSPEFLFYFGPYGKEKKKG |
| | variable domain | RISATLNTKEGYSYLYITDSQPEDSATYLCALYNNYDMRFGAGTRLTVKP |
| 334 | TPP-1298 alpha | ILNVEQSPQSLHVQEGDSTKFTCSFPVKEFQDLHWYRKETAKSPEFLFYFGPYGKEKKKG |
| | full-length | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 335 | TPP-1298 beta | SGHNS |
| | CDR1 | |
| 336 | TPP-1298 beta | FQNTAV |
| | CDR2 | |
| 337 | TPP-1298 beta | ASSAGSTDAQY |
| | CDR3 | |
| 338 | TPP-1298 beta | GVIQSPRHEVTEMGQEVTLRCKPISGHNSLFWYRETPMQGLELLIYFQNTAVIDDSGMPE |
| | variable domain | DRFSAKMPNDSFSTLKIQPSEPRDSAVYFCASSAGSTDAQYFGPGTRLTVL |
| 339 | TPP-1298 beta | QIQMTQSPSSLSASVGDRVTITCSATSSVSYMHWYQQKPGKAPKRWIYDTSKLASGVPSR |
| | full-length | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSAGSTDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 340 | TPP-230 alpha | VKEFQD |
| | CDR1 | |
| 341 | TPP-230 alpha | FGPYGKE |
| | CDR2 | |
| 342 | TPP-230 alpha | ALYNNYDMR |
| | CDR3 | |
| 343 | TPP-230 alpha | ILNVEQSPQSLHVQEGDSTKFTCSFPVKEFQDLHWYRKETAKSPEFLFYFGPYGKEKKKG |
| | variable domain | RISATLNTKEGYSYLYITDSQPEDSATYLCALYNNYDMRFGAGTRLTVKP |
| 344 | TPP-230 alpha | ILNVEQSPQSLHVQEGDSTKFTCSFPVKEFQDLHWYRKETAKSPEFLFYFGPYGKEKKKG |
| | full-length | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGNSYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 345 | TPP-230 beta | SGHNS |
| | CDR1 | |
| 346 | TPP-230 beta | FQNTAV |
| | CDR2 | |
| 347 | TPP-230 beta | ASSPGATDKQY |
| | CDR3 | |
| 348 | TPP-230 beta | GVIQSPRHEVTEMGQEVTLRCKPISGHNSLFWYRETPMQGLELLIYFQNTAVIDDSGMPE |
| | variable domain | DRFSAKMPNASFSTLKIQPSEPRDSAVYFCASSPGATDKQYFGPGTRLTVL |
| 349 | TPP-230 beta | QTVVTQEPSLTVSPGGTVTLTCGSSTGAVTSGYYPNWVQQKPGQAPRGLIGGTKFLAPGT |
| | full-length | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNASFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 350 | TPP-669 alpha | VKEFQD |
| | CDR1 | |
| 351 | TPP-669 alpha | FGPYGKE |
| | CDR2 | |
| 352 | TPP-669 alpha | ALYNNYDMR |
| | CDR3 | |
| 353 | TPP-669 alpha | ILNVEQSPQSLHVQEGDSTKFTCSFPVKEFQDLHWYRKETAKSPEFLFYFGPYGKEKKKG |
| | variable domain | RISATLNTKEGYSYLYITDSQPEDSATYLCALYNNYDMRFGAGTRLTVKP |
| 354 | TPP-669 alpha | ILNVEQSPQSLHVQEGDSTKFTCSFPVKEFQDLHWYRKETAKSPEFLFYFGPYGKEKKKG |
| | full-length | RISATLNTKEGYSYLYITDSQPEDSATYLCALYNNYDMRFGAGTRLTVKPGGGSGGGGEV |
| | | QLVQSGAEVKKPGASVKVSCKASGYKFTSYVMHWVRQAPGQGLEWMGYINPRNDVTKYAE |
| | | KFQGRVTLTSDTSTSTAYMELSSLRSEDTAVHYCARGSYYDYEGFVYWGQGTLVTVSSEP |
| | | KSSDKTHTCPPCPAPPVAGPSVFLFPPKPKDTLMISRTPEVTCVVVDVSHEDPEVKFNWY |
| | | VDGVEVHNAKTKPREEQYQSTYRVVSVLTVLHQDWLNGKEYKCKVSNKALPASIEKTISK |
| | | AKGQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSP |
| 355 | TPP-669 beta | SGHNS |
| | CDR1 | |
| 356 | TPP-669 beta | FQNTAV |
| | CDR2 | |
| 357 | TPP-669 beta | ASSPGSTDAQY |
| | CDR3 | |
| 358 | TPP-669 beta | GVIQSPRHEVTEMGQEVTLRCKPISGHNSLFWYRETPMQGLELLIYFQNTAVIDDSGMPE |
| | variable domain | DRFSAKMPNDSFSTLKIQPSEPRDSAVYFCASSPGSTDAQYFGPGTRLTVL |
| 359 | TPP-669 beta | QIQMTQSPSSLSASVGDRVTITCSATSSVSYMHWYQQKPGKAPKRWIYDTSKLASGVPSR |
| | full-length | FSGSGSGTDYTLTISSLQPEDAATYYCQQWSSNPLTFGGGTKVEIKGGGSGGGGGVIQSP |
| | | RHEVTEMGQEVTLRCKPISGHNSLFWYRETPMQGLELLIYFQNTAVIDDSGMPEDRFSAK |
| | | MPNDSFSTLKIQPSEPRDSAVYFCASSPGSTDAQYFGPGTRLTVLEPKSSDKTHTCPPCP |
| | | APPVAGPSVFLFPPKPKDTLMISRTPEVTCVVVDVSHEDPEVKFNWYVDGVEVHNAKTKP |
| | | REEQYQSTYRVVSVLTVLHQDWLNGKEYKCKVSNKALPASIEKTISKAKGQPREPQVYTL |
| | | PPCRDELTKNQVSLWCLVKGFYPSDIAVEWESNGQPENNYKTTPPVLDSDGSFFLYSKLT |
| | | VDKSRWQQGNVFSCSVMHEALHNHYTQKSLSLSP |
| 360 | TPP-1333 alpha | VKEFQD |
| | CDR1 | |
| 361 | TPP-1333 alpha | FGPYGKE |
| | CDR2 | |
| 362 | TPP-1333 alpha | ALYNNYDMR |
| | CDR3 | |
| 363 | TPP-1333 alpha | ILNVEQSPQSLHVQEGDSTKFTCSFPVKEFQDLHWYRKETAKSPEFLFYFGPYGKEKKKG |
| | variable domain | RISATLNTKEGYSYLYITDSQPEDSATYLCALYNNYDMRFGAGTRLTVKP |
| 364 | TPP-1333 alpha | ILNVEQSPQSLHVQEGDSTKFTCSFPVKEFQDLHWYRKETAKSPEFLFYFGPYGKEKKKG |
| | full-length | RISATLNTKEGYSYLYITDSQPEDSATYLCALYNNYDMRFGAGTRLTVKPGGGSGGGGEV |
| | | QLVESGGGLVQPGGSLKLSCAASGFTFNKYAMNWVRQAPGKGLEWVARIRSKYNNYATYY |
| | | ADSVKDRFTISRDDSKNTAYLQMNNLKTEDTAVYYCVRHGNFGESYISYWAYWGQGTLVT |
| | | VSSEPKSSDKTHTCPPCPAPPVAGPSVFLFPPKPKDTLMISRTPEVTCVVVDVSHEDPEV |
| | | KFNWYVDGVEVHNAKTKPREEQYQSTYRVVSVLTVLHQDWLNGKEYKCKVSNKALPASIE |
| | | KTISKAKGQPREPQVCTLPPSRDELTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| 365 | TPP-1333 beta | SGHNS |
| | CDR1 | |
| 366 | TPP-1333 beta | FQNTAV |
| | CDR2 | |
| 367 | TPP-1333 beta | ASSPGATDKQY |
| | CDR3 | |
| 368 | TPP-1333 beta | GVIQSPRHEVTEMGQEVTLRCKPISGHNSLFWYRETPMQGLELLIYFQNTAVIDDSGMPE |
| | variable domain | DRFSAKMPNASFSTLKIQPSEPRDSAVYFCASSPGATDKQYFGPGTRLTVL |
| 369 | TPP-1333 beta | QTVVTQEPSLTVSPGGTVTLTCGSSTGAVTSGYYPNWVQQKPGQAPRGLIGGTKFLAPGT |
| | full-length | PARFSGSLLGGKAALTLSGVQPEDEAEYYCALWYSNRWVFGGGTKLTVLGGGSGGGGGVI |
| | | QSPRHEVTEMGQEVTLRCKPISGHNSLFWYRETPMQGLELLIYFQNTAVIDDSGMPEDRF |
| | | SAKMPNASFSTLKIQPSEPRDSAVYFCASSPGATDKQYFGPGTRLTVLEPKSSDKTHTCP |
| | | PCPAPPVAGPSVFLFPPKPKDTLMISRTPEVTCVVVDVSHEDPEVKFNWYVDGVEVHNAK |
| | | TKPREEQYQSTYRVVSVLTVLHQDWLNGKEYKCKVSNKALPASIEKTISKAKGQPREPQV |
| | | YTLPPCRDELTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| 370 | SMARCD1-001 | IIINHVISV |
| 371 | VIM-009 | SLNLRETNL |
| 372 | FARSA-001 | LTLGHLMGV |
| 373 | GIMAP8-001 | KLLKNLIGI |
| 374 | TPP-1109 full | EVQLVQSGAEVKKPGASVKVSCKASGYSFTGYTMNWVRQAPGQGLEWMGLINPYKGVSTY |
| | length 1 | AQKFQDRVTLTVDKSTSTAYMELSSLRSEDTAVYYCARSGYYGDSDWYFDVWGQGTLVTV |
| | | SSGGGSGGGGKAGVTQTPRYLIKTRGQQVTLSCSPIPGHRAVSWYQQTPGQGLQFLFEYV |
| | | HGEERNKGNFPGRFSGRQFSNSSSEMNISNLELGDSALYLCASSPWDSPNVQYFGPGTRL |
| | | TVTEDLKNEPKSSDKTHTCPPCPAPPVAGPSVFLFPPKPKDTLMISRTPEVTCVVVDVSH |
| | | EDPEVKFNWYVDGVEVHNAKTKPREEQYQSTYRVVSVLTVLHQDWLNGKEYKCKVSNKAL |
| | | PASIEKTISKAKGQPREPQVCTLPPSRDELTKNQVSLSCAVKGFYPSDIAVEWESNGQPE |
| | | NNYKTTPPVLDSDGSFFLVSKLTVDKSRWQQGNVFSCSVMHEALHNHYTQKSLSLSPGK |
| 375 | TPP-1109 full | QKEVEQNSGPLSVPEGAIASLNCTYSDRGSQSFFWYRQYSGKSPELIMSIYSNGDKEDGR |
| | length 1 | FTAQLNKASQYVSLLIRDSQPSDSATYLCAAVIDNDQGGILTFGTGTRLTIIPNIQNGGG |
| | | SGGGGDIQMTQSPSSLSASVGDRVTITCRASQDIRNYLNWYQQKPGKAPKLLIYYTSRLH |
| | | SGVPSRFSGSGSGTDYTLTISSLQPEDIATYFCQQGQTLPWTFGQGTKVEIKEPKSSDKT |
| | | HTCPPCPAPPVAGPSVFLFPPKPKDTLMISRTPEVTCVVVDVSHEDPEVKFNWYVDGVEV |
| | | HNAKTKPREEQYQSTYRVVSVLTVLHQDWLNGKEYKCKVSNKALPASIEKTISKAKGQPR |
| | | EPQVYTLPPCRDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 376 | PSMA₂₈₈₋₂₉₇ | GLPSIPVHPI |
| 377 | PSMA₂₈₈₋₂₉₇ I297V | GLPSIPVHPV |

What is claimed is:

1. A method of treating a patient who has metastasis or a metastatic lesion that presents a peptide comprising SLLQHLIGL (SEQ ID NO: 310) on the cell surface, comprising
identifying a metastatic lesion and
treating the metastatic lesion with a population of T lymphocytes that kills the metastasis or metastatic lesion that presents a peptide comprising SLLQHLIGL (SEQ ID NO: 310) on the cell surface,
wherein the population of T lymphocytes comprises a T cell receptor (TCR) that binds a peptide comprising SLLQHLIGL (SEQ ID NO: 310) in a complex with a class I MHC molecule,
wherein the TCR comprises
(1) a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
(2) a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
(3) a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
(4) a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
(5) a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
(6) a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
(7) a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, uterine carcinoma, uterine carcinosarcoma, head and neck squamous cell carcinomas, head and neck adenocarcinoma, colon cancer, gastro-intestinal cancer, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

2. A method of eliciting an immune response in a patient who has metastasis or a metastatic lesion that presents a peptide comprising SLLQHLIGL (SEQ ID NO: 310) on the cell surface, comprising:
identifying a metastatic lesion and
treating the metastatic lesion with a population of T lymphocytes that kills the metastasis or metastatic lesion that presents a peptide comprising SLLQHLIGL (SEQ ID NO: 310) on the cell surface,
wherein the population of T lymphocytes comprises a T cell receptor (TCR) that binds a peptide comprising SLLQHLIGL (SEQ ID NO: 310) in a complex with a class I MHC molecule,
wherein the TCR comprises
(1) a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
(2) a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
(3) a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
(4) a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
(5) a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
(6) a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR11 chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
(7) a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
wherein the metastasis or metastatic lesion originates from a cancer selected from the group consisting of adrenocortical carcinoma, lung cancer, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, uterine carcinoma, uterine carcinosarcoma, head and neck squamous cell carcinomas, head and neck adenocarcinoma, colon cancer, gastro-intestinal cancer, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin's lymphoma, glioblastoma, cervical carcinoma, cervical squamous cell carcinoma and endocervical adenocarcinoma, hepatocellular carcinoma, liver hepatocellular carcinoma, Ewing's sarcoma, endometrial cancer, epithelial cancer of the larynx, esophageal carcinoma, oral carcinoma, atypical meningioma, papillary thyroid carcinoma, thymoma, brain tumors, salivary duct carcinoma, and extranodal T/NK-cell lymphomas.

3. The method of claim 1, wherein the population of T lymphocyte is autologous or allogenic to the patient.

4. The method of claim 1, wherein the TCR comprises
(1) an α chain variable domain comprising SEQ ID NO: 15, and a β chain variable domain comprising SEQ ID NO: 21, or
(2) an α chain variable domain comprising SEQ ID NO: 27, and a β chain variable domain comprising SEQ ID NO: 33, or
(3) an α chain variable domain comprising SEQ ID NO: 39, and a β chain variable domain comprising SEQ ID NO: 45, or
(4) an α chain variable domain comprising SEQ ID NO: 51, and a β chain variable domain comprising SEQ ID NO: 57, or
(5) an α chain variable domain comprising SEQ ID NO: 63, and a β chain variable domain comprising SEQ ID NO: 69, or
(6) an α chain variable domain comprising SEQ ID NO: 75, and a β chain variable domain comprising SEQ ID NO: 81, or
(7) an α chain variable domain comprising SEQ ID NO: 87, and a β chain variable domain comprising SEQ ID NO: 93, or
(8) an α chain variable domain comprising SEQ ID NO: 111, and a β chain variable domain comprising SEQ ID NO: 117.

5. The method of claim 1, wherein the TCR comprises a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20.

6. The method of claim 1, wherein the TCR comprises a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32.

7. The method of claim 1, wherein the TCR comprises a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44.

8. The method of claim 1, wherein the TCR comprises a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56.

9. The method of claim 1, wherein the TCR comprises a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68.

10. The method of claim 1, wherein the TCR comprises a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80.

11. The method of claim 1, wherein the TCR comprises a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92.

12. The method of claim 2, wherein the recombinant T lymphocyte are autologous or allogenic to the patient.

13. The method of claim 2, wherein the TCR comprises
(1) an α chain variable domain comprising SEQ ID NO: 15, and a β chain variable domain comprising SEQ ID NO: 21, or
(2) an α chain variable domain comprising SEQ ID NO: 27, and a β chain variable domain comprising SEQ ID NO: 33, or
(3) an α chain variable domain comprising SEQ ID NO: 39, and a β chain variable domain comprising SEQ ID NO: 45, or
(4) an α chain variable domain comprising SEQ ID NO: 51, and a β chain variable domain comprising SEQ ID NO: 57, or
(5) an α chain variable domain comprising SEQ ID NO: 63, and a β chain variable domain comprising SEQ ID NO: 69, or
(6) an α chain variable domain comprising SEQ ID NO: 75, and a β chain variable domain comprising SEQ ID NO: 81, or
(7) an α chain variable domain comprising SEQ ID NO: 87, and a β chain variable domain comprising SEQ ID NO: 93, or
(8) an α chain variable domain comprising SEQ ID NO: 111, and a β chain variable domain comprising SEQ ID NO: 117.

14. The method of claim 2, wherein the TCR comprises a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20.

15. The method of claim 2, wherein the TCR comprises a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32.

16. The method of claim 2, wherein the TCR comprises a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44.

17. The method of claim 2, wherein the TCR comprises a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56.

18. The method of claim 2, wherein the TCR comprises a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68.

19. The method of claim 2, wherein the TCR comprises a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80.

20. The method of claim 2, wherein the TCR comprises a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92.
